SEQUENCE LISTING (566 sequences)

SEQ ID NO: 1 - DNA, 69 bp, Artificial Sequence
  Synthetic polynucleotide
tcttgtggaaagccagaaacatggactacttaccagggacacctgtttcagagctaagcacaagagtgc

SEQ ID NO: 2 - DNA, 69 bp, Artificial Sequence
  Synthetic polynucleotide
tcttgtggaaagccagaaacatggcacacacagagaacttaggggtttcagagctaagcacaagagtgc

SEQ ID NO: 3 - DNA, 69 bp, Artificial Sequence
  Synthetic polynucleotide
tcttgtggaaagccagaaacatggctcacctttggaaaacacaggtttcagagctaagcacaagagtgc

SEQ ID NO: 4 - DNA, 69 bp, Artificial Sequence
  Synthetic polynucleotide
tcttgtggaaagccagaaacatggactgaccattcagaacagacgtttcagagctaagcacaagagtgc

SEQ ID NO: 5 - DNA, 69 bp, Artificial Sequence
  Synthetic polynucleotide
tcttgtggaaagccagaaacatggcacctgcggatcttacgggcgtttcagagctaagcacaagagtgc

SEQ ID NO: 6 - DNA, 69 bp, Artificial Sequence
  Synthetic polynucleotide
tcttgtggaaagccagaaacatggcctccctctaggccggaagggtttcagagctaagcacaagagtgc

SEQ ID NO: 7 - DNA, 69 bp, Artificial Sequence
  Synthetic polynucleotide
tcttgtggaaagccagaaacatgggaagattgaagccagagaaagtttcagagctaagcacaagagtgc

SEQ ID NO: 8 - DNA, 69 bp, Artificial Sequence
  Synthetic polynucleotide
tcttgtggaaagccagaaacatggacaacctcttcaacacaaccgtttcagagctaagcacaagagtgc

SEQ ID NO: 9 - DNA, 69 bp, Artificial Sequence
  Synthetic polynucleotide
tcttgtggaaagccagaaacatggctacgagagtacaaggtagtgtttcagagctaagcacaagagtgc

SEQ ID NO: 10 - DNA, 69 bp, Artificial Sequence
  Synthetic polynucleotide
tcttgtggaaagccagaaacatggagaccttcttcttgccacaggtttcagagctaagcacaagagtgc

SEQ ID NO: 11 - DNA, 69 bp, Artificial Sequence
  Synthetic polynucleotide
tcttgtggaaagccagaaacatggatagcatagtggtcaagcctgtttcagagctaagcacaagagtgc

SEQ ID NO: 12 - DNA, 69 bp, Artificial Sequence
  Synthetic polynucleotide
tcttgtggaaagccagaaacatggagtcggctttcctttccaaggtttcagagctaagcacaagagtgc

SEQ ID NO: 13 - DNA, 69 bp, Artificial Sequence
  Synthetic polynucleotide
tcttgtggaaagccagaaacatggatcttccttcgaatgtgggagtttcagagctaagcacaagagtgc

SEQ ID NO: 14 - DNA, 69 bp, Artificial Sequence
  Synthetic polynucleotide
tcttgtggaaagccagaaacatggacgcaaagctgtcatcgtgagtttcagagctaagcacaagagtgc

SEQ ID NO: 15 - DNA, 69 bp, Artificial Sequence
  Synthetic polynucleotide
tcttgtggaaagccagaaacatggccaccgaggcctgcgcaagggtttcagagctaagcacaagagtgc

SEQ ID NO: 16 - DNA, 69 bp, Artificial Sequence
  Synthetic polynucleotide
tcttgtggaaagccagaaacatggaaagtgggaagtacgtcctggtttcagagctaagcacaagagtgc

SEQ ID NO: 17 - DNA, 69 bp, Artificial Sequence
  Synthetic polynucleotide
tcttgtggaaagccagaaacatggagagggatactcacactccagtttcagagctaagcacaagagtgc

SEQ ID NO: 18 - DNA, 69 bp, Artificial Sequence
  Synthetic polynucleotide
tcttgtggaaagccagaaacatggagggttcgtagaagattcaagtttcagagctaagcacaagagtgc

SEQ ID NO: 19 - DNA, 69 bp, Artificial Sequence
  Synthetic polynucleotide
tcttgtggaaagccagaaacatggaacaaagaaacatgtcagcagtttcagagctaagcacaagagtgc

SEQ ID NO: 20 - DNA, 69 bp, Artificial Sequence
  Synthetic polynucleotide
tcttgtggaaagccagaaacatggcaatggatttccggacgactgtttcagagctaagcacaagagtgc

SEQ ID NO: 21 - DNA, 69 bp, Artificial Sequence
  Synthetic polynucleotide
tcttgtggaaagccagaaacatggaacaaaaccagagtcatctggtttcagagctaagcacaagagtgc

SEQ ID NO: 22 - DNA, 69 bp, Artificial Sequence
  Synthetic polynucleotide
tcttgtggaaagccagaaacatggcatggccctacgctaccctagtttcagagctaagcacaagagtgc

SEQ ID NO: 23 - DNA, 69 bp, Artificial Sequence
  Synthetic polynucleotide
tcttgtggaaagccagaaacatggaggccttagagccacagcgggtttcagagctaagcacaagagtgc

SEQ ID NO: 24 - DNA, 69 bp, Artificial Sequence
  Synthetic polynucleotide
tcttgtggaaagccagaaacatggagcaagtgtacttggcgtgcgtttcagagctaagcacaagagtgc

SEQ ID NO: 25 - DNA, 69 bp, Artificial Sequence
  Synthetic polynucleotide
tcttgtggaaagccagaaacatggcagcagatacctttacacccgtttcagagctaagcacaagagtgc

SEQ ID NO: 26 - DNA, 69 bp, Artificial Sequence
  Synthetic polynucleotide
tcttgtggaaagccagaaacatggattaccagggatgtttctgagtttcagagctaagcacaagagtgc

SEQ ID NO: 27 - DNA, 69 bp, Artificial Sequence
  Synthetic polynucleotide
tcttgtggaaagccagaaacatgggactggtgatgaatacaatggtttcagagctaagcacaagagtgc

SEQ ID NO: 28 - DNA, 69 bp, Artificial Sequence
  Synthetic polynucleotide
tcttgtggaaagccagaaacatggacagggttgcggctgcaatggtttcagagctaagcacaagagtgc

SEQ ID NO: 29 - DNA, 69 bp, Artificial Sequence
  Synthetic polynucleotide
tcttgtggaaagccagaaacatggcgtttgtcatcagaatcagagtttcagagctaagcacaagagtgc

SEQ ID NO: 30 - DNA, 69 bp, Artificial Sequence
  Synthetic polynucleotide
tcttgtggaaagccagaaacatggcaccctttacttctcaggacgtttcagagctaagcacaagagtgc

SEQ ID NO: 31 - DNA, 69 bp, Artificial Sequence
  Synthetic polynucleotide
tcttgtggaaagccagaaacatggagactgctgcctcatatccggtttcagagctaagcacaagagtgc

SEQ ID NO: 32 - DNA, 69 bp, Artificial Sequence
  Synthetic polynucleotide
tcttgtggaaagccagaaacatggagccccaccagcaggtacacgtttcagagctaagcacaagagtgc

SEQ ID NO: 33 - DNA, 69 bp, Artificial Sequence
  Synthetic polynucleotide
tcttgtggaaagccagaaacatggccgatgttcttgtagtaccggtttcagagctaagcacaagagtgc

SEQ ID NO: 34 - DNA, 69 bp, Artificial Sequence
  Synthetic polynucleotide
tcttgtggaaagccagaaacatggaaacactcaccgatctgtgagtttcagagctaagcacaagagtgc

SEQ ID NO: 35 - DNA, 69 bp, Artificial Sequence
  Synthetic polynucleotide
tcttgtggaaagccagaaacatggcaacatagcagcatatggtggtttcagagctaagcacaagagtgc

SEQ ID NO: 36 - DNA, 69 bp, Artificial Sequence
  Synthetic polynucleotide
tcttgtggaaagccagaaacatggatcatcctacccgagatggtgtttcagagctaagcacaagagtgc

SEQ ID NO: 37 - DNA, 69 bp, Artificial Sequence
  Synthetic polynucleotide
tcttgtggaaagccagaaacatggatcaacaatgccgaaaagaggtttcagagctaagcacaagagtgc

SEQ ID NO: 38 - DNA, 69 bp, Artificial Sequence
  Synthetic polynucleotide
tcttgtggaaagccagaaacatggagctccgcaacaagatagcagtttcagagctaagcacaagagtgc

SEQ ID NO: 39 - DNA, 69 bp, Artificial Sequence
  Synthetic polynucleotide
tcttgtggaaagccagaaacatggaaaggatggaaaatacagccgtttcagagctaagcacaagagtgc

SEQ ID NO: 40 - DNA, 69 bp, Artificial Sequence
  Synthetic polynucleotide
tcttgtggaaagccagaaacatggaactggttctacacgcgagcgtttcagagctaagcacaagagtgc

SEQ ID NO: 41 - DNA, 69 bp, Artificial Sequence
  Synthetic polynucleotide
tcttgtggaaagccagaaacatggaaaaacacccgtggagccgggtttcagagctaagcacaagagtgc

SEQ ID NO: 42 - DNA, 69 bp, Artificial Sequence
  Synthetic polynucleotide
tcttgtggaaagccagaaacatggtacaaagatgacatccggtggtttcagagctaagcacaagagtgc

SEQ ID NO: 43 - DNA, 69 bp, Artificial Sequence
  Synthetic polynucleotide
tcttgtggaaagccagaaacatggcgtcattcgaaacatagtgggtttcagagctaagcacaagagtgc

SEQ ID NO: 44 - DNA, 69 bp, Artificial Sequence
  Synthetic polynucleotide
tcttgtggaaagccagaaacatggatcagtctctgctgatcagggtttcagagctaagcacaagagtgc

SEQ ID NO: 45 - DNA, 69 bp, Artificial Sequence
  Synthetic polynucleotide
tcttgtggaaagccagaaacatggaaatcattatcttagccaccgtttcagagctaagcacaagagtgc

SEQ ID NO: 46 - DNA, 69 bp, Artificial Sequence
  Synthetic polynucleotide
tcttgtggaaagccagaaacatggaaaaacgcaacaatcagatagtttcagagctaagcacaagagtgc

SEQ ID NO: 47 - DNA, 69 bp, Artificial Sequence
  Synthetic polynucleotide
tcttgtggaaagccagaaacatgggttcattaaaatcgatggcagtttcagagctaagcacaagagtgc

SEQ ID NO: 48 - DNA, 69 bp, Artificial Sequence
  Synthetic polynucleotide
tcttgtggaaagccagaaacatggacctgcctcacagtgcaggggtttcagagctaagcacaagagtgc

SEQ ID NO: 49 - DNA, 69 bp, Artificial Sequence
  Synthetic polynucleotide
tcttgtggaaagccagaaacatggactgtagtatcagtcagtccgtttcagagctaagcacaagagtgc

SEQ ID NO: 50 - DNA, 69 bp, Artificial Sequence
  Synthetic polynucleotide
tcttgtggaaagccagaaacatggaaattgaagttggtggtggtgtttcagagctaagcacaagagtgc

SEQ ID NO: 51 - DNA, 69 bp, Artificial Sequence
  Synthetic polynucleotide
tcttgtggaaagccagaaacatggacaagaaataccgtgccctggtttcagagctaagcacaagagtgc

SEQ ID NO: 52 - DNA, 69 bp, Artificial Sequence
  Synthetic polynucleotide
tcttgtggaaagccagaaacatgggcaaaacttatgtgaccccggtttcagagctaagcacaagagtgc

SEQ ID NO: 53 - DNA, 69 bp, Artificial Sequence
  Synthetic polynucleotide
tcttgtggaaagccagaaacatggcataaatctcaagaggacctgtttcagagctaagcacaagagtgc

SEQ ID NO: 54 - DNA, 69 bp, Artificial Sequence
  Synthetic polynucleotide
tcttgtggaaagccagaaacatgggacaccatgtggccacaaaggtttcagagctaagcacaagagtgc

SEQ ID NO: 55 - DNA, 69 bp, Artificial Sequence
  Synthetic polynucleotide
tcttgtggaaagccagaaacatgggaagaactatgatccccagagtttcagagctaagcacaagagtgc

SEQ ID NO: 56 - DNA, 69 bp, Artificial Sequence
  Synthetic polynucleotide
tcttgtggaaagccagaaacatggggatgcgaagttcccgcatggtttcagagctaagcacaagagtgc

SEQ ID NO: 57 - DNA, 69 bp, Artificial Sequence
  Synthetic polynucleotide
tcttgtggaaagccagaaacatgggaccccaacgagatcaaagtgtttcagagctaagcacaagagtgc

SEQ ID NO: 58 - DNA, 69 bp, Artificial Sequence
  Synthetic polynucleotide
tcttgtggaaagccagaaacatgggccggatgggacccgacgcggtttcagagctaagcacaagagtgc

SEQ ID NO: 59 - DNA, 69 bp, Artificial Sequence
  Synthetic polynucleotide
tcttgtggaaagccagaaacatggctgccccacaaaaccaagcggtttcagagctaagcacaagagtgc

SEQ ID NO: 60 - DNA, 69 bp, Artificial Sequence
  Synthetic polynucleotide
tcttgtggaaagccagaaacatgggtctcctttggacctcatgcgtttcagagctaagcacaagagtgc

SEQ ID NO: 61 - DNA, 69 bp, Artificial Sequence
  Synthetic polynucleotide
tcttgtggaaagccagaaacatggccgccataacaaggaccgaagtttcagagctaagcacaagagtgc

SEQ ID NO: 62 - DNA, 69 bp, Artificial Sequence
  Synthetic polynucleotide
tcttgtggaaagccagaaacatggctgcgctatgactcccggaggtttcagagctaagcacaagagtgc

SEQ ID NO: 63 - DNA, 69 bp, Artificial Sequence
  Synthetic polynucleotide
tcttgtggaaagccagaaacatgggaatgccagagaaggtcacagtttcagagctaagcacaagagtgc

SEQ ID NO: 64 - DNA, 69 bp, Artificial Sequence
  Synthetic polynucleotide
tcttgtggaaagccagaaacatgggcacgtcacccaccttccgggtttcagagctaagcacaagagtgc

SEQ ID NO: 65 - DNA, 69 bp, Artificial Sequence
  Synthetic polynucleotide
tcttgtggaaagccagaaacatggcgtagcgcctcccgtgtccggtttcagagctaagcacaagagtgc

SEQ ID NO: 66 - DNA, 69 bp, Artificial Sequence
  Synthetic polynucleotide
tcttgtggaaagccagaaacatggatttgctcttaggttgtacggtttcagagctaagcacaagagtgc

SEQ ID NO: 67 - DNA, 69 bp, Artificial Sequence
  Synthetic polynucleotide
tcttgtggaaagccagaaacatgggggatatccacagagtacctgtttcagagctaagcacaagagtgc

SEQ ID NO: 68 - DNA, 69 bp, Artificial Sequence
  Synthetic polynucleotide
tcttgtggaaagccagaaacatggccgtgtcattgcccacacccgtttcagagctaagcacaagagtgc

SEQ ID NO: 69 - DNA, 69 bp, Artificial Sequence
  Synthetic polynucleotide
tcttgtggaaagccagaaacatggaatagagtactatgctatgtgtttcagagctaagcacaagagtgc

SEQ ID NO: 70 - DNA, 69 bp, Artificial Sequence
  Synthetic polynucleotide
tcttgtggaaagccagaaacatggcagatgtgcgcattgacaccgtttcagagctaagcacaagagtgc

SEQ ID NO: 71 - DNA, 69 bp, Artificial Sequence
  Synthetic polynucleotide
tcttgtggaaagccagaaacatggatttccttcagcgtaactgggtttcagagctaagcacaagagtgc

SEQ ID NO: 72 - DNA, 69 bp, Artificial Sequence
  Synthetic polynucleotide
tcttgtggaaagccagaaacatgggtaaacaattctattaccaggtttcagagctaagcacaagagtgc

SEQ ID NO: 73 - DNA, 69 bp, Artificial Sequence
  Synthetic polynucleotide
tcttgtggaaagccagaaacatgggttaataactgtgagaacacgtttcagagctaagcacaagagtgc

SEQ ID NO: 74 - DNA, 69 bp, Artificial Sequence
  Synthetic polynucleotide
tcttgtggaaagccagaaacatggagcaacacagtcactcctgggtttcagagctaagcacaagagtgc

SEQ ID NO: 75 - DNA, 69 bp, Artificial Sequence
  Synthetic polynucleotide
tcttgtggaaagccagaaacatggcccaggcacagccgacgccggtttcagagctaagcacaagagtgc

SEQ ID NO: 76 - DNA, 69 bp, Artificial Sequence
  Synthetic polynucleotide
tcttgtggaaagccagaaacatggcctcattcgaccagttccgggtttcagagctaagcacaagagtgc

SEQ ID NO: 77 - DNA, 69 bp, Artificial Sequence
  Synthetic polynucleotide
tcttgtggaaagccagaaacatgggccaaacgtaccaagaaagtgtttcagagctaagcacaagagtgc

SEQ ID NO: 78 - DNA, 69 bp, Artificial Sequence
  Synthetic polynucleotide
tcttgtggaaagccagaaacatggcctgctcacagcccgacgaagtttcagagctaagcacaagagtgc

SEQ ID NO: 79 - DNA, 69 bp, Artificial Sequence
  Synthetic polynucleotide
tcttgtggaaagccagaaacatggcagactagtgctgagtcttggtttcagagctaagcacaagagtgc

SEQ ID NO: 80 - DNA, 69 bp, Artificial Sequence
  Synthetic polynucleotide
tcttgtggaaagccagaaacatgggaggcttgtctatccctcacgtttcagagctaagcacaagagtgc

SEQ ID NO: 81 - DNA, 69 bp, Artificial Sequence
  Synthetic polynucleotide
tcttgtggaaagccagaaacatgggccaagaaggttgatgctgggtttcagagctaagcacaagagtgc

SEQ ID NO: 82 - DNA, 69 bp, Artificial Sequence
  Synthetic polynucleotide
tcttgtggaaagccagaaacatgggaactgaaattcgaatggcggtttcagagctaagcacaagagtgc

SEQ ID NO: 83 - DNA, 69 bp, Artificial Sequence
  Synthetic polynucleotide
tcttgtggaaagccagaaacatggagagagccatcctctataaggtttcagagctaagcacaagagtgc

SEQ ID NO: 84 - DNA, 69 bp, Artificial Sequence
  Synthetic polynucleotide
tcttgtggaaagccagaaacatggcaccacagccttcccgcgaagtttcagagctaagcacaagagtgc

SEQ ID NO: 85 - DNA, 69 bp, Artificial Sequence
  Synthetic polynucleotide
tcttgtggaaagccagaaacatggattgaagacgtcattgcccagtttcagagctaagcacaagagtgc

SEQ ID NO: 86 - DNA, 69 bp, Artificial Sequence
  Synthetic polynucleotide
tcttgtggaaagccagaaacatggggatgtagtgcagatagtctgtttcagagctaagcacaagagtgc

SEQ ID NO: 87 - DNA, 69 bp, Artificial Sequence
  Synthetic polynucleotide
tcttgtggaaagccagaaacatggccagcgcgctacttacagtggtttcagagctaagcacaagagtgc

SEQ ID NO: 88 - DNA, 69 bp, Artificial Sequence
  Synthetic polynucleotide
tcttgtggaaagccagaaacatggccggagtttgatgtgtaggggtttcagagctaagcacaagagtgc

SEQ ID NO: 89 - DNA, 69 bp, Artificial Sequence
  Synthetic polynucleotide
tcttgtggaaagccagaaacatggcttgcgcaggcgcttcagcagtttcagagctaagcacaagagtgc

SEQ ID NO: 90 - DNA, 69 bp, Artificial Sequence
  Synthetic polynucleotide
tcttgtggaaagccagaaacatggcctcacaggcaggctaaacagtttcagagctaagcacaagagtgc

SEQ ID NO: 91 - DNA, 69 bp, Artificial Sequence
  Synthetic polynucleotide
tcttgtggaaagccagaaacatggatatccagaaagtttacctcgtttcagagctaagcacaagagtgc

SEQ ID NO: 92 - DNA, 69 bp, Artificial Sequence
  Synthetic polynucleotide
tcttgtggaaagccagaaacatggagacattgacctcaccaagagtttcagagctaagcacaagagtgc

SEQ ID NO: 93 - DNA, 69 bp, Artificial Sequence
  Synthetic polynucleotide
tcttgtggaaagccagaaacatgggcgcggcacctgtacctccggtttcagagctaagcacaagagtgc

SEQ ID NO: 94 - DNA, 69 bp, Artificial Sequence
  Synthetic polynucleotide
tcttgtggaaagccagaaacatggaccagttcgaatgcctaccagtttcagagctaagcacaagagtgc

SEQ ID NO: 95 - DNA, 69 bp, Artificial Sequence
  Synthetic polynucleotide
tcttgtggaaagccagaaacatggtaggcactgtcgccttcccggtttcagagctaagcacaagagtgc

SEQ ID NO: 96 - DNA, 69 bp, Artificial Sequence
  Synthetic polynucleotide
tcttgtggaaagccagaaacatggctgtgcccgatgcgtgcccagtttcagagctaagcacaagagtgc

SEQ ID NO: 97 - DNA, 69 bp, Artificial Sequence
  Synthetic polynucleotide
tcttgtggaaagccagaaacatggccacccggcccgtacctcgagtttcagagctaagcacaagagtgc

SEQ ID NO: 98 - DNA, 69 bp, Artificial Sequence
  Synthetic polynucleotide
tcttgtggaaagccagaaacatggagatggctactctggagttggtttcagagctaagcacaagagtgc

SEQ ID NO: 99 - DNA, 69 bp, Artificial Sequence
  Synthetic polynucleotide
tcttgtggaaagccagaaacatggcagacgaagcaagtaaccatgtttcagagctaagcacaagagtgc

SEQ ID NO: 100 - DNA, 69 bp, Artificial Sequence
  Synthetic polynucleotide
tcttgtggaaagccagaaacatggtaagatacttacaaacacacgtttcagagctaagcacaagagtgc

SEQ ID NO: 101 - DNA, 69 bp, Artificial Sequence
  Synthetic polynucleotide
tcttgtggaaagccagaaacatggcatacacctgctcacaggcggtttcagagctaagcacaagagtgc

SEQ ID NO: 102 - DNA, 69 bp, Artificial Sequence
  Synthetic polynucleotide
tcttgtggaaagccagaaacatggagtggtgggaacgacaaacagtttcagagctaagcacaagagtgc

SEQ ID NO: 103 - DNA, 69 bp, Artificial Sequence
  Synthetic polynucleotide
tcttgtggaaagccagaaacatggataagcaaaagcgtcccagggtttcagagctaagcacaagagtgc

SEQ ID NO: 104 - DNA, 69 bp, Artificial Sequence
  Synthetic polynucleotide
tcttgtggaaagccagaaacatggacagcacaccgtaccgacaggtttcagagctaagcacaagagtgc

SEQ ID NO: 105 - DNA, 69 bp, Artificial Sequence
  Synthetic polynucleotide
tcttgtggaaagccagaaacatgggcggaccagccgccgcagcagtttcagagctaagcacaagagtgc

SEQ ID NO: 106 - DNA, 69 bp, Artificial Sequence
  Synthetic polynucleotide
tcttgtggaaagccagaaacatggtacacagcgcaatggtaggtgtttcagagctaagcacaagagtgc

SEQ ID NO: 107 - DNA, 69 bp, Artificial Sequence
  Synthetic polynucleotide
tcttgtggaaagccagaaacatggtcttgttgatgcggatgacggtttcagagctaagcacaagagtgc

SEQ ID NO: 108 - DNA, 69 bp, Artificial Sequence
  Synthetic polynucleotide
tcttgtggaaagccagaaacatggtcgcgacaccctgtacgagggtttcagagctaagcacaagagtgc

SEQ ID NO: 109 - DNA, 69 bp, Artificial Sequence
  Synthetic polynucleotide
tcttgtggaaagccagaaacatggttcatttctccggatgccaagtttcagagctaagcacaagagtgc

SEQ ID NO: 110 - DNA, 69 bp, Artificial Sequence
  Synthetic polynucleotide
tcttgtggaaagccagaaacatggtcttctgcagttaaacacaggtttcagagctaagcacaagagtgc

SEQ ID NO: 111 - DNA, 69 bp, Artificial Sequence
  Synthetic polynucleotide
tcttgtggaaagccagaaacatggtaccacacgaaggtgcgcgcgtttcagagctaagcacaagagtgc

SEQ ID NO: 112 - DNA, 69 bp, Artificial Sequence
  Synthetic polynucleotide
tcttgtggaaagccagaaacatggtctttctctaacagaaaaaggtttcagagctaagcacaagagtgc

SEQ ID NO: 113 - DNA, 69 bp, Artificial Sequence
  Synthetic polynucleotide
tcttgtggaaagccagaaacatggttgtcggacatacttctggtgtttcagagctaagcacaagagtgc

SEQ ID NO: 114 - DNA, 69 bp, Artificial Sequence
  Synthetic polynucleotide
tcttgtggaaagccagaaacatggtcctggccgggaaaacaagagtttcagagctaagcacaagagtgc

SEQ ID NO: 115 - DNA, 69 bp, Artificial Sequence
  Synthetic polynucleotide
tcttgtggaaagccagaaacatgggaagttcttcacccgcaggggtttcagagctaagcacaagagtgc

SEQ ID NO: 116 - DNA, 69 bp, Artificial Sequence
  Synthetic polynucleotide
tcttgtggaaagccagaaacatggtgtacccttccgcttacctggtttcagagctaagcacaagagtgc

SEQ ID NO: 117 - DNA, 69 bp, Artificial Sequence
  Synthetic polynucleotide
tcttgtggaaagccagaaacatggtaaagctgaagccaaagcgagtttcagagctaagcacaagagtgc

SEQ ID NO: 118 - DNA, 69 bp, Artificial Sequence
  Synthetic polynucleotide
tcttgtggaaagccagaaacatgggccgagcagtcaaattccaggtttcagagctaagcacaagagtgc

SEQ ID NO: 119 - DNA, 69 bp, Artificial Sequence
  Synthetic polynucleotide
tcttgtggaaagccagaaacatggtttgcgattcttgcttcggtgtttcagagctaagcacaagagtgc

SEQ ID NO: 120 - DNA, 69 bp, Artificial Sequence
  Synthetic polynucleotide
tcttgtggaaagccagaaacatggtggcagctgtcactttgcgggtttcagagctaagcacaagagtgc

SEQ ID NO: 121 - DNA, 69 bp, Artificial Sequence
  Synthetic polynucleotide
tcttgtggaaagccagaaacatggtgagatgatcgacgtcatcggtttcagagctaagcacaagagtgc

SEQ ID NO: 122 - DNA, 69 bp, Artificial Sequence
  Synthetic polynucleotide
tcttgtggaaagccagaaacatggtgctttgtagaaaaagtcgcgtttcagagctaagcacaagagtgc

SEQ ID NO: 123 - DNA, 69 bp, Artificial Sequence
  Synthetic polynucleotide
tcttgtggaaagccagaaacatgggcttcaagaagcgtgcacctgtttcagagctaagcacaagagtgc

SEQ ID NO: 124 - DNA, 69 bp, Artificial Sequence
  Synthetic polynucleotide
tcttgtggaaagccagaaacatggcttccagctccttgacgttggtttcagagctaagcacaagagtgc

SEQ ID NO: 125 - DNA, 69 bp, Artificial Sequence
  Synthetic polynucleotide
tcttgtggaaagccagaaacatggtattgtaggaaagcctacgtgtttcagagctaagcacaagagtgc

SEQ ID NO: 126 - DNA, 69 bp, Artificial Sequence
  Synthetic polynucleotide
tcttgtggaaagccagaaacatggttacatcttagagagcttgggtttcagagctaagcacaagagtgc

SEQ ID NO: 127 - DNA, 69 bp, Artificial Sequence
  Synthetic polynucleotide
tcttgtggaaagccagaaacatgggtgtcttaggctgtggtccagtttcagagctaagcacaagagtgc

SEQ ID NO: 128 - DNA, 69 bp, Artificial Sequence
  Synthetic polynucleotide
tcttgtggaaagccagaaacatgggtggctttgccccgtacgaggtttcagagctaagcacaagagtgc

SEQ ID NO: 129 - DNA, 69 bp, Artificial Sequence
  Synthetic polynucleotide
tcttgtggaaagccagaaacatggtttagataactggagtgccagtttcagagctaagcacaagagtgc

SEQ ID NO: 130 - DNA, 69 bp, Artificial Sequence
  Synthetic polynucleotide
tcttgtggaaagccagaaacatggtcgggatcgtcggtaaatacgtttcagagctaagcacaagagtgc

SEQ ID NO: 131 - DNA, 69 bp, Artificial Sequence
  Synthetic polynucleotide
tcttgtggaaagccagaaacatgggttgcagcctcggaaaattggtttcagagctaagcacaagagtgc

SEQ ID NO: 132 - DNA, 69 bp, Artificial Sequence
  Synthetic polynucleotide
tcttgtggaaagccagaaacatggtttgttaggtcatcgtattggtttcagagctaagcacaagagtgc

SEQ ID NO: 133 - DNA, 69 bp, Artificial Sequence
  Synthetic polynucleotide
tcttgtggaaagccagaaacatggtcatcttgtacataccctgcgtttcagagctaagcacaagagtgc

SEQ ID NO: 134 - DNA, 69 bp, Artificial Sequence
  Synthetic polynucleotide
tcttgtggaaagccagaaacatgggttggtggtgacaagaacgggtttcagagctaagcacaagagtgc

SEQ ID NO: 135 - DNA, 69 bp, Artificial Sequence
  Synthetic polynucleotide
tcttgtggaaagccagaaacatggttcagcttcgaaaggcaagggtttcagagctaagcacaagagtgc

SEQ ID NO: 136 - DNA, 69 bp, Artificial Sequence
  Synthetic polynucleotide
tcttgtggaaagccagaaacatgggggcctactcacctgctcgagtttcagagctaagcacaagagtgc

SEQ ID NO: 137 - DNA, 69 bp, Artificial Sequence
  Synthetic polynucleotide
tcttgtggaaagccagaaacatggttatccgaaatgtttcattggtttcagagctaagcacaagagtgc

SEQ ID NO: 138 - DNA, 69 bp, Artificial Sequence
  Synthetic polynucleotide
tcttgtggaaagccagaaacatggggacgacgaccggctcaacagtttcagagctaagcacaagagtgc

SEQ ID NO: 139 - DNA, 69 bp, Artificial Sequence
  Synthetic polynucleotide
tcttgtggaaagccagaaacatgggtaatgtgtccattcgaggggtttcagagctaagcacaagagtgc

SEQ ID NO: 140 - DNA, 69 bp, Artificial Sequence
  Synthetic polynucleotide
tcttgtggaaagccagaaacatgggttgacatctgacgacgtgagtttcagagctaagcacaagagtgc

SEQ ID NO: 141 - DNA, 69 bp, Artificial Sequence
  Synthetic polynucleotide
tcttgtggaaagccagaaacatgggactggtgggatgaaggtaagtttcagagctaagcacaagagtgc

SEQ ID NO: 142 - DNA, 69 bp, Artificial Sequence
  Synthetic polynucleotide
tcttgtggaaagccagaaacatgggctggtcgaggtccacgccggtttcagagctaagcacaagagtgc

SEQ ID NO: 143 - DNA, 69 bp, Artificial Sequence
  Synthetic polynucleotide
tcttgtggaaagccagaaacatggtgacgtgcaactcaaagaccgtttcagagctaagcacaagagtgc

SEQ ID NO: 144 - DNA, 69 bp, Artificial Sequence
  Synthetic polynucleotide
tcttgtggaaagccagaaacatgggaaaagtactacacgcgcctgtttcagagctaagcacaagagtgc

SEQ ID NO: 145 - DNA, 69 bp, Artificial Sequence
  Synthetic polynucleotide
tcttgtggaaagccagaaacatggtcaacaccaacatcgatggggtttcagagctaagcacaagagtgc

SEQ ID NO: 146 - DNA, 69 bp, Artificial Sequence
  Synthetic polynucleotide
tcttgtggaaagccagaaacatggtgacggtatccacccattcggtttcagagctaagcacaagagtgc

SEQ ID NO: 147 - DNA, 69 bp, Artificial Sequence
  Synthetic polynucleotide
tcttgtggaaagccagaaacatgggtgtgtgctgacttgataaggtttcagagctaagcacaagagtgc

SEQ ID NO: 148 - DNA, 69 bp, Artificial Sequence
  Synthetic polynucleotide
tcttgtggaaagccagaaacatggtggtcatgaactttctagtggtttcagagctaagcacaagagtgc

SEQ ID NO: 149 - DNA, 69 bp, Artificial Sequence
  Synthetic polynucleotide
tcttgtggaaagccagaaacatggtgcagcgaataggctgcacggtttcagagctaagcacaagagtgc

SEQ ID NO: 150 - DNA, 69 bp, Artificial Sequence
  Synthetic polynucleotide
tcttgtggaaagccagaaacatggggatacgatagaaaatgtaagtttcagagctaagcacaagagtgc

SEQ ID NO: 151 - DNA, 69 bp, Artificial Sequence
  Synthetic polynucleotide
tcttgtggaaagccagaaacatgggctgaaaaggtggccactaggtttcagagctaagcacaagagtgc

SEQ ID NO: 152 - DNA, 69 bp, Artificial Sequence
  Synthetic polynucleotide
tcttgtggaaagccagaaacatggttccatggtcaaaaaatggcgtttcagagctaagcacaagagtgc

SEQ ID NO: 153 - DNA, 69 bp, Artificial Sequence
  Synthetic polynucleotide
tcttgtggaaagccagaaacatggtgttcctcaggaaaatgatggtttcagagctaagcacaagagtgc

SEQ ID NO: 154 - DNA, 69 bp, Artificial Sequence
  Synthetic polynucleotide
tcttgtggaaagccagaaacatggttcactgcaatgtaatcctggtttcagagctaagcacaagagtgc

SEQ ID NO: 155 - DNA, 69 bp, Artificial Sequence
  Synthetic polynucleotide
tcttgtggaaagccagaaacatggtactttctatgagaagcgtagtttcagagctaagcacaagagtgc

SEQ ID NO: 156 - DNA, 69 bp, Artificial Sequence
  Synthetic polynucleotide
tcttgtggaaagccagaaacatgggccaagatcgtgaagcccaagtttcagagctaagcacaagagtgc

SEQ ID NO: 157 - DNA, 69 bp, Artificial Sequence
  Synthetic polynucleotide
tcttgtggaaagccagaaacatgggctggttcgtaccaagacccgtttcagagctaagcacaagagtgc

SEQ ID NO: 158 - DNA, 69 bp, Artificial Sequence
  Synthetic polynucleotide
tcttgtggaaagccagaaacatggtgggctccggaacaaacgtggtttcagagctaagcacaagagtgc

SEQ ID NO: 159 - DNA, 69 bp, Artificial Sequence
  Synthetic polynucleotide
tcttgtggaaagccagaaacatggttggcagggagctcactcaggtttcagagctaagcacaagagtgc

SEQ ID NO: 160 - DNA, 69 bp, Artificial Sequence
  Synthetic polynucleotide
tcttgtggaaagccagaaacatggtccgtctgcttcatgagcgggtttcagagctaagcacaagagtgc

SEQ ID NO: 161 - DNA, 69 bp, Artificial Sequence
  Synthetic polynucleotide
tcttgtggaaagccagaaacatggtcctcacctaaagtgcaatagtttcagagctaagcacaagagtgc

SEQ ID NO: 162 - DNA, 69 bp, Artificial Sequence
  Synthetic polynucleotide
tcttgtggaaagccagaaacatggtcctcgatagctggaatccagtttcagagctaagcacaagagtgc

SEQ ID NO: 163 - DNA, 69 bp, Artificial Sequence
  Synthetic polynucleotide
tcttgtggaaagccagaaacatggtcctgccaagaaacacccttgtttcagagctaagcacaagagtgc

SEQ ID NO: 164 - DNA, 69 bp, Artificial Sequence
  Synthetic polynucleotide
tcttgtggaaagccagaaacatggtcctggataccgcgtggttagtttcagagctaagcacaagagtgc

SEQ ID NO: 165 - DNA, 69 bp, Artificial Sequence
  Synthetic polynucleotide
tcttgtggaaagccagaaacatggtcgagaggaaaaacacactggtttcagagctaagcacaagagtgc

SEQ ID NO: 166 - DNA, 69 bp, Artificial Sequence
  Synthetic polynucleotide
tcttgtggaaagccagaaacatggtcgagatgcgcagcagatgagtttcagagctaagcacaagagtgc

SEQ ID NO: 167 - DNA, 69 bp, Artificial Sequence
  Synthetic polynucleotide
tcttgtggaaagccagaaacatggtcgatcgaggttgcattcgggtttcagagctaagcacaagagtgc

SEQ ID NO: 168 - DNA, 69 bp, Artificial Sequence
  Synthetic polynucleotide
tcttgtggaaagccagaaacatggtcgatgtagccccgcccaaggtttcagagctaagcacaagagtgc

SEQ ID NO: 169 - DNA, 69 bp, Artificial Sequence
  Synthetic polynucleotide
tcttgtggaaagccagaaacatggtcgcaaggaagccagctaaggtttcagagctaagcacaagagtgc

SEQ ID NO: 170 - DNA, 69 bp, Artificial Sequence
  Synthetic polynucleotide
tcttgtggaaagccagaaacatggtcgcagcggcgtgggatcgggtttcagagctaagcacaagagtgc

SEQ ID NO: 171 - DNA, 69 bp, Artificial Sequence
  Synthetic polynucleotide
tcttgtggaaagccagaaacatggtcgcgcttgggttatacgctgtttcagagctaagcacaagagtgc

SEQ ID NO: 172 - DNA, 69 bp, Artificial Sequence
  Synthetic polynucleotide
tcttgtggaaagccagaaacatggtcgcggacatagggctctaagtttcagagctaagcacaagagtgc

SEQ ID NO: 173 - DNA, 69 bp, Artificial Sequence
  Synthetic polynucleotide
tcttgtggaaagccagaaacatggtcggaagcaaacttctggaggtttcagagctaagcacaagagtgc

SEQ ID NO: 174 - DNA, 69 bp, Artificial Sequence
  Synthetic polynucleotide
tcttgtggaaagccagaaacatggtcggcatacgggacacacgcgtttcagagctaagcacaagagtgc

SEQ ID NO: 175 - DNA, 69 bp, Artificial Sequence
  Synthetic polynucleotide
tcttgtggaaagccagaaacatggtcggctacaatctttggcatgtttcagagctaagcacaagagtgc

SEQ ID NO: 176 - DNA, 69 bp, Artificial Sequence
  Synthetic polynucleotide
tcttgtggaaagccagaaacatggtcggctacggcgtggagaaggtttcagagctaagcacaagagtgc

SEQ ID NO: 177 - DNA, 69 bp, Artificial Sequence
  Synthetic polynucleotide
tcttgtggaaagccagaaacatggtcggctcctgaagccagtatgtttcagagctaagcacaagagtgc

SEQ ID NO: 178 - DNA, 69 bp, Artificial Sequence
  Synthetic polynucleotide
tcttgtggaaagccagaaacatggtcgggcagtgagtacaatacgtttcagagctaagcacaagagtgc

SEQ ID NO: 179 - DNA, 69 bp, Artificial Sequence
  Synthetic polynucleotide
tcttgtggaaagccagaaacatggtcggggaccacccacgatccgtttcagagctaagcacaagagtgc

SEQ ID NO: 180 - DNA, 69 bp, Artificial Sequence
  Synthetic polynucleotide
tcttgtggaaagccagaaacatggtcgtaaacacacgaccaagtgtttcagagctaagcacaagagtgc

SEQ ID NO: 181 - DNA, 69 bp, Artificial Sequence
  Synthetic polynucleotide
tcttgtggaaagccagaaacatggtctaaagccgtcctgatgttgtttcagagctaagcacaagagtgc

SEQ ID NO: 182 - DNA, 69 bp, Artificial Sequence
  Synthetic polynucleotide
tcttgtggaaagccagaaacatggtctacctattgtggaatttggtttcagagctaagcacaagagtgc

SEQ ID NO: 183 - DNA, 69 bp, Artificial Sequence
  Synthetic polynucleotide
tcttgtggaaagccagaaacatggtctacgtgtagttgtacatagtttcagagctaagcacaagagtgc

SEQ ID NO: 184 - DNA, 69 bp, Artificial Sequence
  Synthetic polynucleotide
tcttgtggaaagccagaaacatggtctattttgtctgcgcagaagtttcagagctaagcacaagagtgc

SEQ ID NO: 185 - DNA, 69 bp, Artificial Sequence
  Synthetic polynucleotide
tcttgtggaaagccagaaacatggtctcgtagcctaatgcgccagtttcagagctaagcacaagagtgc

SEQ ID NO: 186 - DNA, 69 bp, Artificial Sequence
  Synthetic polynucleotide
tcttgtggaaagccagaaacatggtctctcggagtggagcaacagtttcagagctaagcacaagagtgc

SEQ ID NO: 187 - DNA, 69 bp, Artificial Sequence
  Synthetic polynucleotide
tcttgtggaaagccagaaacatggtctgaaaaataggcccaaccgtttcagagctaagcacaagagtgc

SEQ ID NO: 188 - DNA, 69 bp, Artificial Sequence
  Synthetic polynucleotide
tcttgtggaaagccagaaacatggtctgacgattaatgcttctagtttcagagctaagcacaagagtgc

SEQ ID NO: 189 - DNA, 69 bp, Artificial Sequence
  Synthetic polynucleotide
tcttgtggaaagccagaaacatggtctggcttgacacgaccgttgtttcagagctaagcacaagagtgc

SEQ ID NO: 190 - DNA, 69 bp, Artificial Sequence
  Synthetic polynucleotide
tcttgtggaaagccagaaacatggtctggcttgcaccgtgtcatgtttcagagctaagcacaagagtgc

SEQ ID NO: 191 - DNA, 69 bp, Artificial Sequence
  Synthetic polynucleotide
tcttgtggaaagccagaaacatggtgaacgcgtgtttccttgcagtttcagagctaagcacaagagtgc

SEQ ID NO: 192 - DNA, 69 bp, Artificial Sequence
  Synthetic polynucleotide
tcttgtggaaagccagaaacatggtgaacggtgaagagataggggtttcagagctaagcacaagagtgc

SEQ ID NO: 193 - DNA, 69 bp, Artificial Sequence
  Synthetic polynucleotide
tcttgtggaaagccagaaacatggtgaagtggggcgtcggacacgtttcagagctaagcacaagagtgc

SEQ ID NO: 194 - DNA, 69 bp, Artificial Sequence
  Synthetic polynucleotide
tcttgtggaaagccagaaacatggtgaatcgaatacaaacgatggtttcagagctaagcacaagagtgc

SEQ ID NO: 195 - DNA, 69 bp, Artificial Sequence
  Synthetic polynucleotide
tcttgtggaaagccagaaacatggtgaatcgtaacctcgccattgtttcagagctaagcacaagagtgc

SEQ ID NO: 196 - DNA, 69 bp, Artificial Sequence
  Synthetic polynucleotide
tcttgtggaaagccagaaacatggtgacacattggctgggtgttgtttcagagctaagcacaagagtgc

SEQ ID NO: 197 - DNA, 69 bp, Artificial Sequence
  Synthetic polynucleotide
tcttgtggaaagccagaaacatggtgacctctgaggaattcacagtttcagagctaagcacaagagtgc

SEQ ID NO: 198 - DNA, 69 bp, Artificial Sequence
  Synthetic polynucleotide
tcttgtggaaagccagaaacatggtgacgcgatagagttggcttgtttcagagctaagcacaagagtgc

SEQ ID NO: 199 - DNA, 69 bp, Artificial Sequence
  Synthetic polynucleotide
tcttgtggaaagccagaaacatggtgacgctccacgtccggaccgtttcagagctaagcacaagagtgc

SEQ ID NO: 200 - DNA, 69 bp, Artificial Sequence
  Synthetic polynucleotide
tcttgtggaaagccagaaacatggtgactagctcttacatattcgtttcagagctaagcacaagagtgc

SEQ ID NO: 201 - DNA, 69 bp, Artificial Sequence
  Synthetic polynucleotide
tcttgtggaaagccagaaacatggtgactcgggcaatatcggttgtttcagagctaagcacaagagtgc

SEQ ID NO: 202 - DNA, 69 bp, Artificial Sequence
  Synthetic polynucleotide
tcttgtggaaagccagaaacatggtgagcatgtcgggagtaactgtttcagagctaagcacaagagtgc

SEQ ID NO: 203 - DNA, 69 bp, Artificial Sequence
  Synthetic polynucleotide
tcttgtggaaagccagaaacatggtgagcattcgtagcccagcagtttcagagctaagcacaagagtgc

SEQ ID NO: 204 - DNA, 69 bp, Artificial Sequence
  Synthetic polynucleotide
tcttgtggaaagccagaaacatggtgagcggcctctaattaatcgtttcagagctaagcacaagagtgc

SEQ ID NO: 205 - DNA, 69 bp, Artificial Sequence
  Synthetic polynucleotide
tcttgtggaaagccagaaacatggtgaggatcatgtcgagcgccgtttcagagctaagcacaagagtgc

SEQ ID NO: 206 - DNA, 69 bp, Artificial Sequence
  Synthetic polynucleotide
tcttgtggaaagccagaaacatggtgagtcttactaggtcctgtgtttcagagctaagcacaagagtgc

SEQ ID NO: 207 - DNA, 69 bp, Artificial Sequence
  Synthetic polynucleotide
tcttgtggaaagccagaaacatggtgcaaccttccttttcaggagtttcagagctaagcacaagagtgc

SEQ ID NO: 208 - DNA, 69 bp, Artificial Sequence
  Synthetic polynucleotide
tcttgtggaaagccagaaacatggtgcaaggacctggtatgaacgtttcagagctaagcacaagagtgc

SEQ ID NO: 209 - DNA, 69 bp, Artificial Sequence
  Synthetic polynucleotide
tcttgtggaaagccagaaacatggtgcaggtctaggtcccaaaggtttcagagctaagcacaagagtgc

SEQ ID NO: 210 - DNA, 69 bp, Artificial Sequence
  Synthetic polynucleotide
tcttgtggaaagccagaaacatggtgcagtcgcgctgagcgtcagtttcagagctaagcacaagagtgc

SEQ ID NO: 211 - DNA, 69 bp, Artificial Sequence
  Synthetic polynucleotide
tcttgtggaaagccagaaacatggatcaacgactaccagcccgggtttcagagctaagcacaagagtgc

SEQ ID NO: 212 - DNA, 69 bp, Artificial Sequence
  Synthetic polynucleotide
tcttgtggaaagccagaaacatggagacgtgcactttgagcggagtttcagagctaagcacaagagtgc

SEQ ID NO: 213 - DNA, 69 bp, Artificial Sequence
  Synthetic polynucleotide
tcttgtggaaagccagaaacatggagtgcaaggatagaaacaccgtttcagagctaagcacaagagtgc

SEQ ID NO: 214 - DNA, 69 bp, Artificial Sequence
  Synthetic polynucleotide
tcttgtggaaagccagaaacatggaaacaaaagcccatgaatgtgtttcagagctaagcacaagagtgc

SEQ ID NO: 215 - DNA, 69 bp, Artificial Sequence
  Synthetic polynucleotide
tcttgtggaaagccagaaacatggcaacattaaatacccaatgagtttcagagctaagcacaagagtgc

SEQ ID NO: 216 - DNA, 69 bp, Artificial Sequence
  Synthetic polynucleotide
tcttgtggaaagccagaaacatggcaaccacaatctcaggacgagtttcagagctaagcacaagagtgc

SEQ ID NO: 217 - DNA, 69 bp, Artificial Sequence
  Synthetic polynucleotide
tcttgtggaaagccagaaacatgggcaacttgtcacagacgcaggtttcagagctaagcacaagagtgc

SEQ ID NO: 218 - DNA, 69 bp, Artificial Sequence
  Synthetic polynucleotide
tcttgtggaaagccagaaacatggcctgccaagaaggatgtcaagtttcagagctaagcacaagagtgc

SEQ ID NO: 219 - DNA, 69 bp, Artificial Sequence
  Synthetic polynucleotide
tcttgtggaaagccagaaacatggcgtggacggagtgattacgagtttcagagctaagcacaagagtgc

SEQ ID NO: 220 - DNA, 69 bp, Artificial Sequence
  Synthetic polynucleotide
tcttgtggaaagccagaaacatggagtgattgtgtgtcctaccagtttcagagctaagcacaagagtgc

SEQ ID NO: 221 - DNA, 69 bp, Artificial Sequence
  Synthetic polynucleotide
tcttgtggaaagccagaaacatggaagtctacttgtatctacgggtttcagagctaagcacaagagtgc

SEQ ID NO: 222 - DNA, 69 bp, Artificial Sequence
  Synthetic polynucleotide
tcttgtggaaagccagaaacatggaagatttaatagctcaagcagtttcagagctaagcacaagagtgc

SEQ ID NO: 223 - DNA, 69 bp, Artificial Sequence
  Synthetic polynucleotide
tcttgtggaaagccagaaacatggacgtcagggatgtcctcgatgtttcagagctaagcacaagagtgc

SEQ ID NO: 224 - DNA, 69 bp, Artificial Sequence
  Synthetic polynucleotide
tcttgtggaaagccagaaacatggaaacacaaaattcacgtccagtttcagagctaagcacaagagtgc

SEQ ID NO: 225 - DNA, 69 bp, Artificial Sequence
  Synthetic polynucleotide
tcttgtggaaagccagaaacatggaggtgccaacacccatccaggtttcagagctaagcacaagagtgc

SEQ ID NO: 226 - DNA, 69 bp, Artificial Sequence
  Synthetic polynucleotide
tcttgtggaaagccagaaacatggatcatctcacagttcaaccagtttcagagctaagcacaagagtgc

SEQ ID NO: 227 - DNA, 69 bp, Artificial Sequence
  Synthetic polynucleotide
tcttgtggaaagccagaaacatggcgcccaggaatagttaggaggtttcagagctaagcacaagagtgc

SEQ ID NO: 228 - DNA, 69 bp, Artificial Sequence
  Synthetic polynucleotide
tcttgtggaaagccagaaacatggcccaggtcgaacatatccaggtttcagagctaagcacaagagtgc

SEQ ID NO: 229 - DNA, 69 bp, Artificial Sequence
  Synthetic polynucleotide
tcttgtggaaagccagaaacatggcaaaacattatactggcatggtttcagagctaagcacaagagtgc

SEQ ID NO: 230 - DNA, 69 bp, Artificial Sequence
  Synthetic polynucleotide
tcttgtggaaagccagaaacatggaacaccaccgtaccttcccggtttcagagctaagcacaagagtgc

SEQ ID NO: 231 - DNA, 69 bp, Artificial Sequence
  Synthetic polynucleotide
tcttgtggaaagccagaaacatggcttcgtgacctactccaatggtttcagagctaagcacaagagtgc

SEQ ID NO: 232 - DNA, 69 bp, Artificial Sequence
  Synthetic polynucleotide
tcttgtggaaagccagaaacatggcttactgacaatcttatccagtttcagagctaagcacaagagtgc

SEQ ID NO: 233 - DNA, 69 bp, Artificial Sequence
  Synthetic polynucleotide
tcttgtggaaagccagaaacatggactctcccatcaattgaatggtttcagagctaagcacaagagtgc

SEQ ID NO: 234 - DNA, 69 bp, Artificial Sequence
  Synthetic polynucleotide
tcttgtggaaagccagaaacatggaatgtgtgctcgaccacacagtttcagagctaagcacaagagtgc

SEQ ID NO: 235 - DNA, 69 bp, Artificial Sequence
  Synthetic polynucleotide
tcttgtggaaagccagaaacatggccaacactggacggtcaagagtttcagagctaagcacaagagtgc

SEQ ID NO: 236 - DNA, 69 bp, Artificial Sequence
  Synthetic polynucleotide
tcttgtggaaagccagaaacatggaaattcacttacctaaaaccgtttcagagctaagcacaagagtgc

SEQ ID NO: 237 - DNA, 69 bp, Artificial Sequence
  Synthetic polynucleotide
tcttgtggaaagccagaaacatggaattctaagagtggaaagcggtttcagagctaagcacaagagtgc

SEQ ID NO: 238 - DNA, 69 bp, Artificial Sequence
  Synthetic polynucleotide
tcttgtggaaagccagaaacatggacaagaccaataagaggcgtgtttcagagctaagcacaagagtgc

SEQ ID NO: 239 - DNA, 69 bp, Artificial Sequence
  Synthetic polynucleotide
tcttgtggaaagccagaaacatggagagtacttgtctcgatttggtttcagagctaagcacaagagtgc

SEQ ID NO: 240 - DNA, 69 bp, Artificial Sequence
  Synthetic polynucleotide
tcttgtggaaagccagaaacatggactcccatttggatgcacaagtttcagagctaagcacaagagtgc

SEQ ID NO: 241 - DNA, 69 bp, Artificial Sequence
  Synthetic polynucleotide
tcttgtggaaagccagaaacatggaaatgggataacattgccgggtttcagagctaagcacaagagtgc

SEQ ID NO: 242 - DNA, 69 bp, Artificial Sequence
  Synthetic polynucleotide
tcttgtggaaagccagaaacatggaggtccctatgataggccgggtttcagagctaagcacaagagtgc

SEQ ID NO: 243 - DNA, 69 bp, Artificial Sequence
  Synthetic polynucleotide
tcttgtggaaagccagaaacatggacattgacgatggactaccagtttcagagctaagcacaagagtgc

SEQ ID NO: 244 - DNA, 69 bp, Artificial Sequence
  Synthetic polynucleotide
tcttgtggaaagccagaaacatggatgatgtttgatgaccgtcggtttcagagctaagcacaagagtgc

SEQ ID NO: 245 - DNA, 69 bp, Artificial Sequence
  Synthetic polynucleotide
tcttgtggaaagccagaaacatggactcagttcaaagtgcccaggtttcagagctaagcacaagagtgc

SEQ ID NO: 246 - DNA, 69 bp, Artificial Sequence
  Synthetic polynucleotide
tcttgtggaaagccagaaacatggaatactgatgatccatcagggtttcagagctaagcacaagagtgc

SEQ ID NO: 247 - DNA, 69 bp, Artificial Sequence
  Synthetic polynucleotide
tcttgtggaaagccagaaacatggaccccatgccaataccaccagtttcagagctaagcacaagagtgc

SEQ ID NO: 248 - DNA, 69 bp, Artificial Sequence
  Synthetic polynucleotide
tcttgtggaaagccagaaacatggacactccaaggtgtttaccagtttcagagctaagcacaagagtgc

SEQ ID NO: 249 - DNA, 69 bp, Artificial Sequence
  Synthetic polynucleotide
tcttgtggaaagccagaaacatggatcgagttagaggaccaaaggtttcagagctaagcacaagagtgc

SEQ ID NO: 250 - DNA, 69 bp, Artificial Sequence
  Synthetic polynucleotide
tcttgtggaaagccagaaacatggccagccgatacggaccacgtgtttcagagctaagcacaagagtgc

SEQ ID NO: 251 - DNA, 69 bp, Artificial Sequence
  Synthetic polynucleotide
tcttgtggaaagccagaaacatggcaagtgagcaaagaccgctagtttcagagctaagcacaagagtgc

SEQ ID NO: 252 - DNA, 69 bp, Artificial Sequence
  Synthetic polynucleotide
tcttgtggaaagccagaaacatggagatggtatagctgcatgctgtttcagagctaagcacaagagtgc

SEQ ID NO: 253 - DNA, 69 bp, Artificial Sequence
  Synthetic polynucleotide
tcttgtggaaagccagaaacatggagtggaccgcaacatcaagggtttcagagctaagcacaagagtgc

SEQ ID NO: 254 - DNA, 69 bp, Artificial Sequence
  Synthetic polynucleotide
tcttgtggaaagccagaaacatggacacactcggtgacagactggtttcagagctaagcacaagagtgc

SEQ ID NO: 255 - DNA, 69 bp, Artificial Sequence
  Synthetic polynucleotide
tcttgtggaaagccagaaacatggagttggcagtatcatcggaagtttcagagctaagcacaagagtgc

SEQ ID NO: 256 - DNA, 69 bp, Artificial Sequence
  Synthetic polynucleotide
tcttgtggaaagccagaaacatggcatggtgaactactacacctgtttcagagctaagcacaagagtgc

SEQ ID NO: 257 - DNA, 69 bp, Artificial Sequence
  Synthetic polynucleotide
tcttgtggaaagccagaaacatggaaatatggacgaatagtggagtttcagagctaagcacaagagtgc

SEQ ID NO: 258 - DNA, 69 bp, Artificial Sequence
  Synthetic polynucleotide
tcttgtggaaagccagaaacatggatgatcgtggaagatctacagtttcagagctaagcacaagagtgc

SEQ ID NO: 259 - DNA, 69 bp, Artificial Sequence
  Synthetic polynucleotide
tcttgtggaaagccagaaacatgggatgacaagaaaaaaaacaggtttcagagctaagcacaagagtgc

SEQ ID NO: 260 - DNA, 69 bp, Artificial Sequence
  Synthetic polynucleotide
tcttgtggaaagccagaaacatggatgagtgctaaaatgctggggtttcagagctaagcacaagagtgc

SEQ ID NO: 261 - DNA, 69 bp, Artificial Sequence
  Synthetic polynucleotide
tcttgtggaaagccagaaacatggatggagggactgtagtaactgtttcagagctaagcacaagagtgc

SEQ ID NO: 262 - DNA, 69 bp, Artificial Sequence
  Synthetic polynucleotide
tcttgtggaaagccagaaacatggattctgtgactatggaaccagtttcagagctaagcacaagagtgc

SEQ ID NO: 263 - DNA, 69 bp, Artificial Sequence
  Synthetic polynucleotide
tcttgtggaaagccagaaacatggaccatcttaccactcttccagtttcagagctaagcacaagagtgc

SEQ ID NO: 264 - DNA, 69 bp, Artificial Sequence
  Synthetic polynucleotide
tcttgtggaaagccagaaacatgggccagttgtaacaatgtcaggtttcagagctaagcacaagagtgc

SEQ ID NO: 265 - DNA, 69 bp, Artificial Sequence
  Synthetic polynucleotide
tcttgtggaaagccagaaacatggcctgtacaacgagcacacgggtttcagagctaagcacaagagtgc

SEQ ID NO: 266 - DNA, 69 bp, Artificial Sequence
  Synthetic polynucleotide
tcttgtggaaagccagaaacatgggcctcgattcactcgcgcgggtttcagagctaagcacaagagtgc

SEQ ID NO: 267 - DNA, 69 bp, Artificial Sequence
  Synthetic polynucleotide
tcttgtggaaagccagaaacatggcatgaacccagacatactcagtttcagagctaagcacaagagtgc

SEQ ID NO: 268 - DNA, 69 bp, Artificial Sequence
  Synthetic polynucleotide
tcttgtggaaagccagaaacatggctgatcgtatcttggatgtggtttcagagctaagcacaagagtgc

SEQ ID NO: 269 - DNA, 69 bp, Artificial Sequence
  Synthetic polynucleotide
tcttgtggaaagccagaaacatggcatcacaaaaaagctgtcaggtttcagagctaagcacaagagtgc

SEQ ID NO: 270 - DNA, 69 bp, Artificial Sequence
  Synthetic polynucleotide
tcttgtggaaagccagaaacatggcatggtttgtgatgatccgggtttcagagctaagcacaagagtgc

SEQ ID NO: 271 - DNA, 69 bp, Artificial Sequence
  Synthetic polynucleotide
tcttgtggaaagccagaaacatggcttaatcggaaccataggcggtttcagagctaagcacaagagtgc

SEQ ID NO: 272 - DNA, 69 bp, Artificial Sequence
  Synthetic polynucleotide
tcttgtggaaagccagaaacatggctcaaagccacagtcgacaagtttcagagctaagcacaagagtgc

SEQ ID NO: 273 - DNA, 69 bp, Artificial Sequence
  Synthetic polynucleotide
tcttgtggaaagccagaaacatgggcaccaccataaaccacgcagtttcagagctaagcacaagagtgc

SEQ ID NO: 274 - DNA, 69 bp, Artificial Sequence
  Synthetic polynucleotide
tcttgtggaaagccagaaacatggcctgatcgaccctattcgaggtttcagagctaagcacaagagtgc

SEQ ID NO: 275 - DNA, 69 bp, Artificial Sequence
  Synthetic polynucleotide
tcttgtggaaagccagaaacatggcattcctagagagaggcgatgtttcagagctaagcacaagagtgc

SEQ ID NO: 276 - DNA, 69 bp, Artificial Sequence
  Synthetic polynucleotide
tcttgtggaaagccagaaacatggaagtgataataaactagagggtttcagagctaagcacaagagtgc

SEQ ID NO: 277 - DNA, 69 bp, Artificial Sequence
  Synthetic polynucleotide
tcttgtggaaagccagaaacatggctgcctcgcagaaagcaacggtttcagagctaagcacaagagtgc

SEQ ID NO: 278 - DNA, 69 bp, Artificial Sequence
  Synthetic polynucleotide
tcttgtggaaagccagaaacatggtcagtgtgtccattggagcagtttcagagctaagcacaagagtgc

SEQ ID NO: 279 - DNA, 69 bp, Artificial Sequence
  Synthetic polynucleotide
tcttgtggaaagccagaaacatggcaggtaggggatttcatcaggtttcagagctaagcacaagagtgc

SEQ ID NO: 280 - DNA, 69 bp, Artificial Sequence
  Synthetic polynucleotide
tcttgtggaaagccagaaacatggccagttacagcagtttcagggtttcagagctaagcacaagagtgc

SEQ ID NO: 281 - DNA, 69 bp, Artificial Sequence
  Synthetic polynucleotide
tcttgtggaaagccagaaacatggtcatagtatcgaggagtatggtttcagagctaagcacaagagtgc

SEQ ID NO: 282 - DNA, 69 bp, Artificial Sequence
  Synthetic polynucleotide
tcttgtggaaagccagaaacatggtagacggcgaaatcttgtcggtttcagagctaagcacaagagtgc

SEQ ID NO: 283 - DNA, 69 bp, Artificial Sequence
  Synthetic polynucleotide
tcttgtggaaagccagaaacatggggagatttaccaacaaccatgtttcagagctaagcacaagagtgc

SEQ ID NO: 284 - DNA, 69 bp, Artificial Sequence
  Synthetic polynucleotide
tcttgtggaaagccagaaacatggcaaagctataatccccctcagtttcagagctaagcacaagagtgc

SEQ ID NO: 285 - DNA, 69 bp, Artificial Sequence
  Synthetic polynucleotide
tcttgtggaaagccagaaacatggggaggatatctactccggtggtttcagagctaagcacaagagtgc

SEQ ID NO: 286 - DNA, 69 bp, Artificial Sequence
  Synthetic polynucleotide
tcttgtggaaagccagaaacatgggggaacgctcacggactgtggtttcagagctaagcacaagagtgc

SEQ ID NO: 287 - DNA, 69 bp, Artificial Sequence
  Synthetic polynucleotide
tcttgtggaaagccagaaacatggaggaactactacgaacaatggtttcagagctaagcacaagagtgc

SEQ ID NO: 288 - DNA, 69 bp, Artificial Sequence
  Synthetic polynucleotide
tcttgtggaaagccagaaacatggctaatacctcctccccccgggtttcagagctaagcacaagagtgc

SEQ ID NO: 289 - DNA, 69 bp, Artificial Sequence
  Synthetic polynucleotide
tcttgtggaaagccagaaacatggcgccaccgagaacggacatggtttcagagctaagcacaagagtgc

SEQ ID NO: 290 - DNA, 69 bp, Artificial Sequence
  Synthetic polynucleotide
tcttgtggaaagccagaaacatggaggtgtgaaacgatctgcaggtttcagagctaagcacaagagtgc

SEQ ID NO: 291 - DNA, 69 bp, Artificial Sequence
  Synthetic polynucleotide
tcttgtggaaagccagaaacatggaggtgtgaaacgatcagcaggtttcagagctaagcacaagagtgc

SEQ ID NO: 292 - DNA, 69 bp, Artificial Sequence
  Synthetic polynucleotide
tcttgtggaaagccagaaacatggccatggtggcggcgacacaggtttcagagctaagcacaagagtgc

SEQ ID NO: 293 - DNA, 69 bp, Artificial Sequence
  Synthetic polynucleotide
tcttgtggaaagccagaaacatggcaacgcgagcaagaatcagcgtttcagagctaagcacaagagtgc

SEQ ID NO: 294 - DNA, 69 bp, Artificial Sequence
  Synthetic polynucleotide
tcttgtggaaagccagaaacatggccatttcatctacactagaggtttcagagctaagcacaagagtgc

SEQ ID NO: 295 - DNA, 69 bp, Artificial Sequence
  Synthetic polynucleotide
tcttgtggaaagccagaaacatggaatgtctgatcacagatacggtttcagagctaagcacaagagtgc

SEQ ID NO: 296 - DNA, 69 bp, Artificial Sequence
  Synthetic polynucleotide
tcttgtggaaagccagaaacatggctgtgaagcaaactgcacaagtttcagagctaagcacaagagtgc

SEQ ID NO: 297 - DNA, 69 bp, Artificial Sequence
  Synthetic polynucleotide
tcttgtggaaagccagaaacatggagatagcagaaaatgctctggtttcagagctaagcacaagagtgc

SEQ ID NO: 298 - DNA, 69 bp, Artificial Sequence
  Synthetic polynucleotide
tcttgtggaaagccagaaacatggctgttgggacataccgctcggtttcagagctaagcacaagagtgc

SEQ ID NO: 299 - DNA, 69 bp, Artificial Sequence
  Synthetic polynucleotide
tcttgtggaaagccagaaacatggaggatgggtccaccagtggggtttcagagctaagcacaagagtgc

SEQ ID NO: 300 - DNA, 69 bp, Artificial Sequence
  Synthetic polynucleotide
tcttgtggaaagccagaaacatggaggactctgtgaatctgtgggtttcagagctaagcacaagagtgc

SEQ ID NO: 301 - DNA, 69 bp, Artificial Sequence
  Synthetic polynucleotide
tcttgtggaaagccagaaacatggaggtgatggctacgactggtgtttcagagctaagcacaagagtgc

SEQ ID NO: 302 - DNA, 69 bp, Artificial Sequence
  Synthetic polynucleotide
tcttgtggaaagccagaaacatggagaaccagatccagaagtcagtttcagagctaagcacaagagtgc

SEQ ID NO: 303 - DNA, 69 bp, Artificial Sequence
  Synthetic polynucleotide
tcttgtggaaagccagaaacatggcaccgtcaccgcgaacagcggtttcagagctaagcacaagagtgc

SEQ ID NO: 304 - DNA, 69 bp, Artificial Sequence
  Synthetic polynucleotide
tcttgtggaaagccagaaacatggccccggatacgctcactaaggtttcagagctaagcacaagagtgc

SEQ ID NO: 305 - DNA, 69 bp, Artificial Sequence
  Synthetic polynucleotide
tcttgtggaaagccagaaacatggtcttacggtttcgatggacggtttcagagctaagcacaagagtgc

SEQ ID NO: 306 - DNA, 69 bp, Artificial Sequence
  Synthetic polynucleotide
tcttgtggaaagccagaaacatggcagcataaacgagttccctggtttcagagctaagcacaagagtgc

SEQ ID NO: 307 - DNA, 69 bp, Artificial Sequence
  Synthetic polynucleotide
tcttgtggaaagccagaaacatggctggacaatatgccactccggtttcagagctaagcacaagagtgc

SEQ ID NO: 308 - DNA, 69 bp, Artificial Sequence
  Synthetic polynucleotide
tcttgtggaaagccagaaacatggagagatccgcgagagtacgggtttcagagctaagcacaagagtgc

SEQ ID NO: 309 - DNA, 69 bp, Artificial Sequence
  Synthetic polynucleotide
tcttgtggaaagccagaaacatggcaagatcaaggaaatacgaggtttcagagctaagcacaagagtgc

SEQ ID NO: 310 - DNA, 69 bp, Artificial Sequence
  Synthetic polynucleotide
tcttgtggaaagccagaaacatggccacgatgatcctgagcacggtttcagagctaagcacaagagtgc

SEQ ID NO: 311 - DNA, 69 bp, Artificial Sequence
  Synthetic polynucleotide
tcttgtggaaagccagaaacatggacatctctacgagagggcaggtttcagagctaagcacaagagtgc

SEQ ID NO: 312 - DNA, 69 bp, Artificial Sequence
  Synthetic polynucleotide
tcttgtggaaagccagaaacatggatgggcctcaatcagaatcggtttcagagctaagcacaagagtgc

SEQ ID NO: 313 - DNA, 69 bp, Artificial Sequence
  Synthetic polynucleotide
tcttgtggaaagccagaaacatggtattcctaagagtaaaaccagtttcagagctaagcacaagagtgc

SEQ ID NO: 314 - DNA, 69 bp, Artificial Sequence
  Synthetic polynucleotide
tcttgtggaaagccagaaacatggattcttataaggttctctgggtttcagagctaagcacaagagtgc

SEQ ID NO: 315 - DNA, 69 bp, Artificial Sequence
  Synthetic polynucleotide
tcttgtggaaagccagaaacatgggaatactatagaccaagggagtttcagagctaagcacaagagtgc

SEQ ID NO: 316 - DNA, 69 bp, Artificial Sequence
  Synthetic polynucleotide
tcttgtggaaagccagaaacatggcttgctcttacacagagtgtgtttcagagctaagcacaagagtgc

SEQ ID NO: 317 - DNA, 69 bp, Artificial Sequence
  Synthetic polynucleotide
tcttgtggaaagccagaaacatggtaacacggcaccaatgctgtgtttcagagctaagcacaagagtgc

SEQ ID NO: 318 - DNA, 69 bp, Artificial Sequence
  Synthetic polynucleotide
tcttgtggaaagccagaaacatggggacatcatagagcgctcgtgtttcagagctaagcacaagagtgc

SEQ ID NO: 319 - DNA, 69 bp, Artificial Sequence
  Synthetic polynucleotide
tcttgtggaaagccagaaacatgggaggcgcgcaaggtgaagaggtttcagagctaagcacaagagtgc

SEQ ID NO: 320 - DNA, 69 bp, Artificial Sequence
  Synthetic polynucleotide
tcttgtggaaagccagaaacatggtttcccacctgtctccacgcgtttcagagctaagcacaagagtgc

SEQ ID NO: 321 - DNA, 69 bp, Artificial Sequence
  Synthetic polynucleotide
tcttgtggaaagccagaaacatggtgataggccagttctctcgtgtttcagagctaagcacaagagtgc

SEQ ID NO: 322 - DNA, 69 bp, Artificial Sequence
  Synthetic polynucleotide
tcttgtggaaagccagaaacatggtgataaagcgaatgcaacaggtttcagagctaagcacaagagtgc

SEQ ID NO: 323 - DNA, 69 bp, Artificial Sequence
  Synthetic polynucleotide
tcttgtggaaagccagaaacatgggatgtcatccgagtatataggtttcagagctaagcacaagagtgc

SEQ ID NO: 324 - DNA, 69 bp, Artificial Sequence
  Synthetic polynucleotide
tcttgtggaaagccagaaacatggtgagactagatcaccaggcagtttcagagctaagcacaagagtgc

SEQ ID NO: 325 - DNA, 69 bp, Artificial Sequence
  Synthetic polynucleotide
tcttgtggaaagccagaaacatggtgatgagtagtcagtctccggtttcagagctaagcacaagagtgc

SEQ ID NO: 326 - DNA, 69 bp, Artificial Sequence
  Synthetic polynucleotide
tcttgtggaaagccagaaacatgggataagatgcttgaacagctgtttcagagctaagcacaagagtgc

SEQ ID NO: 327 - DNA, 69 bp, Artificial Sequence
  Synthetic polynucleotide
tcttgtggaaagccagaaacatggtctcggttccttaaatgagggtttcagagctaagcacaagagtgc

SEQ ID NO: 328 - DNA, 69 bp, Artificial Sequence
  Synthetic polynucleotide
tcttgtggaaagccagaaacatggtctacaaaattggatctaaggtttcagagctaagcacaagagtgc

SEQ ID NO: 329 - DNA, 69 bp, Artificial Sequence
  Synthetic polynucleotide
tcttgtggaaagccagaaacatggctgataggcaaactctaatggtttcagagctaagcacaagagtgc

SEQ ID NO: 330 - DNA, 69 bp, Artificial Sequence
  Synthetic polynucleotide
tcttgtggaaagccagaaacatggtataactaggaaactcagcggtttcagagctaagcacaagagtgc

SEQ ID NO: 331 - DNA, 69 bp, Artificial Sequence
  Synthetic polynucleotide
tcttgtggaaagccagaaacatggttgacagcatgcatcagtccgtttcagagctaagcacaagagtgc

SEQ ID NO: 332 - DNA, 69 bp, Artificial Sequence
  Synthetic polynucleotide
tcttgtggaaagccagaaacatggttattaaagcaagatcccccgtttcagagctaagcacaagagtgc

SEQ ID NO: 333 - DNA, 69 bp, Artificial Sequence
  Synthetic polynucleotide
tcttgtggaaagccagaaacatgggaggcatcgctggagctacggtttcagagctaagcacaagagtgc

SEQ ID NO: 334 - DNA, 69 bp, Artificial Sequence
  Synthetic polynucleotide
tcttgtggaaagccagaaacatggttgctccataggaaggcggtgtttcagagctaagcacaagagtgc

SEQ ID NO: 335 - DNA, 69 bp, Artificial Sequence
  Synthetic polynucleotide
tcttgtggaaagccagaaacatggttacaccataacgctccagggtttcagagctaagcacaagagtgc

SEQ ID NO: 336 - DNA, 69 bp, Artificial Sequence
  Synthetic polynucleotide
tcttgtggaaagccagaaacatggtgatgatcgcgtactgagtggtttcagagctaagcacaagagtgc

SEQ ID NO: 337 - DNA, 69 bp, Artificial Sequence
  Synthetic polynucleotide
tcttgtggaaagccagaaacatggtggatgtgggaaaaccacacgtttcagagctaagcacaagagtgc

SEQ ID NO: 338 - DNA, 69 bp, Artificial Sequence
  Synthetic polynucleotide
tcttgtggaaagccagaaacatgggccaccaccactactcatgggtttcagagctaagcacaagagtgc

SEQ ID NO: 339 - DNA, 69 bp, Artificial Sequence
  Synthetic polynucleotide
tcttgtggaaagccagaaacatggtgggactctgacggactgcggtttcagagctaagcacaagagtgc

SEQ ID NO: 340 - DNA, 69 bp, Artificial Sequence
  Synthetic polynucleotide
tcttgtggaaagccagaaacatggttcatgtcttaaggtcgaaggtttcagagctaagcacaagagtgc

SEQ ID NO: 341 - DNA, 69 bp, Artificial Sequence
  Synthetic polynucleotide
tcttgtggaaagccagaaacatggtttaggaatctggaaaaccagtttcagagctaagcacaagagtgc

SEQ ID NO: 342 - DNA, 69 bp, Artificial Sequence
  Synthetic polynucleotide
tcttgtggaaagccagaaacatggttccaccaaagtttccaccagtttcagagctaagcacaagagtgc

SEQ ID NO: 343 - DNA, 69 bp, Artificial Sequence
  Synthetic polynucleotide
tcttgtggaaagccagaaacatgggtgaagaaaatcttcgttgggtttcagagctaagcacaagagtgc

SEQ ID NO: 344 - DNA, 69 bp, Artificial Sequence
  Synthetic polynucleotide
tcttgtggaaagccagaaacatggtatcaggaaacacttcacgagtttcagagctaagcacaagagtgc

SEQ ID NO: 345 - DNA, 69 bp, Artificial Sequence
  Synthetic polynucleotide
tcttgtggaaagccagaaacatggtgtagatattaacctggctggtttcagagctaagcacaagagtgc

SEQ ID NO: 346 - DNA, 69 bp, Artificial Sequence
  Synthetic polynucleotide
tcttgtggaaagccagaaacatgggtttataggaggccttagctgtttcagagctaagcacaagagtgc

SEQ ID NO: 347 - DNA, 69 bp, Artificial Sequence
  Synthetic polynucleotide
tcttgtggaaagccagaaacatggtggagctggatttaaaatgggtttcagagctaagcacaagagtgc

SEQ ID NO: 348 - DNA, 69 bp, Artificial Sequence
  Synthetic polynucleotide
tcttgtggaaagccagaaacatggtgaagccgtagccatcactggtttcagagctaagcacaagagtgc

SEQ ID NO: 349 - DNA, 69 bp, Artificial Sequence
  Synthetic polynucleotide
tcttgtggaaagccagaaacatggtggtccaaatagtcctgacagtttcagagctaagcacaagagtgc

SEQ ID NO: 350 - DNA, 69 bp, Artificial Sequence
  Synthetic polynucleotide
tcttgtggaaagccagaaacatggtctgatcatagatacggagagtttcagagctaagcacaagagtgc

SEQ ID NO: 351 - DNA, 69 bp, Artificial Sequence
  Synthetic polynucleotide
tcttgtggaaagccagaaacatggtatgacagaatgcgacgagggtttcagagctaagcacaagagtgc

SEQ ID NO: 352 - DNA, 69 bp, Artificial Sequence
  Synthetic polynucleotide
tcttgtggaaagccagaaacatggtaaaatcaaagaacttcgaggtttcagagctaagcacaagagtgc

SEQ ID NO: 353 - DNA, 69 bp, Artificial Sequence
  Synthetic polynucleotide
tcttgtggaaagccagaaacatggtgccctcaccatattctgcggtttcagagctaagcacaagagtgc

SEQ ID NO: 354 - DNA, 69 bp, Artificial Sequence
  Synthetic polynucleotide
tcttgtggaaagccagaaacatggttaggaatgacaatgacagtgtttcagagctaagcacaagagtgc

SEQ ID NO: 355 - DNA, 69 bp, Artificial Sequence
  Synthetic polynucleotide
tcttgtggaaagccagaaacatggggtggtggtatggaaaacatgtttcagagctaagcacaagagtgc

SEQ ID NO: 356 - DNA, 69 bp, Artificial Sequence
  Synthetic polynucleotide
tcttgtggaaagccagaaacatggggtccggccttctcaaaaaggtttcagagctaagcacaagagtgc

SEQ ID NO: 357 - DNA, 69 bp, Artificial Sequence
  Synthetic polynucleotide
tcttgtggaaagccagaaacatggggctggtcactaactacaaggtttcagagctaagcacaagagtgc

SEQ ID NO: 358 - DNA, 69 bp, Artificial Sequence
  Synthetic polynucleotide
tcttgtggaaagccagaaacatggggggtactttgagcaccgaggtttcagagctaagcacaagagtgc

SEQ ID NO: 359 - DNA, 69 bp, Artificial Sequence
  Synthetic polynucleotide
tcttgtggaaagccagaaacatggttactatgaattccgagagggtttcagagctaagcacaagagtgc

SEQ ID NO: 360 - DNA, 69 bp, Artificial Sequence
  Synthetic polynucleotide
tcttgtggaaagccagaaacatggtctgacgtgtactctcctaagtttcagagctaagcacaagagtgc

SEQ ID NO: 361 - DNA, 69 bp, Artificial Sequence
  Synthetic polynucleotide
tcttgtggaaagccagaaacatgggctctggacagatgcagtgagtttcagagctaagcacaagagtgc

SEQ ID NO: 362 - DNA, 69 bp, Artificial Sequence
  Synthetic polynucleotide
tcttgtggaaagccagaaacatggatggtcatgactctcccttggtttcagagctaagcacaagagtgc

SEQ ID NO: 363 - DNA, 69 bp, Artificial Sequence
  Synthetic polynucleotide
tcttgtggaaagccagaaacatgggtgtgtcaaacagatctgcggtttcagagctaagcacaagagtgc

SEQ ID NO: 364 - DNA, 69 bp, Artificial Sequence
  Synthetic polynucleotide
tcttgtggaaagccagaaacatggcgtggacgttcggaacggaagtttcagagctaagcacaagagtgc

SEQ ID NO: 365 - DNA, 69 bp, Artificial Sequence
  Synthetic polynucleotide
tcttgtggaaagccagaaacatggctctagtatcacgagaacttgtttcagagctaagcacaagagtgc

SEQ ID NO: 366 - DNA, 69 bp, Artificial Sequence
  Synthetic polynucleotide
tcttgtggaaagccagaaacatggtctacctgctgatatcacgggtttcagagctaagcacaagagtgc

SEQ ID NO: 367 - DNA, 69 bp, Artificial Sequence
  Synthetic polynucleotide
tcttgtggaaagccagaaacatggtgagcgagatggtgctgtcagtttcagagctaagcacaagagtgc

SEQ ID NO: 368 - DNA, 69 bp, Artificial Sequence
  Synthetic polynucleotide
tcttgtggaaagccagaaacatggtgattatgacactcgaagtggtttcagagctaagcacaagagtgc

SEQ ID NO: 369 - DNA, 69 bp, Artificial Sequence
  Synthetic polynucleotide
tcttgtggaaagccagaaacatggtgggttcggtcataatgggggtttcagagctaagcacaagagtgc

SEQ ID NO: 370 - DNA, 69 bp, Artificial Sequence
  Synthetic polynucleotide
tcttgtggaaagccagaaacatggtatctccagatcaacatctggtttcagagctaagcacaagagtgc

SEQ ID NO: 371 - DNA, 69 bp, Artificial Sequence
  Synthetic polynucleotide
tcttgtggaaagccagaaacatggttgaagaatatcctaaactggtttcagagctaagcacaagagtgc

SEQ ID NO: 372 - DNA, 69 bp, Artificial Sequence
  Synthetic polynucleotide
tcttgtggaaagccagaaacatggggttgaaggactgacaccgcgtttcagagctaagcacaagagtgc

SEQ ID NO: 373 - DNA, 69 bp, Artificial Sequence
  Synthetic polynucleotide
tcttgtggaaagccagaaacatggatcttcagggtaactacgaagtttcagagctaagcacaagagtgc

SEQ ID NO: 374 - DNA, 69 bp, Artificial Sequence
  Synthetic polynucleotide
tcttgtggaaagccagaaacatggatcttctcgacgaaaatgcggtttcagagctaagcacaagagtgc

SEQ ID NO: 375 - DNA, 69 bp, Artificial Sequence
  Synthetic polynucleotide
tcttgtggaaagccagaaacatggatgacattgcgcgtctacgggtttcagagctaagcacaagagtgc

SEQ ID NO: 376 - DNA, 69 bp, Artificial Sequence
  Synthetic polynucleotide
tcttgtggaaagccagaaacatggatgatatctgacatgcagcggtttcagagctaagcacaagagtgc

SEQ ID NO: 377 - DNA, 69 bp, Artificial Sequence
  Synthetic polynucleotide
tcttgtggaaagccagaaacatggatgcaagacagcctcccagcgtttcagagctaagcacaagagtgc

SEQ ID NO: 378 - DNA, 87 bp, Artificial Sequence
  Synthetic polynucleotide
cagaaacatggaagacgtgacccggagatccgattggaaccgtcccaagcgttgcgaaacgagacggtcaatcgcgctgcatacttg

SEQ ID NO: 379 - DNA, 87 bp, Artificial Sequence
  Synthetic polynucleotide
cagaaacatggaagacgtgacccggagatccgattggaaccgtcccaagcgttgcgaagtgaattgctgcgcatcggcgtacgactt

SEQ ID NO: 380 - DNA, 87 bp, Artificial Sequence
  Synthetic polynucleotide
cagaaacatggaagacgtgacccggagatccgattggaaccgtcccaagcgttgcgaaagttcaacgggaccgtggccgatgtttcg

SEQ ID NO: 381 - DNA, 87 bp, Artificial Sequence
  Synthetic polynucleotide
cagaaacatggaagacgtgacccggagctatcgttcgttcgaggccagagcattcgaaacgagacggtcaatcgcgctgcatacttg

SEQ ID NO: 382 - DNA, 87 bp, Artificial Sequence
  Synthetic polynucleotide
cagaaacatggaagacgtgacccggagctatcgttcgttcgaggccagagcattcgaagtgaattgctgcgcatcggcgtacgactt

SEQ ID NO: 383 - DNA, 87 bp, Artificial Sequence
  Synthetic polynucleotide
cagaaacatggaagacgtgacccggagctatcgttcgttcgaggccagagcattcgaaagttcaacgggaccgtggccgatgtttcg

SEQ ID NO: 384 - DNA, 87 bp, Artificial Sequence
  Synthetic polynucleotide
cagaaacatggaagacgtgacccggacccatgatcgtccgatctggtcggatttgtaaacgagacggtcaatcgcgctgcatacttg

SEQ ID NO: 385 - DNA, 87 bp, Artificial Sequence
  Synthetic polynucleotide
cagaaacatggaagacgtgacccggacccatgatcgtccgatctggtcggatttgtaagtgaattgctgcgcatcggcgtacgactt

SEQ ID NO: 386 - DNA, 87 bp, Artificial Sequence
  Synthetic polynucleotide
cagaaacatggaagacgtgacccggacccatgatcgtccgatctggtcggatttgtaaagttcaacgggaccgtggccgatgtttcg

SEQ ID NO: 387 - DNA, 62 bp, Artificial Sequence
  Synthetic polynucleotide
ccggttcgactgaaggtttactaggcgaggtcaagtatgcagcgcgattgaccgtctcgttt

SEQ ID NO: 388 - DNA, 62 bp, Artificial Sequence
  Synthetic polynucleotide
gctcgtcgggcaacaatagctctacgcacctcaagtatgcagcgcgattgaccgtctcgttt

SEQ ID NO: 389 - DNA, 62 bp, Artificial Sequence
  Synthetic polynucleotide
gcatgagttgcctggcgttgcgacgactaatcaagtatgcagcgcgattgaccgtctcgttt

SEQ ID NO: 390 - DNA, 62 bp, Artificial Sequence
  Synthetic polynucleotide
ccggttcgactgaaggtttactaggcgaggtaagtcgtacgccgatgcgcagcaattcactt

SEQ ID NO: 391 - DNA, 62 bp, Artificial Sequence
  Synthetic polynucleotide
gctcgtcgggcaacaatagctctacgcacctaagtcgtacgccgatgcgcagcaattcactt

SEQ ID NO: 392 - DNA, 62 bp, Artificial Sequence
  Synthetic polynucleotide
gcatgagttgcctggcgttgcgacgactaataagtcgtacgccgatgcgcagcaattcactt

SEQ ID NO: 393 - DNA, 62 bp, Artificial Sequence
  Synthetic polynucleotide
ccggttcgactgaaggtttactaggcgaggtcgaaacatcggccacggtcccgttgaacttt

SEQ ID NO: 394 - DNA, 62 bp, Artificial Sequence
  Synthetic polynucleotide
gctcgtcgggcaacaatagctctacgcacctcgaaacatcggccacggtcccgttgaacttt

SEQ ID NO: 395 - DNA, 62 bp, Artificial Sequence
  Synthetic polynucleotide
gcatgagttgcctggcgttgcgacgactaatcgaaacatcggccacggtcccgttgaacttt

SEQ ID NO: 396 - DNA, 62 bp, Artificial Sequence
  Synthetic polynucleotide
acctcgcctagtaaaccttcagtcgaaccggagtaagcgcaacggtggaccggagacgacgg

SEQ ID NO: 397 - DNA, 62 bp, Artificial Sequence
  Synthetic polynucleotide
acctcgcctagtaaaccttcagtcgaaccggaaaattgcgtgacggacctgggccattggcc

SEQ ID NO: 398 - DNA, 62 bp, Artificial Sequence
  Synthetic polynucleotide
acctcgcctagtaaaccttcagtcgaaccggagcagattccgctacgctccgattcgatcaa

SEQ ID NO: 399 - DNA, 62 bp, Artificial Sequence
  Synthetic polynucleotide
aggtgcgtagagctattgttgcccgacgagcagtaagcgcaacggtggaccggagacgacgg

SEQ ID NO: 400 - DNA, 62 bp, Artificial Sequence
  Synthetic polynucleotide
aggtgcgtagagctattgttgcccgacgagcaaaattgcgtgacggacctgggccattggcc

SEQ ID NO: 401 - DNA, 62 bp, Artificial Sequence
  Synthetic polynucleotide
aggtgcgtagagctattgttgcccgacgagcagcagattccgctacgctccgattcgatcaa

SEQ ID NO: 402 - DNA, 62 bp, Artificial Sequence
  Synthetic polynucleotide
attagtcgtcgcaacgccaggcaactcatgcagtaagcgcaacggtggaccggagacgacgg

SEQ ID NO: 403 - DNA, 62 bp, Artificial Sequence
  Synthetic polynucleotide
attagtcgtcgcaacgccaggcaactcatgcaaaattgcgtgacggacctgggccattggcc

SEQ ID NO: 404 - DNA, 62 bp, Artificial Sequence
  Synthetic polynucleotide
attagtcgtcgcaacgccaggcaactcatgcagcagattccgctacgctccgattcgatcaa

SEQ ID NO: 405 - DNA, 62 bp, Artificial Sequence
  Synthetic polynucleotide
ttaggtccggcgattagcgctcgtgcgcgatccgtcgtctccggtccaccgttgcgcttact

SEQ ID NO: 406 - DNA, 62 bp, Artificial Sequence
  Synthetic polynucleotide
gcctcgattacgacggatgtaattcggccgtccgtcgtctccggtccaccgttgcgcttact

SEQ ID NO: 407 - DNA, 62 bp, Artificial Sequence
  Synthetic polynucleotide
gcccgtattcccgcttgcgagtagggcaattccgtcgtctccggtccaccgttgcgcttact

SEQ ID NO: 408 - DNA, 62 bp, Artificial Sequence
  Synthetic polynucleotide
ttaggtccggcgattagcgctcgtgcgcgatggccaatggcccaggtccgtcacgcaatttt

SEQ ID NO: 409 - DNA, 62 bp, Artificial Sequence
  Synthetic polynucleotide
gcctcgattacgacggatgtaattcggccgtggccaatggcccaggtccgtcacgcaatttt

SEQ ID NO: 410 - DNA, 62 bp, Artificial Sequence
  Synthetic polynucleotide
gcccgtattcccgcttgcgagtagggcaattggccaatggcccaggtccgtcacgcaatttt

SEQ ID NO: 411 - DNA, 62 bp, Artificial Sequence
  Synthetic polynucleotide
ttaggtccggcgattagcgctcgtgcgcgatttgatcgaatcggagcgtagcggaatctgct

SEQ ID NO: 412 - DNA, 62 bp, Artificial Sequence
  Synthetic polynucleotide
gcctcgattacgacggatgtaattcggccgtttgatcgaatcggagcgtagcggaatctgct

SEQ ID NO: 413 - DNA, 62 bp, Artificial Sequence
  Synthetic polynucleotide
gcccgtattcccgcttgcgagtagggcaattttgatcgaatcggagcgtagcggaatctgct

SEQ ID NO: 414 - DNA, 62 bp, Artificial Sequence
  Synthetic polynucleotide
atcgcgcacgagcgctaatcgccggacctaaaggtcgatgccctaatccacgtgcttcccgc

SEQ ID NO: 415 - DNA, 62 bp, Artificial Sequence
  Synthetic polynucleotide
atcgcgcacgagcgctaatcgccggacctaaaccataacaacggctagcacggcacgcaaat

SEQ ID NO: 416 - DNA, 62 bp, Artificial Sequence
  Synthetic polynucleotide
atcgcgcacgagcgctaatcgccggacctaaaccctcggtcccaccttacggcgatgaccct

SEQ ID NO: 417 - DNA, 62 bp, Artificial Sequence
  Synthetic polynucleotide
acggccgaattacatccgtcgtaatcgaggcaggtcgatgccctaatccacgtgcttcccgc

SEQ ID NO: 418 - DNA, 62 bp, Artificial Sequence
  Synthetic polynucleotide
acggccgaattacatccgtcgtaatcgaggcaccataacaacggctagcacggcacgcaaat

SEQ ID NO: 419 - DNA, 62 bp, Artificial Sequence
  Synthetic polynucleotide
acggccgaattacatccgtcgtaatcgaggcaccctcggtcccaccttacggcgatgaccct

SEQ ID NO: 420 - DNA, 62 bp, Artificial Sequence
  Synthetic polynucleotide
aattgccctactcgcaagcgggaatacgggcaggtcgatgccctaatccacgtgcttcccgc

SEQ ID NO: 421 - DNA, 62 bp, Artificial Sequence
  Synthetic polynucleotide
aattgccctactcgcaagcgggaatacgggcaccataacaacggctagcacggcacgcaaat

SEQ ID NO: 422 - DNA, 62 bp, Artificial Sequence
  Synthetic polynucleotide
aattgccctactcgcaagcgggaatacgggcaccctcggtcccaccttacggcgatgaccct

SEQ ID NO: 423 - DNA, 62 bp, Artificial Sequence
  Synthetic polynucleotide
caacgactggcgaccttagggttggctcgctgcgggaagcacgtggattagggcatcgacct

SEQ ID NO: 424 - DNA, 62 bp, Artificial Sequence
  Synthetic polynucleotide
cgagtaggccgcctgaacaactagccggagtgcgggaagcacgtggattagggcatcgacct

SEQ ID NO: 425 - DNA, 62 bp, Artificial Sequence
  Synthetic polynucleotide
ggacacgggatcgacggtagtggatcgatttgcgggaagcacgtggattagggcatcgacct

SEQ ID NO: 426 - DNA, 62 bp, Artificial Sequence
  Synthetic polynucleotide
caacgactggcgaccttagggttggctcgctatttgcgtgccgtgctagccgttgttatggt

SEQ ID NO: 427 - DNA, 62 bp, Artificial Sequence
  Synthetic polynucleotide
cgagtaggccgcctgaacaactagccggagtatttgcgtgccgtgctagccgttgttatggt

SEQ ID NO: 428 - DNA, 62 bp, Artificial Sequence
  Synthetic polynucleotide
ggacacgggatcgacggtagtggatcgatttatttgcgtgccgtgctagccgttgttatggt

SEQ ID NO: 429 - DNA, 62 bp, Artificial Sequence
  Synthetic polynucleotide
caacgactggcgaccttagggttggctcgctagggtcatcgccgtaaggtgggaccgagggt

SEQ ID NO: 430 - DNA, 62 bp, Artificial Sequence
  Synthetic polynucleotide
cgagtaggccgcctgaacaactagccggagtagggtcatcgccgtaaggtgggaccgagggt

SEQ ID NO: 431 - DNA, 62 bp, Artificial Sequence
  Synthetic polynucleotide
ggacacgggatcgacggtagtggatcgatttagggtcatcgccgtaaggtgggaccgagggt

SEQ ID NO: 432 - DNA, 62 bp, Artificial Sequence
  Synthetic polynucleotide
agcgagccaaccctaaggtcgccagtcgttgaaaccggtacatgacgcggaacctaaggtcg

SEQ ID NO: 433 - DNA, 62 bp, Artificial Sequence
  Synthetic polynucleotide
agcgagccaaccctaaggtcgccagtcgttgagtgaaatcgcagcagcccacgtcgtaaaca

SEQ ID NO: 434 - DNA, 62 bp, Artificial Sequence
  Synthetic polynucleotide
agcgagccaaccctaaggtcgccagtcgttgatagcaaagccggtagcgacaaccgtttccc

SEQ ID NO: 435 - DNA, 62 bp, Artificial Sequence
  Synthetic polynucleotide
actccggctagttgttcaggcggcctactcgaaaccggtacatgacgcggaacctaaggtcg

SEQ ID NO: 436 - DNA, 62 bp, Artificial Sequence
  Synthetic polynucleotide
actccggctagttgttcaggcggcctactcgagtgaaatcgcagcagcccacgtcgtaaaca

SEQ ID NO: 437 - DNA, 62 bp, Artificial Sequence
  Synthetic polynucleotide
actccggctagttgttcaggcggcctactcgatagcaaagccggtagcgacaaccgtttccc

SEQ ID NO: 438 - DNA, 62 bp, Artificial Sequence
  Synthetic polynucleotide
aaatcgatccactaccgtcgatcccgtgtccaaaccggtacatgacgcggaacctaaggtcg

SEQ ID NO: 439 - DNA, 62 bp, Artificial Sequence
  Synthetic polynucleotide
aaatcgatccactaccgtcgatcccgtgtccagtgaaatcgcagcagcccacgtcgtaaaca

SEQ ID NO: 440 - DNA, 62 bp, Artificial Sequence
  Synthetic polynucleotide
aaatcgatccactaccgtcgatcccgtgtccatagcaaagccggtagcgacaaccgtttccc

SEQ ID NO: 441 - DNA, 62 bp, Artificial Sequence
  Synthetic polynucleotide
gatggtcgactggcggtcttaatatgcccatcgaccttaggttccgcgtcatgtaccggttt

SEQ ID NO: 442 - DNA, 62 bp, Artificial Sequence
  Synthetic polynucleotide
atccatatgaccggcggccttttctcggactcgaccttaggttccgcgtcatgtaccggttt

SEQ ID NO: 443 - DNA, 62 bp, Artificial Sequence
  Synthetic polynucleotide
atgatccacgaccgagcaggttagttgacgtcgaccttaggttccgcgtcatgtaccggttt

SEQ ID NO: 444 - DNA, 62 bp, Artificial Sequence
  Synthetic polynucleotide
gatggtcgactggcggtcttaatatgcccattgtttacgacgtgggctgctgcgatttcact

SEQ ID NO: 445 - DNA, 62 bp, Artificial Sequence
  Synthetic polynucleotide
atccatatgaccggcggccttttctcggacttgtttacgacgtgggctgctgcgatttcact

SEQ ID NO: 446 - DNA, 62 bp, Artificial Sequence
  Synthetic polynucleotide
atgatccacgaccgagcaggttagttgacgttgtttacgacgtgggctgctgcgatttcact

SEQ ID NO: 447 - DNA, 62 bp, Artificial Sequence
  Synthetic polynucleotide
gatggtcgactggcggtcttaatatgcccatgggaaacggttgtcgctaccggctttgctat

SEQ ID NO: 448 - DNA, 62 bp, Artificial Sequence
  Synthetic polynucleotide
atccatatgaccggcggccttttctcggactgggaaacggttgtcgctaccggctttgctat

SEQ ID NO: 449 - DNA, 62 bp, Artificial Sequence
  Synthetic polynucleotide
atgatccacgaccgagcaggttagttgacgtgggaaacggttgtcgctaccggctttgctat

SEQ ID NO: 450 - DNA, 62 bp, Artificial Sequence
  Synthetic polynucleotide
atgggcatattaagaccgccagtcgaccatcagtcctgttcttgtcgagcgtggtgcgtcta

SEQ ID NO: 451 - DNA, 62 bp, Artificial Sequence
  Synthetic polynucleotide
atgggcatattaagaccgccagtcgaccatcaatccgtagaccaaccggccgttaaacgagt

SEQ ID NO: 452 - DNA, 62 bp, Artificial Sequence
  Synthetic polynucleotide
atgggcatattaagaccgccagtcgaccatcactgaatccgtcgttccagacgcgagtaccc

SEQ ID NO: 453 - DNA, 62 bp, Artificial Sequence
  Synthetic polynucleotide
agtccgagaaaaggccgccggtcatatggatagtcctgttcttgtcgagcgtggtgcgtcta

SEQ ID NO: 454 - DNA, 62 bp, Artificial Sequence
  Synthetic polynucleotide
agtccgagaaaaggccgccggtcatatggataatccgtagaccaaccggccgttaaacgagt

SEQ ID NO: 455 - DNA, 62 bp, Artificial Sequence
  Synthetic polynucleotide
agtccgagaaaaggccgccggtcatatggatactgaatccgtcgttccagacgcgagtaccc

SEQ ID NO: 456 - DNA, 62 bp, Artificial Sequence
  Synthetic polynucleotide
acgtcaactaacctgctcggtcgtggatcatagtcctgttcttgtcgagcgtggtgcgtcta

SEQ ID NO: 457 - DNA, 62 bp, Artificial Sequence
  Synthetic polynucleotide
acgtcaactaacctgctcggtcgtggatcataatccgtagaccaaccggccgttaaacgagt

SEQ ID NO: 458 - DNA, 62 bp, Artificial Sequence
  Synthetic polynucleotide
acgtcaactaacctgctcggtcgtggatcatactgaatccgtcgttccagacgcgagtaccc

SEQ ID NO: 459 - DNA, 62 bp, Artificial Sequence
  Synthetic polynucleotide
aacatcggatcggtgcggtgggatggataattagacgcaccacgctcgacaagaacaggact

SEQ ID NO: 460 - DNA, 62 bp, Artificial Sequence
  Synthetic polynucleotide
agacgacgcacgttcgtaccgcgtacttcgttagacgcaccacgctcgacaagaacaggact

SEQ ID NO: 461 - DNA, 62 bp, Artificial Sequence
  Synthetic polynucleotide
tttgctcgcaagtgcgcacgagttgaactgttagacgcaccacgctcgacaagaacaggact

SEQ ID NO: 462 - DNA, 62 bp, Artificial Sequence
  Synthetic polynucleotide
aacatcggatcggtgcggtgggatggataatactcgtttaacggccggttggtctacggatt

SEQ ID NO: 463 - DNA, 62 bp, Artificial Sequence
  Synthetic polynucleotide
agacgacgcacgttcgtaccgcgtacttcgtactcgtttaacggccggttggtctacggatt

SEQ ID NO: 464 - DNA, 62 bp, Artificial Sequence
  Synthetic polynucleotide
tttgctcgcaagtgcgcacgagttgaactgtactcgtttaacggccggttggtctacggatt

SEQ ID NO: 465 - DNA, 62 bp, Artificial Sequence
  Synthetic polynucleotide
aacatcggatcggtgcggtgggatggataatgggtactcgcgtctggaacgacggattcagt

SEQ ID NO: 466 - DNA, 62 bp, Artificial Sequence
  Synthetic polynucleotide
agacgacgcacgttcgtaccgcgtacttcgtgggtactcgcgtctggaacgacggattcagt

SEQ ID NO: 467 - DNA, 62 bp, Artificial Sequence
  Synthetic polynucleotide
tttgctcgcaagtgcgcacgagttgaactgtgggtactcgcgtctggaacgacggattcagt

SEQ ID NO: 468 - DNA, 87 bp, Artificial Sequence
  Synthetic polynucleotide
attatccatcccaccgcaccgatccgatgttaagcccgtctaaacgcggcgaggtacattgggccgtccgtcatctcgttttcgatg

SEQ ID NO: 469 - DNA, 87 bp, Artificial Sequence
  Synthetic polynucleotide
attatccatcccaccgcaccgatccgatgttagccgcgatattcacgccgcgtatggaaactgccgtccgtcatctcgttttcgatg

SEQ ID NO: 470 - DNA, 87 bp, Artificial Sequence
  Synthetic polynucleotide
attatccatcccaccgcaccgatccgatgttaatgaaacgtctgggcccgtaacgctgtagcgccgtccgtcatctcgttttcgatg

SEQ ID NO: 471 - DNA, 87 bp, Artificial Sequence
  Synthetic polynucleotide
acgaagtacgcggtacgaacgtgcgtcgtctaagcccgtctaaacgcggcgaggtacattgggccgtccgtcatctcgttttcgatg

SEQ ID NO: 472 - DNA, 87 bp, Artificial Sequence
  Synthetic polynucleotide
acgaagtacgcggtacgaacgtgcgtcgtctagccgcgatattcacgccgcgtatggaaactgccgtccgtcatctcgttttcgatg

SEQ ID NO: 473 - DNA, 87 bp, Artificial Sequence
  Synthetic polynucleotide
acgaagtacgcggtacgaacgtgcgtcgtctaatgaaacgtctgggcccgtaacgctgtagcgccgtccgtcatctcgttttcgatg

SEQ ID NO: 474 - DNA, 87 bp, Artificial Sequence
  Synthetic polynucleotide
acagttcaactcgtgcgcacttgcgagcaaaaagcccgtctaaacgcggcgaggtacattgggccgtccgtcatctcgttttcgatg

SEQ ID NO: 475 - DNA, 87 bp, Artificial Sequence
  Synthetic polynucleotide
acagttcaactcgtgcgcacttgcgagcaaaagccgcgatattcacgccgcgtatggaaactgccgtccgtcatctcgttttcgatg

SEQ ID NO: 476 - DNA, 87 bp, Artificial Sequence
  Synthetic polynucleotide
acagttcaactcgtgcgcacttgcgagcaaaaatgaaacgtctgggcccgtaacgctgtagcgccgtccgtcatctcgttttcgatg

SEQ ID NO: 477 - DNA, 25 bp, Artificial Sequence
  Synthetic polynucleotide
catcgaaaacgagatgacggacggc

SEQ ID NO: 478 - DNA, 49 bp, Artificial Sequence
  Synthetic polynucleotide
ggctttatatatcttgtggaaagccagaaacatggaagacgtgacccgg

SEQ ID NO: 479 - DNA, 79 bp, Artificial Sequence
  Synthetic polynucleotide
  misc_feature (29)..(48): n is a, c, g, or t
ccagaggttgattatcgtaatggatcctnnnnnnnnnnnnnnnnnnnntctagacatcgaaaacgagatgacggacggc

SEQ ID NO: 480 - DNA, 79 bp, Artificial Sequence
  Synthetic polynucleotide
ggctttatatatcttgtggaaagccagaaacaaggaccaggatgggcaccacccgtttcagagctaagcacaagagtgc

SEQ ID NO: 481 - DNA, 56 bp, Artificial Sequence
  Synthetic polynucleotide
ccgggtcacgtcttccatgtttctggctatcattacaccttgcgcttcttcttggg

SEQ ID NO: 482 - DNA, 98 bp, Artificial Sequence
  Synthetic polynucleotide
gtggcacccgagtcgggtgcttttttttcgggtaccagatccgattggaaccgtcccaagcgttgcgaaacgagacggtcaatcgcgctgcatacttg

SEQ ID NO: 483 - DNA, 98 bp, Artificial Sequence
  Synthetic polynucleotide
gtggcacccgagtcgggtgcttttttttcgggtaccagatccgattggaaccgtcccaagcgttgcgaagtgaattgctgcgcatcggcgtacgactt

SEQ ID NO: 484 - DNA, 98 bp, Artificial Sequence
  Synthetic polynucleotide
gtggcacccgagtcgggtgcttttttttcgggtaccagatccgattggaaccgtcccaagcgttgcgaaagttcaacgggaccgtggccgatgtttcg

SEQ ID NO: 485 - DNA, 98 bp, Artificial Sequence
  Synthetic polynucleotide
gtggcacccgagtcgggtgcttttttttcgggtaccagctatcgttcgttcgaggccagagcattcgaaacgagacggtcaatcgcgctgcatacttg

SEQ ID NO: 486 - DNA, 98 bp, Artificial Sequence
  Synthetic polynucleotide
gtggcacccgagtcgggtgcttttttttcgggtaccagctatcgttcgttcgaggccagagcattcgaagtgaattgctgcgcatcggcgtacgactt

SEQ ID NO: 487 - DNA, 98 bp, Artificial Sequence
  Synthetic polynucleotide
gtggcacccgagtcgggtgcttttttttcgggtaccagctatcgttcgttcgaggccagagcattcgaaagttcaacgggaccgtggccgatgtttcg

SEQ ID NO: 488 - DNA, 98 bp, Artificial Sequence
  Synthetic polynucleotide
gtggcacccgagtcgggtgcttttttttcgggtaccacccatgatcgtccgatctggtcggatttgtaaacgagacggtcaatcgcgctgcatacttg

SEQ ID NO: 489 - DNA, 98 bp, Artificial Sequence
  Synthetic polynucleotide
gtggcacccgagtcgggtgcttttttttcgggtaccacccatgatcgtccgatctggtcggatttgtaagtgaattgctgcgcatcggcgtacgactt

SEQ ID NO: 490 - DNA, 98 bp, Artificial Sequence
  Synthetic polynucleotide
gtggcacccgagtcgggtgcttttttttcgggtaccacccatgatcgtccgatctggtcggatttgtaaagttcaacgggaccgtggccgatgtttcg

SEQ ID NO: 491 - DNA, 25 bp, Artificial Sequence
  Synthetic polynucleotide
gtttcagagctaagcacaagagtgc

SEQ ID NO: 492 - DNA, 20 bp, Artificial Sequence
  Synthetic polynucleotide
gcacccgactcgggtgccac

SEQ ID NO: 493 - DNA, 34 bp, Artificial Sequence
  Synthetic polynucleotide
gctttatatatcttgtggaaagccagaaacatgg

SEQ ID NO: 494 - DNA, 25 bp, Artificial Sequence
  Synthetic polynucleotide
gcactcttgtgcttagctctgaaac

SEQ ID NO: 495 - DNA, 79 bp, Artificial Sequence
  Synthetic polynucleotide
  misc_feature (29)..(48): n is a, c, g, or t
gtcgacggtatttatcgtaatggatcctnnnnnnnnnnnnnnnnnnnntctagacatcgaaaacgagatgacggacggc

SEQ ID NO: 496 - DNA, 106 bp, Artificial Sequence
  Synthetic polynucleotide
  misc_feature (71)..(82): n is a, c, g, or t
aatgatacggcgaccaccgagatctacactatagcctacactctttccctacacgacgctcttccgatctnnnnnnnnnnnnccagaaacatgggcatgtggatcc

SEQ ID NO: 497 - DNA, 105 bp, Artificial Sequence
  Synthetic polynucleotide
  misc_feature (71)..(81): n is a, c, g, or t
aatgatacggcgaccaccgagatctacactatagcctacactctttccctacacgacgctcttccgatctnnnnnnnnnnnccagaaacatgggcatgtggatcc

SEQ ID NO: 498 - DNA, 104 bp, Artificial Sequence
  Synthetic polynucleotide
  misc_feature (71)..(80): n is a, c, g, or t
aatgatacggcgaccaccgagatctacactatagcctacactctttccctacacgacgctcttccgatctnnnnnnnnnnccagaaacatgggcatgtggatcc

SEQ ID NO: 499 - DNA, 102 bp, Artificial Sequence
  Synthetic polynucleotide
  misc_feature (67)..(78): n is a, c, g, or t
caagcagaagacggcatacgagatcgagtaatgtgactggagttcagacgtgtgctcttccgatctnnnnnnnnnnnnggatccacatgcccatgtttctgg

SEQ ID NO: 500 - DNA, 101 bp, Artificial Sequence
  Synthetic polynucleotide
  misc_feature (67)..(77): n is a, c, g, or t
caagcagaagacggcatacgagatcgagtaatgtgactggagttcagacgtgtgctcttccgatctnnnnnnnnnnnggatccacatgcccatgtttctgg

SEQ ID NO: 501 - DNA, 100 bp, Artificial Sequence
  Synthetic polynucleotide
  misc_feature (67)..(76): n is a, c, g, or t
caagcagaagacggcatacgagatcgagtaatgtgactggagttcagacgtgtgctcttccgatctnnnnnnnnnnggatccacatgcccatgtttctgg

SEQ ID NO: 502 - DNA, 106 bp, Artificial Sequence
  Synthetic polynucleotide
  misc_feature (71)..(82): n is a, c, g, or t
aatgatacggcgaccaccgagatctacacatagaggcacactctttccctacacgacgctcttccgatctnnnnnnnnnnnnccagaaacatgggcatgtggatcc

SEQ ID NO: 503 - DNA, 105 bp, Artificial Sequence
  Synthetic polynucleotide
  misc_feature (71)..(81): n is a, c, g, or t
aatgatacggcgaccaccgagatctacacatagaggcacactctttccctacacgacgctcttccgatctnnnnnnnnnnnccagaaacatgggcatgtggatcc

SEQ ID NO: 504 - DNA, 104 bp, Artificial Sequence
  Synthetic polynucleotide
  misc_feature (71)..(80): n is a, c, g, or t
aatgatacggcgaccaccgagatctacacatagaggcacactctttccctacacgacgctcttccgatctnnnnnnnnnnccagaaacatgggcatgtggatcc

SEQ ID NO: 505 - DNA, 102 bp, Artificial Sequence
  Synthetic polynucleotide
  misc_feature (67)..(78): n is a, c, g, or t
caagcagaagacggcatacgagattctccggagtgactggagttcagacgtgtgctcttccgatctnnnnnnnnnnnnggatccacatgcccatgtttctgg

SEQ ID NO: 506 - DNA, 101 bp, Artificial Sequence
  Synthetic polynucleotide
  misc_feature (67)..(77): n is a, c, g, or t
caagcagaagacggcatacgagattctccggagtgactggagttcagacgtgtgctcttccgatctnnnnnnnnnnnggatccacatgcccatgtttctgg

SEQ ID NO: 507 - DNA, 100 bp, Artificial Sequence
  Synthetic polynucleotide
  misc_feature (67)..(76): n is a, c, g, or t
caagcagaagacggcatacgagattctccggagtgactggagttcagacgtgtgctcttccgatctnnnnnnnnnnggatccacatgcccatgtttctgg

SEQ ID NO: 508 - DNA, 108 bp, Artificial Sequence
  Synthetic polynucleotide
  misc_feature (71)..(82): n is a, c, g, or t
aatgatacggcgaccaccgagatctacactatagcctacactctttccctacacgacgctcttccgatctnnnnnnnnnnnncacggtaccgtactgcaggatcctgc

SEQ ID NO: 509 - DNA, 107 bp, Artificial Sequence
  Synthetic polynucleotide
  misc_feature (71)..(81): n is a, c, g, or t
aatgatacggcgaccaccgagatctacactatagcctacactctttccctacacgacgctcttccgatctnnnnnnnnnnncacggtaccgtactgcaggatcctgc

SEQ ID NO: 510 - DNA, 106 bp, Artificial Sequence
  Synthetic polynucleotide
  misc_feature (71)..(80): n is a, c, g, or t
aatgatacggcgaccaccgagatctacactatagcctacactctttccctacacgacgctcttccgatctnnnnnnnnnncacggtaccgtactgcaggatcctgc

SEQ ID NO: 511 - DNA, 104 bp, Artificial Sequence
  Synthetic polynucleotide
  misc_feature (67)..(78): n is a, c, g, or t
caagcagaagacggcatacgagatcgagtaatgtgactggagttcagacgtgtgctcttccgatctnnnnnnnnnnnngcaggatcctgcagtacggtaccgtg

SEQ ID NO: 512 - DNA, 103 bp, Artificial Sequence
  Synthetic polynucleotide
  misc_feature (67)..(77): n is a, c, g, or t
caagcagaagacggcatacgagatcgagtaatgtgactggagttcagacgtgtgctcttccgatctnnnnnnnnnnngcaggatcctgcagtacggtaccgtg

SEQ ID NO: 513 - DNA, 102 bp, Artificial Sequence
  Synthetic polynucleotide
  misc_feature (67)..(76): n is a, c, g, or t
caagcagaagacggcatacgagatcgagtaatgtgactggagttcagacgtgtgctcttccgatctnnnnnnnnnngcaggatcctgcagtacggtaccgtg

SEQ ID NO: 514 - DNA, 116 bp, Artificial Sequence
  Synthetic polynucleotide
  misc_feature (71)..(82): n is a, c, g, or t
aatgatacggcgaccaccgagatctacactatagcctacactctttccctacacgacgctcttccgatctnnnnnnnnnnnngctttatatatcttgtggaaagccagaaacatgg

SEQ ID NO: 515 - DNA, 115 bp, Artificial Sequence
  Synthetic polynucleotide
  misc_feature (71)..(81): n is a, c, g, or t
aatgatacggcgaccaccgagatctacactatagcctacactctttccctacacgacgctcttccgatctnnnnnnnnnnngctttatatatcttgtggaaagccagaaacatgg

SEQ ID NO: 516 - DNA, 114 bp, Artificial Sequence
  Synthetic polynucleotide
  misc_feature (71)..(80): n is a, c, g, or t
aatgatacggcgaccaccgagatctacactatagcctacactctttccctacacgacgctcttccgatctnnnnnnnnnngctttatatatcttgtggaaagccagaaacatgg

SEQ ID NO: 517 - DNA, 98 bp, Artificial Sequence
  Synthetic polynucleotide
  misc_feature (67)..(78): n is a, c, g, or t
caagcagaagacggcatacgagatcgagtaatgtgactggagttcagacgtgtgctcttccgatctnnnnnnnnnnnngcacccgactcgggtgccac

SEQ ID NO: 518 - DNA, 104 bp, Artificial Sequence
  Synthetic polynucleotide
  misc_feature (71)..(82): n is a, c, g, or t
aatgatacggcgaccaccgagatctacacatagaggcacactctttccctacacgacgctcttccgatctnnnnnnnnnnnngccgtccgtcatctcgttttcg

SEQ ID NO: 519 - DNA, 103 bp, Artificial Sequence
  Synthetic polynucleotide
  misc_feature (71)..(81): n is a, c, g, or t
aatgatacggcgaccaccgagatctacacatagaggcacactctttccctacacgacgctcttccgatctnnnnnnnnnnngccgtccgtcatctcgttttcg

SEQ ID NO: 520 - DNA, 102 bp, Artificial Sequence
  Synthetic polynucleotide
  misc_feature (71)..(80): n is a, c, g, or t
aatgatacggcgaccaccgagatctacacatagaggcacactctttccctacacgacgctcttccgatctnnnnnnnnnngccgtccgtcatctcgttttcg

SEQ ID NO: 521 - DNA, 101 bp, Artificial Sequence
  Synthetic polynucleotide
  misc_feature (67)..(78): n is a, c, g, or t
caagcagaagacggcatacgagattctccggagtgactggagttcagacgtgtgctcttccgatctnnnnnnnnnnnntccctagttagccagagagctcc

SEQ ID NO: 522 - DNA, 20 bp, Artificial Sequence
  Synthetic polynucleotide
atcctccttcaatacatccc

SEQ ID NO: 523 - DNA, 20 bp, Artificial Sequence
  Synthetic polynucleotide
acactaccaccatttcctat

SEQ ID NO: 524 - DNA, 20 bp, Artificial Sequence
  Synthetic polynucleotide
actccactactactcactct

SEQ ID NO: 525 - DNA, 20 bp, Artificial Sequence
  Synthetic polynucleotide
accctctaacttccatcaca

SEQ ID NO: 526 - DNA, 20 bp, Artificial Sequence
  Synthetic polynucleotide
accacaacccattcctttca

SEQ ID NO: 527 - DNA, 20 bp, Artificial Sequence
  Synthetic polynucleotide
tttctaccactaatcaaccc

SEQ ID NO: 528 - DNA, 20 bp, Artificial Sequence
  Synthetic polynucleotide
accctttacaaacacaccct

SEQ ID NO: 529 - DNA, 20 bp, Artificial Sequence
  Synthetic polynucleotide
tcctattctcaacctaacct

SEQ ID NO: 530 - DNA, 20 bp, Artificial Sequence
  Synthetic polynucleotide
tatccttcaatccctccaca

SEQ ID NO: 531 - DNA, 20 bp, Artificial Sequence
  Synthetic polynucleotide
acattacacctcattctccc

SEQ ID NO: 532 - DNA, 20 bp, Artificial Sequence
  Synthetic polynucleotide
tttactccctacacctccaa

SEQ ID NO: 533 - DNA, 20 bp, Artificial Sequence
  Synthetic polynucleotide
ttctccctctatcaactcta

SEQ ID NO: 534 - DNA, 20 bp, Artificial Sequence
  Synthetic polynucleotide
acccttactactacatcatc

SEQ ID NO: 535 - DNA, 20 bp, Artificial Sequence
  Synthetic polynucleotide
tcctaacaaccaactactcc

SEQ ID NO: 536 - DNA, 20 bp, Artificial Sequence
  Synthetic polynucleotide
tctatcattaccctcctcct

SEQ ID NO: 537 - DNA, 20 bp, Artificial Sequence
  Synthetic polynucleotide
tattcaccttacaaaccctc

SEQ ID NO: 538 - DNA, 20 bp, Artificial Sequence
  Synthetic polynucleotide
aaacacacactaaaccaccc

SEQ ID NO: 539 - DNA, 20 bp, Artificial Sequence
  Synthetic polynucleotide
aactcatctcaatcctccca

SEQ ID NO: 540 - DNA, 20 bp, Artificial Sequence
  Synthetic polynucleotide
tatctcatcaatcccacact

SEQ ID NO: 541 - DNA, 20 bp, Artificial Sequence
  Synthetic polynucleotide
tctatcatctccaaaccaca

SEQ ID NO: 542 - DNA, 20 bp, Artificial Sequence
  Synthetic polynucleotide
tccaactcatctctaatctc

SEQ ID NO: 543 - DNA, 20 bp, Artificial Sequence
  Synthetic polynucleotide
aatactctcccacctcaact

SEQ ID NO: 544 - DNA, 20 bp, Artificial Sequence
  Synthetic polynucleotide
ataaatcattcccactaccc

SEQ ID NO: 545 - DNA, 20 bp, Artificial Sequence
  Synthetic polynucleotide
acccaacactcataacatcc

SEQ ID NO: 546 - DNA, 20 bp, Artificial Sequence
  Synthetic polynucleotide
ttcctaacaaatcacatccc

SEQ ID NO: 547 - DNA, 20 bp, Artificial Sequence
  Synthetic polynucleotide
ttcttccctcaatcttcatc

SEQ ID NO: 548 - DNA, 20 bp, Artificial Sequence
  Synthetic polynucleotide
tactacaaacccataatccc

SEQ ID NO: 549 - DNA, 20 bp, Artificial Sequence
  Synthetic polynucleotide
tcctcatcttactccctcta

SEQ ID NO: 550 - DNA, 20 bp, Artificial Sequence
  Synthetic polynucleotide
aatctcaccttccacttcac

SEQ ID NO: 551 - DNA, 20 bp, Artificial Sequence
  Synthetic polynucleotide
ttacctctaaccctccattc

SEQ ID NO: 552 - DNA, 20 bp, Artificial Sequence
  Synthetic polynucleotide
actttccacatactatccca

SEQ ID NO: 553 - DNA, 20 bp, Artificial Sequence
  Synthetic polynucleotide
acctttctccatacccaact

SEQ ID NO: 554 - DNA, 20 bp, Artificial Sequence
  Synthetic polynucleotide
tcaaactttccaaccacctc

SEQ ID NO: 555 - DNA, 20 bp, Artificial Sequence
  Synthetic polynucleotide
acaccatttatccactcctc

SEQ ID NO: 556 - DNA, 20 bp, Artificial Sequence
  Synthetic polynucleotide
tcccaactaacctaacattc

SEQ ID NO: 557 - DNA, 20 bp, Artificial Sequence
  Synthetic polynucleotide
acatcctaactacaaccttc

SEQ ID NO: 558 - DNA, 20 bp, Artificial Sequence
  Synthetic polynucleotide
ttccacaatcacttccacaa

SEQ ID NO: 559 - DNA, 20 bp, Artificial Sequence
  Synthetic polynucleotide
tcccatacaaatccaaacct

SEQ ID NO: 560 - DNA, 20 bp, Artificial Sequence
  Synthetic polynucleotide
ttctctactcattccctcaa

SEQ ID NO: 561 - DNA, 20 bp, Artificial Sequence
  Synthetic polynucleotide
aaactccacatccatctcat

SEQ ID NO: 562 - DNA, 20 bp, Artificial Sequence
  Synthetic polynucleotide
aatctcctccaacacttcta

SEQ ID NO: 563 - DNA, 20 bp, Artificial Sequence
  Synthetic polynucleotide
taaacataacaccctttccc

SEQ ID NO: 564 - DNA, 20 bp, Artificial Sequence
  Synthetic polynucleotide
ttatccatccctcttcctac

SEQ ID NO: 565 - DNA, 20 bp, Artificial Sequence
  Synthetic polynucleotide
aactacatactccctacctc

SEQ ID NO: 566 - DNA, 20 bp, Artificial Sequence
  Synthetic polynucleotide
attacactccatccactcaa

(19) United States
(12) Patent Application Publication
(10) Pub. No.: US 2022/0205983 A1
(43) Pub. Date: Jun. 30, 2022

(54) IMAGING-BASED POOLED CRISPR SCREENING

(71) Applicant: President and Fellows of Harvard College, Cambridge, MA (US)

(72) Inventors: Xiaowei Zhuang, Cambridge, MA (US); Chong Wang, Cambridge, MA (US); Tian Lu, Cambridge, MA (US)

(73) Assignee: President and Fellows of Harvard College, Cambridge, MA (US)

(21) Appl. No.: 17/604,686

(22) PCT Filed: Apr. 17, 2020
(86) PCT No.: PCT/US2020/028632

§ 371 Date: Oct. 18, 2021

Related U.S. Application Data
(60) Provisional application No. 62/841,715, filed on May 1, 2019.
(60) Provisional application No. 62/836,578, filed on Apr. 19, 2019.

(51) Int. Cl. G01N 33/52; C12N 15/113; C12N 15/11; C12N 9/22; G01N 21/64

(52) U.S. Cl. CPC G01N 33/52; C12N 15/113; C12N 15/111; G01N 2021/6439; G01N 21/6428; C12N 2310/20; C12N 2320/12; C12N 9/22

(57) ABSTRACT

The present invention generally relates to imaging cells, for example, to determine phenotypes and/or genotypes in populations of cells, e.g., to build genotype-phenotype corresponse for high-throughput screening. In some cases, the cells may be manipulated, e.g., using CRISPR or other techniques. In certain embodiments, nucleic acids may be introduced to the cell, e.g., using a lentivirus. The nucleic acids may contain a guide portion comprising a DNA or RNA recognition sequence, a reporter portion, and an identification portion comprising one or more read sequences. The guide portion may be used to alter the phenotype of the cells, e.g., using a sequence, e.g., an sgRNA sequence, that can be targeted using CRISPR or other techniques, and in some cases, the phenotype of the cells may be determined using various imaging approaches. The identification portion may be determined using MERFISH or other suitable techniques. In addition, in some cases, association or colocalization between determination of the reporter and the read sequences may substantially improve decoding accuracy, e.g., due to lowered misidentification of background signals. Other aspects are generally directed to compositions or devices for use in such methods, kits for use in such methods, or the like.

### RELATED APPLICATIONS

This application claims the benefit of U.S. Provisional Patent Application Ser. No. 62/836,578, filed Apr. 19, 2019, entitled “Imaging-Based Pooled CRISPR Screening,” by Zhuang, et al., and U.S. Provisional Patent Application Ser. No. 62/841,715, filed May 1, 2019, entitled “Imaging-Based Pooled CRISPR Screening,” by Zhuang, et al. Each of these is incorporated herein by reference in its entirety.

### GOVERNMENT FUNDING

This invention was made with government support under Grant. No. MH113094 awarded by National Institutes of Health. The government has certain rights in the invention.

### FIELD

The present invention generally relates to imaging cells, for example, to determine phenotypes and/or genotypes in populations of cells. In some cases, the cells may be manipulated, e.g., using CRISPR or other techniques.

### BACKGROUND

The development of CRISPR-based gene editing systems has greatly advanced our ability to manipulate genes and probe molecular mechanisms underlying cellular functions through genetic perturbations. Facilitated by the ability to generate high-diversity nucleic acid libraries, CRISPR-based pooled-library screening can substantially accelerate discoveries of genes involved in cellular processes. However, the phenotypes that are accessible in pooled-library screenings are limited primarily to cell viability and marker expression. Recently, single-cell RNA sequencing and mass cytometry have been combined with CRISPR screening to expand the phenotype space accessible to pooled-library screening, allowing genetic screening based on the single-cell profiles of RNA and protein expression.

However, many important cellular phenotypes are still beyond the reach of high-throughput pooled-library screening. These include morphology of cellular structures and intracellular molecular organization, as well as their dynamics, which can only be measured by techniques such as high-resolution imaging. High-content imaging further allows these properties to be measured simultaneously for many molecular species in a parallelized manner—for example, the recent development of single-cell transcriptome imaging methods has increased the number of molecular phenotypes that could be imaged in individual cells in a single experiment to the genomic scale. Despite the power of imaging in assessing cellular phenotypes, imaging-based pooled-library screening remains challenging, primarily because of the difficulty associated with determining the genotypes of individual phenotype-imaged cells in a pooled-library screening. Approaches have been developed to allow genotype determination by sequencing after physically isolating cells with certain phenotypes. However, in order to determine the full genotype-phenotype correspondence, an all imaging-based pooled-library screen approach is in demand, in which both genotypes and phenotypes are imaged for individual cells in situ.

### SUMMARY

The present invention generally relates to imaging cells, for example, to determine phenotypes and/or genotypes in populations of cells. In some cases, the cells may be manipulated, e.g., using CRISPR or other techniques. The subject matter of the present disclosure involves, in some cases, interrelated products, alternative solutions to a particular problem, and/or a plurality of different uses of one or more systems and/or articles.

In one aspect, the present invention is generally directed to a method. According to one set of embodiments, the method comprises (a) introducing, into a plurality of cells, DNA comprising a guide portion comprising a recognition sequence, a reporter portion, and an identification portion comprising read sequences, (b) determining positions of RNA molecules expressed from the reporter portion of the introduced DNA within the plurality of cells by determining the reporter portions, (c) determining a read sequence on the RNA molecules expressed from the introduced DNA comprising the reporter portion and the identification portion within the plurality of cells by exposing the cells to a readout probe able to bind to the read sequence, (d) colocalizing the binding of the readout probe with the positions of the RNA molecules expressed from the reporter portion of the introduced DNA, (e) repeating (b), (c), and (d) a plurality of times using different read sequences, and (f) creating codewords corresponding to the binding of the colocalized readout probes, wherein the values of the digits of the codewords are based on the binding of the readout probes to the read sequences.

The method, according to another set of embodiments, comprises introducing, into a plurality of cells, DNA comprising a guide portion comprising a recognition sequence, a reporter portion, and an identification portion comprising read sequences, determining positions of RNA molecules expressed from the reporter portion of the introduced DNA within the plurality of cells by determining the reporter portions, determining the read sequences within the plurality of cells by exposing the cells to a plurality of readout probes each able to bind to a read sequence, colocalizing the binding of the readout probes with the positions of the RNA molecules expressed from the reporter portion of the introduced DNA, and creating codewords corresponding to the binding of the colocalized readout probes, wherein the values of the digits of the codewords are based on the binding of the readout probes to the read sequences.

In yet another set of embodiments, the method includes introducing nucleic acids into a plurality of cells, wherein the nucleic acids comprise a guide portion comprising a recognition sequence, a reporter portion, and an identification portion comprising read sequences, imaging the plurality of cells, wherein the cells exhibit imagable differences in phenotype due to expression of the guide portion, and acquiring a plurality of images of the plurality of cells, wherein the images of the cells exhibit differences due to differences in the identification portions of the nucleic acids within the cells.

The method, in still another set of embodiments, comprises introducing DNA into a plurality of cells using a lentivirus, wherein the DNA comprises a guide portion comprising a recognition sequence, a reporter portion, and an identification portion comprising read sequences, determining phenotype of the plurality of cells, and determining genotype of the plurality of cells, and determining the correspondence between the genotype and the phenotype.

According to yet another set of embodiments, the method comprises introducing DNA into a plurality of cells using a lentivirus, wherein the DNA comprises a guide portion comprising a recognition sequence and an identification portion comprising read sequences, determining phenotype of the plurality of cells, determining genotype of the plurality of cells, and determining the correspondence between genotypes and phenotypes.

In another aspect, the present invention encompasses methods of making one or more of the embodiments described herein. In still another aspect, the present invention encompasses methods of using one or more of the embodiments described herein.

Other advantages and novel features of the present invention will become apparent from the following detailed description of various non-limiting embodiments of the invention when considered in conjunction with the accompanying figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1F illustrate imaging-based barcode detection for genotype determination, in accordance with one embodiment of the invention;

FIGS. 2A-2D illustrate barcode misidentification rates, in another embodiment of the invention;

FIGS. 3A-3E illustrate the design of a lentivirus, in still another embodiment of the invention;

FIGS. 4A-4D illustrate imaging-based pooled CRISPR screening, in yet another embodiment of the invention;

FIGS. 5A-5C illustrate genetic factors involved in regulation, in accordance with one embodiment of the invention;

FIGS. 6A-6B illustrate certain genes used for transcription inhibition, in another embodiment of the invention;

FIG. 7 illustrates the cloning strategy for a library, in one embodiment of the invention;

FIG. 8 illustrates a colocalization ratio analysis, in another embodiment of the invention;

FIG. 9 illustrates a cloning strategy for a library, in still another embodiment of the invention;

FIGS. 10A-10D illustrate knockdown of certain genes, in one embodiment of the invention; and

FIG. 11 illustrates changes of MALAT1 nuclear speckle enrichment, in another embodiment of the invention.

### DETAILED DESCRIPTION

The present invention generally relates to imaging cells, for example, to determine phenotypes and/or genotypes in populations of cells, e.g., to build genotype-phenotype corresponse for high-throughput screening. In some cases, the cells may be manipulated, e.g., using CRISPR or other techniques. In certain embodiments, nucleic acids may be introduced to the cell, e.g., using a lentivirus. The nucleic acids may contain a guide portion comprising a DNA or RNA recognition sequence, a reporter portion, and an identification portion comprising one or more read sequences. The guide portion may be used to alter the phenotype of the cells, e.g., using a sequence, e.g., an sgRNA sequence, that can be targeted using CRISPR or other techniques, and in some cases, the phenotype of the cells may be determined using various imaging approaches. The identification portion may be determined using MERFISH or other suitable techniques. In addition, in some cases, association or colocalization between determination of the reporter and the read sequences may substantially improve decoding accuracy, e.g., due to lowered misidentification of background signals. Other aspects are generally directed to compositions or devices for use in such methods, kits for use in such methods, or the like.

One example aspect of the present invention is generally directed to systems and methods for manipulating the genetic material of a cell, e.g., using CRISPR or other techniques, and determining the resulting phenotype of the cell as a result of that manipulation. The genotype of the cell may also be determined, e.g., using read sequences encoding codewords, such as is used in MERFISH or similar techniques. By determining both the genotype of the cell and how the phenotype of the cell is modified, certain embodiments as discussed herein may be useful for understanding complex questions, e.g., in regards to cellular morphology, subcellular molecular organization, and the like, for example, spatially within a cell, such as a mammalian cell.

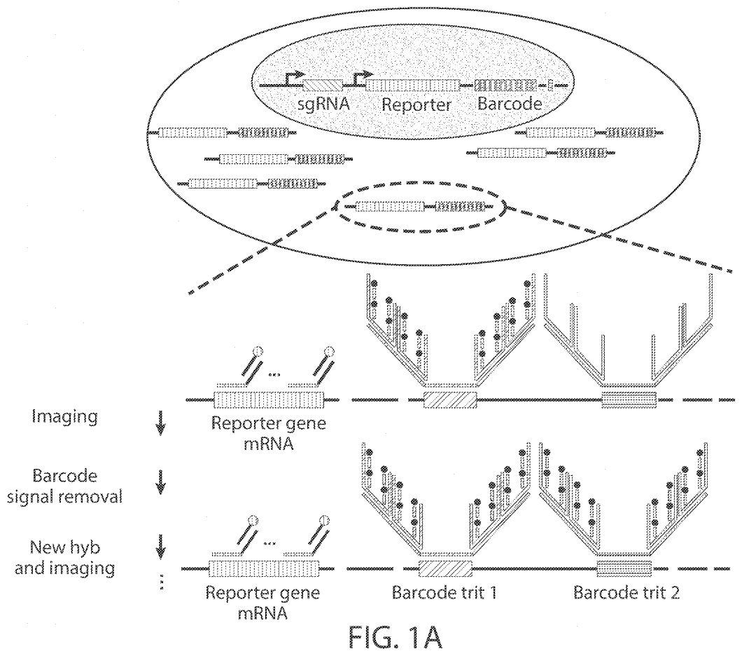

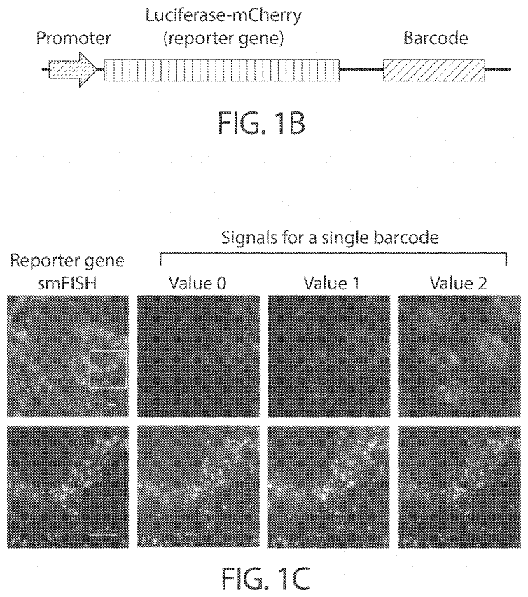

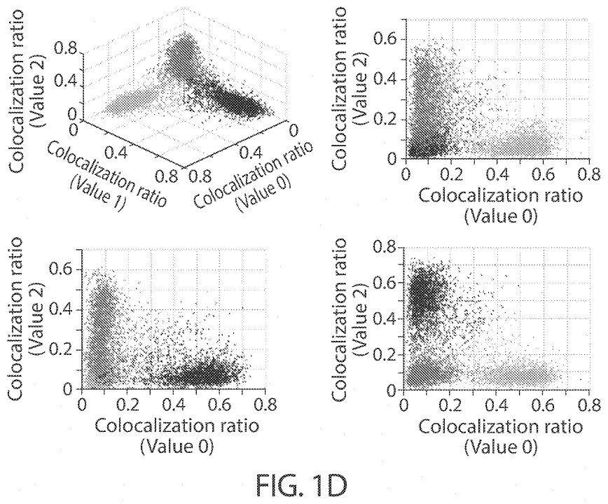

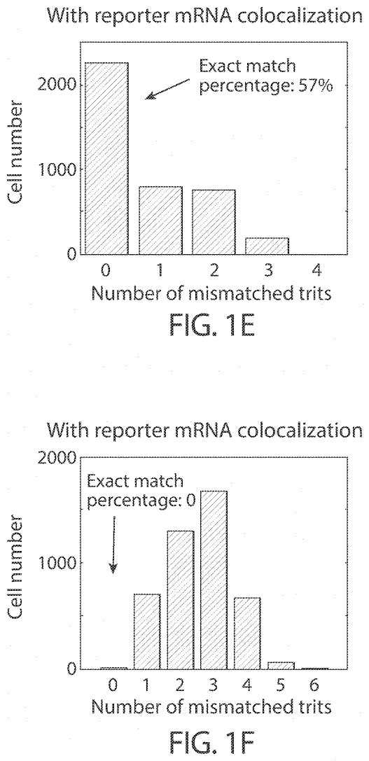

An example embodiment of the invention is now described with reference to FIG. 1A. In this figure, a member of a library of nucleic acid may be introduced into a cell, such as a mammalian cell. The nucleic acid, in one set of embodiments, comprises a guide portion (for example, containing sgRNA or another recognition sequence that can be used to recognize a target site), a reporter portion (for example, that can produce a signal such as a fluorescent or an immunoprecipitant signal, directly or indirectly), and an identification or “barcode” portion (for example, containing read sequences which can be used to distinguish various nucleic acids containing different guide portions from each other).

A variety of methods may be used to introduce the nucleic acid into the cell. These include, for example, viral delivery (e.g., using lentiviruses, retrovriuses, adenoviruses, adeno-associated viruses, etc.), electroporation, ballistic delivery, or the like. In some cases, lentiviruses may be useful because they allow for stable integration of the nucleic acid into the genome of the cell. In addition, in certain embodiments, the introduction rate of the nucleic acid into the cells may be controlled that most of the cells contain only one such nucleic acid. For example, at least 50%, at least 60%, at least 70%, at least 80%, at least 90%, or at least 95% of the cells may have only one such nucleic acid that was introduced therein.

In this example, for lentiviruses generated from pooled library, each lentivirus may contain two members of the library. During the lentivirus infection process, the guide portion and the identification portion can be recombined. Such recombination can result in misidentification of the guide portion based on the measurement of the identification portion. Accordingly, in one set of embodiments, the guide portion and the identification portion can be placed in adjacent to each other in the 3′LTR region of lentivirus, i.e. after the polypurine tract (PPT) sequence, so that the distance between the guide protion and the identification partition is minimal, e.g., 100 bases or less for the constant region the sgRNA for Cas9. In this way, the recombination rate can be reduced to improve accurate association between guide portion and identification portion. In some cases, the guide portion is duplicated within the 5′ region of the proviral DNA, e.g., of the lentivirus. This may allow the guide portion to be integrated into host cell genome to provide expression of the guide portion.

After introduction, the cells may be studied to determine the phenotype of the cells and the genotype of the cells (e.g., using the identification portion). For example, the phenotypes can be measured using imaging approaches that detect protein, RNA or DNA in the cell or in subcompartments of the cell, etc. The phenotype can also be related to cell growth, morphology or cell-cell interactions in certain embodiments. In some cases, the phenotype can be temporal changes, dynamics of cellular properties, or the like. In some cases, the phenotype can comprise multiplexed features, i.e., a multi-dimensional readout.

The identification portion may be determined, for example, using MERFISH (multiplexed error-robust fluorescence in situ hybridization) or other techniques. Those of ordinary skill in the art will be familiar with MERFISH and related techniques; see, e.g., Int. Pat. Apl. Pub. Nos. WO 2016/018960, WO 2016/018963, WO 2018/089445, WO 2018/218150, WO 2018/089438. In some cases, the identification portion can contain various “read sequences” or nucleic acid sequences that can be specifically identified using corresponding nucleic acid probes (e.g., “readout probes”), in some embodiments sequentially. In techniques such as MERFISH, the presence or absence of a read sequence can be encoded as a digit, and the sequence of readout probes can thus be encoded as a codeword. In addition, in some cases, various error detection and/or correction techniques, such as Hamming codes or Golay codes, can be applied to the codewords.

In some instances, the determination of the reporter portion may be interspersed with the determination of various portions of the identification portion (for example, using one or more readout probes). In addition, in some embodiments, the association or colocalization between the locations of the reporter portions and the determinations of the identification portions may be used to substantially improve decoding accuracy. For example, binding events or codewords that do not sufficiently correspond to locations where the reporter portion is present may be ignored as being background noise, non-specific labeling, or the like. Such association or colocalization between the reporter portions and the identification portions may substantially improve the detection accuracy.

The above discussion is a non-limiting example of one embodiment of the present invention that can be used to image cells, for example, to determine phenotypes and/or genotypes in populations of cells. However, other embodiments are also possible. Accordingly, more generally, various aspects of the invention are directed to various systems and methods for determine phenotypes and/or genotypes in populations of cells, e.g., via imaging, and/or to manipulating the cells using CRISPR or other techniques.

According to one aspect, the present invention is generally directed to systems and methods for determining the phenotypes and/or genotypes of populations of cells using imaging. In addition, the genomes of the cells may be manipulated, e.g., using CRISPR or other techniques. In some cases, relatively large numbers of cells may be studied, e.g., using suitable imaging techniques such as those described herein, to determine their phenotypes and genotypes, e.g., after manipulation. In some embodiments, due to the use of such imaging techniques, relatively large number of cells may be determined, allowing for relatively large-scale or high-throughput screening, as discussed herein. For instance, a plurality of cells may be determined for specific phenotypes (for example, after editing by CRISPR), and cells with a certain or desirable phenotype may also be determined genotypically.

In some cases, relatively large numbers of cells may be determined. For example, depending on the magnification, a single field of view may contain relatively large numbers of cells (for example, at least 10, at least 100, at least 1,000, at least 10,000, at least 100,000, etc. cells). In addition, a sample may be larger than a single field of view (e.g., especially at relatively high magnifications), and multiple images of different portions of a sample may be acquired, e.g., manually or automatically (for example, using computer control). This may allow even larger numbers of cells to be studied via the use of more than one field of view, for example, at least 10, at least 100, at least 1,000, at least 10,000, at least 100,000, at least 1,000,000, at least 10,000,000, etc. cells. For instance, an overall image of a sample may be assembled using multiple fields of views (for example, taken simultaneously or near-simultaneously) to produce an image; for example, at least 2, at least 3, at least 5, at least 7, at least 10, at least 15, at least 20, at least 30, at least 50, at least 75, or at least 100 images may be acquired at different fields of views (e.g., corresponding to different portions of a sample) to produce the overall image. Thus, the sample may, in some cases, be substantially larger than a single field of view. For example, a sample may have an area of at least about 0.01 cm2, at least about 0.03 cm2, at least about 0.1 cm2, at least about 0.3 cm2, at least about 1 cm2, at least about 3 cm2, or at least about 10 cm2, etc.

In addition in some embodiments, multiple images may be taken for the same field of view. For example, at least 2, at least 3, at least 5, at least 7, at least 10, at least 15, at least 20, at least 30, at least 50, at least 75, or at least 100 images may be acquired for the same field of view.

In some cases, multiple images may be taken at each of the fields of view imaged within a sample, in one set of embodiments. In some embodiments, different wavelengths may be used. For example, in some cases, images may be collected, for example, with different illumination sources, and captured using different optical filters so as to produce different colors of images that probe the presence of different fluorescent compounds. Thus, in some embodiments, multiple images may be taken at different wavelengths, e.g., to view the images in different colors (for example, red-green-blue, red-yellow-blue, cyan-magenta-yellow, or the like).

In some embodiments, these images may be collected at defined time intervals so as to create time-lapse images of the sample. This may be useful, for example, to determine properties that change with time, e.g., the growth of cells. For example, an image (or a plurality of images) may be acquired at different points in time, e.g., with a periodicity of about 5 seconds, about 10 seconds, about 15 seconds, about 30 seconds, about 1 minute, about 2 minutes, about 3 minutes, about 5 minutes, about 10 minutes, about 15 minutes, about 20 minutes, about 30 minutes, about 1 hour, about 2 hours, about 3 hours, about 4 hours, about 1 day, or the like. Similarly, in some embodiments, images may be collected after different treatments of the same sample.

Also, in some embodiments, multiple images may be collected with different imaging modalities, e.g. super-resolution optical microscopy, conventional epi-fluorescence microscopy, confocal microscopy, etc., including those described herein. Such images may be combined, in some cases, to create high content optical measurements of the properties of the cells.

The cells may be any suitable cells, for example, mammalian cells (e.g., human or non-human cells), bacterial cells (e.g., E. coli), eukaryotic cells, prokaryotic cells, yeast cells, or other types of cells. The cells may arise from any suitable source, for example, a cell culture. In some cases, the cells may be taken from a tissue sample, e.g., from a biopsy, artificially grown or cultured, etc. In some cases, the cells are genetically engineered. In some cases, a tissue sample may be analyzed. In certain embodiments, a plurality of cells may be transfected as discussed herein, and the resulting phenotypes of the cells determined.

In certain aspects, nucleic acids are introduced into cells, which can be used to modify the genetic material of a cell, for example, its genome. Techniques such as CRISPR or other related techniques may be used to modify the genetic material of the cell, e.g., as guided by the nucleic acids. This may allow, in some embodiments, for the accurate identification of genetic manipulations of the cells, and their corresponding phenotypes, using identification portions to identify the genotypes that lead to the observed phenotypes.

For example, in one set of embodiments, a nucleic acid that is delivered to a cell may include a guide portion, and/or a reporter portion, and/or an identification portion. The guide portion may contain sgRNA or another recognition sequence that can be used to recognize a target site, e.g., within the genome of a cell. The reporter portion may be able to produce a signal, such as a fluorescent signal, directly or indirectly. For example, the reporter portion may encode a fluorescent protein (for example, GFP), an enzyme that can be used to cause another molecule to become fluorescent (e.g., luciferase), an enzyme that produces a detectable chemical reaction, or the like. The identification portion may include sequences that can be used to distinguish various nucleic acids containing different guide portions from each other. For example, the identification portion may include one or more sequences (e.g., “read sequences”) that can be read using a corresponding nucleic acid probe (e.g., a “readout probe”).

The guide portion, and/or a reporter portion, and/or an identification portion, if present, may be arranged in any suitable order on the nucleic acid that is to be introduced to the cell. In some cases, these portions can be relatively close to each other (e.g., separated by less than 5,000, less than 3,000, less than 1,000, less than 500, less than 300, less than 100, less than 50, less than 30, or less than 10 bases away from each other, e.g., within the nucleic acid. In addition, in some cases, one or more of these portions may at least partially overlap, e.g., within the nucleic acid. Furthermore, in some embodiments, other portions or sequences may also be present within the nucleic acid. For example, one or more of these portions may contain a promoter sequence, such as those discussed herein.

In one set of embodiments, the nucleic acid includes an expression portion or a guide portion. The guide portion may include any suitable nucleic acid sequence that is suspected of being able to alter the phenotype of a cell, and/or can be used to intentionally alter or manipulate the genome of the cell, e.g., which may lead to an alteration of the phenotype of the cell that can be observed. For example, the guide portion may encode a gene, a protein, a regulatory sequence (for example, an operon, a promoter such as a CMV promoter, a repressor, a transcription factor binding site, etc.), a sequence encoding non-coding RNA (for example, miRNA, siRNA, rRNA, tRNA, lncRNA, snoRNA, snRNAs, exRNAs, piRNA, tsRNA, rsRNA, shRNA, Cas9 guide RNA, sgRNA, etc.), or the like. In some cases, the guide portion may be part of the same nucleic acid comprising an identification portion; in other cases, however, the expression portion may be part of a different nucleic acid.

Thus for example, the guide portion may include a sequence, such as an RNA sequence, that recognizes a target region of interest, e.g., on DNA (for example, on the genome of the cell). In some cases, the guide portion may also include a binding sequence, such as a Cas binding sequence, that Cas or another nuclease is able to recognize. For instance, in certain cases, the guide portion may be suitable for allowing CRISPR editing of the genome to occur. For example, the guide portion may include gRNA (guide RNA) or sgRNA (single guide RNA). In some embodiments, the sgRNA may include a crispr RNA portion (crRNA), which is a sequence complementary to a target sequence (e.g., to a target DNA), and a tracrRNA portion, which the Cas nuclease, or another nuclease, can recognize. In some cases, the crRNA portion may have 17, 18, 19, or 20 nucleotides. A variety of different Cas nucleases, such as Cas9 (from Streptococcus pyogenes), Cas14, CasX, CasY, Cas12a, Cas13a, Cas13b, Cas13d, Cas14a, etc. can be used. Variant forms of such Cas nucleases are also contemplated, e.g., High-Fidelity Cas9, eSpCas9, SpCas9-HF1, HypaCas9, FokI-Fused dCas9, xCas9, dCas9, etc. Non-limiting examples of suitable binding sequences for Cas are provided below. In addition, those of ordinary skill in the art will be aware of CRISPR and related techniques, and kits useful for conducting CRISPR experiments are readily available commercially.

In certain embodiments, more than one possibility may be present for the guide portion. For instance, a library of nucleic acids may be prepared, e.g., having different crRNA portions, e.g., for binding to different target sequences in a genome. In certain cases, there may be at least 10, at least 10², at least 10³, at least 10⁴, at least 10⁵, etc. possibilities for the guide portion, e.g., able to bind to different target sites within a genome, and/or able to cause different changes or manipulations of the genome, etc. Thus, a plurality of distinguishable nucleic acids may be prepared using one or more identification portions (such as those described herein) and one or more guide portions in certain embodiments. It should be understood, however, that the number of possible identification portions need not equal the number of possible guide portions, i.e., there may be some redundancy involved, e.g., as discussed below.

In some embodiments, the nucleic acid may include a reporter portion that can be determined, e.g., using fluorescence or other detection techniques. For example, the reporter portion may comprise a gene encoding a fluorescent protein, such as GFP (Green Fluorescent Protein), red fluorescent protein from dsRed, PAGFP, PSCFP, PSCFP2, Dendra, Dendra2, EosFP, tdEos, mEos2, mEos3, PAmCherry, PAtagRFP, mMaple, mMaple2, and mMaple3. Other suitable fluorescent proteins are known to those of ordinary skill in the art. See, e.g., U.S. Pat. No. 7,838,302 or U.S. Pat. Apl. Ser. No. 61/979,436, each incorporated herein by reference in its entirety.

In another set of embodiments, the reporter portion may encode an enzyme that can be used to cause another molecule to become fluorescent (e.g., luciferase). When expressed within a cell, a suitable substrate (e.g., luciferin) may be added, that can be converted into a fluorescent form upon exposure to the enzyme. However, in regions where the nucleic acid is not present, then no such fluorescence occurs. In this way, the nucleic acid may be localized or determined positionally in a cell (or in a portion of the cell).

It should be understood that the reporter portion need not be determinable only through fluorescence. Other reporter portions may be used in other embodiments. For example, in one embodiment an enzyme that produces a detectable chemical reaction or the like may be encoded within the reporter portion. Still other examples of reporters that may be used include, but are not limited to, proteins detectable by immunoprecipitation, immunofluorescence, or the like. Non-limiting examples of suitable proteins include the Myc tag or the HA tag.

Any suitable technique may be used to determine the reporter portion, and the exact method may depend on the type of reporter. Examples include, but are not limited to, in situ hybridization techniques such as smFISH (single-molecule fluorescent in situ hybridization), multiplexed FISH, CASFISH, or other techniques known to those of ordinary skill in the art. In one embodiment, smFISH is used to localize the reporter portion, e.g., within a cell.

Furthermore, as discussed herein, the positions of identification portions may also be determined, and associated or colocalized with the reporter portions, which may be useful, for example, for reducing background noise and/or improving decoding accuracy. For example, a reporter portion of the nucleic acid may produce a first signal (e.g., a first fluorescence), and an identification portion may produce a second signal (e.g., a second fluorescence, which may be at the same or different wavelength than the first fluorescence), which can be associated or colocalized with each other.

In some embodiments, the nucleic acid may include an identification portion or a “barcode” of nucleotides, which may be used to distinguish nucleic acids from each other. The identification portion may be present in any suitable location on the nucleic acid. For example, in one embodiment, the identification portion may be present within a 3′ UTR of the reporter gene.

In some cases, other sequences may be present in the identification portion. For example, in some cases, the identification portion may include a promoter or another regulatory sequence (for example, an operon, a promoter such as a CMV promoter, a repressor, a transcription factor binding site, etc.). The promoter may drive transcription. In some embodiments, the promoter of the identification portion may be the same or different than the promoter of the guide portion.

A library of identification portions may be used in certain embodiments, e.g., containing at least 10, at least 10², at least 10³, at least 10⁴, at least 10⁵, at least 10⁶, at least 10⁷, at least 10⁸, etc. unique sequences. The unique sequences may be all individually determined (e.g., randomly), although in some cases, the identification portion may be defined as a plurality of variable portions (or “bits”), e.g., in sequence. For example, an identification portion may include at least 2, at least 3, at least 5, at least 7, at least 10, at least 15, at least 20, at least 25, at least 30, at least 40, or at least 50 variable portions. Each of the variable portions may include at least 2, at least 3, at least 4, at least 5, at least 7, at least 10, at least 15, at least 20, at least 25, at least 30, at least 40, at least 50, or more possibilities.

Thus, for example, an identification portion defined with 22 variable regions and 2 unique possibilities per variable region would define a library of identification portions with 2²²=4,194,304 members. As another non-limiting example, an identification portion may be defined with 10 variable regions and 7 unique possibilities per variable region to define a library of identification portions with 7¹⁰ members. It should be understood that a variable portion may include any suitable number of nucleotides, and different variable portions within an identification portion may independently have the same or different numbers of nucleotides. Different variable regions also may have the same or different numbers of unique possibilities.

For example, a variable portion may be defined having a length of at least 2, at least 3, at least 4, at least 5, at least 7, at least 10, at least 15, at least 20, at least 25, at least 30, at least 40, at least 50, or more nucleotides, and/or a maximum length of no more than 50, no more than 40, no more than 30, no more than 25, no more than 20, no more than 15, no more than 10, no more than 7, no more than 5, no more than 4, no more than 3, or no more than 2 nucleotides. Combinations of these are also possible, e.g., a variable portion may have a length of between 5 and 50 nt, or between 15 and 25 nt, etc.

Each readout sequence position may be thought of as a “bit” (e.g., 1 or 0 in this example), although it should be understood that the number of possibilities for each “bit” is not necessarily limited to only 2, unlike in a computer. In other embodiments, there may be 3 possibilities (i.e., a “trit”), 4 possibilities (i.e., a “quad-bit”), 5 possibilities, etc., instead of only 2 possibilities. For instance, various trits are used in the examples below. However, the use of bits (of any number of possibilities) to form an identification portion can allow, in some but not all embodiments, the use of codewords, error-detecting codes, error-correcting codes, or the like within the identification portion, for example, as discussed in detail herein.

In some cases, the variable portions of the identification portion may be concatenated together to produce the identification portion. In other cases, however, one or more variable portions may be separated, for example, with constant portions of nucleotides, to produce the identification portion. In addition, in some cases, some or all of the possible variable portions within a library may be unique, e.g., to minimize confusion. Any method may be used for the concatenation. For example, the portions may be concatenated together using ligation, overlap PCR, oligonucleotide pool synthesis, or other techniques known to those of ordinary skill in the art for joining or concatenating nucleic acids together.

In certain embodiments, all members of a library are produced and/or are used. In other embodiments, however, not all members of a library are necessarily produced and/or used. For example, in some embodiments, e.g., to reduce or eliminate ambiguity or inadvertent reuse, a smaller subset of the library may be used, e.g., less than 75%, less than 50%, less than 40%, less than 30%, less than 20%, less than 10%, less than 5%, less than 3%, less than 2%, less than 1%, less than 0.5%, less than 0.3%, or less than 0.1% of all possible members of a library are produced and/or are used.

In some embodiments, the genotype of the cells can be determined, e.g., using the identification portion. A variety of different techniques for determining the genotype of cells may be used, for example, FISH, smFISH, MERFISH, in situ hybridization, multiplexed FISH, CASFISH, or other techniques known to those of ordinary skill in the art. These approaches can involve, in some embodiments, the direct hybridization to the identification portion, or molecules generated via the cell from that portion. It can also involve, in certain instances, binding of separate adaptor entities, which in turn bind directly to the identification portion or molecules generated from it. Additional non-limiting examples of techniques include those disclosed in U.S. patent application Ser. No. 15/329,683 or Int. Pat. Apl. Pub. No. WO 2016/018960, each incorporated herein by reference in its entirety.

In one set of embodiments, the determination of the genotype of the cells may be facilitated by determining an identification portion of a nucleic acid within the cells. For example, nucleic acids comprising an identification portion and an guide portion may have been introduced into the cells; the guide portion may have led to different phenotypes as discussed above, for example, by allowing editing of a target sequence to occur, e.g., on a genome. However, it would also be important to know which nucleic acids were introduced into which cells, thereby allowing an understanding between the observed phenotypes (e.g., altered phenotypes) and the genotypes leading to those phenotypes. By determining the identification portion within the cells, as discussed herein, the identity of the nucleic acid contained within each cell may be determined, and thus a specific guide portion may also be determined, e.g., if the nucleic acid comprises the identification portion and the guide portion on the same individual nucleic acid.

As a non-limiting example, in one set of embodiments, the cells may be sequentially exposed to nucleic acid probes able to bind to different portions of the identification portion, or molecules, such as RNA, expressed by the cell from this identification portion, for example, nucleic acid probes comprising a target sequence (e.g., that is able to bind to at least a portion of the identification portion, in some cases specifically) and a read sequence (e.g., which may be “read” in some fashion to determine binding), and binding of the nucleic acid probes within the cells may be determined. For example, the cells may be exposed to secondary nucleic acid probe may contain a recognition sequence able to bind to or hybridize with a read sequence, and which may contain a signaling entity. By determining signaling entities within images (and in some cases, inactivating the signaling entities between images and exposure to different nucleic acid probes), the identification portions of the cells may be determined.

As discussed herein, a variety of nucleic acid probes may be used to determine one or more nucleic acids within a cell. The probes may comprise nucleic acids (or entities that can hybridize to a nucleic acid, e.g., specifically) such as DNA, RNA, LNA (locked nucleic acids), PNA (peptide nucleic acids), or combinations thereof. In some cases, additional components may also be present within the nucleic acid probes, e.g., as discussed below. In some embodiments, the nucleic acid probes can be created from other components, e.g. protein or other small molecules, or may represent a combination of these components with nucleic acids such as DNA, RNA, LNA, PNA, or the like.

The nucleic acid probes may be introduced into the cells using any suitable method. In some cases, the cells may be sufficiently permeabilized such that the nucleic acid probes may be introduced into the cells by flowing a fluid containing the nucleic acid probes around the cells. In some cases, the cells may be sufficiently permeabilized as part of a fixation process; in other embodiments, cells may be permeabilized by exposure to certain chemicals such as ethanol, methanol, Triton, or the like. In addition, in some embodiments, techniques such as electroporation or microinjection may be used to introduce nucleic acid probes into the cells.

The determination of nucleic acids within the cells may be qualitative and/or quantitative. In addition, the determination may also be spatial, e.g., the position of the nucleic acid within the cells may be determined in two or three dimensions. In some embodiments, the positions, number, and/or concentrations of nucleic acids within the cells may be determined.

As mentioned, in certain embodiments, association or colocalization between the reporter gene locations and the detection of read sequences when reading codewords may substantially improve decoding accuracy, e.g., due to lowered misidentification of background signals introduced by non-specific labeling. In some cases, for example, a codeword readout portion may contain only one sequence for readout, so that the readout signal may be more difficult to identify, e.g., relative to the background. For instance, in certain cases, the reporter portion may be determined as discussed herein, e.g., locally or spatially, and portions of the identification sequence may be determined as discussed herein. In some cases, apparent portions of the identification sequence that are not colocalized with a reporter portion may be deleted from further consideration. For example, the apparent identification sequence may be an incorrect signal, background noise, or the like. In addition, in some cases, the reporter portion may be determined between different determinations of the identification sequence. Such an approach may improve accuracy, e.g., reducing errors due to movement of the sample, stage drift, or the like. Accordingly, association or colocalization between the reporter gene location and the detection of the read sequence may be used to determine whether a purported signal of the read sequence is a read sequence or is background noise, etc. (and hence not worth further consideration).

It should be understood that although the number of guide portions and/or identification portions may have a relatively large number of possibilities (for example, millions), this is readily achievable by one of ordinary skill in the art using technologies such as computers and automated nucleic acid synthesis machines (many of which are commercially available), as well as techniques such as solid-phase synthesis and/or isothermal assembly, and/or error-prone PCR and/or ligating or otherwise assembling by for example overlap PCR multiple variable regions combinatorially. Similarly, a correspondingly relatively large number of unique identification portions may be correlated with such large numbers of possibilities for the guide portions, for example, through the use of relatively small numbers of suitable variable regions and unique “bits” that can be produced for each. Accordingly, a library of nucleic acids (e.g., each containing an identification portion and a guide portion) may be prepared, e.g., containing at least 10, at least 10², at least 10³, at least 10⁴, at least 10⁵, at least 10⁶, at least 10⁷, at least 10⁸, etc. unique members.

In certain aspects, nucleic acids from the library of nucleic acids may be introduced into a cell. Any suitable technique may be used to introduce the nucleic acid. For example, in one set of embodiments, a nucleic acid may be delivered to a cell using a virus, such as a lentivirus, a retrovirus, an adenovirus, or an adeno-associated virus. In some cases, the virus may be able transfect or deliver the nucleic acid into genome of the cell, and in some cases, stably within the genome.

For example, in one set embodiments, a lentiviral delivery system may be used to introduce a nucleic acid into a cell. A lentiviral system may allow the number of nucleic acids introduced into a cell to be controlled. For example, by controlling the titer of the lentivirus used for transduction, the number of members of the library delivered to individual cells can be controlled to be one, or more than one. In some embodiments, the guide portion and the identification portion can be placed in adjacent to each other within the 3′LTR region of the lentivirus, i.e. after the polypurine tract (PPT) sequence of the lentivirus so that the distance between the guide protion and the identification partition is minimal, e.g., 100 bases or less for the constant region the sgRNA fopr Cas9. The distance may also be less than 500 bases, less than 300 bases, less than 200 bases, less than 100 bases, less than 50 bases, less than 30 bases, or less than 10 bases in certain embodiments. Such a lentiviral construct may reduce the genomic distance between guide portion and identification portion. This may result in reduced recombination effects, which may allow for more accurate identification of the guide portion by the measurement of the identification portion. Those of ordinary skill in the art will be familiar with lentiviruses and other virus-based delivery systems for introducing nucleic acids into cells. Many kits allowing for such delivery of nucleic acids into cells using viruses can be readily obtained commercially.

In addition, in some embodiments, other techniques may be used to introduce a nucleic acid into a cell. For example, the nucleic acids may be incorporated into plasmids that may be taken up by the cells. Other methods of introducing nucleic acids into cells include, but are not limited to, calcium phosphate (e.g., tricalcium phosphate), electroporation, cell squeezing, mixing a cationic lipid with the material to produce liposomes which fuse with the cell membrane, or the like. Additional non-limiting examples of suitable methods include dendrimers, cationic polymers, lipofection, FuGENE, sonoporation, optical transfection, protoplast fusion, impalefection, the gene gun, magnetofection, particle bombardment, viral infection, or the like.

In certain embodiments, the nucleic acids may be introduced or transfected into the cells such that at least 50% of the cells have only 0 or 1 nucleic acids introduced therein. In some cases, at least 60%, at least 70%, at least 80%, at least 85%, at least 90%, at least 95%, at least 97%, at least 99%, etc. of the cells may have only 0 and/or 1 nucleic acids introduced therein. This may be achieved, for example, using lentiviruses such as discussed above, suitable dilution techniques, cell sorting techniques, or through the use of other techniques such as microfluidic droplets. In other cases, the percent of transfected cells may be smaller, such as less than 50%, less than 20%, less than 10%, less than 1%. In some embodiments, cells where no such nucleic acids were introduced may be removed. Non-limiting examples of cell removal include treatment with a chemical (such as an antibiotic) that, for example, kills or prevents from dividing the non-transfected cells. In another example, some or all of the cells not containing introduced nucleic acids may be removed from the sample, for example, using fluorescence activated cell sorting and/or other suitable cell sorting or microfluidic techniques.

In certain embodiments, the identification portion and the guide portion may be combined within a single source, e.g. a nucleic acid contained within a single virus. In other embodiments, these portions may be provided to a cell in separate sources, e.g. two different viral delivery vehicles. Other examples of introducing a nucleic acid into a cell are disclosed herein, and the methods of introduction may be the same or different.

The combination of the identification portion and the guide portion, whether it is on the same or different vehicles, e.g., viruses, can be determined, for example, randomly or deterministically. For example, a given CRISPR edit can be assigned to a given barcode, and expressed within a cell. In some embodiments, the specific association between the identification and guide portions can be measured with any of a variety of techniques. For example, PCR may be used to amplify a portion of a nucleic acid containing both the identification and the guide portions, and then sequencing approaches, included next-generation sequencing methods, can be used to identify which identification region occurs with which guide portion via direct sequencing of this PCR product. Those of ordinary skill in the art will be aware of other techniques that can be used to sequence the nucleic acids, e.g., containing the identification portion and the guide portion. Any technique may be used for sequencing, for example, Sanger sequencing, high-throughput sequencing, next generation sequencing, nanopore sequencing, sequencing by ligation, sequencing by synthesis, etc. Those of ordinary skill in the art will be aware of different techniques for sequencing nucleic acids.

The cells may be analyzed to determine their phenotype in certain aspects. In some cases, the phenotypes may be altered in some embodiments, for example, through the use of CRISPR or other techniques, e.g., which can interact with the genome of the cell as discussed herein. The phenotype may be determined using any suitable technique, for example, using optical techniques, through analysis of cell behavior, or the like. Specific examples include, but are not limited to, microscopy or other optical techniques such as light microscopy, fluorescence microscopy, confocal microscopy, near-field microscopy, two-photon microscopy, or phase contrast microscopy, or other techniques described herein. In some cases, super-resolution techniques may be used, including any of those described herein. In some embodiments, the phenotype can be probed by other techniques, such as atomic force microscopy or patch clamping. In addition, in some embodiments, the phenotype may be determined using a protein. For example, a protein may be determining using fluorescence, immunofluorescence, etc. Specific non-limiting examples include fluorescence labelling approaches such as fluorescent proteins or organic dyes. In some cases, both microscopy and another technique can be used in combination for determining the phenotype.

Examples of phenotype that may be determined include, but are not limited to, the morphology of a cell (e.g., shape, size, visual appearance, organelles, subcompartments, state (for example, during the cell cycle), etc.), certain characteristics of cell motility (for example, speed, persistence, chemotaxis behavior, etc.), certain characteristics of inter-cellular interactions (e.g. cell to cell adhesion, cell to cell avoidance, cell to cell interaction, etc.), or certain subcellular characteristics (for example position of a protein or nucleic acid, diffusion of protein or nucleic acids, binding of two or more proteins and/or nucleic acids, etc.). The morphology may include whole cell morphology or subcompartment morphology. In one embodiment, smFISH is used to determine the phenotypes of the cells.

In some cases, the phenotype may be determined dynamically, e.g., as temporal changes in the cells.

In certain embodiments, the cells are present on a substrate, for example, suitable for culturing and/or imaging cells. For example, the substrate may be glass, silicon, plastic (for example, polystyrene, polypropylene, polycarbonate, etc.), or the like. In some cases, at least a portion of the substrate may be at least partially optically transparent. The substrate may also be untreated or treated in some fashion to facilitate cell attachment.

In some embodiments, phenotypes that may be determined include all, or at least a portion, of the transcriptome of the cells. A variety of techniques may be used to determine transcriptomes including, but not limited to, smFISH, MERFISH, or other techniques such as those described herein. See also U.S. patent application Ser. No. 15/329,683 or Int. Pat. Apl. Pub. No. WO 2016/018960, each incorporated herein by reference in its entirety. In some cases, the transcriptome may be determined spatially within one or more cells.

In addition, in some cases, phenotypes that may be determined include all, or at least a portion, of the chromosome of the cells, and/or agents such as proteins or RNA that may be bound to or otherwise associated with the chromosome of the cells. For example, concentrations, spatial positions, activities, associations, etc. of the chromosomes and/or other associated agents may be determined, according to certain embodiments of the invention. In some cases, the chromosomes may be determined spatially within one or more cells. Non-limiting examples of techniques that may be used to determine chromosomes include multiplexed DNA FISH or CASFISH. As yet another example, an epigenetic modification of a cell may be determined.

In addition, in some cases, phenotypes that may be determined include all, or at least a portion, of the proteome of the cells. A variety of techniques may be used to determine proteomes include antibody labeling, sequential antibody labeling, multiplexed antibody imaging, or other multiplexed protein imaging techniques. For example, concentrations, spatial positions, activities, associations, etc. of the proteins and/or other associated agents may be determined.

In certain embodiments, one or more markers may be determined within the cell to determine a phenotype. For example, the marker may be indicative for a certain cell protein, nucleic acid, morphological characteristic, or the like, or the marker may be indicative of cell behavior. In addition, the marker may be one that can be visually determined in some cases. For example, the marker may be fluorescent, or may alter fluorescence of another fluorescent entity within the cell (for example, via enhancement or quenching). The marker may also be a dye or may change color in some embodiments. Accordingly, differences in intensity, wavelength, frequency, position, distribution, or the like between cells in an image may be determined to determine phenotypes of the cells. Other methods of determining a marker may also be used in some cases; for example, the marker may be radioactive. Many such markers may be obtained commercially.

Moreover, it should be understood that these measurements are not mutually exclusive. Any combination of these measurements can be performed in a single sample. Moreover, such measurements may be repeated in some embodiments, e.g., for the same sample. For instance, the measurements may be repeated to ensure validity or reduce potential errors (e.g., measurement errors), or the measurements may be repeated after exposure to various stimuli or conditions, such as treatment with different nutritional sources, small molecules, or other suitable agents that may interact with the cells.

In some cases, the phenotype of a cell may be altered by application of a guide portion, e.g., as discussed above, that may be expressed in some form by the cell to alter its phenotype. For example, a guide portion may be used to induce an alteration of the genome of the cell, e.g., through CRISPR or other suitable techniques, including those described herein. As another example, a guide portion that encodes a protein to the cell may be added, and the cell may express the protein. If different proteins are encoded in different cells, then the cells may exhibit different phenotypes, which can be determined as noted above. Thus, for instance, a plurality of cells may be transfected or otherwise introduced to a plurality of different guide portions, and then the cells studied to determine the effects the different guide portions have had on their phenotype.

Certain aspects are thus generally directed to nucleic acid probes that are introduced into a cell (or other sample). The probes may comprise any of a variety of entities that can hybridize to a nucleic acid, e.g., a target site, typically by Watson-Crick base pairing, such as DNA, RNA, LNA, PNA, etc., depending on the application. The nucleic acid probe typically contains a target sequence that is able to bind to at least a portion of a target, e.g., a target site. In some cases, the binding may be specific binding (e.g., via complementary binding). When introduced into a cell or other system, the target sequence may be able to bind to a specific target (e.g., an mRNA, or other nucleic acids as discussed herein). The nucleic acid probe may also contain one or more read sequences, as discussed below.

In some cases, more than one type of nucleic acid probe may be applied to a sample, e.g., sequentially or simultaneously. For example, there may be at least 2, at least 5, at least 10, at least 25, at least 50, at least 75, at least 100, at least 300, at least 1,000, at least 3,000, at least 10,000, or at least 30,000 distinguishable nucleic acid probes that are applied to a sample. In some cases, the nucleic acid probes may be added sequentially. However, in some cases, more than one nucleic acid probe may be added simultaneously.

The nucleic acid probe may include one or more target sequences, which may be positioned anywhere within the nucleic acid probe. The target sequence may contain a region that is substantially complementary to a portion of a target, e.g., a target nucleic acid. For instance, in some cases, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary, e.g., to produce specific binding. Typically, complementarity is determined on the basis of Watson-Crick nucleotide base pairing.

In some cases, the target sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the target sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the target sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

The target sequence of a nucleic acid probe may be determined with reference to a target suspected of being present within a cell or other sample. For example, a target nucleic acid to a protein may be determined using the protein's sequence, e.g., by determining the nucleic acids that are expressed to form the protein. In some cases, only a portion of the nucleic acids encoding the protein are used, e.g., having the lengths as discussed above. In addition, in some cases, more than one target sequence that can be used to identify a particular target may be used. For instance, multiple probes can be used, sequentially and/or simultaneously, that can bind to or hybridize to the same or different regions of the same target. Hybridization typically refers to an annealing process by which complementary single-stranded nucleic acids associate through Watson-Crick nucleotide base pairing (e.g., hydrogen bonding, guanine-cytosine and adenine-thymine) to form double-stranded nucleic acid.

In some embodiments, a nucleic acid probe may also comprise one or more “read” sequences, as previously discussed. The read sequences may be used, to identify the nucleic acid probe, e.g., through association with signaling entities, as discussed below. In some embodiments, the nucleic acid probe may comprise 1, 2, 3, 4, 5, 6, 7, 8, 9, 10, 11, 12, 13, 14, 15, 16 or more, 20 or more, 24 or more, 32 or more, 40 or more, 48 or more, 50 or more, 64 or more, 75 or more, 100 or more, 128 or more read sequences. The read sequences may be positioned anywhere within the nucleic acid probe. If more than one read sequence is present, the read sequences may be positioned next to each other, and/or interspersed with other sequences.

The read sequences may be of any length. If more than one read sequence is used, the read sequences may independently have the same or different lengths. For instance, the read sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the read sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the read sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

The read sequence may be arbitrary or random in some embodiments. In certain cases, the read sequences are chosen so as to reduce or minimize homology with other components of the cell or other sample, e.g., such that the read sequences do not themselves bind to or hybridize with other nucleic acids suspected of being within the cell or other sample. In some cases, the homology may be less than 10%, less than 8%, less than 7%, less than 6%, less than 5%, less than 4%, less than 3%, less than 2%, or less than 1%. In some cases, there may be a homology of less than 20 basepairs, less than 18 basepairs, less than 15 basepairs, less than 14 basepairs, less than 13 basepairs, less than 12 basepairs, less than 11 basepairs, or less than 10 basepairs. In some cases, such basepairs are sequential.

In one set of embodiments, a population of nucleic acid probes may contain a certain number of read sequences, which may be less than the number of targets of the nucleic acid probes in some cases. Those of ordinary skill in the art will be aware that if there is one signaling entity and n read sequences, then in general 2ⁿ−1 different nucleic acid targets may be uniquely identified. However, not all possible combinations need be used. For instance, a population of nucleic acid probes may target 12 different nucleic acid sequences, yet contain no more than 8 read sequences. As another example, a population of nucleic acids may target 140 different nucleic acid species, yet contain no more than 16 read sequences. Different nucleic acid sequence targets may be separately identified by using different combinations of read sequences within each probe. For instance, each probe may contain 1, 2, 3, 4, 5, 6, 7, 8, 9, 10, 11, 12, 13, 14, 15, 16, etc. or more read sequences. In some cases, a population of nucleic acid probes may each contain the same number of read sequences, although in other cases, there may be different numbers of read sequences present on the various probes.

As a non-limiting example, a first nucleic acid probe may contain a first target sequence, a first read sequence, and a second read sequence, while a second, different nucleic acid probe may contain a second target sequence, the same first read sequence, but a third read sequence instead of the second read sequence. Such probes may thereby be distinguished by determining the various read sequences present or associated with a given probe or location, as discussed herein. For example, the probes can be sequentially identified and encoded using “codewords,” as discussed below. Optionally, the codewords may also be subjected to error detection and/or correction.

In addition, the population of nucleic acid probes (and their corresponding, complimentary sites on the encoding probes), in certain embodiments, may be made using only 2 or only 3 of the 4 naturally occurring nucleotide bases, such as leaving out all the “G”s or leaving out all of the “C”s within the population of probes. Sequences lacking either “G”s or “C”s may form very little secondary structure in certain embodiments, and can contribute to more uniform, faster hybridization. Thus, in some cases, the nucleic acid probes may contain only A, T, and G; only A, T, and C; only A, C, and G; or only T, C, and G.

In one aspect, the read sequences on the nucleic acid probes may be able to bind (e.g., specifically) to corresponding recognition sequences on the primary amplifier nucleic acids. Thus, when a nucleic acid probe recognizes a target within a biological sample, e.g., a DNA or RNA target, the primary amplifier nucleic acid are also able to associate with the target via the nucleic acid probe, with interactions between the read sequences of the nucleic acid probes and corresponding recognition sequences on the primary amplifier nucleic acids, e.g., complementary binding. For instance, the recognition sequence may be able to recognize a target read sequence, but not substantially recognize or bind to other, non-target read sequence. The primary amplifier nucleic acids may also comprise any of a variety of entities able to hybridize a nucleic acid, e.g., DNA, RNA, LNA, and/or PNA, etc., depending on the application. For instance, such entities may form some or all of the recognition sequence. Thus, the recognition sequence may recognize a nucleic acid sequence, such as DNA or RNA.

In some cases, the recognition sequence may be substantially complementary to the target read sequence. In some cases, the sequences may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary. Typically, complementarity is determined on the basis of Watson-Crick nucleotide base pairing. The structures of the target read sequence may include those previously described.

In some cases, the recognition sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the recognition sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the recognition sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

In some embodiments, a primary amplifier nucleic acid may also comprise one or more read sequences able to bind to secondary amplifier nucleic acids, as discussed below. For example, a primary amplifier nucleic acid may comprise 1, 2, 3, 4, 5, 6, 7, 8, 9, 10, 11, 12, 13, 14, 15, 16 or more, 20 or more, 32 or more, 40 or more, 50 or more, 64 or more, 75 or more, 100 or more, 128 or more read sequences. The read sequences may be positioned anywhere within the primary amplifier nucleic acid. If more than one read sequence is present, the read sequence may be positioned next to each other, and/or interspersed with other sequences. In one embodiment, the primary amplifier nucleic acid comprises a recognition sequence at a first end and a plurality of read sequences at a second end.

In some cases, a read sequence within the primary amplifier nucleic acid may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the read sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the read sequence may have a length of between 10 and 20 nucleotides, between 10 and 30 nucleotides, between 20 and 40 nucleotides, between 5 and 50 nucleotides, between 10 and 200 nucleotides, or between 25 and 35 nucleotides, between 10 and 300 nucleotides, etc.

There may be any number of read sequences within a primary amplifier nucleic acid. For example, there may be 1, 2, 3, 4, 5, 6, 7, 8, 9, 10, 11, 12, 13, 14, 15, 16, 17, 18, 19, 20, or more read sequences present within a primary amplifier nucleic acid. If more than one read sequence is present within a primary amplifier nucleic acid, the read sequences may be the same or different. In some cases, for example, the read sequences may all be identical.

In some embodiments, the population of primary amplifier nucleic acids may be made using only 2 or only 3 of the 4 naturally occurring nucleotide bases, such as leaving out all the “G”s or leaving out all of the “C”s within the population of nucleic acids. Sequences lacking either “G”s or “C”s may form very little secondary structure in certain embodiments, and can contribute to more uniform, faster hybridization. Thus, in some cases, the primary amplifier nucleic acids may contain only A, T, and G; only A, T, and C; only A, C, and G; or only T, C, and G.

In some cases, more than one type of primary amplifier nucleic acid may be applied to a sample, e.g., sequentially or simultaneously. For example, there may be at least 2, at least 5, at least 10, at least 25, at least 50, at least 75, at least 100, at least 300, at least 1,000, at least 3,000, at least 10,000, or at least 30,000 distinguishable primary amplifier nucleic acids that are applied to a sample. In some cases, the primary amplifier nucleic acids may be added sequentially. However, in some cases, more than one primary amplifier nucleic acid may be added simultaneously.

In one set of embodiments, the read sequences on the primary amplifier nucleic acids may be able to bind (e.g., specifically) to corresponding recognition sequences on the secondary amplifier nucleic acids. Thus, when a nucleic acid probe recognizes a target within a biological sample, e.g., a DNA or RNA target, the secondary amplifier nucleic acids are also able to associate with the target, via the primary amplifier nucleic acids, with interactions between the read sequences of the primary amplifier nucleic acids and corresponding recognition sequences on the secondary amplifier nucleic acids, e.g., complementary binding. For instance, the recognition sequence on a secondary amplifier nucleic acid may be able to recognize a read sequence on a primary amplifier nucleic acid, but not substantially recognize or bind to other, non-target read sequence. The secondary amplifier nucleic acids may also comprise any of a variety of entities able to hybridize a nucleic acid, e.g., DNA, RNA, LNA, and/or PNA, etc., depending on the application. For instance, such entities may form some or all of the recognition sequence.

In some cases, the recognition sequence on the secondary amplifier nucleic acid may be substantially complementary to a read sequence on a primary amplifier nucleic acid. In some cases, the sequences may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary.

In some cases, the recognition sequence on the secondary amplifier nucleic acid may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the recognition sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the recognition sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

In some embodiments, a secondary amplifier nucleic acid may also comprise one or more read sequences able to bind to a signaling entity, as discussed herein. For example, a secondary amplifier nucleic acid may comprise 1, 2, 3, 4, 5, 6, 7, 8, 9, 10, 11, 12, 13, 14, 15, 16 or more, 20 or more, 32 or more, 40 or more, 50 or more, 64 or more, 75 or more, 100 or more, 128 or more read sequences able to bind to a signaling entity. The read sequences may be positioned anywhere within the secondary amplifier nucleic acid. If more than one read sequences is present, the read sequences may be positioned next to each other, and/or interspersed with other sequences. In one embodiment, the secondary amplifier nucleic acid comprises a recognition sequence at a first end and a plurality of read sequences at a second end. This structure may also be the same or different than the structure of the primary amplifier nucleic acid.

In some cases, the read sequence within the secondary amplifier nucleic acid may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the read sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the read sequence within the secondary amplifier nucleic acid may have a length of between 10 and 20 nucleotides, between 10 and 30 nucleotides, between 20 and 40 nucleotides, between 5 and 50 nucleotides, between 10 and 200 nucleotides, or between 25 and 35 nucleotides, between 10 and 300 nucleotides, etc.

There may be any number of read sequences within a secondary amplifier nucleic acid. For example, there may be 1, 2, 3, 4, 5, 6, 7, 8, 9, 10, 11, 12, 13, 14, 15, 16, 17, 18, 19, 20, or more read sequences present within a secondary amplifier nucleic acid. If more than one read sequence is present within a secondary amplifier nucleic acid, the read sequences may be the same or different. In some cases, for example, the read sequences may all be identical. In addition, there may independently be the same or different numbers of read sequences in the primary and in the secondary amplifier nucleic acids.

The population of secondary amplifier nucleic acids may be made using only 2 or only 3 of the 4 naturally occurring nucleotide bases, in certain embodiments such as leaving out all the “G”s or leaving out all of the “C”s within the population of nucleic acids. Sequences lacking either “G”s or “C”s may form very little secondary structure in certain embodiments, and can contribute to more uniform, faster hybridization. Thus, in some cases, the secondary amplifier nucleic acids may contain only A, T, and G; only A, T, and C; only A, C, and G; or only T, C, and G.

In some cases, more than one type of secondary amplifier nucleic acid may be applied to a sample, e.g., sequentially or simultaneously. For example, there may be at least 2, at least 5, at least 10, at least 25, at least 50, at least 75, at least 100, at least 300, at least 1,000, at least 3,000, at least 10,000, or at least 30,000 distinguishable secondary amplifier nucleic acids that are applied to a sample. In some cases, the secondary amplifier nucleic acids may be added sequentially. However, in some cases, more than one secondary amplifier nucleic acid may be added simultaneously.

In addition, in certain embodiments, this pattern can instead be repeated prior to the signaling entity, e.g., with tertiary amplifier nucleic acids, quaternary nucleic acids, etc., similar to the above discussion. The signaling entities may thus be bound to the ending amplifier nucleic acid. Thus, as non-limiting examples, to a target may be bound an encoding nucleic acid probe, to which a primary amplifier nucleic acid is bound, to which a secondary amplifier nucleic acid is bound, to which a tertiary amplifier nucleic acid is bound, to which a signaling entity is bound, or to a target may be bound an encoding nucleic acid probe, to which a primary amplifier nucleic acid is bound, to which a secondary amplifier nucleic acid is bound, to which a tertiary amplifier nucleic acid is bound, to which a quaternary amplifier nucleic acid is bound, to which a signaling entity is bound, etc. Accordingly, the ending amplifier nucleic acid need not necessarily be the secondary amplifier nucleic acid in all embodiments.

In some aspects, cells may be immobilized or fixed to a substrate, e.g., prior to determining genotype as discussed below. In some cases, immobilization or fixing of the cells may occur after determination of phenotype. This may be useful according to certain embodiments, for example, to correlate the phenotype of the cells within an image with the subsequent genotype of the cells (e.g., determined as discussed below). The cells can also be fixed in some embodiments before measuring the phenotype instead of after measuring the phenotype and before measuring the genotype.

Those of ordinary skill in the art will be aware of systems and methods for fixing or otherwise immobilizing cells on a substrate. As non-limiting examples, a cell may be fixed using chemicals such as formaldehyde, paraformaldehyde, glutaraldehyde, ethanol, methanol, acetone, acetic acid, or the like. In one embodiment, a cell may be fixed using Hepes-glutamic acid buffer-mediated organic solvent (HOPE). See also U.S. Pat. Apl. Ser. No. 62/419,033, incorporated herein by reference in its entirety.

Certain aspects of the invention are directed to determining a sample, which may include a cell culture, a suspension of cells, a biological tissue, a biopsy, an organism, or the like. The sample can also be cell-free but nevertheless contain nucleic acids in some cases. If the sample contains a cell, the cell may be a human cell, or any other suitable cell, e.g., a mammalian cell, a fish cell, an insect cell, a plant cell, or the like. More than one cell may be present in some cases.

Within the sample, the targets to be determined can include nucleic acids, proteins, or the like. Nucleic acids to be determined may include, for example, DNA (for example, genomic DNA), RNA, or other nucleic acids that are present within a cell (or other sample). The nucleic acids may be endogenous to the cell, or added to the cell. For instance, the nucleic acid may be viral, or artificially created. In some cases, the nucleic acid to be determined may be expressed by the cell. The nucleic acid is RNA in some embodiments. The RNA may be coding and/or non-coding RNA. For example, the RNA may encode a protein. Non-limiting examples of RNA that may be studied within the cell include mRNA, siRNA, rRNA, miRNA, tRNA, lncRNA, snoRNAs, snRNAs, exRNAs, piRNAs, or the like.

In some cases, a significant portion of the nucleic acid within the cell may be studied. For instance, in some cases, enough of the RNA present within a cell may be determined so as to produce a partial or complete transcriptome of the cell. In some cases, at least 4 types of mRNAs are determined within a cell, and in some cases, at least 3, at least 4, at least 7, at least 8, at least 12, at least 14, at least 15, at least 16, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000 types of mRNAs may be determined within a cell.

In some cases, the transcriptome of a cell may be determined. It should be understood that the transcriptome generally encompasses all RNA molecules produced within a cell, not just mRNA. Thus, for instance, the transcriptome may also include rRNA, tRNA, siRNA, etc. in certain instances. In some embodiments, at least 5%, at least 10%, at least 15%, at least 20%, at least 25%, at least 30%, at least 40%, at least 50%, at least 60%, at least 70%, at least 80%, at least 90%, or 100% of the transcriptome of a cell may be determined.

In some embodiments, other targets to be determined can include targets that are linked to nucleic acids, proteins, or the like. For instance, in one set of embodiments, a binding entity able to recognize a target may be conjugated to a nucleic acid probe. The binding entity may be any entity that can recognize a target, e.g., specifically or non-specifically. Non-limiting examples include enzymes, antibodies, receptors, complementary nucleic acid strands, aptamers, or the like. For example, an oligonucleotide-linked antibody may be used to determine a target. The target may bind to the oligonucleotide-linked antibody, and the oligonucleotides determined as discussed herein.

The determination of targets, such as nucleic acids within the cell or other sample, may be qualitative and/or quantitative. In addition, the determination may also be spatial, e.g., the position of the nucleic acids, or other targets, within the cell or other sample may be determined in two or three dimensions. In some embodiments, the positions, number, and/or concentrations of nucleic acids, or other targets, within the cell or other sample may be determined.

In some cases, a significant portion of the genome of a cell may be determined. The determined genomic segments may be continuous or interspersed on the genome. For example, in some cases, at least 4 genomic segments are determined within a cell, and in some cases, at least 3, at least 4, at least 7, at least 8, at least 12, at least 14, at least 15, at least 16, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000 genomic segments may be determined within a cell.

In some cases, the entire genome of a cell may be determined. It should be understood that the genome generally encompasses all DNA molecules produced within a cell, not just chromosome DNA. Thus, for instance, the genome may also include, in some cases, mitochondria DNA, chloroplast DNA, plasmid DNA, etc., e.g., in addition to (or instead of) chromosome DNA. In some embodiments, at least about 5%, at least about 10%, at least about 15%, at least about 20%, at least about 25%, at least about 30%, at least about 40%, at least about 50%, at least about 60%, at least about 70%, at least about 80%, at least about 90%, or 100% of the genome of a cell may be determined.

As discussed herein, a variety of nucleic acid probes may be used to determine one or more targets within a cell or other sample, according to certain aspects. The probes may comprise nucleic acids (or entities that can hybridize to a nucleic acid, e.g., specifically) such as DNA, RNA, LNA (locked nucleic acids), PNA (peptide nucleic acids), and/or combinations thereof. In some cases, additional components may also be present within the nucleic acid probes, e.g., as discussed herein. In addition, any suitable method may be used to introduce nucleic acid probes into a cell.

Other components may also be present within a nucleic acid probe or an amplifier nucleic acid as well. For example, in one set of embodiments, one or more primer sequences may be present, e.g., to facilitate enzymatic amplification. Those of ordinary skill in the art will be aware of primer sequences suitable for applications such as amplification (e.g., using PCR or other suitable techniques). Many such primer sequences are available commercially. Other examples of sequences that may be present within a primary nucleic acid probe include, but are not limited to promoter sequences, operons, identification sequences, nonsense sequences, or the like.

Typically, a primer is a single-stranded or partially double-stranded nucleic acid (e.g., DNA) that serves as a starting point for nucleic acid synthesis, allowing polymerase enzymes such as nucleic acid polymerase to extend the primer and replicate the complementary strand. A primer is (e.g., is designed to be) complementary to and to hybridize to a target nucleic acid. In some embodiments, a primer is a synthetic primer. In some embodiments, a primer is a non-naturally-occurring primer. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides.

In some embodiments, one or more signaling entities may be bound to the recognition entities on the secondary amplifier nucleic acids (or other ending amplifier nucleic acid). Non-limiting examples of signaling entities include fluorescent entities (fluorophores) or phosphorescent entities, e.g., as discussed below. The signaling entities may then be determined, e.g., to determine the nucleic acid probes or the targets. In some cases, the determination may be spatial, e.g., in two or three dimensions. In addition, in some cases, the determination may be quantitative, e.g., the amount or concentration of signaling entity and/or of a target may be determined.

In one set of embodiments, the signaling entities may be attached to the secondary amplifier nucleic acid (or other ending amplifier nucleic acid). The signaling entities may be attached to the secondary amplifier nucleic acid (or other ending amplifier nucleic acid) before or after association of the secondary amplifier nucleic acid to targets within the sample. For example, the signaling entities may be attached to the secondary amplifier nucleic acid initially, or after the secondary amplifier nucleic acids have been applied to a sample. In some cases, the signaling entities are added, then reacted to attach them to the amplifier nucleic acids.

In one set of embodiments, the signaling entities may be attached to a nucleotide sequence via a bond that can be cleaved to release the signaling entity. For example, after determination the distribution of nucleic acid probes within a sample, the signaling entities may be released or inactivated, prior to another round of nucleic acid probes and/or amplifier nucleic acids. Thus, in some embodiments, the bond may be a cleavable bond, such as a disulfide bond or a photocleavable bond. Examples of photocleavable bonds are discussed in detail herein. In some cases, such bonds may be cleaved, for example, upon exposure to reducing agents or light (e.g., ultraviolet light). See below for additional details. Other examples of systems and methods for inactivating and/or removing the signaling entity are discussed in more detail herein.

In certain embodiments, the use of primary and secondary amplifier nucleic acids suggests that there is a maximum number of signaling entities that can be bound to a given nucleic acid probe. For instance, there may be a maximum number of primary amplifier nucleic acids is able to bind to a nucleic acid probe, e.g., due to a maximum number of secondary amplifier nucleic acids that are able to bind to a finite number of primary amplifier nucleic acids, and/or due to a maximum number of primary amplifier nucleic acids that are able to bind to the finite number of read sequences on the nucleic acid probes. While each potential location need not actually be filled with a signaling entity, this structure suggests that there is a saturation limit of signaling entities, beyond which any additional signaling entities that may happen to be present are unable to associate with a nucleic acid probe or its target.

Accordingly, certain embodiments of the invention are generally directed to systems and methods of amplifying a signal indicating a nucleic acid probe or its target that are saturatable, i.e., such that there is an upper, saturation limit of how many signaling entities can associate with the nucleic acid probe or its target. Typically, that number is greater than 1. For instance, the upper limit of signaling entities may be at least 2, at least 3, at least 5, at least 10, at least 15, at least 20, at least 25, at least 30, at least 40, at least 50, at least 75, at least 100, at least 125, at least 150, at least 175, at least 200, at least 250, at least 300, at least 400, at least 500, etc. In some cases, the upper limit may be less than 500, less than 400, less than 300, less than 250, less than 200, less than 175, less than 150, less than 125, less than 100, less than 75, less than 50, less than 40, less than 30, less than 25, less than 20, less than 15, less than 10, less than 5, etc. In some cases, the upper limit may be determined as the maximum number of signaling entities that can bind to a secondary amplifier nucleic acid, multiplied by the maximum number of secondary amplifier nucleic acids that can bind to a primary amplifier nucleic acid, multiplied by the maximum number of primary amplifier nucleic acids that can bind to a nucleic acid probe that binds to a target. In contrast, techniques such as rolling circle amplification or hairpin unfolding allow the amplification of a signal in an uncontrolled manner, i.e., when sufficient reagents are present, amplification can continue without a predetermined endpoint or saturation limit. Thus, such techniques have no theoretical upper limit as to the number of signaling entities that can associate with the nucleic acid probe or its target.

It should be understood, however, that the average number of signaling entities actually bound to a nucleic acid probe or its target need not actually be the same as its upper limit, i.e., the signaling entities may not actually be at full saturation (although they can be). For instance, the amount of saturation (or the number of signaling entities bound, relative to the maximum number that can bind) may be less than 97%, less than 95%, less than 90%, less than 85%, less than 80%, less than 75%, etc., and/or at least 50%, at least 75%, at least 80%, at least 85%, at least 90%, at least 95%, at least 97%, etc. In some cases, allowing more time for binding to occur and/or increasing the concentration of reagents may increase the amount of saturation.

Because of the potential upper limit on the number of signaling entities actually bound to a nucleic acid probe or its target, the binding events distributed within a sample, e.g., spatially, may present substantially uniform sizes and/or brightnesses, in contrast to uncontrolled amplifications, such as those discussed above. For instance, due to the specific number of secondary amplifier nucleic acids that can bind to a primary amplifier nucleic acids, the secondary amplifier nucleic acids cannot be found greater than a fixed distance from the nucleic acid probe or its target, which may limit the “spot size” or diameter of fluorescence from the signaling entities, indicating binding.

In certain embodiments, at least 50%, at least 60%, at least 70%, at least 75%, at least 80%, at least 85%, at least 90%, or at least 95% of the binding events may exhibit substantially the same brightnesses, sizes (e.g., apparent diameters), colors, or the like, which may make it easier to distinguish binding events from other events, such as nonspecific binding, noise, or the like.

In addition, as previously discussed, certain aspects of the invention use code spaces that encode the various binding events, and optionally can use error detection and/or correction to determine the binding of nucleic acid probes to their targets. In some cases, a population of nucleic acid probes may contain certain “read sequences” which can bind certain amplifier nucleic acids, as discussed above, and the locations of the nucleic acid probes or targets can be determined within the sample using signaling entities associated with the amplifier nucleic acids, for example, within a certain code space, e.g., as discussed herein. See also Int. Pat. Apl. Pub. Nos. WO 2016/018960 and WO 2016/018963, each incorporated herein by reference in its entirety. As mentioned, in some cases, a population of read sequences within the nucleic acid probes may be combined in various combinations, e.g., such that a relatively small number of read sequences may be used to determine a relatively large number of different nucleic acid probes, as discussed herein.

Thus, in some cases, a population of nucleic acid probes may each contain a certain number of read sequences, some of which are shared between different nucleic acid probes such that the total population of nucleic acid probes may contain a certain number of read sequences. A population of nucleic acid probes may have any suitable number of read sequences. For example, a population of nucleic acid probes may have 1, 2, 3, 4, 5, 6, 7, 8, 9, 10, 11, 12, 13, 14, 15, 16, 17, 18, 19, 20, etc. read sequences. More than 20 are also possible in some embodiments. In addition, in some cases, a population of nucleic acid probes may, in total, have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 20 or more, 24 or more, 32 or more, 40 or more, 50 or more, 60 or more, 64 or more, 100 or more, 128 or more, etc. of possible read sequences present, although some or all of the probes may each contain more than one read sequence, as discussed herein. In addition, in some embodiments, the population of nucleic acid probes may have no more than 100, no more than 80, no more than 64, no more than 60, no more than 50, no more than 40, no more than 32, no more than 24, no more than 20, no more than 16, no more than 15, no more than 14, no more than 13, no more than 12, no more than 11, no more than 10, no more than 9, no more than 8, no more than 7, no more than 6, no more than 5, no more than 4, no more than 3, or no more than two read sequences present. Combinations of any of these are also possible, e.g., a population of nucleic acid probes may comprise between 10 and 15 read sequences in total.

As a non-limiting example of an approach to combinatorially identifying a relatively large number of nucleic acid probes from a relatively small number of read sequences contained within the nucleic acid probes, in a population of 6 different types of nucleic acid probes, each comprising one or more read sequences, the total number of read sequences within the population may be no greater than 4. It should be understood that although 4 read sequences are used in this example for ease of explanation, in other embodiments, larger numbers of nucleic acid probes may be realized, for example, using 5, 8, 10, 16, 32, etc. or more read sequences, or any other suitable number of read sequences described herein, depending on the application. For example, if each of the nucleic acid probes contains two different read sequences, then by using 4 such read sequences (A, B, C, and D), up to 6 probes may be separately identified. It should be noted that in this example, the ordering of read sequences on a nucleic acid probe is not essential, i.e., “AB” and “BA” may be treated as being synonymous (although in other embodiments, the ordering of read sequences may be essential and “AB” and “BA” may not necessarily be synonymous). Similarly, if 5 read sequences are used (A, B, C, D, and E) in the population of nucleic acid probes, up to 10 probes may be separately identified (e.g., AB, AC, AD, AE, BC, BD, BE, CD, CE, DE). For example, one of ordinary skill in the art would understand that, for k read sequences in a population with n read sequences on each probe, up to

$$( \begin{matrix}n \\ k\end{matrix} )$$

different probes may be produced, assuming that the ordering of read sequences is not essential; because not all of the probes need to have the same number of read sequences and not all combinations of read sequences need to be used in every embodiment, either more or less than this number of different probes may also be used in certain embodiments. In addition, it should also be understood that the number of read sequences on each probe need not be identical in some embodiments. For instance example, some probes may contain 2 read sequences while other probes may contain 3 read sequences.

In some aspects, the read sequences and/or the pattern of binding of nucleic acid probes within a sample may be used to define an error-detecting and/or an error-correcting code, for example, to reduce or prevent misidentification or errors of the nucleic acids. Thus, for example, if binding is indicated (e.g., as determined using a signaling entity), then the location may be identified with a “1”; conversely, if no binding is indicated, then the location may be identified with a “0” (or vice versa, in some cases). Multiple rounds of binding determinations, e.g., using different nucleic acid probes, can then be used to create a “codeword,” e.g., for that spatial location. In some embodiments, the codeword may be subjected to error detection and/or correction. For instance, the codewords may be organized such that, if no match is found for a given set of read sequences or binding pattern of nucleic acid probes, then the match may be identified as an error, and optionally, error correction may be applied sequences to determine the correct target for the nucleic acid probes. In some cases, the codewords may have fewer “letters” or positions that the total number of nucleic acids encoded by the codewords, e.g. where each codeword encodes a different nucleic acid.

Such error-detecting and/or the error-correction code may take a variety of forms. A variety of such codes have previously been developed in other contexts such as the telecommunications industry, such as Golay codes or Hamming codes. In one set of embodiments, the read sequences or binding patterns of the nucleic acid probes are assigned such that not every possible combination is assigned.

For example, if 4 read sequences are possible and a nucleic acid probe contains 2 read sequences, then up to 6 nucleic acid probes could be identified; but the number of nucleic acid probes used may be less than 6. Similarly, for k read sequences in a population with n read sequences on each nucleic acid probe,

$$( \begin{matrix}n \\ k\end{matrix} )$$

different probes may be produced, but the number of nucleic acid probes that are used may be any number more or less than

$$( \begin{matrix}n \\ k\end{matrix} ) .$$

In addition, these may be randomly assigned, or assigned in specific ways to increase the ability to detect and/or correct errors.

As another example, if multiple rounds of nucleic acid probes are used, the number of rounds may be arbitrarily chosen. If in each round, each target can give two possible outcomes, such as being detected or not being detected, up to 2ⁿ different targets may be possible for n rounds of probes, but the number of targets that are actually used may be any number less than 2ⁿ. For example, if in each round, each target can give more than two possible outcomes, such as being detected in different color channels, more than 2ⁿ (e.g. 3ⁿ, 4ⁿ, . . . ) different targets may be possible for n rounds of probes. In some cases, the number of targets that are actually used may be any number less than this number. In addition, these may be randomly assigned, or assigned in specific ways to increase the ability to detect and/or correct errors.

The codewords may be used to define various code spaces. For example, in one set of embodiments, the codewords or nucleic acid probes may be assigned within a code space such that the assignments are separated by a Hamming distance, which measures the number of incorrect “reads” in a given pattern that cause the nucleic acid probe to be misinterpreted as a different valid nucleic acid probe. In certain cases, the Hamming distance may be at least 2, at least 3, at least 4, at least 5, at least 6, or the like. In addition, in one set of embodiments, the assignments may be formed as a Hamming code, for instance, a Hamming(7, 4) code, a Hamming(15, 11) code, a Hamming(31, 26) code, a Hamming(63, 57) code, a Hamming(127, 120) code, etc. In another set of embodiments, the assignments may form a SECDED code, e.g., a SECDED(8,4) code, a SECDED(16,4) code, a SCEDED(16, 11) code, a SCEDED(22, 16) code, a SCEDED(39, 32) code, a SCEDED(72, 64) code, etc. In yet another set of embodiments, the assignments may form an extended binary Golay code, a perfect binary Golay code, or a ternary Golay code. In another set of embodiments, the assignments may represent a subset of the possible values taken from any of the codes described above.

For example, an error-detecting code may be formed by limiting the number of used codewords to less than 10%, less than 5%, less than 2%, less than 1%, less than 0.1%, less than 0.01%, less than 0.001% of the total number of the possible codewords, so that the incorrect codewords are unlikely to be present as another used codeword. Therefore, any detected codewords that do not match a used codeword is more likely to be incorrect.

For example, an error-correcting code may be formed by using only binary words that contain a fixed or constant number of “1” bits (or “0” bits) to encode the targets. For example, the code space may only include 1, 2, 3, 4, 5, 6, 7, 8, 9, 10, 11, 12, 13, 14, 15, 16, etc. “1” bits (or “0” bits), e.g., all of the codes have the same number of “1” bits or “0” bits, etc. In another set of embodiments, the assignments may represent a subset of the possible values taken from codes described above for the purpose of addressing asymmetric readout errors. For example, in some cases, a code in which the number of “1” bits may be fixed for all used binary words may eliminate the biased measurement of words with different numbers of “1”s when the rate at which “0” bits are measured as “1”s or “1” bits are measured as “0”s are different.

Accordingly, in some embodiments, once the codeword is determined (e.g., as discussed herein), the codeword may be compared to the known nucleic acid codewords. If a match is found, then the nucleic acid target can be identified or determined. If no match is found, then an error in the reading of the codeword may be identified. In some cases, error correction can also be applied to determine the correct codeword, and thus resulting in the correct identity of the nucleic acid target. In some cases, the codewords may be selected such that, assuming that there is only one error present, only one possible correct codeword is available, and thus, only one correct identity of the nucleic acid target is possible. In some cases, this may also be generalized to larger codeword spacings or Hamming distances; for instance, the codewords may be selected such that if two, three, or four errors are present (or more in some cases), only one possible correct codeword is available, and thus, only one correct identity of the nucleic acid targets is possible.

The error-correcting code may be a binary error-correcting code, or it may be based on other numbering systems, e.g., ternary or quaternary error-correcting codes. For instance, in one set of embodiments, more than one type of signaling entity may be used and assigned to different numbers within the error-correcting code. Thus, as a non-limiting example, a first signaling entity (or more than one signaling entity, in some cases) may be assigned as “1” and a second signaling entity (or more than one signaling entity, in some cases) may be assigned as “2” (with “0” indicating no signaling entity present), and the codewords distributed to define a ternary error-correcting code. Similarly, a third signaling entity may additionally be assigned as “3” to make a quaternary error-correcting code, etc. Non-limiting examples of such codes include the Reed-Solomon erasure codes and generalizations thereof.

In addition, the code can also be selected in some embodiments through random selection of a sub-set of all possible codewords. For example, a random subset of binary codewords of length n code be selected. In some cases, these codewords can be separated by Hamming distances, i.e. the number of bits that must be flipped to convert one into another, so that some of the used codewords maintain some error robust or correcting abilities. In some embodiments, approaches such as next-generations sequencing can be used to measure the random subset of codewords used and error robustness and error correction could be applied selectively on the codewords that satisfy the constraints necessary for these properties.

As discussed herein, in certain aspects, signaling entities are determined, e.g., by imaging, to determine nucleic acid probes and/or to create codewords. Examples of signaling entities include those discussed herein. In some cases, signaling entities within a sample may be determined, e.g., spatially, using a variety of techniques. In some embodiments, the signaling entities may be fluorescent, and techniques for determining fluorescence within a sample, such as fluorescence microscopy or confocal microscopy, may be used to spatially identify the positions of signaling entities within a cell. In some cases, the positions of entities within the sample may be determined in two or even three dimensions. In addition, in some embodiments, more than one signaling entity may be determined at a time (e.g., signaling entities with different colors or emissions), and/or sequentially.

In addition, in some embodiments, a confidence level for a target, e.g., a nucleic acid target, may be determined. For example, the confidence level may be determined using a ratio of the number of exact matches to the number of matches having one or more one-bit errors. In some cases, only matches having a confidence ratio greater than a certain value may be used. For instance, in certain embodiments, matches may be accepted only if the confidence ratio for the match is greater than about 0.01, greater than about 0.03, greater than about 0.05, greater than about 0.1, greater than about 0.3, greater than about 0.5, greater than about 1, greater than about 3, greater than about 5, greater than about 10, greater than about 30, greater than about 50, greater than about 100, greater than about 300, greater than about 500, greater than about 1000, or any other suitable value. In addition, in some embodiments, matches may be accepted only if the confidence ratio for the target is greater than an internal standard or false positive control by about 0.01, about 0.03, about 0.05, about 0.1, about 0.3, about 0.5, about 1, about 3, about 5, about 10, about 30, about 50, about 100, about 300, about 500, about 1000, or any other suitable value

In some embodiments, the spatial positions of the entities (and thus, nucleic acid probes that the entities may be associated with) may be determined at relatively high resolutions. For instance, the positions may be determined at spatial resolutions of better than about 100 micrometers, better than about 30 micrometers, better than about 10 micrometers, better than about 3 micrometers, better than about 1 micrometer, better than about 800 nm, better than about 600 nm, better than about 500 nm, better than about 400 nm, better than about 300 nm, better than about 200 nm, better than about 100 nm, better than about 90 nm, better than about 80 nm, better than about 70 nm, better than about 60 nm, better than about 50 nm, better than about 40 nm, better than about 30 nm, better than about 20 nm, or better than about 10 nm, etc.

There are a variety of techniques able to determine or image the spatial positions of entities optically, e.g., using fluorescence microscopy. More than one color can be used in some embodiments. In some cases, the spatial positions may be determined at super resolutions, or at resolutions better than the wavelength of light or the diffraction limit. Non-limiting examples include STORM (stochastic optical reconstruction microscopy), STED (stimulated emission depletion microscopy), NSOM (Near-field Scanning Optical Microscopy), 4Pi microscopy, SIM (Structured Illumination Microscopy), SMI (Spatially Modulated Illumination) microscopy, RESOLFT (Reversible Saturable Optically Linear Fluorescence Transition Microscopy), GSD (Ground State Depletion Microscopy), SSIM (Saturated Structured-Illumination Microscopy), SPDM (Spectral Precision Distance Microscopy), Photo-Activated Localization Microscopy (PALM), Fluorescence Photoactivation Localization Microscopy (FPALM), LIMON (3D Light Microscopical Nanosizing Microscopy), Super-resolution optical fluctuation imaging (SOFI), or the like. See, e.g., U.S. Pat. No. 7,838,302, issued Nov. 23, 2010, entitled “Sub-Diffraction Limit Image Resolution and Other Imaging Techniques,” by Zhuang, et al.; U.S. Pat. No. 8,564,792, issued Oct. 22, 2013, entitled “Sub-diffraction Limit Image Resolution in Three Dimensions,” by Zhuang, et al.; or Int. Pat. Apl. Pub. No. WO 2013/090360, published Jun. 20, 2013, entitled “High Resolution Dual-Objective Microscopy,” by Zhuang, et al., each incorporated herein by reference in their entireties.

As an illustrative non-limiting example, in one set of embodiments, the sample may be imaged with a high numerical aperture, oil immersion objective with 100× magnification and light collected on an electron-multiplying CCD camera. In another example, the sample could be imaged with a high numerical aperture, oil immersion lens with 40× magnification and light collected with a wide-field scientific CMOS camera. With different combinations of objectives and cameras, a single field of view may correspond to no less than 40×40 microns, 80×80 microns, 120×120 microns, 240×240 microns, 340×340 microns, or 500×500 microns, etc. in various non-limiting embodiments. Similarly, a single camera pixel may correspond, in some embodiments, to regions of the sample of no less than 80×80 nm, 120×120 nm, 160×160 nm, 240×240 nm, or 300×300 nm, etc. In another example, the sample may be imaged with a low numerical aperture, air lens with 10× magnification and light collected with a sCMOS camera. In additional embodiments, the sample may be optically sectioned by illuminating it via a single or multiple scanned diffraction limited foci generated either by scanning mirrors or a spinning disk and the collected passed through a single or multiple pinholes. In another embodiment, the sample may also be illuminated via thin sheet of light generated via any one of multiple methods known to those versed in the art.

In one embodiment, the sample may be illuminated by single Gaussian mode laser lines. In some embodiments, the illumination profiled may be flattened by passing these laser lines through a multimode fiber that is vibrated via piezo-electric or other mechanical means. In some embodiments, the illumination profile may be flattened by passing single-mode, Gaussian beams through a variety of refractive beam shapers, such as the piShaper or a series of stacked Powell lenses. In yet another set of embodiments, the Gaussian beams may be passed through a variety of different diffusing elements, such as ground glass or engineered diffusers, which may be spun in some cases at high speeds to remove residual laser speckle. In yet another embodiment, laser illumination may be passed through a series of lenslet arrays to produce overlapping images of the illumination that approximate a flat illumination field.

In some embodiments, the centroids of the spatial positions of the entities may be determined. For example, a centroid of a signaling entity may be determined within an image or series of images using image analysis algorithms known to those of ordinary skill in the art. In some cases, the algorithms may be selected to determine non-overlapping single emitters and/or partially overlapping single emitters in a sample. Non-limiting examples of suitable techniques include a maximum likelihood algorithm, a least squares algorithm, a Bayesian algorithm, a compressed sensing algorithm, or the like. Combinations of these techniques may also be used in some cases.

In addition, the signaling entity may be inactivated in some cases. For example, in some embodiments, a first secondary nucleic acid probe that can associate with a signaling entity (e.g., using amplifier nucleic acids) may be applied to a sample that can recognize a first read sequence (e.g., on the nucleic acid probe), then the signaling entity can be inactivated before a second secondary nucleic acid probe is applied to the sample, e.g., that can associate with a signaling entity (e.g., using amplifier nucleic acids). If multiple signaling entities are used, the same or different techniques may be used to inactivate the signaling entities, and some or all of the multiple signaling entities may be inactivated, e.g., sequentially or simultaneously.

Inactivation may be caused by removal of the signaling entity (e.g., from the sample, or from the nucleic acid probe, etc.), and/or by chemically altering the signaling entity in some fashion (e.g., by photobleaching the signaling entity, bleaching or chemically altering the structure of the signaling entity, for example, by reduction, etc.). For instance, in one set of embodiments, a fluorescent signaling entity may be inactivated by chemical or optical techniques such as oxidation, photobleaching, chemically bleaching, stringent washing or enzymatic digestion or reaction by exposure to an enzyme, dissociating the signaling entity from other components (e.g., a probe), chemical reaction of the signaling entity (e.g., to a reactant able to alter the structure of the signaling entity) or the like. For instance, bleaching may occur by exposure to oxygen, reducing agents, or the signaling entity could be chemically cleaved from the nucleic acid probe (for example, using tris(2-carboxyethyl)phosphine) and washed away via fluid flow.

In some embodiments, various nucleic acid probes (including primary and/or secondary nucleic acid probes) may be associated with one or more signaling entities, e.g., using amplifier nucleic acids as discussed herein. If more than one nucleic acid probe is used, the signaling entities may each by the same or different. In certain embodiments, a signaling entity is any entity able to emit light. For instance, in one embodiment, the signaling entity is fluorescent. In other embodiments, the signaling entity may be phosphorescent, radioactive, absorptive, etc. In some cases, the signaling entity is any entity that can be determined within a sample at relatively high resolutions, e.g., at resolutions better than the wavelength of visible light or the diffraction limit. The signaling entity may be, for example, a dye, a small molecule, a peptide or protein, or the like. The signaling entity may be a single molecule in some cases. If multiple secondary nucleic acid probes are used, the nucleic acid probes may associate with or comprise the same or different signaling entities.

Non-limiting examples of signaling entities include fluorescent entities (fluorophores) or phosphorescent entities, for example, cyanine dyes (e.g., Cy2, Cy3, Cy3B, Cy5, Cy5.5, Cy7, etc.), Alexa Fluor dyes, Atto dyes, photoswitchable dyes, photoactivatable dyes, fluorescent dyes, metal nanoparticles, semiconductor nanoparticles or “quantum dots,” fluorescent proteins such as GFP (Green Fluorescent Protein), or photoactivabale fluorescent proteins, such as PAGFP, PSCFP, PSCFP2, Dendra, Dendra2, EosFP, tdEos, mEos2, mEos3, PAmCherry, PAtagRFP, mMaple, mMaple2, and mMaple3. Other suitable signaling entities are known to those of ordinary skill in the art. See, e.g., U.S. Pat. No. 7,838,302 or Int. Pat Apl. Pub. No. WO 2015/160690, each incorporated herein by reference in its entirety.

In one set of embodiments, the signaling entity may be attached to an oligonucleotide sequence via a bond that can be cleaved to release the signaling entity. In one set of embodiments, a fluorophore may be conjugated to an oligonucleotide via a cleavable bond, such as a photocleavable bond. Non-limiting examples of photocleavable bonds include, but are not limited to, 1-(2-nitrophenyl)ethyl, 2-nitrobenzyl, biotin phosphoramidite, acrylic phosphoramidite, diethylaminocoumarin, 1-(4,5-dimethoxy-2-nitrophenyl)ethyl, cyclo-dodecyl (dimethoxy-2-nitrophenyl)ethyl, 4-aminomethyl-3-nitrobenzyl, (4-nitro-3-(1-chlorocarbonyloxyethyl)phenyl)methyl-S-acetylthioic acid ester, (4-nitro-3-(1-thlorocarbonyloxyethyl)phenyl)methyl-3-(2-pyridyldithiopropionic acid) ester, 3-(4,4′-dimethoxytrityl)-1-(2-nitrophenyl)-propane-1,3-diol-[2-cyanoethyl-(N,N-diisopropyl)]-phosphoramidite, 1-[2-nitro-5-(6-trifluoroacetylcaproamidomethyl)phenyl]-ethyl-[2-cyano-ethyl-(N,N-diisopropyl)]-phosphoramidite, 1-[2-nitro-5-(6-(4,4′-dimethoxytrityloxy)butyramidomethyl)phenyl]-ethyl-[2-cyanoethyl-(N,N-diisopropyl)]-phosphoramidite, 1-[2-nitro-5-(6-(N-(4,4′-dimethoxytrityl))-biotinamidocaproamido-methyl)phenyl]-ethyl-[2-cyanoethyl-(N,N-diisopropyl)]-phosphoramidite, or similar linkers. The oligonucleotide sequence may be, for example, a primary or secondary (or other) amplifier nucleic acid, such as those discussed herein.

In another set of embodiments, the fluorophore may be conjugated to an oligonucleotide via a disulfide bond. The disulfide bond may be cleaved by a variety of reducing agents such as, but not limited to, dithiothreitol, dithioerythritol, beta-mercaptoethanol, sodium borohydride, thioredoxin, glutaredoxin, trypsinogen, hydrazine, diisobutylaluminum hydride, oxalic acid, formic acid, ascorbic acid, phosphorous acid, tin chloride, glutathione, thioglycolate, 2,3-dimercaptopropanol, 2-mercaptoethylamine, 2-aminoethanol, tris(2-carboxyethyl)phosphine, bis(2-mercaptoethyl) sulfone, N,N′-dimethyl-N,N′-bis(mercaptoacetyl)hydrazine, 3-mercaptoproptionate, dimethylformamide, thiopropyl-agarose, tri-n-butylphosphine, cysteine, iron sulfate, sodium sulfite, phosphite, hypophosphite, phosphorothioate, or the like, and/or combinations of any of these. The oligonucleotide sequence may be, for example, a primary or secondary (or other) amplifier nucleic acid, such as those discussed herein.

In another embodiment, the fluorophore may be conjugated to an oligonucleotide via one or more phosphorothioate modified nucleotides in which the sulfur modification replaces the bridging and/or non-bridging oxygen. The fluorophore may be cleaved from the oligonucleotide, in certain embodiments, via addition of compounds such as but not limited to iodoethanol, iodine mixed in ethanol, silver nitrate, or mercury chloride. In yet another set of embodiments, the signaling entity may be chemically inactivated through reduction or oxidation. For example, in one embodiment, a chromophore such as Cy5 or Cy7 may be reduced using sodium borohydride to a stable, non-fluorescence state. In still another set of embodiments, a fluorophore may be conjugated to an oligonucleotide via an azo bond, and the azo bond may be cleaved with 2-[(2-N-arylamino)phenylazo]pyridine. In yet another set of embodiments, a fluorophore may be conjugated to an oligonucleotide via a suitable nucleic acid segment that can be cleaved upon suitable exposure to DNAse, e.g., an exodeoxyribonuclease or an endodeoxyribonuclease. Examples include, but are not limited to, deoxyribonuclease I or deoxyribonuclease II. In one set of embodiments, the cleavage may occur via a restriction endonuclease. Non-limiting examples of potentially suitable restriction endonucleases include BamHI, BsrI, NotI, XmaI, PspAI, DpnI, MboI, MnlI, Eco57I, Ksp632I, DraIII, AhaII, SmaI, MluI, HpaI, ApaI, BelI, BstEII, TaqI, EcoRI, SacI, HindII, HaeII, DraII, Tsp509I, Sau3AI, PacI, etc. Over 3000 restriction enzymes have been studied in detail, and more than 600 of these are available commercially. In yet another set of embodiments, a fluorophore may be conjugated to biotin, and the oligonucleotide conjugated to avidin or streptavidin. An interaction between biotin and avidin or streptavidin allows the fluorophore to be conjugated to the oligonucleotide, while sufficient exposure to an excess of addition, free biotin could “outcompete” the linkage and thereby cause cleavage to occur. In addition, in another set of embodiments, the probes may be removed using corresponding “toe-hold-probes,” which comprise the same sequence as the probe, as well as an extra number of bases of homology to the encoding probes (e.g., 1-20 extra bases, for example, 5 extra bases). These probes may remove the labeled readout probe through a strand-displacement interaction. The oligonucleotide sequence may be, for example, a primary or secondary (or other) amplifier nucleic acid, such as those discussed herein.

As used herein, the term “light” generally refers to electromagnetic radiation, having any suitable wavelength (or equivalently, frequency). For instance, in some embodiments, the light may include wavelengths in the optical or visual range (for example, having a wavelength of between about 400 nm and about 700 nm, i.e., “visible light”), infrared wavelengths (for example, having a wavelength of between about 300 micrometers and 700 nm), ultraviolet wavelengths (for example, having a wavelength of between about 400 nm and about 10 nm), or the like. In certain cases, as discussed in detail below, more than one entity may be used, i.e., entities that are chemically different or distinct, for example, structurally. However, in other cases, the entities may be chemically identical or at least substantially chemically identical.

In one set of embodiments, the signaling entity is “switchable,” i.e., the entity can be switched between two or more states, at least one of which emits light having a desired wavelength. In the other state(s), the entity may emit no light, or emit light at a different wavelength. For instance, an entity may be “activated” to a first state able to produce light having a desired wavelength, and “deactivated” to a second state not able to emit light of the same wavelength. An entity is “photoactivatable” if it can be activated by incident light of a suitable wavelength. As a non-limiting example, Cy5, can be switched between a fluorescent and a dark state in a controlled and reversible manner by light of different wavelengths, i.e., 633 nm (or 642 nm, 647 nm, 656 nm) red light can switch or deactivate Cy5 to a stable dark state, while 405 nm green light can switch or activate the Cy5 back to the fluorescent state. In some cases, the entity can be reversibly switched between the two or more states, e.g., upon exposure to the proper stimuli. For example, a first stimuli (e.g., a first wavelength of light) may be used to activate the switchable entity, while a second stimuli (e.g., a second wavelength of light) may be used to deactivate the switchable entity, for instance, to a non-emitting state. Any suitable method may be used to activate the entity. For example, in one embodiment, incident light of a suitable wavelength may be used to activate the entity to emit light, i.e., the entity is “photoswitchable.” Thus, the photoswitchable entity can be switched between different light-emitting or non-emitting states by incident light, e.g., of different wavelengths. The light may be monochromatic (e.g., produced using a laser) or polychromatic. In another embodiment, the entity may be activated upon stimulation by electric field and/or magnetic field. In other embodiments, the entity may be activated upon exposure to a suitable chemical environment, e.g., by adjusting the pH, or inducing a reversible chemical reaction involving the entity, etc. Similarly, any suitable method may be used to deactivate the entity, and the methods of activating and deactivating the entity need not be the same. For instance, the entity may be deactivated upon exposure to incident light of a suitable wavelength, or the entity may be deactivated by waiting a sufficient time.

Typically, a “switchable” entity can be identified by one of ordinary skill in the art by determining conditions under which an entity in a first state can emit light when exposed to an excitation wavelength, switching the entity from the first state to the second state, e.g., upon exposure to light of a switching wavelength, then showing that the entity, while in the second state can no longer emit light (or emits light at a much reduced intensity) when exposed to the excitation wavelength.

In one set of embodiments, as discussed, a switchable entity may be switched upon exposure to light. In some cases, the light used to activate the switchable entity may come from an external source, e.g., a light source such as a laser light source, another light-emitting entity proximate the switchable entity, etc. The second, light emitting entity, in some cases, may be a fluorescent entity, and in certain embodiments, the second, light-emitting entity may itself also be a switchable entity.

In some embodiments, the switchable entity includes a first, light-emitting portion (e.g., a fluorophore), and a second portion that activates or “switches” the first portion. For example, upon exposure to light, the second portion of the switchable entity may activate the first portion, causing the first portion to emit light. Examples of activator portions include, but are not limited to, Alexa Fluor 405 (Invitrogen), Alexa Fluor 488 (Invitrogen), Cy2 (GE Healthcare), Cy3 (GE Healthcare), Cy3B (GE Healthcare), Cy3.5 (GE Healthcare), or other suitable dyes. Examples of light-emitting portions include, but are not limited to, Cy5, Cy5.5 (GE Healthcare), Cy7 (GE Healthcare), Alexa Fluor 647 (Invitrogen), Alexa Fluor 680 (Invitrogen), Alexa Fluor 700 (Invitrogen), Alexa Fluor 750 (Invitrogen), Alexa Fluor 790 (Invitrogen), DiD, DiR, YOYO-3 (Invitrogen), YO-PRO-3 (Invitrogen), TOT-3 (Invitrogen), TO-PRO-3 (Invitrogen) or other suitable dyes. These may linked together, e.g., covalently, for example, directly, or through a linker, e.g., forming compounds such as, but not limited to, Cy5-Alexa Fluor 405, Cy5-Alexa Fluor 488, Cy5-Cy2, Cy5-Cy3, Cy5-Cy3.5, Cy5.5-Alexa Fluor 405, Cy5.5-Alexa Fluor 488, Cy5.5-Cy2, Cy5.5-Cy3, Cy5.5-Cy3.5, Cy7-Alexa Fluor 405, Cy7-Alexa Fluor 488, Cy7-Cy2, Cy7-Cy3, Cy7-Cy3.5, Alexa Fluor 647-Alexa Fluor 405, Alexa Fluor 647-Alexa Fluor 488, Alexa Fluor 647-Cy2, Alexa Fluor 647-Cy3, Alexa Fluor 647-Cy3.5, Alexa Fluor 750-Alexa Fluor 405, Alexa Fluor 750-Alexa Fluor 488, Alexa Fluor 750-Cy2, Alexa Fluor 750-Cy3, or Alexa Fluor 750-Cy3.5. Those of ordinary skill in the art will be aware of the structures of these and other compounds, many of which are available commercially. The portions may be linked via a covalent bond, or by a linker, such as those described in detail below. Other light-emitting or activator portions may include portions having two quaternized nitrogen atoms joined by a polymethine chain, where each nitrogen is independently part of a heteroaromatic moiety, such as pyrrole, imidazole, thiazole, pyridine, quinoine, indole, benzothiazole, etc., or part of a nonaromatic amine. In some cases, there may be 5, 6, 7, 8, 9, or more carbon atoms between the two nitrogen atoms.

In certain cases, the light-emitting portion and the activator portions, when isolated from each other, may each be fluorophores, i.e., entities that can emit light of a certain, emission wavelength when exposed to a stimulus, for example, an excitation wavelength. However, when a switchable entity is formed that comprises the first fluorophore and the second fluorophore, the first fluorophore forms a first, light-emitting portion and the second fluorophore forms an activator portion that switches that activates or “switches” the first portion in response to a stimulus. For example, the switchable entity may comprise a first fluorophore directly bonded to the second fluorophore, or the first and second entity may be connected via a linker or a common entity. Whether a pair of light-emitting portion and activator portion produces a suitable switchable entity can be tested by methods known to those of ordinary skills in the art. For example, light of various wavelength can be used to stimulate the pair and emission light from the light-emitting portion can be measured to determined wither the pair makes a suitable switch.

As a non-limiting example, Cy3 and Cy5 may be linked together to form such an entity. In this example, Cy3 is an activator portion that is able to activate Cy5, the light-emission portion. Thus, light at or near the absorption maximum (e.g., near 532 nm light for Cy3) of the activation or second portion of the entity may cause that portion to activate the first, light-emitting portion, thereby causing the first portion to emit light (e.g., near 647 nm for Cy5). See, e.g., U.S. Pat. No. 7,838,302, incorporated herein by reference in its entirety. In some cases, the first, light-emitting portion can subsequently be deactivated by any suitable technique (e.g., by directing 647 nm red light to the Cy5 portion of the molecule).

Other non-limiting examples of potentially suitable activator portions include 1,5 IAEDANS, 1,8-ANS, 4-Methylumbelliferone, 5-carboxy-2,7-dichlorofluorescein, 5-Carboxyfluorescein (5-FAM), 5-Carboxynapthofluorescein, 5-Carboxytetramethylrhodamine (5-TAMRA), 5-FAM (5-Carboxyfluorescein), 5-HAT (Hydroxy Tryptamine), 5-Hydroxy Tryptamine (HAT), 5-ROX (carboxy-X-rhodamine), 5-TAMRA (5-Carboxytetramethylrhodamine), 6-Carboxyrhodamine 6G, 6-CR 6G, 6-JOE, 7-Amino-4-methylcoumarin, 7-Aminoactinomycin D (7-AAD), 7-Hydroxy-4-methylcoumarin, 9-Amino-6-chloro-2-methoxyacridine, ABQ, Acid Fuchsin, ACMA (9-Amino-6-chloro-2-methoxyacridine), Acridine Orange, Acridine Red, Acridine Yellow, Acriflavin, Acriflavin Feulgen SITSA, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alizarin Complexon, Alizarin Red, AMC, AMCA-S, AMCA (Aminomethylcoumarin), AMCA-X, Aminoactinomycin D, Aminocoumarin, Aminomethylcoumarin (AMCA), Anilin Blue, Anthrocyl stearate, APTRA-BTC, APTS, Astrazon Brilliant Red 4G, Astrazon Orange R, Astrazon Red 6B, Astrazon Yellow 7 GLL, Atabrine, ATTO 390, ATTO 425, ATTO 465, ATTO 488, ATTO 495, ATTO 520, ATTO 532, ATTO 550, ATTO 565, ATTO 590, ATTO 594, ATTO 610, ATTO 611X, ATTO 620, ATTO 633, ATTO 635, ATTO 647, ATTO 647N, ATTO 655, ATTO 680, ATTO 700, ATTO 725, ATTO 740, ATTO-TAG CBQCA, ATTO-TAG FQ, Auramine, Aurophosphine G, Aurophosphine, BAO 9 (Bisaminophenyloxadiazole), BCECF (high pH), BCECF (low pH), Berberine Sulphate, Bimane, Bisbenzamide, Bisbenzimide (Hoechst), bis-BTC, Blancophor FFG, Blancophor SV, BOBO-1, BOBO-3, Bodipy 492/515, Bodipy 493/503, Bodipy 500/510, Bodipy 505/515, Bodipy 530/550, Bodipy 542/563, Bodipy 558/568, Bodipy 564/570, Bodipy 576/589, Bodipy 581/591, Bodipy 630/650-X, Bodipy 650/665-X, Bodipy 665/676, Bodipy Fl, Bodipy FL ATP, Bodipy Fl-Ceramide, Bodipy R6G, Bodipy TMR, Bodipy TMR-X conjugate, Bodipy TMR-X, SE, Bodipy TR, Bodipy TR ATP, Bodipy TR-X SE, BO-PRO-1, BO-PRO-3, Brilliant Sulphoflavin FF, BTC, BTC-5N, Calcein, Calcein Blue, Calcium Crimson, Calcium Green, Calcium Green-1 Ca²⁺ Dye, Calcium Green-2 Ca²⁺, Calcium Green-5N Ca²⁺, Calcium Green-C18 Ca²⁺, Calcium Orange, Calcofluor White, Carboxy-X-rhodamine (5-ROX), Cascade Blue, Cascade Yellow, Catecholamine, CCF2 (GeneBlazer), CFDA, Chromomycin A, Chromomycin A, CL-NERF, CMFDA, Coumarin Phalloidin, CPM Methylcoumarin, CTC, CTC Formazan, Cy2, Cy3.1 8, Cy3.5, Cy3, Cy5.1 8, cyclic AMP Fluorosensor (FiCRhR), Dabcyl, Dansyl, Dansyl Amine, Dansyl Cadaverine, Dansyl Chloride, Dansyl DHPE, Dansyl fluoride, DAPI, Dapoxyl, Dapoxyl 2, Dapoxyl 3′ DCFDA, DCFH (Dichlorodihydrofluorescein Diacetate), DDAO, DHR (Dihydorhodamine 123), Di-4-ANEPPS, Di-8-ANEPPS (non-ratio), DiA (4-Di-16-ASP), Dichlorodihydrofluorescein Diacetate (DCFH), DiD—Lipophilic Tracer, DiD (DiIC18(5)), DIDS, Dihydorhodamine 123 (DHR), DiI (DiIC18(3)), Dinitrophenol, DiO (DiOC18(3)), DiR, DiR (DiIC18(7)), DM-NERF (high pH), DNP, Dopamine, DTAF, DY-630-NHS, DY-635-NHS, DyLight 405, DyLight 488, DyLight 549, DyLight 633, DyLight 649, DyLight 680, DyLight 800, ELF 97, Eosin, Erythrosin, Erythrosin ITC, Ethidium Bromide, Ethidium homodimer-1 (EthD-1), Euchrysin, EukoLight, Europium (III) chloride, Fast Blue, FDA, Feulgen (Pararosaniline), FIF (Formaldehyd Induced Fluorescence), FITC, Flazo Orange, Fluo-3, Fluo-4, Fluorescein (FITC), Fluorescein Diacetate, Fluoro-Emerald, Fluoro-Gold (Hydroxystilbamidine), Fluor-Ruby, FluorX, FM 1-43, FM 4-46, Fura Red (high pH), Fura Red/Fluo-3, Fura-2, Fura-2/BCECF, Genacryl Brilliant Red B, Genacryl Brilliant Yellow 10GF, Genacryl Pink 3G, Genacryl Yellow 5GF, GeneBlazer (CCF2), Gloxalic Acid, Granular blue, Haematoporphyrin, Hoechst 33258, Hoechst 33342, Hoechst 34580, HPTS, Hydroxycoumarin, Hydroxystilbamidine (FluoroGold), Hydroxytryptamine, Indo-1, high calcium, Indo-1, low calcium, Indodicarbocyanine (DiD), Indotricarbocyanine (DiR), Intrawhite Cf, JC-1, JO-JO-1, JO-PRO-1, LaserPro, Laurodan, LDS 751 (DNA), LDS 751 (RNA), Leucophor PAF, Leucophor SF, Leucophor WS, Lissamine Rhodamine, Lissamine Rhodamine B, Calcein/Ethidium homodimer, LOLO-1, LO-PRO-1, Lucifer Yellow, Lyso Tracker Blue, Lyso Tracker Blue-White, Lyso Tracker Green, Lyso Tracker Red, Lyso Tracker Yellow, LysoSensor Blue, LysoSensor Green, LysoSensor Yellow/Blue, Mag Green, Magdala Red (Phloxin B), Mag-Fura Red, Mag-Fura-2, Mag-Fura-5, Mag-Indo-1, Magnesium Green, Magnesium Orange, Malachite Green, Marina Blue, Maxilon Brilliant Flavin 10 GFF, Maxilon Brilliant Flavin 8 GFF, Merocyanin, Methoxycoumarin, Mitotracker Green FM, Mitotracker Orange, Mitotracker Red, Mitramycin, Monobromobimane, Monobromobimane (mBBr-GSH), Monochlorobimane, MPS (Methyl Green Pyronine Stilbene), NBD, NBD Amine, Nile Red, Nitrobenzoxadidole, Noradrenaline, Nuclear Fast Red, Nuclear Yellow, Nylosan Brilliant Iavin E8G, Oregon Green, Oregon Green 488-X, Oregon Green, Oregon Green 488, Oregon Green 500, Oregon Green 514, Pacific Blue, Pararosaniline (Feulgen), PBFI, Phloxin B (Magdala Red), Phorwite AR, Phorwite BKL, Phorwite Rev, Phorwite RPA, Phosphine 3R, PKH26 (Sigma), PKH67, PMIA, Pontochrome Blue Black, POPO-1, POPO-3, PO-PRO-1, PO-PRO-3, Primuline, Procion Yellow, Propidium Iodid (PI), PyMPO, Pyrene, Pyronine, Pyronine B, Pyrozal Brilliant Flavin 7GF, QSY 7, Quinacrine Mustard, Resorufin, RH 414, Rhod-2, Rhodamine, Rhodamine 110, Rhodamine 123, Rhodamine 5 GLD, Rhodamine 6G, Rhodamine B, Rhodamine B 200, Rhodamine B extra, Rhodamine BB, Rhodamine BG, Rhodamine Green, Rhodamine Phallicidine, Rhodamine Phalloidine, Rhodamine Red, Rhodamine WT, Rose Bengal, S65A, S65C, S65L, S65T, SBFI, Serotonin, Sevron Brilliant Red 2B, Sevron Brilliant Red 4G, Sevron Brilliant Red B, Sevron Orange, Sevron Yellow L, SITS, SITS (Primuline), SITS (Stilbene Isothiosulphonic Acid), SNAFL calcein, SNAFL-1, SNAFL-2, SNARF calcein, SNARFI, Sodium Green, SpectrumAqua, SpectrumGreen, SpectrumOrange, Spectrum Red, SPQ (6-methoxy-N-(3-sulfopropyl)quinolinium), Stilbene, Sulphorhodamine B can C, Sulphorhodamine Extra, SYTO 11, SYTO 12, SYTO 13, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Green, SYTOX Orange, Tetracycline, Tetramethylrhodamine (TAMRA), Texas Red, Texas Red-X conjugate, Thiadicarbocyanine (DiSC3), Thiazine Red R, Thiazole Orange, Thioflavin 5, Thioflavin S, Thioflavin TCN, Thiolyte, Thiozole Orange, Tinopol CBS (Calcofluor White), TMR, TO-PRO-1, TO-PRO-3, TO-PRO-5, TOTO-1, TOTO-3, TRITC (tetramethylrodamine isothiocyanate), True Blue, TruRed, Ultralite, Uranine B, Uvitex SFC, WW 781, X-Rhodamine, XRITC, Xylene Orange, Y66F, Y66H, Y66W, YO-PRO-1, YO-PRO-3, YOYO-1, YOYO-3, SYBR Green, Thiazole orange (interchelating dyes), or combinations thereof.

Another aspect of the invention is directed to a computer-implemented method. For instance, a computer and/or an automated system may be provided that is able to automatically and/or repetitively perform any of the methods described herein. As used herein, “automated” devices refer to devices that are able to operate without human direction, i.e., an automated device can perform a function during a period of time after any human has finished taking any action to promote the function, e.g. by entering instructions into a computer to start the process. Typically, automated equipment can perform repetitive functions after this point in time. The processing steps may also be recorded onto a machine-readable medium in some cases.

For example, in some cases, a computer may be used to control imaging of the sample, e.g., using fluorescence microscopy, STORM or other super-resolution techniques such as those described herein. In some cases, the computer may also control operations such as drift correction, physical registration, hybridization and cluster alignment in image analysis, cluster decoding (e.g., fluorescent cluster decoding), error detection or correction (e.g., as discussed herein), noise reduction, identification of foreground features from background features (such as noise or debris in images), or the like. As an example, the computer may be used to control activation and/or excitation of signaling entities within the sample, and/or the acquisition of images of the signaling entities. In one set of embodiments, a sample may be excited using light having various wavelengths and/or intensities, and the sequence of the wavelengths of light used to excite the sample may be correlated, using a computer, to the images acquired of the sample containing the signaling entities. For instance, the computer may apply light having various wavelengths and/or intensities to a sample to yield different average numbers of signaling entities in each region of interest (e.g., one activated entity per location, two activated entities per location, etc.). In some cases, this information may be used to construct an image and/or determine the locations of the signaling entities, in some cases at high resolutions, as noted above.

In some aspects, the sample is positioned on a microscope. In some cases, the microscope may contain one or more channels, such as microfluidic channels, to direct or control fluid to or from the sample. For instance, in one embodiment, nucleic acid probes such as those discussed herein may be introduced and/or removed from the sample by flowing fluid through one or more channels to or from the sample. In some cases, there may also be one or more chambers or reservoirs for holding fluid, e.g., in fluidic communication with the channel, and/or with the sample. Those of ordinary skill in the art will be familiar with channels, including microfluidic channels, for moving fluid to or from a sample.

The following documents are each incorporated herein by reference in their entireties: U.S. Pat. No. 10,240,146, entitled “Probe Library Construction”; U.S. Pat. Apl. Pub. No. 2017/0220733, entitled “Systems and Methods for Determining Nucleic Acids”; U.S. Pat. Apl. Ser. No. 62/779,333, entitled “Amplification Methods and Systems for MERFISH and Other Applications”; Int. Pat. Apl. Pub. No. WO 2016/018960, entitled “Systems and Methods for Determining Nucleic Acids”; Int. Pat. Apl. Pub. No. WO 2016/018963, entitled “Probe Library Construction”; Int. Pat. Apl. Pub. No. WO 2018/089445, entitled “Matrix imprinting and Clearing”; Int. Pat. Apl. Pub. No. WO 2018/218150, entitled “Systems and Methods for High-Throughput Image-Based Screening”; and Int. Pat. Apl. Pub. No. WO 2018/089438, entitled “Multiplexed Imaging Using MERFISH and Expansion Microscopy.” In addition, each of the following is also incorporated herein by reference in its entirety: U.S. Provisional Patent Application Ser. No. 62/836,578, filed Apr. 19, 2019, entitled “Imaging-Based Pooled CRISPR Screening,” by Zhuang, et al., and U.S. Provisional Patent Application Ser. No. 62/841,715, filed May 1, 2019, entitled “Imaging-Based Pooled CRISPR Screening,” by Zhuang, et al.

The following examples are intended to illustrate certain embodiments of the present invention, but do not exemplify the full scope of the invention.

### Example 1

Pooled-library CRISPR screening provides a powerful means to discover genetic factors involved in cellular processes in a high-throughput manner. However, the phenotypes that are accessible to pooled-library screening are limited. Complex phenotypes such as cellular morphology and subcellular molecular organization, as well as their dynamics, require imaging-based readout and are currently beyond the reach of pooled-library CRISPR screening. These examples show an all imaging-based pooled-library CRISPR screening approach that combines high-content phenotype imaging with high-throughput guide RNA (sgRNA) identification in individual cells. In one such approach, sgRNAs are co-delivered to cells with corresponding barcodes placed at the 3′ untranslated region (3′UTR) of a reporter gene using a lentiviral delivery system with reduced recombination-induced sgRNA-barcode mispairing. Multiplexed Error-Robust Fluorescence In Situ Hybridization (MERFISH) can be used to readout the barcodes and hence identify the sgRNAs with high accuracy. See, e.g., Int. Pat. Apl. Pub. Nos. WO 2016/018960, WO 2016/018963, WO 2018/089445, WO 2018/218150, WO 2018/089438, and WO 2018/089438, each incorporated herein by reference in its entirety. These examples used this approach to screen 162 sgRNAs targeting 54 RNA-binding proteins for their effects on RNA localization to nuclear compartments, and uncover previously unknown regulatory factors for nuclear RNA localization. Notably, these screens revealed both positive and negative regulators for the nuclear speckle localization of a long non-coding RNA (lncRNA), MALAT1, suggesting a dynamic regulation of lncRNA localization in subcellular compartments.

These examples develop an imaging-based pooled-library CRISPR screening approach, which allowed both phenotype and genotype of individual cells to be read out by high-resolution, high-content imaging. This approach promises to substantially expand the phenotype space accessible to pooled genetic screening by allowing complex cellular phenotypes, such as cell morphology, subcellular organization of different molecular species, as well as their dynamics, to be probed. Applying this approach to screen for genetic factors involved in nuclear RNA localization, both positive and negative regulators were identified that controlled lncRNA localization to nuclear speckles.

These examples illustrate an approach for all imaging-based pooled-library CRISPR screening in mammalian cells. This approach allows both high-content phenotype imaging of multiple molecular targets in individual cells and high-accuracy identification of the genotype of each cell, the latter being achieved by associating each sgRNA with unique barcodes and reading out the barcodes using Multiplexed Error-Robust Fluorescence In Situ Hybridization (MERFISH). To illustrate the power of this approach, a genetic screen for factors regulating RNA localization in nuclear compartments was performed. Various nuclear RNAs, such as small nuclear RNAs (snRNAs), small nucleolar RNA (snoRNAs), and long non-coding RNAs (lncRNAs), are associated nuclear compartments formed by liquid-liquid phase separation, such as nucleoli and nuclear speckles. Insights of the spatial regulation of these RNAs are important to understand how they orchestrate diverse nuclear activities and functions, including transcription regulation, transcript processing and genome stability. The effect of 162 sgRNAs (targeting 54 genes) was screened on the localizations of six RNA targets, including the lncRNA MALAT1, the U2 snRNA, and the non-coding RNA 7SK, which are all known to localize to nuclear speckles, the nascent pre-ribosomal RNA and the non-coding RNA MRP, both of which are known to localize to nucleoli, and the poly-A containing RNAs. These results revealed a number of regulators for nuclear RNA localization. In particular, both positive regulators that are essential for the nuclear speckle localization of MALAT1, and negatively regulators that reduce the nuclear speckle localization of MALAT1, were identified, suggesting a dynamic regulation of lncRNA localization.

### Example 2

This example illustrates high-throughput, high-accuracy barcode imaging in mammalian cells. In situ imaging-based pooled-library screening has recently been performed in bacteria, in which the genotypes of individual cells were identified through multiplexed FISH imaging of barcodes associated with the genetic variants. Because of the small volume of bacterial cells, the diffuse signals from barcode RNAs in individual cells are sufficiently strong and can be readily measured. However, the mammalian cell volumes are about a thousand times larger than those of bacteria, making it difficult to achieve a sufficiently high concentration of barcode RNAs to allow a reliable measurement. A new barcode expression and detection scheme is thus needed to both increase the barcode signal and reduce the background for mammalian cells.

To achieve this goal, sgRNAs and a reporter gene were expressed using two independent promoters in the same vector and incorporated a 12-digit ternary barcode in the 3′ untranslated region (3′UTR) of the reporter gene (FIG. 1A). Each digit of the ternary barcode (referred to as a trit hereafter) is made of one of three different readout sequences (30 nucleotides (nt) long) specific to that digit, corresponding to the three possible trit values, 0, 1 and 2. For example, twelve trits have the capacity to encode a total of 3¹²=531,441 barcodes. Because there are a total of 36 different trit sequences (3 different sequences for each of the 12 trits), the barcodes were read using sequential rounds of hybridization to form images with 36 pseudo-color channels (18 rounds of hybridization with 2-color imaging per round, one pseudo-color channel per trit sequence), providing a highly multiplexed detection. To increase the signal from the barcodes, a branched DNA amplification scheme was used to amplify the signal for each trit sequence (FIG. 1A). To reduce interference from background, the mRNA sequence of the reporter gene was co-stained and both the reporter gene sequence and the barcode sequence were detected with single-molecule FISH (smFISH), so that only the barcode signals that colocalized with the reporter gene signals were considered (FIG. 1A). For each specific trit, the trit value (0, 1, or 2) was assigned based on the pseudo-color channel that exhibited the highest fraction of reporter mRNA smFISH signal colocalized with the trit signal. This detection scheme reduced background signals arising from non-specific binding of barcode FISH probes, which is important for decoding accuracy as shown in the following section.

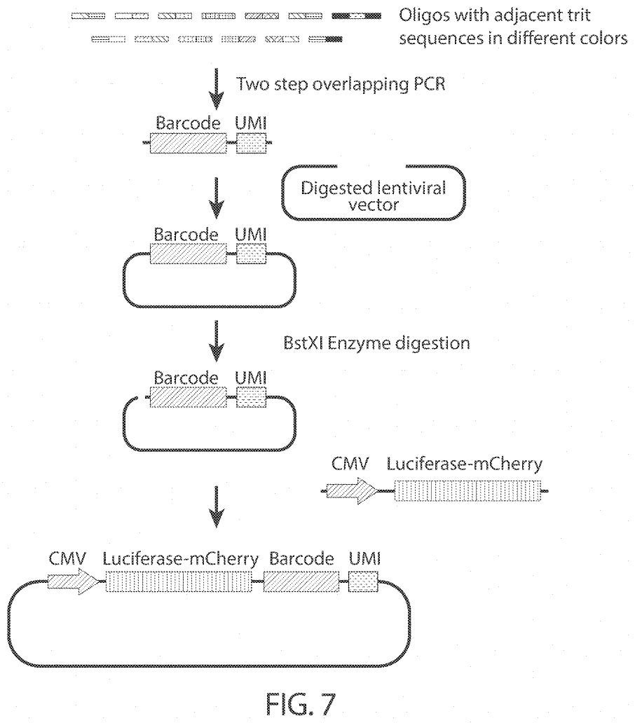

To test this barcode identification scheme, a library of vectors was cloned, each of which contains a common reporter gene, luciferase-mCherry, and a unique barcode under the control of the same promoter, in a pooled manner (FIG. 1B; see below and FIG. 7). Although the total number of possible barcodes exceed 500,000, the library was restricted to only ˜2000 vectors (for error-detection purposes, as described below) and the barcodes in the library were determined by sequencing. The library was delivered into the genome of U-2 OS cells using lentivirus at low multiplicity of infection (MOI) so that most transfected cells received only one barcode. The barcode signals for individual cells were measured using the multiplexed detection scheme as described above.

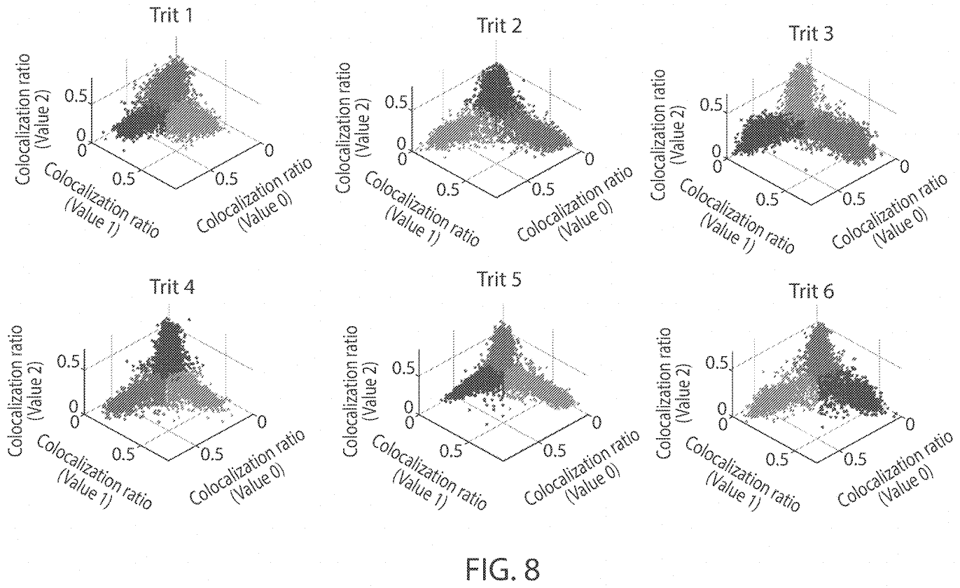

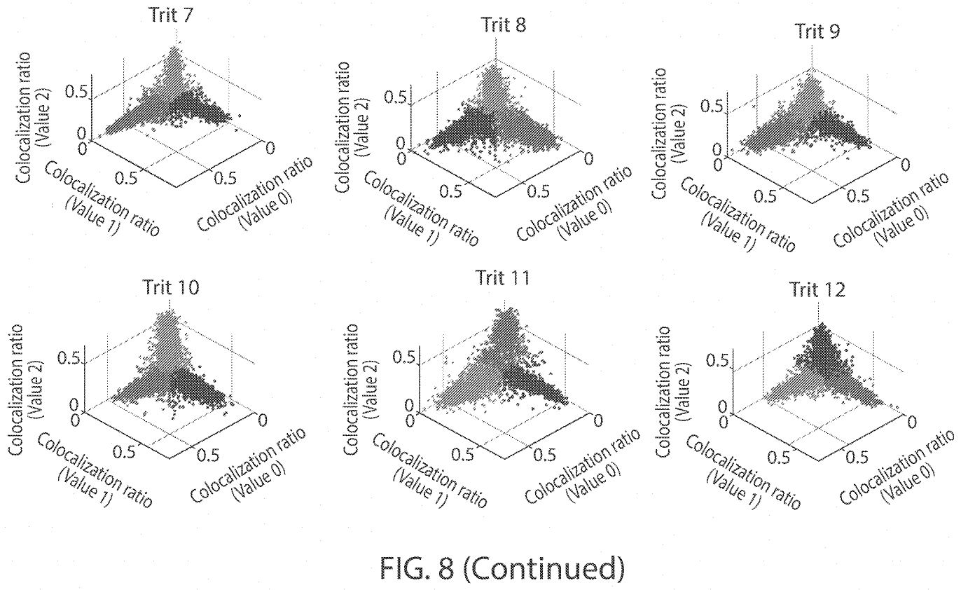

After each round of hybridization, a clear barcode signals colocalizing with the smFISH signals of the reporter gene (luciferase-mCherry) mRNA (FIG. 1C) was observed. For each trit detection, three trit values were separately probed (in different pseudo-color channels as described earlier), and three distinct populations of cells were observed, representing cells expressing barcodes with three different trit values (FIG. 1D; see below and FIG. 8). A k-means clustering algorithm was used to separate the three populations of cells, and a trit value assigned to each population based on which one of the three pseudo-color channels assigned to this trit exhibited the highest fraction of reporter gene mRNA spots that were colocalized the trit signal. The detection of 12 trits using 36 pseudo-color channels allowed a barcode to be assigned each cell. The decoded barcodes for the majority (˜57%) of cells matched the ˜2000 barcodes in the library determined by sequencing (FIG. 1E), and cells with mismatching barcodes were discarded. To assess the improvement in barcode detection accuracy using this reporter gene colocalization approach, the barcode to each cell was also assigned based on the number of FISH spots detected for the barcode signal alone (without considering colocalization with the reporter gene signal). It was found that no decoded barcodes matched the actual barcodes in the library in this case (FIG. 1F), presumably due to background signals introduced by non-specific FISH labeling, illustrating a substantially improved decoding accuracy with the reporter gene colocalization approach.

The bottlenecking strategy that was used (i.e. limiting the total number of vectors in the library to ˜2000, which was only <0.4% of the total possible number of 12-digit ternary barcodes) allowed error detection, since a readout error of any digit would most likely generate an invalid barcode not present in the library. Quantitatively, since only 0.4% of all possible barcodes were present in the libraries, the probability that any erroneously detected barcode would match the barcodes in the libraries is only 0.4%. Thus, among the 57% exact-matched barcodes, only 0.3% could arise from barcode misidentification (see below for a detailed calculation).

FIG. 1 shows imaging-based barcode detection for genotype determination in mammalian cells. FIG. 1 A shows a strategy for high-accuracy imaging-based barcode detection aiming for genotype determination. sgRNA and a reporter gene with an imaging-based barcode are co-delivered into the genome of host cell. The reporter gene portion of mRNA is detected by single-molecule FISH (smFISH) and the barcode is detected by MERFISH, with sequential rounds of hybridization to detect each digit (trit) of the ternary barcode. The barcode signal is amplified using a 4 by 4 branched DNA amplification scheme. FIG. 1B shows a construct design of the reporter gene-barcode library for probing barcode identification accuracy. FIG. 1C, upper panels show example images showing reporter mRNA smFISH signal and the signals for each of the three trit values (0, 1 and 2) for a single trit in the barcode. The lower panels show enlarged views of the white-boxed region of the upper panels, with the reporter gene signal shown on the left and the overlay between the reporter gene signal and the barcode trit signals shown in the right. Trit value 1 has a high colocalization ratio for this cell, whereas Trit values 0 and 2 do not have high colocalization ratios. Scale bars are 10 micrometers. FIG. 1D shows colocalization ratios of the three trit values measured for an example trit for all cells. Each spot in the plots corresponds to a single cell. The colocalization ratio is defined as the number of reporter-gene smFISH spots that are colocalized with trit signal spots divided by total number of reporter-gene smFISH spots within the cell. Cells are partitioned into three clusters (shown in different shadings) based on their colocalization ratios using a k-means clustering algorithm. Each cluster corresponds to cells that have a specific trit value. FIG. 1E shows a histogram of the number of cells with different number of mismatched trits in the decoded barcodes as compared to the valid barcodes in the library. The barcodes were decoded as described above using reporter gene signal and trit signal colocalization. FIG. 1F is the same as FIG. 1E, but with the barcodes decoded by using the number of measured trit signal spots only, without considering reporter gene signal and trit signal colocalization.

FIG. 7 shows the cloning strategy of libraries for evaluation of imaging-based barcode detection accuracy. In brief, the barcode and a UMI (unimolecular identifier) were first assembled from individual pieces of DNA oligos through two-step overlapping PCR (see below). The shadings for different oligos represent different trit sequences. This barcode-UMI library was then inserted into a digested lentiviral plasmid backbone to form a barcode-UMI lentiviral vector library. A reporter gene cassette was further inserted into the barcode-UMI lentiviral vector library to create the final reporter gene-barcode library.

FIG. 8 shows the colocalization ratio analysis of all 12 trits. The colocalization ratio of the three values of individual trits measured for all cells are displayed for all 12 trits. Cell were partitioned into three clusters (shown in different shadings) based on their colocalization ratios using a k-means clustering algorithm. Each cluster corresponds to cells with one trit value.

### Example 3

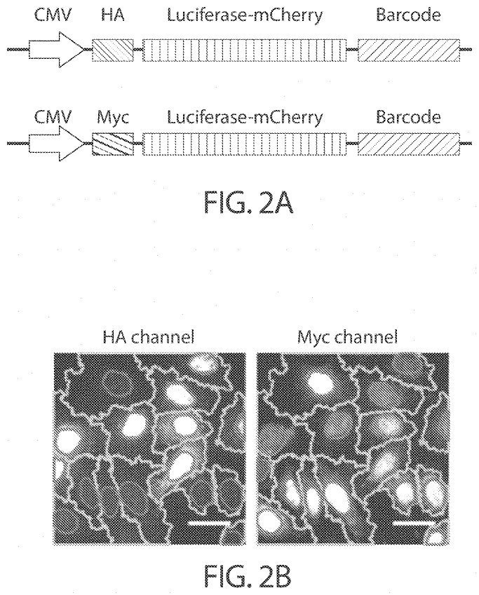

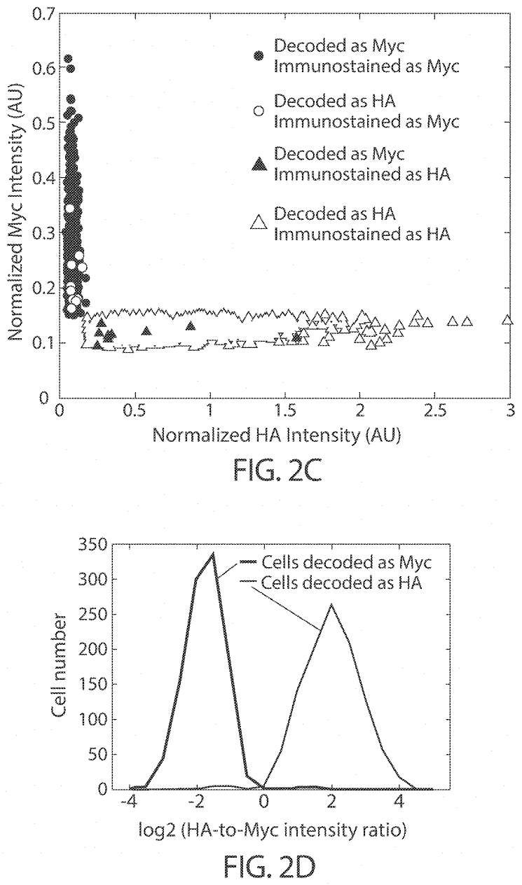

To further validate the low misidentification rate, in this example, two reporter-barcode libraries were designed, each expressing a reporter gene luciferase-mCherry with a distinct epitope tag (a HA tag or a Myc tag) fused to a library of barcodes as described above (FIG. 2A) and the two libraries were cloned separately. Each library was bottlenecked to contain <0.2% of total possible barcodes, so that the same barcodes were highly unlikely to appear in both libraries, and the barcode identities associated with each epitope-tagged reporter gene were determined by sequencing. The two libraries were introduced separately in U-2 OS cells and then the two libraries of cells were pooled together in roughly equal number. The phenotype of each cell, i.e. the expression of HA or Myc tag, was imaged using immunofluorescence (FIG. 2B) and the barcode associated with each cell imaged using sequential rounds of hybridization as described above. The rationale was that determining the phenotype of each cell would allow the deduction of the barcode identity of that cell from the sequencing results, and then comparison with the barcode determined by imaging would allow the determination of the fraction of barcodes that were misidentified.

Only ˜1% of the cells had misidentified barcodes, as judged by barcode-phenotype mismatching (FIGS. 2C and 2D). Even this small error was largely due to errors in cell segmentation, which in turn caused phenotype determination errors, further supporting a very low barcode misidentification rate in these experiments.

FIG. 2 illustrates the evaluation of the barcode misidentification rate using cells with known phenotype-barcode correspondence. FIG. 2A illustrates constructs used to evaluate barcode detection accuracy. The reporter gene luciferase-mCherry is tagged with either a HA or a Myc tag to define two phenotypes, as well as a nuclear localization signal to concentrate HA and Myc signals in the nucleus to facilitate detection. The barcodes are placed at the 3′UTR of the reporter gene, and the correspondence between the barcodes and the HA or Myc tag is determined by sequencing. The reporter gene is driven by a CMV promoter. FIG. 2B illustrates images showing HA and Myc immunostaining signals in two different channels. The nuclei with strong HA signals have weak Myc signals and vice versa. The cell boundaries are highlighted. The nucleus boundaries of HA expression cells and Myc expressing cells are also labeled, respectively. Scale bars are 50 micrometers. FIG. 2C is a scatter plot of the HA and Myc immunostaining intensities of individual cells. Cells assigned to the HA or Myc library based on imaging-based barcode determination are shown. Cells classified as being positive in HA or Myc immunostaining (see below) are shown by triangles or circles, respectively. Only 10 out of 1105 cells with HA-corresponding barcodes were observed as being positive in Myc immunostaining, and only 9 out of 1034 cells with Myc-corresponding barcodes are observed as being positive in HA immunostaining, indicating a low barcode misidentification rate. A small fraction of cells (197 out of 2336 cells) had both their HA and Myc immunostaining signals below a threshold value or both their HA and Myc immunostaining signals above a threshold value, and hence could not be unambiguously identified as being HA-positive or Myc-positive (see below). These cells were excluded from analysis. FIG. 2D shows a histogram of the ratio of HA intensity over Myc intensity for individual cells that were decoded to contain HA tag reporter or Myc tag reporter by barcode imaging.

### Example 4

This example illustrates a lentiviral delivery system with reduced recombination effect for accurate sgRNA identification. Another challenge in sgRNA identification by pooled-barcode imaging arises from the viral system for delivering the sgRNA-reporter gene-barcode vector into the mammalian cells. Lentivirus is a preferred delivery system for mammalian cells because it allows stable genome integration of the vector and the introduction of one sgRNA per cell by transduction at a low MOI (although other delivery systems could also be used in other cases). However, lentivirus has two single-stranded RNA genomes and is prone to recombination, which could lead to mispairing of sgRNA and barcodes during viral transduction. The recombination rate of lentivirus is ˜1 event per kilobase (kb). Because in these experiments, the sgRNA and the reporter gene-barcode combination were separately expressed under two independent promoters, the barcode and sgRNA sequences were separated in these examples by a large genomic distance (>1 kb), and hence the probability for recombination-induced barcode-sgRNA mispairing could be substantial.

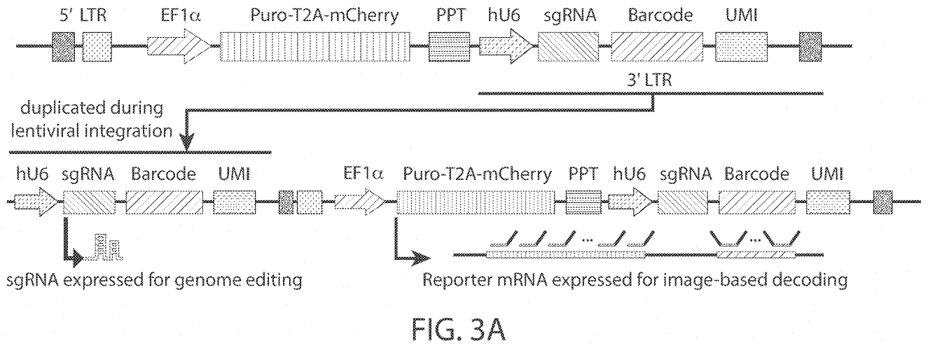

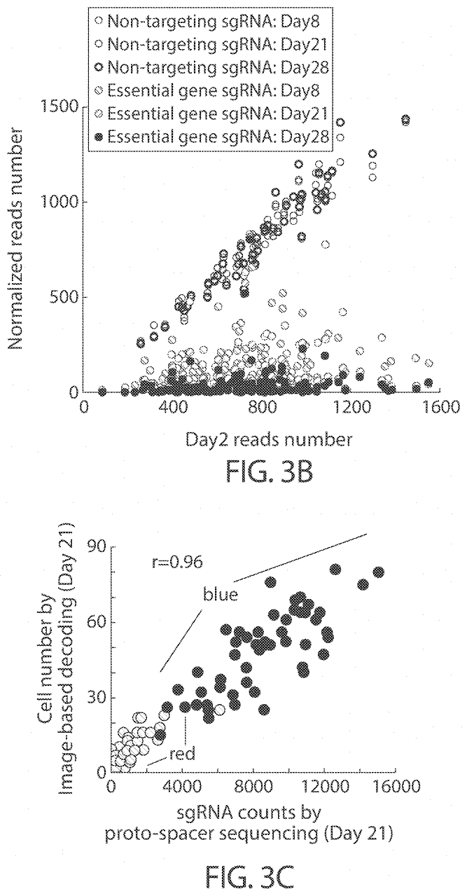

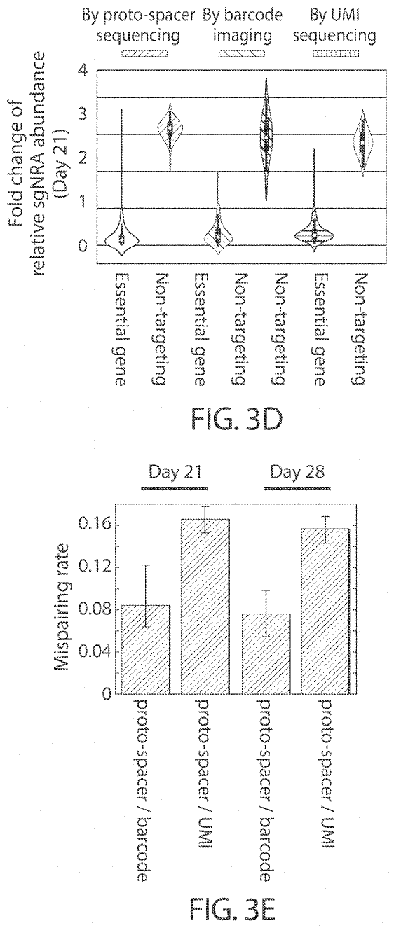

This example illustrates a strategy, modified from the CROP-seq approach, to overcome this recombination problem. Specifically, the reporter gene (puro-T2A-mCherry) was placed under a strong Pol II promoter (EF1α, EF1-alpha), and the sgRNA was placed under a separate promoter (hU6), together with the barcode, downstream of a polypurine tract (PPT) in the lentiviral genome (FIG. 3A). This way, the proto-spacer of sgRNA, a ˜20 nt sequence for specific gene targeting, and the barcode sequence could be separated by a minimal genomic distance (˜100 bases). Although the expression of the sgRNA downstream of the reporter gene could be impaired due to interference from the strong EF1α (EF1-alpha) promoter for the reporter gene expression, the sgRNA expression cassette was duplicated to the 5′ LTR of the proviral genome during genome integration, resulting in an additional functional unit to express sgRNAs that is free of the interference from the EF1α (EF1-alpha) promoter (FIG. 3A). The transcription of reporter gene only stops at 3′ end of the 3′LTR, so the barcode should be expressed in the reporter mRNA 3′UTR for imaging-based barcode identification (FIG. 3A).

To evaluate whether this construct design could support functional lentiviral infection and sgRNA expression, a library containing both sgRNAs targeting genes essential for cell survival and non-targeting control sgRNAs was constructed. An efficient sgRNA expression would cause depletion of cells that express sgRNAs targeting essential genes. 159 sgRNAs targeting 53 essential ribosomal proteins (3 sgRNAs for each gene) as well as 51 non-targeting sgRNAs as controls (Dataset S1) were chosen, and a lentivirus library containing these 210 sgRNAs, together with the reporter gene (puro-T2A-mCherry) and barcodes were generated by pooled cloning (FIG. 3A; see below and FIG. S3). U-2 OS cells stably expressing Cas9-BFP were then infected with this lentivirus library. At day 2 after lentiviral infection, the cells that were both infected by the library and expressed a high level of Cas9 were sorted, based on mCherry and BFP fluorescence, respectively, and these cells were kept for experiments at different time points post infection. The abundance of cells expressing various sgRNAs was determined by sequencing the genomic DNA.

As expected, cells containing sgRNAs targeting essential genes were largely depleted as compared to cells containing non-targeting sgRNAs and the degree of depletion depended on the length of time after lentiviral infection (FIG. 3B), indicating that this viral system could support expression of functional sgRNAs. In addition, the abundance of cells containing different sgRNAs were measured by imaging-based barcode identification, as described above. The abundance of cells containing individual sgRNAs measured by imaging-based barcode identification correlated well with the cell abundance measured by direct sgRNA proto-spacer sequencing (FIG. 3C), further supporting accurate barcode detection.

Next, this experiment was used to evaluate the recombination rate of the constructs. If recombination happens, the barcodes assigned to sgRNAs of essential genes could recombine with non-targeting sgRNAs, which should lead to a higher cell abundance measured by barcode imaging than by proto-spacer sequencing. Similarly, the barcode assigned to non-targeting sgRNAs could recombine with sgRNAs targeting essential genes, leading to a lower cell abundance measured by barcode imaging. Thus, the fold changes of relative cell abundance were measured, between day 2 and day 21 after lentiviral transduction, for both cells containing sgRNAs targeting essential genes and cells containing non-targeting sgRNAs.

As expected, compared to day 2, the relative abundance of cells containing sgRNAs targeting essential genes greatly reduced at day 21, whereas the relative abundance of cells containing non-targeting sgRNAs substantially increased at day 21 (FIG. 3D). Compared to the results obtained by sgRNA sequencing, the fold changes determined by barcode imaging were slightly smaller due to recombination (FIG. 3D). This difference allowed the quantification of the recombination-induced mispairing rate (see below), which was determined to be ˜8% between the sgRNA proto-spacers and barcodes (FIG. 3E).

In addition, the recombination-induced mispairing rate between the sgRNA proto-spacer and a unimolecular identifier (UMI), a 20-nt sequence placed ˜500 bases downstream from the proto-spacer (FIG. 3A), was measured. As expected, due to the larger genomic distance between the UMI and the proto-spacer (˜500 nt), as compared to the genomic distance between the barcode and proto-spacer (˜100 nt), the recombination-induced mispairing rate for the region between the proto-spacer and UMI was larger, ˜16% (FIGS. 3D and 3E). It was noted that, for a random pair of barcodes, the probability that these barcodes share the same sequence at any giving trit position is about ⅓ because there are three possible sequences for any given trit and because the barcodes in the bottlenecked library were a randomly selected subset of all possible barcodes. Thus, it was estimated that the recombination rate in the barcode region should be roughly ⅓ of the recombination rate for the fully homologous sequence of the same length. Based on the ˜8% recombination rate that was measured for the ˜100-nt genomic region between the barcode and proto-spacer (the common sequence of sgRNAs), it was estimated that the recombination rate in the ˜400-nt barcode region was roughly (400/100)×8%/3=10.7%, which would give a recombination rate of ˜8%+10.7%=18.7% for the genomic region between the proto-spacer and UMI, consistent with the measured value of ˜16%. Furthermore, since the barcode library was bottlenecked, the recombination that occurred within the barcode region was unlikely to generate a new barcode that matched with the valid barcodes in the library, and thus would unlikely lead to barcode misidentification.

Together, the low error rate in barcode imaging (<1%) and the low mismatching rate between sgRNA and barcode induced by recombination (˜8%) allowed a high accuracy in sgRNA identification by barcode imaging, which in turn allowed an all imaging-based pooled-library CRISPR screening. It was noted that although the remaining 8% mismatching rate between sgRNA and barcode could potentially generate false positives and negatives in the screening, the error rate would be minimal because hundreds of cells carrying the same sgRNA were typically probed to determine whether a sgRNA had a statistically significant effect; moreover, three sgRNAs targeting each gene were probed, and only genes were considered to be hits when two out of the three sgRNAs exhibited a statistically significant effect. Any remaining false positives could be readily identified by validation experiments.

FIG. 3A illustrates the design of lentiviral delivery approach with a low rate of recombination-induced sgRNA-barcode mismatch. FIG. 3A shows lentiviral constructs used to deliver sgRNA and barcode for sgRNA identification. A sgRNA cassette (hU6 promoter with sgRNA) and barcode array is placed downstream of polypurine tract (PPT). A strong Pol II promoter (EF1α, EF1-alpha) drove the expression of the reporter gene, puro-T2A-mCherry. After genome integration, the sgRNA cassette was duplicated into the 5′LTR for sgRNA expression while the barcode is expressed with the reporter gene at 3′UTR for barcode imaging. UMI: Unique molecular identifiers. FIG. 3B shows proto-spacer counts of each sgRNA at day 8, day 21 and day 28 after lentivirus transduction are plotted against the proto-spacer counts measured at day 2 after transduction. The proto-spacer counts at day 8, day 21 and day 28 were normalized by factors so that the mean counts for the non-targeting sgRNAs for these conditions were the same as the mean counts for the non-targeting sgRNAs at day 2. The proto-spacer counts were determined by sequencing. As expected, the cells expressing sgRNAs targeting essential ribosomal genes were strongly depleted over time and hence the counts of sgRNAs targeting essential genes were much reduced compared to the non-targeting control sgRNAs. FIG. 3C shows correlation between the number of cells expressing certain sgRNAs as measured by imaging-based barcode detection and the sgRNA counts measured by proto-spacer sequencing, at day 21 after lentivirus transduction. sgRNAs targeting essential genes are generally labeled red, and non-targeting control sgRNAs are generally labeled as blue. FIG. 3D are violin plots showing the median fold change of the relative sgRNA abundance between day 2 and day 21 after lentivirus transduction measured by proto-spacer sequencing, imaging-based barcode detection and UMI sequencing. The relative abundance of a certain sgRNA is defined as the fraction of total sgRNA reads that correspond to this specific sgRNA (i.e. the proto-spacer (or UMI) reads determined by sequencing for this particular sgRNA normalized by the total proto-spacer (or UMI) reads for all sgRNAs, or the number of cells expressing the barcode corresponding to this sgRNA determined by imaging normalized by the total cell number). As expected, the relative abundance of sgRNAs targeting essential genes reduced over time and the relative abundances of non-targeting sgRNAs increased. Due to the recombination, the fold changes determined by barcode imaging was slightly smaller than those determined by proto-spacer sequencing, and the fold change determined by UMI sequencing was slightly smaller than those determined by barcode imaging. FIG. 3E shows the median mispairing rates between the proto-spacers and barcodes and between proto-spacers and UMI due to recombination, determined at 21 and 28 days post lentivirus transduction. The error bars show the 95% confidence interval.

### Example 5

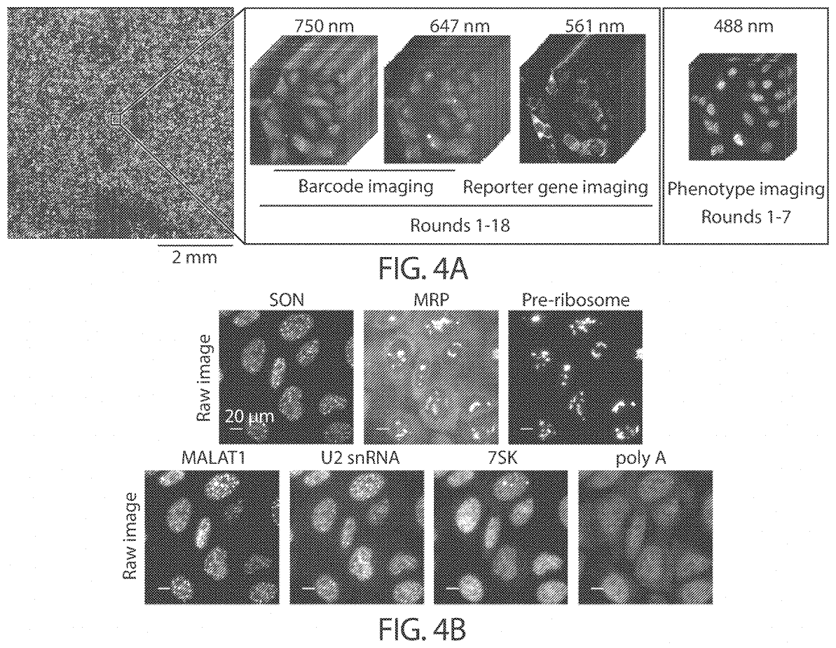

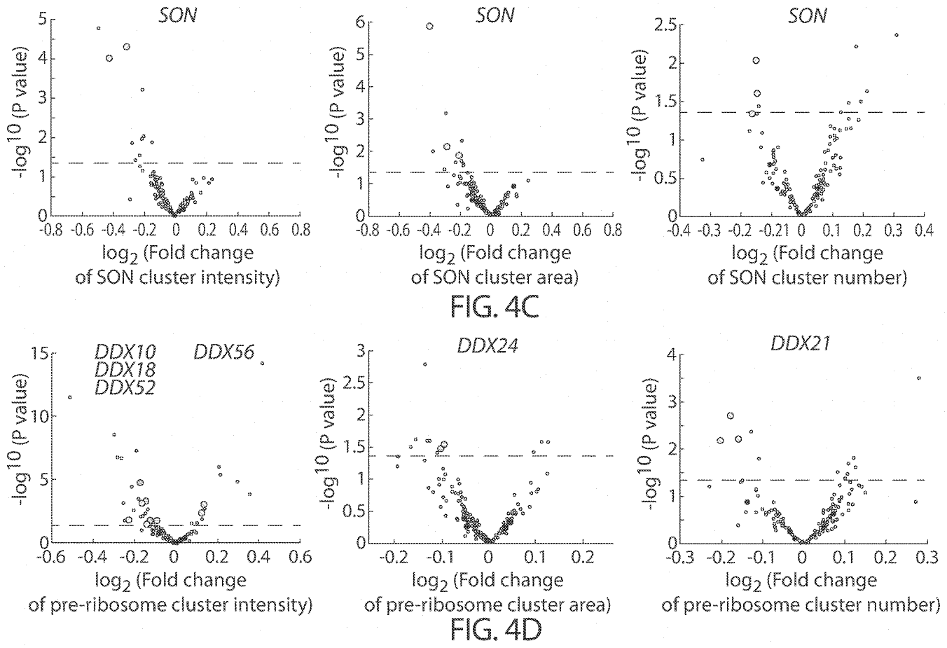

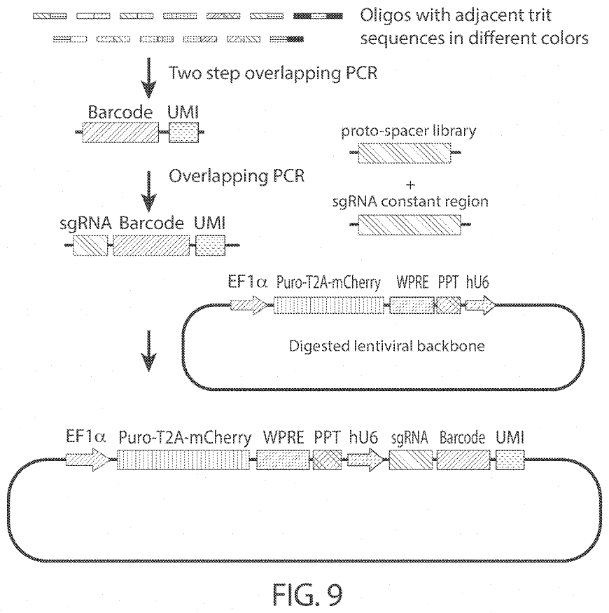

This example illustrates pooled CRISPR screening for factors regulating nuclear RNA localization. To illustrate the power of this screening method, potential regulators of RNA localization were screened in the nucleus (FIG. 4A). 54 candidate genes involved in nuclear RNA regulation were selected, including hnRNP family proteins, DExD/H box RNA helicases, and genes involved in RNA modification (Dataset S2). A library of 167 sgRNAs was designed, containing three sgRNAs for each of the 54 genes and five non-targeting sgRNAs as controls, and a lentivirus library containing these sgRNAs was generated, together with the reporter gene (puro-T2A-mCherry) and barcodes, by pooled cloning (see below and FIG. 9). To demonstrate the ability of this method to assess complex phenotypes, the spatial distributions of five specific RNA species were imaged, the lncRNA MALAT1, the U2 snRNA, 7SK, MRP, and the nascent pre-ribosome, as well as the poly-A containing RNAs, using FISH. In addition, also included in the phenotype imaging was a nuclear speckle protein, SON, using immunolabeling with an oligonucleotide-conjugated antibody. These RNA and protein targets were imaged, along with barcode imaging, using sequential rounds of hybridization with 3-4 different color channels per round (FIG. 4A) (see below for details of the imaging procedure).

As expected, the protein SON exhibited a clustered distribution that marked the nuclear speckles, and the MRP and pre-ribosome signals marked the subnucleolar compartments (FIG. 4B). Based on these images, the boundaries of these structures were identified, and their numbers, the areas covered by them, and their mean signal intensities (i.e. total signals localized within the identified cluster boundaries divided by total area covered by these clusters) were determined in individual cells. Next, the enrichment of MALAT1, U2, 7SK and poly-A containing RNAs in the nuclear speckles identified by the SON staining was quantified (see below).

For each of these feature quantifications, the values determined for cells harboring a targeting sgRNA were compared with the values measured from cells harboring non-targeting control sgRNAs to determine the fold change. 4 biological replicates of experiments were performed and a total of ˜30,000 cells was decoded, and hits based on the criterion that at least two of three sgRNAs targeting the gene exhibited a statistically significant fold change were determined (Dataset S3).

As a positive control, a statistically significant decrease of cluster signal intensity, cluster area and cluster number associated with the SON stain in cells expressing sgRNAs targeting SON (FIG. 4C) was detected. In addition, sgRNAs for several DExD/H box RNA helicases (DDX10, DDX18, DDX21, DDX24, DDX52 and DDX56) caused statistically significant changes in various features of the nascent pre-ribosome stain (FIG. 4D), consistent with the known functions of these genes in ribosome biogenesis. It was noted that the magnitudes of change in these phenotype features were moderate (FIGS. 4C and 4D), potentially because not all cells expressing the sgRNAs had their genome edited. Thus, these quantifications allowed the identification of genetic perturbations that had a statistically significant effect, but the magnitudes of the phenotype changes were less informative. It was also noticed that the perturbation of several genes in the hnRNP family caused significant changes in the pre-ribosome and MRP signals in the nucleoli (Dataset S3), potentially due to indirect effects.

FIG. 4 shows imaging-based pooled CRISPR screening for regulators of nuclear RNA localization. FIG. 4A shows a scheme of imaging-based screening. Cells infected with lentiviruses expressing sgRNAs, barcodes and the reporter gene were fixed and imaged. The barcodes were imaged by MERFISH using 647-nm and 750-nm color channels in 18 rounds of hybridization (rounds 1-18). To increase the accuracy of barcode imaging, the reporter gene mRNA was imaged in every round (rounds 1-18) using the 561-nm color channel to allow the determination of colocalization between barcode and reporter gene mRNA signals. The 7 protein and RNA targets for phenotype measurements are imaged in the 488-nm color channel in the first 7 rounds (rounds 1-7). The mosaic on the left contains 900 fields-of-view from a single screen. FIG. 4B shows phenotype images of SON, MRP, pre-ribosome, MALAT1, U2 snRNA, 7SK and polyA-containing RNAs. SON marks nuclear speckles, pre-ribosome and MRP mark subnucleolar structures. For SON, pre-ribosome, and MRP, the cluster numbers, cluster areas and cluster intensities are quantified. For MALAT1, U2 snRNA, 7SK and polyA-containing RNAs, their enrichments in nuclear speckles are quantified. Scale bars are 20 micrometers. FIG. 4C shows volcano plots for the effect of each sgRNA on SON cluster intensity, cluster area and cluster number. FIG. 4D shows volcano plots for the effect of each sgRNA on pre-ribosome cluster intensity, cluster area and cluster number. In FIGS. 4C and 4D, the fold change induced by each sgRNA is calculated as the mean value from all cells containing this sgRNA divided by the mean value from all cells containing non-targeting sgRNAs. The horizontal dashed lines indicate the p value (0.05) used to define hit of the screen. The data points of the indicated hits (i.e. two of the three sgRNAs targeting the gene show statistically significant fold changes (p<0.05)) are shown in shadings that match the shadings of the gene names shown in the legend and data points for other gene-targeting sgRNAs are shown in grey. Data points for non-targeting sgRNAs are shown in black.

FIG. 9 shows a cloning strategy of lentiviral sgRNA-barcode delivery library. In brief, the barcode and UMI were first assembled from individual pieces of DNA oligos through two-step overlapping PCR and then assembled with the proto-spacer sequences and sgRNA constant region sequence using overlapping PCR to form a sgRNA-barcode-UMI cassette library. The shadings for different oligos represent different trit sequences. This library was then inserted in to a digested, reporter gene containing lentiviral backbone with the hU6 promoter at the site downstream of the polypurine tract (PTT).

### Example 6

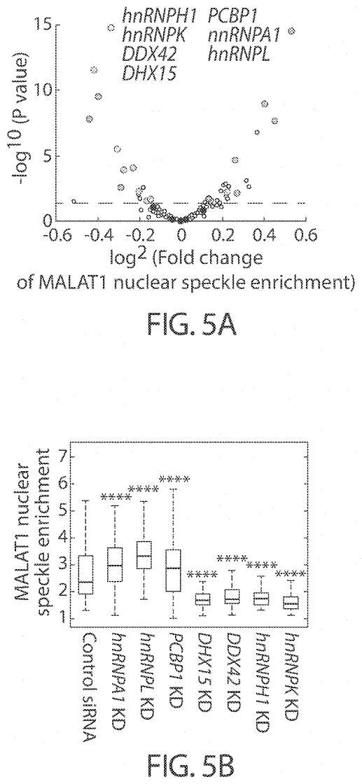

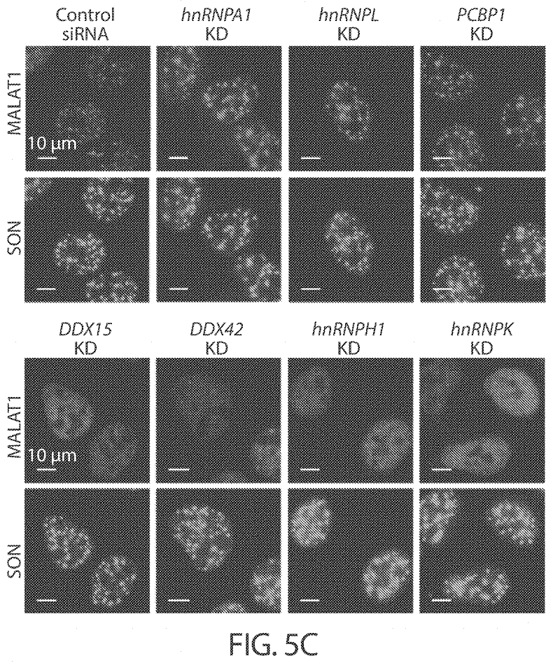

This example illustrates that novel factors are involved in the regulation of MALAT1 nuclear speckle localization. This screening revealed genes involved in regulation of nuclear speckle localization of different RNA species (Dataset S3). Compared to 7SK, U2 snRNA and poly-A containing RNAs, more genes were identified that regulate MALAT1 localization. This discussion focuses on MALAT1. Notably, two groups of genes were identified that regulate the nuclear speckle localization of MALAT1 in opposite directions (FIG. 5A; Dataset S3), which were validated for all but one gene (hnRNPH3) by siRNA-mediated knockdown (FIGS. 5B and 5C). It was not confirmed whether the siRNA for hnRNPH3 was effective due to the lack of an effective antibody for this protein. Depletion of the first group of genes, DHX15, DDX42, hnRNPK and hnRNPH1, caused a statistically significant reduction in the enrichment of MALAT1 in nuclear speckles (FIG. 5A-5C), suggesting that these genes upregulate the nuclear speckle localization of MALAT1. DHX15 and DDX42 are involved in spliceosome recycling and assembly, respectively, which is consistent with the involvement of mRNA splicing factors in recruiting MALAT1 into nuclear speckles. The involvement of the hnRNP family proteins, hnRNPH1 and hnRNPK, in the upregulation of nuclear speckle localization of MALAT1 has not been reported previously. These two genes were also found to affect the localization of other RNA species including U2 snRNA, poly-A containing RNAs, pre-ribosome RNA and MRP (Dataset S3), which could imply a global effect of the perturbations of these two genes.

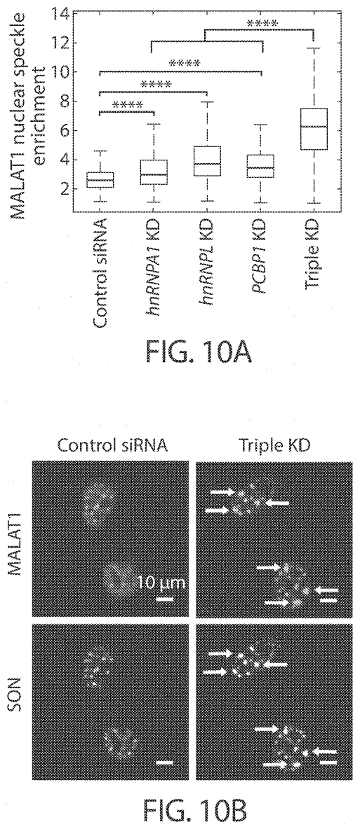

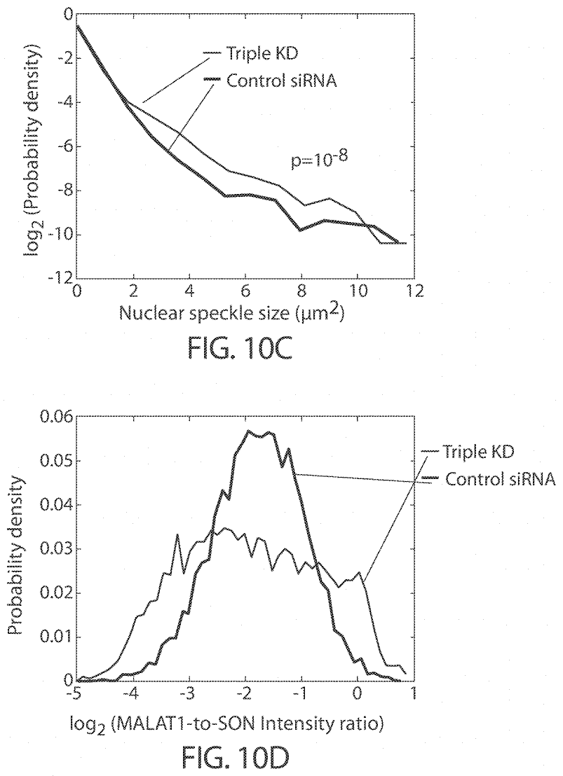

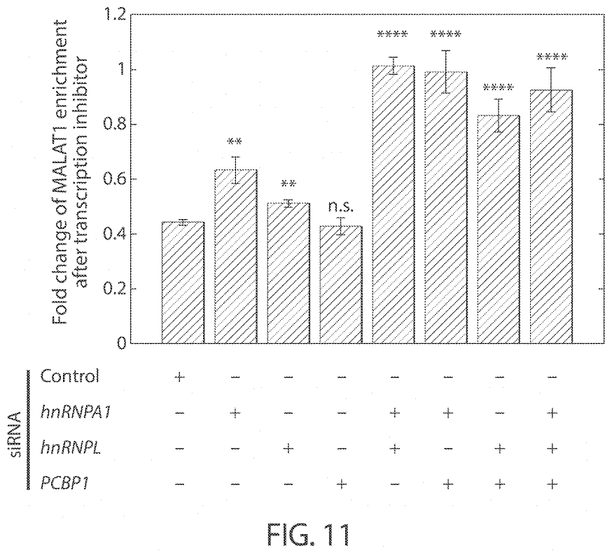

Unexpectedly, three factors, hnRNPA1, hnRNPL and PCBP1, were also identified that negatively regulate the nuclear speckle localization of MALAT1. Their depletion by sgRNA or siRNA induced a statistically significant increase of MALAT1 enrichment in nuclear speckles (FIG. 5A-5C). The fold change of the MALAT1 enrichment induced by siRNA could be an underestimation due to incomplete knockdown. Combined knockdown of all these three factors further increased MALAT1 enrichment in nuclear speckles (see below, FIG. 10A), which, interestingly, also resulted in enlargement of a fraction of nuclear speckles (see below, FIGS. 10B and 10C). The composition of each nuclear speckle, measured by the ratio of MALAT1 and SON levels in the nuclear speckle, also became more heterogeneous in the triple knockdown sample: some speckles had a reduced MALAT1-to-SON ratio, whereas some had an increased ratio (see below, FIG. 10D). These results indicated that the enhanced nuclear speckle localization of MALAT1 upon the knockdown of the three negative regulators is associated with a change of nuclear speckle morphology and composition. This suggested a role of MALAT1 in regulating nuclear speckle structures, which is consistent with the observation that MALAT1 knockdown can lead to a reduction in nuclear speckle size.

FIG. 5 shows genetic factors involved in the regulation of MALAT1 nuclear speckle localization. FIG. 5A shows a volcano plot for the effect of each sgRNA on MALAT1 nuclear speckle enrichment. The fold change is calculated as described in FIG. 4. The horizontal dashed line indicates the p value (0.05) used to define hit of the screen. The hits confirmed by siRNA knockdown are highlighted in shadings that match the shadings of the gene names shown in the legend and data points for other gene-targeting sgRNAs are shown in grey. Data points for non-targeting sgRNAs are shown in black. FIG. 5B shows boxplots showing the effect of siRNA knockdown of the 7 hit genes on MALAT1 localization, alongside data for a control, non-targeting siRNA. The middle lines show the median, the boxes show the 25-75% quartile and the whiskers show the maximum and minimum values. 100-300 cells are quantified for each condition. Student's t tests are performed for each condition in comparison with the control. ****, p<0.0001. FIG. 5C shows images of MALAT1 localization upon siRNA knockdown of the 7 hit genes. Data from a control non-targeting siRNA is also shown. MALAT1 staining is shown in upper images, and SON staining is shown in lower images. Scale bars are 10 micrometers.

FIG. 10 shows triple knockdown of hnRNPA1, hnRNPL and PCBP1 affects the morphology and composition of nuclear speckles. FIG. 10A shows boxplots showing the effect of control siRNA and HNRNPA1, HNRNPL, and PCBP1 single and triple knockdown (KD) on MALAT1 localization. Boxplot elements are as described in FIG. 5. 100-300 cells are quantified for each condition. Student's t tests are performed between each single KD and the non-targeting control and between the triple KD and the hnRNPA1, hnRNPL or PCBP1 single KD. ****, p<0.0001. FIG. 10B shows example images for cells showing that some of the MALAT1-positive nuclear speckles are enlarged (highlighted by arrows) after hnRNPA1, hnRNPL and PCBP1 triple KD, as compared to the cells transfected with control nontargeting siRNA. Scale bars are 10 micrometers. FIG. 10C shows the distribution of nuclear speckle size shows that triple KD of hnRNPA1, hnRNPL and PCBP1 increase the nuclear speckle size. The two-sample Kolmogorov-Smirnov test was used to test the difference between two distributions. FIG. 10D shows distributions of log 2 (MALAT1-to-SON intensity ratio) in each nuclear speckle for control siRNA and hnRNPA1, hnRNPL and PCBP1 triple KD samples. ˜300 cells and ˜7000 speckles are measured for control and triple KD conditions, respectively.

### Example 7

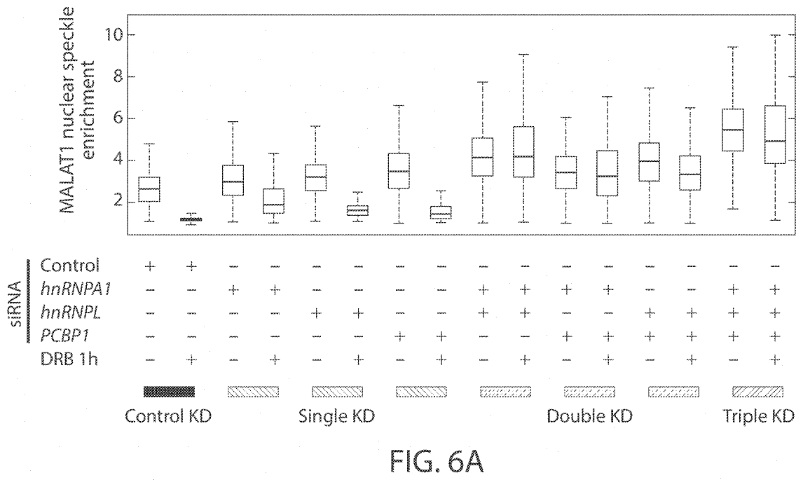

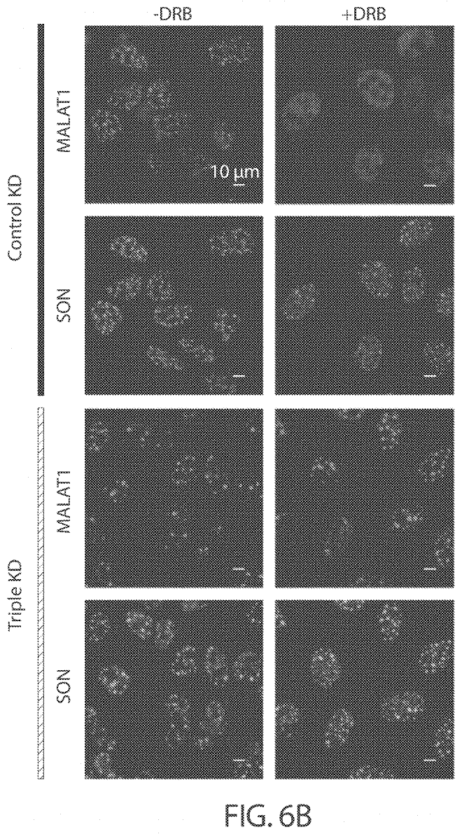

It has been shown previously that nuclear speckle localization of MALAT1 can be impaired under transcription inhibition. However, the genetic factors involved in this process are largely unclear. Thus, whether these three negative regulators play a role in this process were tested. To this end, in this example, the drug 5,6-dichloro-1-β (beta)-D-ribofuranosylbenzimidazole (DRB) was added to inhibit transcription and a substantial reduction in the MALAT1 enrichment in nuclear speckles was observed. Single knockdown of the hnRNPA1, hnRNPL and PCBP1 did not substantially rescue the DRB-induced dissociation of MALAT1 from nuclear speckles (FIGS. 6A and 11, also see below). On the other hand, double knockdown of two of these three factors or triple knockdown of all three factors largely rescued this DRB-induced dissociation effect (FIGS. 6A, 6B, and 11, also see below), suggesting that these hnRNP family proteins are required for transcription inhibition-induced disassociation of MALAT1 from nuclear speckles, and that these factors likely play redundant roles in this process. These results provide a potential mechanism for the dissociation of MALAT1 from nuclear speckles by transcription inhibition. During transcription inhibition, RNA-binding proteins such as hnRNPA1 and hnRNPL are freed from nascent mRNA transcripts to allow their binding to other RNA species. It is thus possible that the freed hnRNPA1 and hnRNPL could bind to MALAT1, which may compete with factors that recruit MALAT1 to nuclear speckles, thereby preventing the nuclear speckle localization of MALAT1 under transcription inhibition.

FIG. 6 shows that hnRNPA1, hnRNPL and PCBP1 are required for transcription inhibition induced dissociation of MALAT1 from nuclear speckles. FIG. 6A shows quantifications of MALAT1 nuclear speckle enrichment with or without transcription inhibitor DRB treatment (50 micromolar, 1 h) for cells transfected by different combination of siRNAs. 100-300 cells are quantified for each condition. The transcription inhibition induced dissociation of MALAT1 from nuclear speckles is not rescued by single knockdowns of hnRNPA1, hnRNPL and PCBP1, but is rescued by the double-knockdown and triple-knockdown of these factors. FIG. 6B shows images showing that in cells transfected by control siRNAs, MALAT1 dissociates from nuclear speckles upon transcription inhibition; whereas in cells co-transfected by siRNAs targeting hnRNPA1, hnRNPL and PCBP1, transcription inhibition fails to dissociate MALAT1 from nuclear speckles. Scale bars are 10 micrometers.

FIG. 11 shows the fold change of MALAT1 nuclear speckle enrichment after transcription inhibition under different knockdown conditions. Bar chart showing the fold change of MALAT1 nuclear speckle enrichment after transcription inhibition under the respective knockdown conditions. This fold change is defined as MALAT1 nuclear speckle enrichment after transcription inhibition divided by the enrichment before transcription inhibition under the same siRNA treatment. Control siRNA, hnRNPA1, hnRNPL and PCBP1 single knockdown as well as hnRNPA1, hnRNPL and PCBP1 double and triple knockdown conditions are shown. Error bar: SD. Student's t tests are performed for each condition with respect to control. **, p<0.01, ****, p<0.0001, n.s., not significant. n=3, each experiment contains 30-100 cells.

### Example 8

These examples developed an imaging-based pooled-library CRISPR screening method, which allowed genotype-phenotype correspondence to be established for individual cells and allowed high-throughput screening of mammalian cells based on complex phenotypes that were previously inaccessible to pooled-library screening. This imaging-based screening is allowed by sgRNA identification through MERFISH-based barcode detection and a barcode misidentification rate as low as ˜1% was demonstrated. A lentiviral delivery scheme was devised with reduced rate of recombination-induced mispairing of sgRNAs and barcodes (mispairing rate<10%). Together, these provided high accuracy sgRNA identification through barcode imaging. These approaches substantially expanded the phenotype space accessible for pooled-library screening. Compared to imaging-based screening using the arrayed format in which individual genetic perturbations are separately assayed in individual wells, a major advantage of performing pooled screening is that the reagents for genetic perturbations, i.e. the DNA plasmids and lentiviruses, can be prepared in a pooled manner with standard molecular biology procedures with reduced labor and cost, which is particularly beneficial for large-scale custom-designed libraries. Reagent preparation for arrayed screening typically requires costly multi-well robotic processing system and more complicated procedures. Another advantage of the pooled approach is that the variation in experimental conditions for different perturbations can be minimized since the measurements for all genetic perturbations are performed in the same experiment. This is particularly desirable when the cells should be treated with concentration or time sensitive conditions. Moreover, the pooled format can also simplify multiplexed phenotype measurements that require sequential rounds of staining and signal removal through buffer exchange. On the other hand, when generating individual genetic perturbation reagents is not especially demanding and when the phenotype measurement is not very sensitive to variations in sample treatment conditions, arrayed screening could be preferred because the MERFISH barcode readout process substantially increases the complexity of the imaging procedure.

The current 12-digit ternary barcode library contains more than half-million barcodes. Even with a stringent 1% bottlenecking strategy to enable error-robust barcode detection, more than 5000 distinct sgRNAs can be included in each library and this capacity can be readily increased by adding more digits to the barcodes. A current limitation for the number of sgRNAs that can be screened is the time required to image a large number of cells. This imaging system utilizes a high magnification (60×) objective to readout the FISH signal on individual single mRNA molecules for barcode detection, limiting the number of cells that can be imaged in each field-of-view. However, the imaging speed could be substantially improved by the following approaches: 1) using greater amplifications for the barcode signal, which allows each field-of-view to be captured with a faster frame rate and/or allows more cells to be imaged in each field-of-view by using lower magnification objectives; 2) using multiple cameras for detection, which allows simultaneous detection of fluorescence signals in different color channels. With these improvements, a more than 10-fold improvement in the number of cells and genotypes that can be screened per experiment can be achieved.

To demonstrate the power of this approach for screening complex phenotypes of mammalian cells, subcellular localizations of 7 different molecular species were imaged, including 6 RNAs and a protein. These screening experiments revealed previously unknown regulators of nuclear RNA localization. Interestingly, both positive and negative regulators of the nuclear-speckle-localization of the lncRNA MALAT1 were identified. The positive regulators included DExD/H box RNA helicases, DHX15 and DDX42, and hnRNP family genes, hnRNPH1 and hnRNPK; whereas the negative regulators included hnRNPA1, hnRNPL and PCBP1. RNAs can be localized to cellular compartments formed by phase separation via at least two mechanisms: 1) RNAs can act as a scaffold, which could nucleate phase separation, such as mRNAs in P body and stress granules and pre-ribosome RNAs in nucleoli; 2) RNAs can be recruited to the phase-separated bodies as clients, which has been shown to be responsible for the localization of MALAT1 in nuclear speckles. It is possible that the negative regulators discovered in this screening could compete with the factors that recruit MALAT1 to nuclear speckles, thereby preventing the nuclear speckle localization of MALAT1. Also identified was a role of these negative regulators in the dissociation of MALAT1 from nuclear speckles induced by transcription inhibition. These results suggest that lncRNA localization could be dynamically regulated by protein factors.

These results demonstrate the ability of this imaging-based screening method to reveal molecular factors involved in cellular processes that can only be assessed by high-resolution imaging. This screening method is broadly applicable to interrogating genetic factors controlling or regulating a broad spectrum of phenotypes, including morphological features, molecular organizations, and dynamics of cellular structures, as well as cell-cell interactions. This screening approach can also be combined with highly multiplexed DNA, RNA and protein imaging approaches, including genomic-scale imaging approaches, to profile factors involved in gene regulation and other genomic functions in a high-throughput manner.

### Example 9

This example illustrates various materials and methods used in these examples.

The cloning of the reporter-barcode libraries and sgRNA-reporter-barcode libraries were performed in pooled manner using oligos ordered from IDT (Datasets S1, S2 and S4). These libraries were cloned into the lentiviral vector pFUGW as described below. The identities of barcodes present in the libraries and the barcode-sgRNA correspondence were established using high-throughput sequencing. Lentivirus were produced in LentiX cells (Takara, 632180) using Lenti-X™ Packaging Single Shots (VSV-G) (Takara, 631276). The lentiviral libraries were used to infect the U-2 OS cells at a low multiplicity of infection (MOI) so that only 10-20% of the cells were infected. The infected cells were sorted based on mCherry expression and Cas9-BFP expression. The sorted cells were fixed, permeabilized and stained for imaging according to detailed methods discussed below.

A custom microscope built around a Nikon Ti-U microscope body with a Nikon CFI Plan Apo Lambda 60× oil immersion objective with 1.4 NA was used for imaging. For sequential rounds of hybridization and imaging, a peristaltic pump (Gilson, MINIPULS 3) pulled liquids (TCEP buffer for dye cleavage, hybridization buffer with readout probes or hybridization buffer for sample wash) into Bioptech's FCS2 flow chamber with sample coverslips, and three valves (Hamilton, MVP and HVXM 8-5) were used to select the input fluid (see details below). The barcode decoding and phenotype quantification based on collected images are also described in detail below.

Cell culture. U-2 OS cells were cultured in EMEM medium (ATCC, HTB-96) supplemented with 10% FBS (Sigma, F4135-1L) and 1% Pen/Strep (Invitrogen, 15140122) antibiotics at 37° C. U-2 OS cells stably expressing Cas9-BFP were generated through lentivirus transduction followed by FACS sorting using BFP signal. To generate the lentivirus vector for Cas9-BFP, the Cas9-BFP sequence was PCR amplified from pLentiCas9-BFP (Addgene #78545) and cloned into pFUGW backbone with SVVF promoter. Two nucleus localization signal sequences were added to enhance the nucleus localization of Cas9.

Vector library cloning. The 12-digit barcodes were each comprised of twelve 30-nt sequences, each of the 30-nt sequence representing a trit, with a nucleotide ‘A’ separating adjacent trits. Oligos encoding each pair of adjacent 30-nt sequences were ordered from IDT in forward and reverse direction alternatively (i.e. trit1+trit2, trit2+trit3 reverse complement, trit3+trit4, trit4+trit5 reverse complement, . . . , trit9+trit10, trit10+trit11 reverse complement, trit11+trit12, see Dataset S4). Each trit has three different values, represented by three distinct 30-nt sequences, hence each pair of adjacent 30-nt sequences has 9 possible different sequences, and total 11×9=99 oligos were needed to cover all possible pairs of adjacent 30-nt sequences. At both ends of the barcodes (represented by Oligos 1-9 and 91-99), two constant primer binding sequences were added for PCR amplification purpose. The sequences of these 99 oligos are described in Dataset S4. The whole barcode library was assembled by two-step overlapping PCR. First, 12 trits were divided into 3 segments, and each segment was generated by the following reactions: Segment 1. Oligos 1-36 as templates, Oligos 1-9 as forward primers, Oligos 28-36 as reverse primers; Segment 2. Oligos 37-72 as templates, Oligos 37-45 as forward primers, Oligos 64-72 as reverse primers; Segment 3. Oligos 73-99 as templates, Oligos 73-81 as forward primers, Oligo 100 as reverse primers. The three PCR products were gel purified. Then, the three segments were mixed and subjected to overlapping PCR using forward primer Oligo 101 and reverse primer Oligo 102. The reverse primer of this step contained a random sequence region of 20 bases which served as the unimolecular identifier (UMI) for the sequencing step. The sequences of oligos 100-102 are also described in Dataset S4. All PCR reactions were performed using real-time qPCR equipment to monitor the reactions so that the reactions were stopped at log-growth phase to reduce library skewing resulted from PCR bias. The PCR products were assembled into a modified pFUGW backbone through isothermal assembly. The assembled library was electroporated into Endura electrocompetent cells (Lucigen, 60242-2) which were then grown under ampicillin selection overnight to amplify the library. The amplified library was purified by mini prep. This library is named pFUGW_barcodes_UMI_backbone library.

The pFUGW_barcodes_UMI_backbone library was then used to generate a library that additionally contain a reporter gene (Luciferease-mCherry) for barcode imaging. The reporter cassettes containing CMV promoter and the reporter open reading frames were first generated in intermediate vectors. The open reading frames contain a luciferase-mCherry, a 2×HA or 2× Myc tag at the N-terminus, and a nuclear localization signal at the C-terminus. The reporter cassettes were PCR amplified from the intermediate vectors using Oligos 103 and 104 (sequence provided in Dataset S4). The pFUGW_barcodes_UMI_backbone library was then digested with BstXI and treated with alkaline phosphatase and assembled with the reporter cassettes PCR products using isothermal assembly. The assembled libraries were electroporated into Endura electrocompetent cells which were then grown under ampicillin selection overnight for amplification. The cells were then diluted to include the desired number of constructs in each library. These bottlenecked libraries were then purified by mini prep. These libraries are named reporter gene_barcodes libraries.

Cloning of sgRNA-barcode libraries. The following strategy was used to clone the sgRNA-barcode libraries: first, the protospacer-sgRNA constant region-barcode cassette library was generated through multi-step overlapping PCR; then, this library was inserted into lentiviral vectors with U6 promoter placed downstream of the PPT sequence through isothermal assembly. To generate the protospacer-sgRNA constant region-barcode cassette library, the barcode segments were first generated using similar approaches as described in the “Cloning of barcode libraries for quantification of barcode decoding accuracy” section. The only difference is that the constant (primer) region in the 5′ end was changed by substituting Oligos 1-9 with 105-113, since the barcodes were placed immediately adjacent to sgRNAs. The sgRNA constant region was PCR amplified using Oligos 114 and 115. The protospacer libraries were ordered from IDT with constant regions on both side of the protospacer for PCR amplification. In this work, two proto-spacer libraries were generated, one for essential ribosome genes and non-targeting sgRNA controls used to measure the recombination rate between sgRNAs and barcodes (Dataset S1) and the other for targeting genes that potentially regulate RNA localizations in the nucleus (Dataset S2). The protospacer libraries were PCR amplified using Oligos 116 and 117, and the PCR products were gel purified. Then, the proto-spacer, sgRNA constant region and barcodes PCR products were mixed and subjected to overlapping PCR using Oligo 116 and 118 as primers. The reverse primer, Oligo 118, contained a random sequence region of 20 bases which served as the unimolecular identifier (UMI) for the sequencing step. The sequences of oligos 105-118 are also described in Dataset S4. All PCR reactions were performed using real-time qPCR equipment to monitor the reactions so that the reactions were stopped at log growth phase. The PCR products were assembled into a modified pFUGW backbone with U6 promoter placed downstream of the PPT sequence through isothermal assembly. The assembled library was electroporated into Endura electrocompetent cells which were then grown on ampicillin selection plate overnight for amplification. Certain number of colonies (˜3800 for essential ribosome gene library and ˜2500 for RNA localization screening library) were scrapped off the plate with LB buffer and cultured in 200 mL LB buffer overnight. The libraries were purified by maxi prep. These libraries are named sgRNA_barcodes libraries.

Sequencing library preparation and analysis. To determine the identity of the barcodes presented in the library as well as to establish the correspondence between sgRNAs and barcodes, the library was analyzed using high-throughput sequencing. It was found that PCR amplification of the barcode region can lead to recombination of the barcodes due to homologous regions among the barcodes. Thus, a ligation-based approach was used to install sequencing adaptors to the barcode library. In this approach, the regions subjected to sequencing were digested from the library and then ligated to adaptors using T4 ligase.

To determine the barcodes in the reporter gene-barcodes libraries, the libraries were digested using BstXI and BamHI at 37° C. for 2 to 3 hours, and the resulting fragments were purified using Zymo DNA purification kit (ZD4002). To generate adaptors with sticky ends for ligation, Oligos 119-124 (sequence provided in Dataset S4) were mixed at 0.5 micromolar each, and subjected to 5 cycle of PCR reaction, and products were purified using Zymo DNA purification kit to produce double-stranded sequences with 5′ and 3′ adaptors separated by BstXI and BamHI digestion sites. The purified products were digested with BstXI and BamHI for 37° C. for 2 to 3 hours, and then purified. The resulting mixture contained adaptors with sticky ends for ligation and was mixed with the purified library fragment mixtures described above. T4 ligase were added and the reactions were kept in room temperature for 2 to 4 hours. The reaction mixtures were directly subjected to 2% agarose gel electrophoresis and a band corresponds to a size of ˜400 bp was excised and purified. The purified DNA samples were used for concentration measurement and high-throughput sequencing using V2-MISeq kit (Illumina, MS-103-1003).

To determine the sgRNA-barcode correspondence of the sgRNA_barcodes libraries, two sequencing libraries were generated because the length from proto-spacer to the end of the barcodes is longer than 500 bps, which exceeds the length range optimal for high quality sequencing by the V2-MISeq kit. In one library, the ligation sites were generated using BstXI and BamHI, which located 5′ to the proto-spacer and 3′ to the UMI, respectively. Sequencing of this library covered the proto-spacer region, part of the barcode region and the UMI region because the middle part the barcode could not be reached by sequencing from either end for this library. In the second library, the ligation sites were generated using KpnI and BamHI (the KpnI site was placed right after sgRNA and before barcodes). Sequencing of this library covered the whole barcode region as well as the UMI region. UMI sequences were used to identify the proto-spacer and barcode in the same construct from these two libraries. Oligos 125-130 and Oligos 131-136 (sequence provided in Dataset S4) were used to generate adaptors for the first and the second library, respectively. The procedures were the same as described for generating sequencing libraries for reporter gene-barcode libraries.

UMI, protospacers and barcode sequences were extracted from sequencing reads. The reads were then grouped by common UMI and barcode to generate a codebook for sgRNA and barcode correspondence. The reads with incorrect protospacers or with barcodes assigned to multiple sgRNAs were excluded from further analysis.

In order to use sequencing to determine the distribution of protospacers and UMIs from cell populations at different time points after lentivirus transduction in the experiments to determine recombination rates, the sequencing libraries were prepared by PCR amplification from purified genomic DNA using Oligos 137-140 and Oligos 141-144 (sequence provided in Dataset S4) as forward and reverse primers, respectively.

Lentivirus production and transduction. Lentivirus were produced in LentiX cells (Takara, 632180) using Lenti-X™ Packaging Single Shots (VSV-G) (Takara, 631276). The produced viruses were concentrated using Lenti-X™ Concentrator (Takara, 631231) and stored at −80° C. For transfection, the amount of virus was controlled so that 10-30% of the cells were transduced to ensure most infected cells were infected by only 1 virus particle. The virus transductions were performed using 10 microgram/mL polybrene (Sigma, TR-1003-G). The virus titer for the construct with U6-sgRNA-barcode array placed after PPT did not show obvious reduction compared to that for the construct without insertion after PPT, indicating that the insertion did not impair the lentivirus transduction.

siRNA knockdown. All siRNAs were purchased from Dharmacon, and siRNA knockdown was performed according to Dharmacon's protocol. Briefly, U-2 OS cells were plated on imaging coverslips in 12-well plate at 30,000 cells per well. For siRNA transfection, 1.5 microliters, 20 micromolar siRNA was added in 100 microliters of serum-free, antibiotics-free medium in one tube and 1 microliter of Dharmacon transfection reagent (Dharmacon, T-2001-01) was added in 100 microliters of serum-free medium in a separate tube. Two tubes were incubated for 5 minutes and then mixed gently to incubate for another 20 minutes at room temperature. 800 microliters of antibiotics-free medium with serum was mixed into the 200 microliter siRNA and transfection reagent mix described above to generate the 1 mL transfection medium. Cell culture medium was replaced with the 1 mL transfection medium. The cells were incubated at 37° C. for 72 hours before phenotype measurements.

Imaging coverslip silanization. Imaging coverslips were first cleaned by 1M KOH and pure methanol, washed by 70% ethanol and dried in the oven. For silanization, coverslips were covered in silanization buffer (500 mL distilled water, 1500 microliters of Bind-silane (Sigma, GE17-1330-01) and pH adjusted to 3.5 by glacier acetic acid) for an hour at room temperature. The coverslips were then washed by water and dried to store. Before plating the cells, the silanized coverslips were coated by 1% poly-D-lysine (Sigma, P0899) in 60 mm diameter cell-culture dishes for 30 min followed by a single one-hour wash with water.

Imaging sample preparation. U-2 OS cells were plated on the coverslips two days before fixation. For phenotype imaging in the experiments that screen for factors involved in regulating nuclear RNA localization, U-2 OS cells were fixed 6 days after lentivirus transduction. The samples were fixed by 4% paraformaldehyde (EMS,15714) in PBS for 15 min and permeabilized in 0.5% Triton-X (Sigma, X100) for 30 mins. Next, samples were incubated in block buffer (500 microliters block buffer: 50 microliters 10×PBS, 200 microliters RNAse free BSA (ThermoFisher, AM2618), 50 microliters 25 mg/ml yeast tRNA (ThermoFisher, 15401029), 5 microliters Murine RNAse inhibitor (NEB, M0314L), 1 microliters 25% Triton-X and RNAse-free water to 500 microliters) for one hour and stained with 1:100 primary antibody, anti-SON (Abcam, ab121759), in block buffer for one hour at room temperature. The samples were washed three times with 1×PBS and incubated with 1:300 oligonucleotide-labeled secondary antibody for one hour. The oligonucleotide-labeled secondary antibody can be later probed by readout probes with sequence complementary to the oligonucleotide sequence on the antibody. The samples were washed three times with 1×PBS and post-fixed with 4% PFA for 30 minutes. Then the samples were equilibrated in 30% formamide in 2×SSC for 5 minutes before FISH staining. The FISH hybridization buffer contains 30% formamide (ThermoFisher, AM9342), 60% stellaris RNA FISH hybridization buffer (Biosearch, SMF-HB1-10), 10% 25 mg/mL Yeast tRNA and 1:100 murine RNase inhibitor. The samples were stained with 300 nM FISH probes for the reporter gene, 300 nM FISH probes for RNA phenotype (i.e, 6 RNA species) imaging, and 100 nM primary amplification probes for barcode imaging at 37° C. overnight. The FISH probes for the reporter gene each contained a 30-nt targeting sequence that can bind to the reporter gene mRNA and three 20-nt readout sequence that allows the binding of complementary, fluorescently labeled readout probes. The FISH probes for each RNA target in phenotype imaging each contained a 30-nt targeting sequence that can bind to the RNA target and one or two 20-nt readout sequences that allows the binding of complementary, fluorescently labeled readout probes. Each primary amplification probe for barcode imaging contained a 30-nt targeting sequence that can bind to one of the 30-nt trit sequence on the barcodes, as well as four additional 30-nt identical sequences that allows the binding of secondary amplification probes (FIG. 1A). Then the samples were washed in 30% formamide in 2×SSC twice and stained with 100 nM secondary amplification probes for barcode imaging in 10% hybridization buffer (10% formamide, 80% stellaris RNA FISH hybridization buffer, 10% 25 mg/mL Yeast tRNA and 1:100 murine RNase inhibitor) for an hour at 37° C. Each secondary amplification probe contained a 30-nt targeting sequence that can bind to the primary amplification probes, and four additional 20-nt identical readout sequences that allows the binding of complementary, fluorescently labeled readout probes. This amplification scheme thus allows a maximum of 16-fold signal amplification. The samples labeled with FISH probes for phenotype imaging and reporter gene mRNA imaging, and primary and secondary amplification probes for barcode imaging were washed twice in 30% formamide in 2×SSC, and then embedded in 4% polyacrylamide gel, followed by incubation with protein digestion buffer (for 50 mL digestion buffer: 5 mL 8M Guanidine-HCL (ThermoFisher, 24115), 2.5 mL 1 M Tris pH 8.0 (ThermoFisher, 15569025), 100 microliters 0.5 M EDTA (ThermoFisher, 15575020), 0.25 mL Triton-X and 1:100 proteinase K (ThermoFisher, AM2548)) at 37° C. overnight to remove proteins and lipids from the sample. This step is referred to as the sample clearing step below. The protease K cleavage led to protein digestion (including the digestion of mCherry protein), and therefore the mCherry fluorescence signal was eliminated after digestion and did not interfere with FISH signal detection using 561 nm channel. The FISH probes for polyA-containing RNAs, 7SK, MRP, U2 snRNA, and the oligonucleotides linked to the secondary antibody for SON staining were conjugated with acrydite, which can crosslink to the polyacrylamide gel and retain these probes as well as their bound RNA within the gel during the sample clearing step. The FISH probes for MALAT1 and pre-ribosome were not labeled by acrydite because both MALAT1 and pre-ribosome are large in size and thus were retained in the gel during sample clearing. The reporter gene mRNAs were linked to the gel through the acrydite labeled poly T probes that can bind to the poly A tails of the reporter mRNAs, thereby allowing the FISH probes for the reporter gene and the FISH probes for barcode imaging to be retained in the gel during clearing. The sample clearing step substantially reduces background signal due to cell autofluorescence and nonspecific binding of FISH probes to proteins and lipids. The samples were then washed by 2×SSC and left in 2×SSC for imaging. Sequences for used FISH probes are listed in Dataset S5.

For experiments that were used to measure the barcoding identification error using two known phenotypes (expression of HA or Myc tagged reporter genes), U-2 OS cells were fixed 6 days after transduction. The tags were stained by primary antibodies (anti-Myc (Abcam, ab9132), anti-HA (Abcam, ab9110)), and then Alexa 405 labeled anti-mouse secondary antibody (Abcam, ab175658) and Alexa 488 labeled anti-rabbit secondary antibody (Invitrogen, A21206). The samples were incubated in 25 mM MA-NHS (Sigma, 730300) in 2×SSC for one hour before gel embedding, therefore, MA-NHS labeled antibodies were linked to the gel during gel polymerization. After sample cleaning, antibodies were digested into fragments and the dyes were linked to gels via crosslinked antibody fragments. The dyes Alexa 405 and Alexa 488 can survive the polymerization reaction during gel embedding. The rest of sample preparation including immunostaining and barcode staining is described as above.

Antibody labeling by oligonucleotide. The following strategy was used to label antibodies with oligonucleotide. Antibodies were first mixed with DBCO-NHS which conjugate DBCO to antibodies and the DBCO-labeled antibodies were then mixed with azide-labeled oligonucleotide to conjugate oligonucleotide to antibodies. Specifically, 100 microgram anti-rabbit antibody (ThermoFisher, 31210) was first buffer exchanged into 100 microliters PBS using 50 KD protein concentrator (Millipore, UFC510024). NaHCO₃ and DBCO-NHS ester (Kerafast, FCC310) were added into the antibody solution so that their final concentrations were 50 mM and 100 micromolar, respectively. The reaction was allowed to proceed for 1 h at room temperature to make DBCO-labeled antibodies and excess DBCO was removed through buffer exchange with PBS using 50 kD protein concentrator. Then PBS buffer was added to DBCO-labeled antibodies to make the solution volume 100 microliters and 25 microliters azide-labeled oligonucleotide (100 micromolar, Dataset S3) was added. The reaction was allowed to proceed at 4° C. overnight. After the reaction finished, the excess oligonucleotide was removed through buffer exchange using PBS and the final oligonucleotide-labeled antibody was aliquoted and stored at −80° C.

Imaging setup and sequential imaging. The imaging setup was as described previously. See, e.g., U.S. Pat. Apl. Pub. No. 2017-0220733 or Int. Pat. Apl. Pub. No. WO 2018/089438, each incorporated herein by reference in its entirety. Briefly, a peristaltic pump (Gilson, MINIPULS 3) pulled liquid into Bioptech's FCS2 flow chamber with sample coverslips and three valves (Hamilton, MVP and HVXM 8-5) were used to select the input fluid. A custom microscope built around a Nikon Ti-U microscope body with a Nikon CFI Plan Apo Lambda 60× oil immersion objective with 1.4 NA was used for imaging. Solid-state single-mode lasers (405 nm laser, Obis 405 nm LX 200 mW, Coherent; 488 nm laser, Genesis MX488-1000, Coherent; 560 nm laser, 2RU-VFL-P-2000-560-B1R, MPB Communications; 647 nm laser, 2RU-VFL-P-1500-647-B1R, MPB Communication; and 750 nm laser, 2RU-VFL-P-500-750-B1R, MPB Communications) were used for illumination. An acousto-optic tunable filter (AOTF) was used to control the intensities of the 488 nm, 560 nm, and 647 nm lasers; the 405 nm laser was modulated by a direct digital signal; the 750 nm laser were switched by mechanical shutters. A custom dichroic (Chroma, zy405/488/561/647/752RP-UF1) and emission filter (Chroma, ZET405/488/461/647-656/752m) were used to separate the excitation illumination from the fluorescence emission. The emission was imaged onto the Hamamatsu digital CMOS camera. During acquisition, the sample was translated using a motorized XY stage (Ludl, BioPrecision2) and kept in focus using a home-built autofocus system.

For experiments that requires higher imaging throughput, a 40× objective (CFI60 Plan Fluor 40× Oil Immersion Objective Lens) was used to acquire more cells per Field-Of-View (FOV). A four-camera system was used for acquiring signals from 750 nm, 647 nm, 561 nm and 488 nm fluorophores separately and simultaneously. In detail, a four-camera mount (QuadCam LS 1.0×, 89 North) was installed on a Nikon Ti-U microscope body, and four Hamamatsu digital CMOS cameras were installed on the mount. Four dichroic filters (T4951pxr, T5621pxr, T6471pxr, T7601pxr, Chroma Tech) which split the signal from different fluorophores and 4 four single-band emission filters (ET 450/50m, ET 525/50m, ET 605/75m and ET 705/70m, Chroma Tech) were installed in the camera mount. To align the signals from the four cameras, multi-color beads (FP-0257-2, Spherotech) were imaged and signals from 647 nm, 561 nm and 488 nm color channel were aligned to signals from 750 nm color channel by cp2tform function in MatLab. Cp2tform inferred a polynomial spatial transformation for x, y and z coordinates and the transformation was applied to barcode and phenotype signals. To image the nucleus on the four-camera setup, 1:1000 647 nm Nucred dye (R37106, ThermoFisher) was used instead of DAPI.

Before loading into the flow chamber, the sample was stained an Atto565-labeled, 20-nt readout probe (Dataset S5) which has a sequence complementary to the readout sequence on the FISH probes for reporter gene mRNA imaging. The staining was performed in hybridization buffer (10% ethylene carbonate (Sigma, E26258) in 2×SSC), with a readout probe concentration of 3 nM. The readout probe for the reporter gene was introduced only once but was imaged repetitively during for all hybridization rounds. The readout probes for the 7 molecular targets (SON protein and 6 RNA targets) for phenotype imaging and the readout probes for barcode imaging were introduced in sequential rounds of hybridizations. For phenotype and barcode imaging, 3 nM 20-nt readout probes (Bio-Synthesis Inc., Dataset S5), complementary to the oligonucleotide sequence on the SON antibody (Abcam, ab121759), or to the readout sequences on the FISH probe for the 6 RNA targets, or to the readout sequences on the secondary amplification probes for barcode imaging, in hybridization buffers (10% ethylene carbonate in 2×SSC) were flowed into the chamber, left for 15 minutes and followed by hybridization buffer wash. The dyes for these probes, Alexa 488, Cy5 or Alexa 750, were linked to the oligos via a cleavable disulfide bond (Biosynthesis, Dataset S5). The sample were imaged in anti-bleach buffer (For 50 mL anti-bleach buffer: 50 mg gluco-oxidase (Sigma, G2133), 50 mg (+/−)-6-hydroxy-2,5,7,8-tetramethylchromane-2-carboxylic acid (Trolox) (Sigma, 238813), 300 microliters catalase (Sigma, C100-500MG), 10% w/v glucose (Sigma, G8270), 5 mL 500 micromolar Trolox quinone and 50 microliters murine RNase inhibitor). For each round of hybridization, fluorescence signals from four color channels (488 nm, 561 nm, 647 nm, and 750 nm, if phenotype imaging was included in the round) or three color channels (561 nm, 647 nm, and 750 nm, if phenotype imaging was not included in the round) were imaged. After each round, the dyes on the readout probes were cleaved by 10% tris (2-carboxyethyl) phosphine (TCEP; Sigma, 646547-10X1ML), followed by hybridization of the readout probes for next round.

Reporter gene mRNA signal was detected using the 561 nm channel in every round for the sake of quantification of the colocalization ratio between reporter gene signal and barcode signal, and for image registration. The barcode signals were measured through sequential rounds of hybridization and imaging using 647 nm and 750 nm channels with cleavable Cy5 and Alexa 750 dyes in rounds 1-18, which allowed all 36 values of the 12-trit barcodes to be imaged. The signals for SON and 6 RNA targets in phenotype imaging were measured through sequential rounds of hybridization and imaging using the 488 nm channel with cleavable Alexa 488 dye in rounds 1-7. For phenotype imaging, the images were collected at a slightly higher focal plane (2-3 micrometer) optimal for signals from interior of the nuclei.

For experiments measuring the barcoding identification error using two known phenotypes (expression of HA or Myc tagged reporter genes), Myc and HA tags were stained with Alexa 405-dye and Alexa 488-dye labeled secondary antibodies and imaged in 405 nm and 488 nm channels, respectively.

DAPI staining was imaged and used for cell segmentation and nucleus identification. For experiments measuring two know phenotypes (HA or Myc tagged reporter genes), DAPI staining was imaged at the last round of imaging. For the experiments to screen for factors regulating nuclear RNA localization, DAPI staining was imaged at the first round of imaging. The sequences for dye labeled readout probes are listed in Dataset S5.

Transcription inhibition. For transcription inhibition, 50 micromolar DRB (Sigma, D1916-10MG) was mixed in EMEM and incubated with the cells for an hour before fixation.

Barcode decoding analysis. To corrected for non-uniformity in illumination, every image for a give color channel was divided by the mean-intensity image for all images for that illumination color. Images of multiple rounds were registered using uncleavable signals of the reporter gene mRNA. Cells were segmented by watershed algorithm using DAPI staining as seed and cell autofluorescence (for the experiments to evaluate barcode decoding accuracy and lentivirus recombination) or poly-A containing RNAs staining (for the experiments to screen for factors regulating nuclear RNA localization) for cell boundary identification. Single-molecule signals for reporter gene mRNA and barcodes across all hybridizations were identified using a spot finding algorithm. For experiments using 4-camera imaging, spots were identified using a segmentation algorithm. In detail, the pixels with an intensity larger than a brightness threshold were selected. The clusters of the selected pixels were identified by the bwareaopen function in MatLab. The clusters within a bounded area range (2-30 pixels) were kept. The area ranges were determined by visual inspection of the raw image. In order to capture spots with varies intensity, this process was iterated using multiple brightness thresholds, e.g., from 0.6×max (pixel intensity in the FOV) to 0.3×max (pixel intensity in the FOV) with the decrement of 0.05× max (pixel intensity in the FOV). The brightness threshold for each trit signals was determined manually. For each iteration, lower brightness threshold will identify two types of clusters: (i) the dim clusters that cannot be detected at higher brightness threshold from the previous round and (ii) the larger clusters that completely include one or more clusters identified from previous round. For any cluster of type (i), it was kept only if its area was within the allowed area range described above. For any cluster of type (ii), if its area was within the allowed area range, it was kept; otherwise, it was removed, and the smaller cluster(s) identified from the previous round that overlapped with this new cluster was kept instead. The center of these clusters was identified by regionprops function in MatLab.

The single-molecule FISH spots were assigned to cells, and the colocalization ratio for each of the three values of a trit in the barcode was calculated as the number of reporter-gene smFISH spots that were colocalized with barcode smFISH signal divided by total number of reporter-gene smFISH spots within the cells. To determine the value of each trit for each cell, cells were clustered based on the three colocalization ratios of that trit by k-means clustering, and the trit value was assigned to each cluster based on which of the three mean colocalization ratio was the highest for that cluster. The same process was repeated for all 12 trits, so that each cell was assigned a 12-trit barcode. For each value of a trit, the average colocalization ratio for the population of cells assigned that value was measured to be 0.4; whereas the average colocalization ratio due to random colocalization with non-specifically bound probes, assessed from the two populations of cells not assigned that trit value, was measured to be 0.1.

To decode the cells based on the barcode signals alone (i.e. without consideration of the colocalization between barcode signals and reporter gene signals), cells were clustered based on the numbers of barcode-signal spots detected for the three trit values within each cell. A k-means clustering algorithm was used to partition the cells into three populations, and the trit value was assigned to each population based on which one of the three trit values had the highest mean spot numbers. This same process was repeated for all 12 trits.

To estimate the barcode misidentification rate, since only 0.4% of all possible barcodes were present in the libraries due to the bottlenecking strategy, the probability that any erroneously decoded barcode would match the barcodes in the libraries is only 0.4%. Thus, among the 57% exact-matched barcodes, only approximately p=0.3% could arise from barcode misidentification (solving from (p*57%)/(1−57%)=0.4%/(1−0.4%)).

Myc and HA signal quantification. To quantify the HA and Myc expression in the nucleus, the nuclear boundary of each cell was used as a mask to measure the intensity of the corresponding Myc or HA channel. To allow unambiguous assignment of HA and Myc expression to individual cells, the threshold values for HA and Myc expression were first determined, above which HA or Myc tag expression can be confidently detected. To determine these threshold values, a k-means clustering algorithm was used to cluster the cells into two groups based on their unthresholded HA and Myc tag staining intensity. This grouping allowed approximated separation of cells into HA- and Myc-expressing cells. To estimate the background stain level for the HA tag, the mean and standard deviation of the HA intensity values for cells in the Myc-expressing cluster was calculated and the threshold for HA signal was calculated as mean plus three standard deviations. The threshold value for Myc expression was determined similarly from the HA-expressing cluster. The cells with HA and Myc intensities that were both lower than their respective thresholds or both higher than their respective thresholds were discarded (197 out of 2336 cells). After removing these ambiguous cells, the remaining cells were clustered again using a k-means algorithm to obtain the final grouping as shown in FIG. 2C.

Recombination rate calculation. Calculation of the recombination rate aᵢ for the ith sgRNA (with the barcode or UMI) is based on the following:

$${B}_{i , day n} = {P}_{i , day 2} * ( ( 1 - {a}_{i} ) * {S}_{i} + {a}_{i} * C )$$

$${S}_{i} = \frac{{P}_{i , day n}}{{P}_{i , day 2}}$$

$$C = {\sum }_{i} ( \frac{{P}_{i , day 2}}{{Σ}_{j} {P}_{j , day 2}} * {S}_{i} ) ,$$

where n is the number of days post transduction, which is equal to 21 or 28 in these experiments. P_{iday} 2 is the normalized proto-spacer reads determined by sequencing for the ith sgRNA on day 2 post transduction (normalized by the total proto-spacer reads measured on day 2 post transduction). P_{i, day n} is the normalized proto-spacer reads determined by sequencing for the ith sgRNA on day n post transduction (normalized by the total proto-spacer reads measured on day n post transduction). B_{i, day n} is normalized cell numbers determined by barcode imaging or normalized UMI reads determined by sequencing (normalized by the total cell number or UMI reads on day n post transduction). Sᵢ is the survival rate of the ith sgRNA. C is the average survival rate for all sgRNAs within the library, calculated by considering the abundance weight of different sgRNAs in the library, which is the mean survival rate if recombination happens.

Phenotype measurement quantification. Nucleus boundary were determined by DAPI signals. The cells whose nuclei were in contact with the edge of the imaging field-of-view were removed from further analysis. To identify the clusters of MRP, pre-ribosome, and SON, the background intensity of the channel was subtracted and the functions regionprops (MatLab) and bwareaopen (MatLab) were used to identify the clusters, which was similar to the spot finding algorithm for experiments with 4-camera imaging. In detail, the pixels with intensity larger than a brightness threshold will be selected. The clusters of the selected pixels were identified by the bwareaopen function. The clusters within a bounded area range (20-3000 pixels for SON, 100-5000 pixels for pre-ribosome and 100-6000 pixels for MRP) were kept. The area ranges were determined by visual inspection of the raw image. In order to capture clusters with relatively wide variations in staining levels, this process was iterated using multiple brightness thresholds (from 0.9×max (pixel intensity in the nucleus) to 0.1×max (pixel intensity in the nucleus) with the decrement of 0.05×max (pixel intensity in the nucleus)). The number of the final identified clusters and the area of each cluster were measured using the regionprops function. For MRP, preribosome, and SON, the number of clusters, the mean area of clusters, and the cluster intensity (defined as the total signal within the cluster boundaries divided by total cluster area) were calculated for each cell. To quantify the nuclear speckle enrichment MALAT1, 7SK, U2 and poly-A containing RNAs, cluster boundaries from the SON staining were used as mask to measure the MALAT1, 7SK, U2 and poly-A containing RNAs signals within the SON cluster boundaries. Nuclear speckle intensity of each of these RNAs was measured as the total signal of the said RNA within the SON cluster boundaries divided by the total area covered by SON clusters. The signal intensity outside the speckle was measured as the total signal of the RNA in the nucleus but outside nuclear speckles divided by the total area of the nucleus that was not in nuclear speckles. The nuclear speckle enrichment was determined as the ratio between the nuclear speckle intensity and the signal intensity outside the speckle.

To identify hits from the screening, four replicates of experiments were combined. The quantified values described above (i.e. cluster number, cluster area, cluster intensity, and nuclear speckle enrichment) of each replicate were normalized by the median values of all cells within each replicate before combination. The Student's t test was used to calculate the p value by testing the measured values for the cells harboring one targeting sgRNA against the values measured from cells harboring all control, non-targeting sgRNAs. When at least two sgRNAs targeting a certain gene showed p values<0.05, the gene was listed as a hit. The sgRNAs that had less than 40 cells were removed from analysis.

### Dataset S1

sgRNA library to evaluate the lentivirus design for reduced recombination effect. This dataset lists the oligo sequences for the proto-spacers of 159 sgRNAs targeting essential ribosomal genes and 51 non-targeting sgRNAs.

| | | SEQ |
|---|---|---|
| | | ID |
| sgRNA | SEQUENCE | NO: |
| RPL10 | tcttgtggaaagCCAGAAACATG | 1 |
| | GACTACTTACCAGGGACACCTGT | |
| | TTCAGAGCTAAGCACAAGAGTGC | |
| RPL10A | tcttgtggaaagCCAGAAACATG | 2 |
| | GCACACACAGAGAACTTAGGGGT | |
| | TTCAGAGCTAAGCACAAGAGTGC | |
| RPL11 | tcttgtggaaagCCAGAAACATG | 3 |
| | GCTCACCTTTGGAAAACACAGGT | |
| | TTCAGAGCTAAGCACAAGAGTGC | |
| RPL12 | tcttgtggaaagCCAGAAACATG | 4 |
| | GACTGACCATTCAGAACAGACGT | |
| | TTCAGAGCTAAGCACAAGAGTGC | |
| RPL13 | tcttgtggaaagCCAGAAACATG | 5 |
| | GCACCTGCGGATCTTACGGGCGT | |
| | TTCAGAGCTAAGCACAAGAGTGC | |
| RPL13A | tcttgtggaaagCCAGAAACATG | 6 |
| | GCCTCCCTCTAGGCCGGAAGGGT | |
| | TTCAGAGCTAAGCACAAGAGTGC | |
| RPL14 | tcttgtggaaagCCAGAAACATG | 7 |
| | GGAAGATTGAAGCCAGAGAAAGT | |
| | TTCAGAGCTAAGCACAAGAGTGC | |
| RPL18 | tcttgtggaaagCCAGAAACATG | 8 |
| | GACAACCTCTTCAACACAACCGT | |
| | TTCAGAGCTAAGCACAAGAGTGC | |
| RPL18A | tcttgtggaaagCCAGAAACATG | 9 |
| | GCTACGAGAGTACAAGGTAGTGT | |
| | TTCAGAGCTAAGCACAAGAGTGC | |
| RPL19 | tcttgtggaaagCCAGAAACATG | 10 |
| | GAGACCTTCTTCTTGCCACAGGT | |
| | TTCAGAGCTAAGCACAAGAGTGC | |
| RPL23A | tcttgtggaaagCCAGAAACATG | 11 |
| | GATAGCATAGTGGTCAAGCCTGT | |
| | TTCAGAGCTAAGCACAAGAGTGC | |
| RPL24 | tcttgtggaaagCCAGAAACATG | 12 |
| | GAGTCGGCTTTCCTTTCCAAGGT | |
| | TTCAGAGCTAAGCACAAGAGTGC | |
| RPL26 | tcttgtggaaagCCAGAAACATG | 13 |
| | GATCTTCCTTCGAATGTGGGAGT | |
| | TTCAGAGCTAAGCACAAGAGTGC | |
| RPL27 | tcttgtggaaagCCAGAAACATG | 14 |
| | GACGCAAAGCTGTCATCGTGAGT | |
| | TTCAGAGCTAAGCACAAGAGTGC | |
| RPL3 | tcttgtggaaagCCAGAAACATG | 15 |
| | GCCACCGAGGCCTGCGCAAGGGT | |
| | TTCAGAGCTAAGCACAAGAGTGC | |
| RPL30 | tcttgtggaaagCCAGAAACATG | 16 |
| | GAAAGTGGGAAGTACGTCCTGGT | |
| | TTCAGAGCTAAGCACAAGAGTGC | |
| RPL31 | tcttgtggaaagCCAGAAACATG | 17 |
| | GAGAGGGATACTCACACTCCAGT | |
| | TTCAGAGCTAAGCACAAGAGTGC | |
| RPL32 | tcttgtggaaagCCAGAAACATG | 18 |
| | GAGGGTTCGTAGAAGATTCAAGT | |
| | TTCAGAGCTAAGCACAAGAGTGC | |
| RPL34 | tcttgtggaaagCCAGAAACATG | 19 |
| | GAACAAAGAAACATGTCAGCAGT | |
| | TTCAGAGCTAAGCACAAGAGTGC | |
| RPL35 | tcttgtggaaagCCAGAAACATG | 20 |
| | GCAATGGATTTCCGGACGACTGT | |
| | TTCAGAGCTAAGCACAAGAGTGC | |
| RPL35A | tcttgtggaaagCCAGAAACATG | 21 |
| | GAACAAAACCAGAGTCATCTGGT | |
| | TTCAGAGCTAAGCACAAGAGTGC | |
| RPL36 | tcttgtggaaagCCAGAAACATG | 22 |
| | GCATGGCCCTACGCTACCCTAGT | |
| | TTCAGAGCTAAGCACAAGAGTGC | |
| RPL37 | tcttgtggaaagCCAGAAACATG | 23 |
| | GAGGCCTTAGAGCCACAGCGGGT | |
| | TTCAGAGCTAAGCACAAGAGTGC | |
| RPL37A | tcttgtggaaagCCAGAAACATG | 24 |
| | GAGCAAGTGTACTTGGCGTGCGT | |
| | TTCAGAGCTAAGCACAAGAGTGC | |
| RPL38 | tcttgtggaaagCCAGAAACATG | 25 |
| | GCAGCAGATACCTTTACACCCGT | |
| | TTCAGAGCTAAGCACAAGAGTGC | |
| RPL4 | tcttgtggaaagCCAGAAACATG | 26 |
| | GATTACCAGGGATGTTTCTGAGT | |
| | TTCAGAGCTAAGCACAAGAGTGC | |
| RPL5 | tcttgtggaaagCCAGAAACATG | 27 |
| | GGACTGGTGATGAATACAATGGT | |
| | TTCAGAGCTAAGCACAAGAGTGC | |
| RPL6 | tcttgtggaaagCCAGAAACATG | 28 |
| | GACAGGGTTGCGGCTGCAATGGT | |
| | TTCAGAGCTAAGCACAAGAGTGC | |
| RPL7 | tcttgtggaaagCCAGAAACATG | 29 |
| | GCGTTTGTCATCAGAATCAGAGT | |
| | TTCAGAGCTAAGCACAAGAGTGC | |
| RPL7A | tcttgtggaaagCCAGAAACATG | 30 |
| | GCACCCTTTACTTCTCAGGACGT | |
| | TTCAGAGCTAAGCACAAGAGTGC | |
| RPLP0 | tcttgtggaaagCCAGAAACATG | 31 |
| | GAGACTGCTGCCTCATATCCGGT | |
| | TTCAGAGCTAAGCACAAGAGTGC | |
| RPLP2 | tcttgtggaaagCCAGAAACATG | 32 |
| | GAGCCCCACCAGCAGGTACACGT | |
| | TTCAGAGCTAAGCACAAGAGTGC | |
| RPS11 | tcttgtggaaagCCAGAAACATG | 33 |
| | GCCGATGTTCTTGTAGTACCGGT | |
| | TTCAGAGCTAAGCACAAGAGTGC | |
| RPS13 | tcttgtggaaagCCAGAAACATG | 34 |
| | GAAACACTCACCGATCTGTGAGT | |
| | TTCAGAGCTAAGCACAAGAGTGC | |
| RPS14 | tcttgtggaaagCCAGAAACATG | 35 |
| | GCAACATAGCAGCATATGGTGGT | |
| | TTCAGAGCTAAGCACAAGAGTGC | |
| RPS15 | tcttgtggaaagCCAGAAACATG | 36 |
| | GATCATCCTACCCGAGATGGTGT | |
| | TTCAGAGCTAAGCACAAGAGTGC | |
| RPS15A | tcttgtggaaagCCAGAAACATG | 37 |
| | GATCAACAATGCCGAAAAGAGGT | |
| | TTCAGAGCTAAGCACAAGAGTGC | |
| RPS17 | tcttgtggaaagCCAGAAACATG | 38 |
| | GAGCTCCGCAACAAGATAGCAGT | |
| | TTCAGAGCTAAGCACAAGAGTGC | |
| RPS18 | tcttgtggaaagCCAGAAACATG | 39 |
| | C.AAAGGATGGAAAATACAGCCG | |
| | TTTCAGAGCTAAGCACAAGAGTG | |
| | C | |
| RPS19 | tcttgtggaaagCCAGAAACATG | 40 |
| | GAACTGGTTCTACACGCGAGCGT | |
| | TTCAGAGCTAAGCACAAGAGTGC | |
| RPS20 | tcttgtggaaagCCAGAAACATG | 41 |
| | GAAAAACACCCGTGGAGCCGGGT | |
| | TTCAGAGCTAAGCACAAGAGTGC | |
| RPS24 | tcttgtggaaagCCAGAAACATG | 42 |
| | GTACAAAGATGACATCCGGTGGT | |
| | TTCAGAGCTAAGCACAAGAGTGC | |
| RPS26 | tcttgtggaaagCCAGAAACATG | 43 |
| | GCGTCATTCGAAACATAGTGGGT | |
| | TTCAGAGCTAAGCACAAGAGTGC | |
| RPS27A | tcttgtggaaagCCAGAAACATG | 44 |
| | GATCAGTCTCTGCTGATCAGGGT | |
| | TTCAGAGCTAAGCACAAGAGTGC | |
| RPS3 | tcttgtggaaagCCAGAAACATG | 45 |
| | GAAATCATTATCTTAGCCACCGT | |
| | TTCAGAGCTAAGCACAAGAGTGC | |
| RPS3A | tcttgtggaaagCCAGAAACATG | 46 |
| | GAAAAACGCAACAATCAGATAGT | |
| | TTCAGAGCTAAGCACAAGAGTGC | |
| RPS4X | tcttgtggaaagCCAGAAACATG | 47 |
| | GGTTCATTAAAATCGATGGCAGT | |
| | TTCAGAGCTAAGCACAAGAGTGC | |
| RPS5 | tcttgtggaaagCCAGAAACATG | 48 |
| | GACCTGCCTCACAGTGCAGGGGT | |
| | TTCAGAGCTAAGCACAAGAGTGC | |
| RPS6 | tcttgtggaaagCCAGAAACATG | 49 |
| | GACTGTAGTATCAGTCAGTCCGT | |
| | TTCAGAGCTAAGCACAAGAGTGC | |
| RPS7 | tcttgtggaaagCCAGAAACATG | 50 |
| | GAAATTGAAGTTGGTGGTGGTGT | |
| | TTCAGAGCTAAGCACAAGAGTGC | |
| RPS8 | tcttgtggaaagCCAGAAACATG | 51 |
| | GACAAGAAATACCGTGCCCTGGT | |
| | TTCAGAGCTAAGCACAAGAGTGC | |
| RPS9 | tcttgtggaaagCCAGAAACATG | 52 |
| | GGCAAAACTTATGTGACCCCGGT | |
| | TTCAGAGCTAAGCACAAGAGTGC | |
| RPSA | tcttgtggaaagCCAGAAACATG | 53 |
| | GCATAAATCTCAAGAGGACCTGT | |
| | TTCAGAGCTAAGCACAAGAGTGC | |
| RPL10 | tcttgtggaaagCCAGAAACATG | 54 |
| | GGACACCATGTGGCCACAAAGGT | |
| | TTCAGAGCTAAGCACAAGAGTGC | |
| RPL10A | tcttgtggaaagCCAGAAACATG | 55 |
| | GGAAGAACTATGATCCCCAGAGT | |
| | TTCAGAGCTAAGCACAAGAGTGC | |
| RPL11 | tcttgtggaaagCCAGAAACATG | 56 |
| | GGGATGCGAAGTTCCCGCATGGT | |
| | TTCAGAGCTAAGCACAAGAGTGC | |
| RPL12 | tcttgtggaaagCCAGAAACATG | 57 |
| | GGACCCCAACGAGATCAAAGTGT | |
| | TTCAGAGCTAAGCACAAGAGTGC | |
| RPL13 | tcttgtggaaagCCAGAAACATG | 58 |
| | GGCCGGATGGGACCCGACGCGGT | |
| | TTCAGAGCTAAGCACAAGAGTGC | |
| RPL13A | tcttgtggaaagCCAGAAACATG | 59 |
| | GCTGCCCCACAAAACCAAGCGGT | |
| | TTCAGAGCTAAGCACAAGAGTGC | |
| RPL14 | tcttgtggaaagCCAGAAACATG | 60 |
| | GGTCTCCTTTGGACCTCATGCGT | |
| | TTCAGAGCTAAGCACAAGAGTGC | |
| RPL18 | tcttgtggaaagCCAGAAACATG | 61 |
| | GCCGCCATAACAAGGACCGAAGT | |
| | TTCAGAGCTAAGCACAAGAGTGC | |
| RPL18A | tcttgtggaaagCCAGAAACATG | 62 |
| | GCTGCGCTATGACTCCCGGAGGT | |
| | TTCAGAGCTAAGCACAAGAGTGC | |
| RPL19 | tcttgtggaaagCCAGAAACATG | 63 |
| | GGAATGCCAGAGAAGGTCACAGT | |
| | TTCAGAGCTAAGCACAAGAGTGC | |
| RPL23A | tcttgtggaaagCCAGAAACATG | 64 |
| | GGCACGTCACCCACCTTCCGGGT | |
| | TTCAGAGCTAAGCACAAGAGTGC | |
| RPL24 | tcttgtggaaagCCAGAAACATG | 65 |
| | GCGTAGCGCCTCCCGTGTCCGGT | |
| | TTCAGAGCTAAGCACAAGAGTGC | |
| RPL26 | tcttgtggaaagCCAGAAACATG | 66 |
| | GATTTGCTCTTAGGTTGTACGGT | |
| | TTCAGAGCTAAGCACAAGAGTGC | |
| RPL27 | tcttgtggaaagCCAGAAACATG | 67 |
| | GGGGATATCCACAGAGTACCTGT | |
| | TTCAGAGCTAAGCACAAGAGTGC | |
| RPL3 | tcttgtggaaagCCAGAAACATG | 68 |
| | GCCGTGTCATTGCCCACACCCGT | |
| | TTCAGAGCTAAGCACAAGAGTGC | |
| RPL30 | tcttgtggaaagCCAGAAACATG | 69 |
| | GAATAGAGTACTATGCTATGTGT | |
| | TTCAGAGCTAAGCACAAGAGTGC | |
| RPL31 | tcttgtggaaagCCAGAAACATG | 70 |
| | GCAGATGTGCGCATTGACACCGT | |
| | TTCAGAGCTAAGCACAAGAGTGC | |
| RPL32 | tcttgtggaaagCCAGAAACATG | 71 |
| | GATTTCCTTCAGCGTAACTGGGT | |
| | TTCAGAGCTAAGCACAAGAGTGC | |
| RPL34 | tcttgtggaaagCCAGAAACATG | 72 |
| | GGTAAACAATTCTATTACCAGGT | |
| | TTCAGAGCTAAGCACAAGAGTGC | |
| RPL35 | tcttgtggaaagCCAGAAACATG | 73 |
| | GGTTAATAACTGTGAGAACACGT | |
| | TTCAGAGCTAAGCACAAGAGTGC | |
| RPL35A | tcttgtggaaagCCAGAAACATG | 74 |
| | GAGCAACACAGTCACTCCTGGGT | |
| | TTCAGAGCTAAGCACAAGAGTGC | |
| RPL36 | tcttgtggaaagCCAGAAACATG | 75 |
| | GCCCAGGCACAGCCGACGCCGGT | |
| | TTCAGAGCTAAGCACAAGAGTGC | |
| RPL37 | tcttgtggaaagCCAGAAACATG | 76 |
| | GCCTCATTCGACCAGTTCCGGGT | |
| | TTCAGAGCTAAGCACAAGAGTGC | |
| RPL37A | tcttgtggaaagCCAGAAACATG | 77 |
| | GGCCAAACGTACCAAGAAAGTGT | |
| | TTCAGAGCTAAGCACAAGAGTGC | |
| RPL38 | tcttgtggaaagCCAGAAACATG | 78 |
| | GCCTGCTCACAGCCCGACGAAGT | |
| | TTCAGAGCTAAGCACAAGAGTGC | |
| RPL4 | tcttgtggaaagCCAGAAACATG | 79 |
| | GCAGACTAGTGCTGAGTCTTGGT | |
| | TTCAGAGCTAAGCACAAGAGTGC | |
| RPL5 | tcttgtggaaagCCAGAAACATG | 80 |
| | GGAGGCTTGTCTATCCCTCACGT | |
| | TTCAGAGCTAAGCACAAGAGTGC | |
| RPL6 | tcttgtggaaagCCAGAAACATG | 81 |
| | GGCCAAGAAGGTTGATGCTGGGT | |
| | TTCAGAGCTAAGCACAAGAGTGC | |
| RPL7 | tcttgtggaaagCCAGAAACATG | 82 |
| | GGAACTGAAATTCGAATGGCGGT | |
| | TTCAGAGCTAAGCACAAGAGTGC | |
| RPL7A | tcttgtggaaagCCAGAAACATG | 83 |
| | GAGAGAGCCATCCTCTATAAGGT | |
| | TTCAGAGCTAAGCACAAGAGTGC | |
| RPLP0 | tcttgtggaaagCCAGAAACATG | 84 |
| | GCACCACAGCCTTCCCGCGAAGT | |
| | TTCAGAGCTAAGCACAAC.AGTG | |
| | C | |
| RPLP2 | tcttgtggaaagCCAGAAACATG | 85 |
| | GATTGAAGACGTCATTGCCCAGT | |
| | TTCAGAGCTAAGCACAAGAGTGC | |
| RPS11 | tcttgtggaaagCCAGAAACATG | 86 |
| | GGGATGTAGTGCAGATAGTCTGT | |
| | TTCAGAGCTAAGCACAAGAGTGC | |
| RPS13 | tcttgtggaaagCCAGAAACATG | 87 |
| | GCCAGCGCGCTACTTACAGTGGT | |
| | TTCAGAGCTAAGCACAAGAGTGC | |
| RPS14 | tcttgtggaaagCCAGAAACATG | 88 |
| | GCCGGAGTTTGATGTGTAGGGGT | |
| | TTCAGAGCTAAGCACAAGAGTGC | |
| RPS15 | tcttgtggaaagCCAGAAACATG | 89 |
| | GCTTGCGCAGGCGCTTCAGCAGT | |
| | TTCAGAGCTAAGCACAAGAGTGC | |
| RPS15A | tcttgtggaaagCCAGAAACATG | 90 |
| | GCCTCACAGGCAGGCTAAACAGT | |
| | TTCAGAGCTAAGCACAAGAGTGC | |
| RPS17 | tcttgtggaaagCCAGAAACATG | 91 |
| | GATATCCAGAAAGTTTACCTCGT | |
| | TTCAGAGCTAAGCACAAGAGTGC | |
| RPS18 | tcttgtggaaagCCAGAAACATG | 92 |
| | GAGACATTGACCTCACCAAGAGT | |
| | TTCAGAGCTAAGCACAAGAGTGC | |
| RPS19 | tcttgtggaaagCCAGAAACATG | 93 |
| | GGCGCGGCACCTGTACCTCCGGT | |
| | TTCAGAGCTAAGCACAAGAGTGC | |
| RPS20 | tcttgtggaaagCCAGAAACATG | 94 |
| | GACCAGTTCGAATGCCTACCAGT | |
| | TTCAGAGCTAAGCACAAGAGTGC | |
| RPS24 | tcttgtggaaagCCAGAAACATG | 95 |
| | GTAGGCACTGTCGCCTTCCCGGT | |
| | TTCAGAGCTAAGCACAAGAGTGC | |
| RPS26 | tcttgtggaaagCCAGAAACATG | 96 |
| | GCTGTGCCCGATGCGTGCCCAGT | |
| | TTCAGAGCTAAGCACAAGAGTGC | |
| RPS27A | tcttgtggaaagCCAGAAACATG | 97 |
| | GCCACCCGGCCCGTACCTCGAGT | |
| | TTCAGAGCTAAGCACAAGAGTGC | |
| RPS3 | tcttgtggaaagCCAGAAACATG | 98 |
| | GAGATGGCTACTCTGGAGTTGGT | |
| | TTCAGAGCTAAGCACAAGAGTGC | |
| RPS3A | tcttgtggaaagCCAGAAACATG | 99 |
| | GCAGACGAAGCAAGTAACCATGT | |
| | TTCAGAGCTAAGCACAAGAGTGC | |
| RPS4X | tcttgtggaaagCCAGAAACATG | 100 |
| | GTAAGATACTTACAAACACACGT | |
| | TTCAGAGCTAAGCACAAGAGTGC | |
| RPS5 | tcttgtggaaagCCAGAAACATG | 101 |
| | GCATACACCTGCTCACAGGCGGT | |
| | TTCAGAGCTAAGCACAAGAGTGC | |
| RPS6 | tcttgtggaaagCCAGAAACATG | 102 |
| | GAGTGGTGGGAACGACAAACAGT | |
| | TTCAGAGCTAAGCACAAGAGTGC | |
| RPS7 | tcttgtggaaagCCAGAAACATG | 103 |
| | GATAAGCAAAAGCGTCCCAGGGT | |
| | TTCAGAGCTAAGCACAAGAGTGC | |
| RPS8 | tcttgtggaaagCCAGAAACATG | 104 |
| | GACAGCACACCGTACCGACAGGT | |
| | TTCAGAGCTAAGCACAAGAGTGC | |
| RPS9 | tcttgtggaaagCCAGAAACATG | 105 |
| | GGCGGACCAGCCGCCGCAGCAGT | |
| | TTCAGAGCTAAGCACAAGAGTGC | |
| RPSA | tcttgtggaaagCCAGAAACATG | 106 |
| | GTACACAGCGCAATGGTAGGTGT | |
| | TTCAGAGCTAAGCACAAGAGTGC | |
| RPL10 | tcttgtggaaagCCAGAAACATG | 107 |
| | GTCTTGTTGATGCGGATGACGGT | |
| | TTCAGAGCTAAGCACAAGAGTGC | |
| RPL10A | tcttgtggaaagCCAGAAACATG | 108 |
| | GTCGCGACACCCTGTACGAGGGT | |
| | TTCAGAGCTAAGCACAAGAGTGC | |
| RPL11 | tcttgtggaaagCCAGAAACATG | 109 |
| | GTTCATTTCTCCGGATGCCAAGT | |
| | TTCAGAGCTAAGCACAAGAGTGC | |
| RPL12 | tcttgtggaaagCCAGAAACATG | 110 |
| | GTCTTCTGCAGTTAAACACAGGT | |
| | ITCAGAGCTAAGCACAAGAGTGC | |
| RPL13 | tcttgtggaaagCCAGAAACATG | 111 |
| | GTACCACACGAAGGTGCGCGCGT | |
| | ITCAGAGCTAAGCACAAGAGTGC | |
| RPL13A | tcttgtggaaagCCAGAAACATG | 112 |
| | GTCTTTCTCTAACAGAAAAAGGT | |
| | TTCAGAGCTAAGCACAAGAGTGC | |
| RPL14 | tcttgtggaaagCCAGAAACATG | 113 |
| | GTTGTCGGACATACTTCTGGTGT | |
| | TTCAGAGCTAAGCACAAGAGTGC | |
| RPL18 | tcttgtggaaagCCAGAAACATG | 114 |
| | GTCCTGGCCGGGAAAACAAGAGT | |
| | TTCAGAGCTAAGCACAAGAGTGC | |
| RPL18A | tcttgtggaaagCCAGAAACATG | 115 |
| | GGAAGTTCTTCACCCGCAGGGGT | |
| | TTCAGAGCTAAGCACAAGAGTGC | |
| RPL19 | tcttgtggaaagCCAGAAACATG | 116 |
| | GTGTACCCTTCCGCTTACCTGGT | |
| | TTCAGAGCTAAGCACAAGAGTGC | |
| RPL23A | tcttgtggaaagCCAGAAACATG | 117 |
| | GTAAAGCTGAAGCCAAAGCGAGT | |
| | TTCAGAGCTAAGCACAAGAGTGC | |
| RPL24 | tcttgtggaaagCCAGAAACATG | 118 |
| | GGCCGAGCAGTCAAATTCCAGGT | |
| | TTCAGAGCTAAGCACAAGAGTGC | |
| RPL26 | tcttgtggaaagCCAGAAACATG | 119 |
| | GTTTGCGATTCTTGCTTCGGTGT | |
| | TCAGAGCTAAGCACAAGAGTGC | |
| RPL27 | tcttgtggaaagCCAGAAACATG | 120 |
| | GTGGCAGCTGTCACTTTGCGGGT | |
| | TTCAGAGCTAAGCACAAGAGTGC | |
| RPL3 | tcttgtggaaagCCAGAAACATG | 121 |
| | GTGAGATGATCGACGTCATCGGT | |
| | TTCAGAGCTAAGCACAAGAGTGC | |
| RPL30 | tcttgtggaaagCCAGAAACATG | 122 |
| | GTGCTTTGTAGAAAAAGTCGCGT | |
| | TTCAGAGCTAAGCACAAGAGTGC | |
| RPL31 | tcttgtggaaagCCAGAAACATG | 123 |
| | GGCTTCAAGAAGCGTGCACCTGT | |
| | TTCAGAGCTAAGCACAAGAGTGC | |
| RPL32 | tcttgtggaaagCCAGAAACATG | 124 |
| | GCTTCCAGCTCCTTGACGTTGGT | |
| | TTCAGAGCTAAGCACAAGAGTGC | |
| RPL34 | tcttgtggaaagCCAGAAACATG | 125 |
| | GTATTGTAGGAAAGCCTACGTGT | |
| | TTCAGAGCTAAGCACAAGAGTGC | |
| RPL35 | tcttgtggaaagCCAGAAACATG | 126 |
| | GTTACATCTTAGAGAGCTTGGGT | |
| | TTCAGAGCTAAGCACAAGAGTGC | |
| RPL35A | tcttgtggaaagCCAGAAACATG | 127 |
| | GGTGTCTTAGGCTGTGGTCCAGT | |
| | TTCAGAGCTAAGCACAAGAGTGC | |
| RPL36 | tcttgtggaaagCCAGAAACATG | 128 |
| | GGTGGCTTTGCCCCGTACGAGGT | |
| | TTCAGAGCTAAGCACAAGAGTGC | |
| RPL37 | tcttgtggaaagCCAGAAACATG | 129 |
| | GTTTAGATAACTGGAGTGCCAGT | |
| | TTCAGAGCTAAGCACAAGAGTGC | |
| RPL37A | tcttgtggaaagCCAGAAACATG | 130 |
| | GTCGGGATCGTCGGTAAATACGT | |
| | TTCAGAGCTAAGCACAAGAGTGC | |
| RPL38 | tcttgtggaaagCCAGAAACATG | 131 |
| | GGTTGCAGCCTCGGAAAATTGGT | |
| | TTCAGAGCTAAGCACAAGAGTGC | |
| RPL4 | tcttgtggaaagCCAGAAACATG | 132 |
| | GTTTGTTAGGTCATCGTATTGGT | |
| | TTCAGAGCTAAGCACAAGAGTGC | |
| RPL5 | tcttgtggaaagCCAGAAACATG | 133 |
| | GTCATCTTGTACATACCCTGCGT | |
| | TTCAGAGCTAAGCACAAGAGTGC | |
| RPL6 | tcttgtggaaagCCAGAAACATG | 134 |
| | GGTTGGTGGTGACAAGAACGGGT | |
| | TTCAGAGCTAAGCACAAGAGTGC | |
| RPL7 | tcttgtggaaagCCAGAAACATG | 135 |
| | GTTCAGCTTCGAAAGGCAAGGGT | |
| | TTCAGAGCTAAGCACAAGAGTGC | |
| RPL7A | tcttgtggaaagCCAGAAACATG | 136 |
| | GGGGCCTACTCACCTGCTCGAGT | |
| | TTCAGAGCTAAGCACAAGAGTGC | |
| RPLP0 | tcttgtggaaagCCAGAAACATG | 137 |
| | GTTATCCGAAATGTTTCATTGGT | |
| | TTCAGAGCTAAGCACAAGAGTGC | |
| RPLP2 | tcttgtggaaagCCAGAAACATG | 138 |
| | GGGACGACGACCGGCTCAACAGT | |
| | TTCAGAGCTAAGCACAAGAGTGC | |
| RPS11 | tcttgtggaaagCCAGAAACATG | 139 |
| | GGTAATGTGTCCATTCGAGGGGT | |
| | TTCAGAGCTAAGCACAAGAGTGC | |
| RPS13 | tcttgtggaaagCCAGAAACATG | 140 |
| | GGTTGACATCTGACGACGTGAGT | |
| | TTCAGAGCTAAGCACAAGAGTGC | |
| RPS14 | tcttgtggaaagCCAGAAACATG | 141 |
| | GGACTGGTGGGATGAAGGTAAGT | |
| | TTCAGAGCTAAGCACAAGAGTGC | |
| RPS15 | tcttgtggaaagCCAGAAACATG | 142 |
| | GGCTGGTCGAGGTCCACGCCGGT | |
| | TTCAGAGCTAAGCACAAGAGTGC | |
| RPS15A | tcttgtggaaagCCAGAAACATG | 143 |
| | GTGACGTGCAACTCAAAGACCGT | |
| | TTCAGAGCTAAGCACAAGAGTGC | |
| RPS17 | tcttgtggaaagCCAGAAACATG | 144 |
| | GGAAAAGTACTACACGCGCCTGT | |
| | TTCAGAGCTAAGCACAAGAGTGC | |
| RPS18 | tcttgtggaaagCCAGAAACATG | 145 |
| | GTCAACACCAACATCGATGGGGT | |
| | TTCAGAGCTAAGCACAAGAGTGC | |
| RPS19 | tcttgtggaaagCCAGAAACATG | 146 |
| | GTGACGGTATCCACCCATTCGGT | |
| | TTCAGAGCTAAGCACAAGAGTGC | |
| RPS20 | tcttgtggaaagCCAGAAACATG | 147 |
| | GGTGTGTGCTGACTTGATAAGGT | |
| | TTCAGAGCTAAGCACAAGAGTGC | |
| RPS24 | tcttgtggaaagCCAGAAACATG | 148 |
| | GTGGTCATGAACTTTCTAGTGGT | |
| | TTCAGAGCTAAGCACAAGAGTGC | |
| RPS26 | tcttgtggaaagCCAGAAACATG | 149 |
| | GTGCAGCGAATAGGCTGCACGGT | |
| | TTCAGAGCTAAGCACAAGAGTGC | |
| RPS27A | tcttgtggaaagCCAGAAACATG | 150 |
| | GGGATACGATAGAAAATGTAAGT | |
| | TTCAGAGCTAAGCACAAGAGTGC | |
| RPS3 | tcttgtggaaagCCAGAAACATG | 151 |
| | GGCTGAAAAGGTGGCCACTAGGT | |
| | TTCAGAGCTAAGCACAAGAGTGC | |
| RPS3A | tcttgtggaaagCCAGAAACATG | 152 |
| | GITCCATGGTCAAAAAAIGGCGT | |
| | TTCAGAGCTAAGCACAAGAGTGC | |
| RPS4X | tcttgtggaaagCCAGAAACATG | 153 |
| | GTGTTCCTCAGGAAAATGATGGT | |
| | TTCAGAGCTAAGCACAAGAGTGC | |
| RPS5 | tcttgtggaaagCCAGAAACATG | 154 |
| | GTTCACTGCAATGTAATCCTGGT | |
| | TTCAGAGCTAAGCACAAGAGTGC | |
| RPS6 | tcttgtggaaagCCAGAAACATG | 155 |
| | GTACTTTCTATGAGAAGCGTAGT | |
| | TTCAGAGCTAAGCACAAGAGTGC | |
| RPS7 | tcttgtggaaagCCAGAAACATG | 156 |
| | GGCCAAGATCGTGAAGCCCAAGT | |
| | TTCAGAGCTAAGCACAAGAGTGC | |
| RPS8 | tcttgtggaaagCCAGAAACATG | 157 |
| | GGCTGGTTCGTACCAAGACCCGT | |
| | TTCAGAGCTAAGCACAAGAGTGC | |
| RPS9 | tcttgtggaaagCCAGAAACATG | 158 |
| | GTGGGCTCCGGAACAAACGTGGT | |
| | TTCAGAGCTAAGCACAAGAGTGC | |
| RPSA | tcttgtggaaagCCAGAAACATG | 159 |
| | GTTGGCAGGGAGCTCACTCAGGT | |
| | TTCAGAGCTAAGCACAAGAGTGC | |
| non- | tcttgtggaaagCCAGAAACATG | 160 |
| targeting1 | GTCCGTCTGCTTCATGAGCGGGT | |
| | TTCAGAGCTAAGCACAAGAGTGC | |
| non- | tcttgtggaaagCCAGAAACATG | 161 |
| targeting2 | GTCCTCACCTAAAGTGCAATAGT | |
| | TTCAGAGCTAAGCACAAGAGTGC | |
| non- | tcttgtggaaagCCAGAAACATG | 162 |
| targeting3 | GTCCTCGATAGCTGGAATCCAGT | |
| | TTCAGAGCTAAGCACAAGAGTGC | |
| non- | tcttgtggaaagCCAGAAACATG | 163 |
| targeting4 | GTCCTGCCAAGAAACACCCTTGT | |
| | TTCAGAGCTAAGCACAAGAGTGC | |
| non- | tcttgtggaaagCCAGAAACATG | 164 |
| targeting5 | GTCCTGGATACCGCGTGGTTAGT | |
| | TTCAGAGCTAAGCACAAGAGTGC | |
| non- | tcttgtggaaagCCAGAAACATG | 165 |
| targeting6 | GTCGAGAGGAAAAACACACTGGT | |
| | TTCAGAGCTAAGCACAAGAGTGC | |
| non- | tcttgtggaaagCCAGAAACATG | 166 |
| targeting7 | GTCGAGATGCGCAGCAGATGAGT | |
| | TTCAGAGCTAAGCACAAGAGTGC | |
| non- | tcttgtggaaagCCAGAAACATG | 167 |
| targeting8 | GTCGATCGAGGTTGCATTCGGGT | |
| | TTCAGAGCTAAGCACAAGAGTGC | |
| non- | tcttgtggaaagCCAGAAACATG | 168 |
| targeting9 | GTCGATGTAGCCCCGCCCAAGGT | |
| | TTCAGAGCTAAGCACAAGAGTGC | |
| non- | tcttgtggaaagCCAGAAACATG | 169 |
| targeting10 | GTCGCAAGGAAGCCAGCTAAGGT | |
| | TTCAGAGCTAAGCACAAGAGTGC | |
| non- | tcttgtggaaagCCAGAAACATG | 170 |
| targeting11 | GTCGCAGCGGCGTGGGATCGGGT | |
| | TTCAGAGCTAAGCACAAGAGTGC | |
| non- | tcttgtggaaagCCAGAAACATG | 171 |
| targeting12 | GTCGCGCTTGGGTTATACGCTGT | |
| | TTCAGAGCTAAGCACAAGAGTGC | |
| non- | tcttgtggaaagCCAGAAACATG | 172 |
| targetingl3 | GTCGCGGACATAGGGCTCTAAGT | |
| | TTCAGAGCTAAGCACAAGAGTGC | |
| non- | tcttgtggaaagCCAGAAACATG | 173 |
| targeting14 | GTCGGAAGCAAACTTCTGGAGGT | |
| | TTCAGAGCTAAGCACAAGAGTGC | |
| non- | tcttgtggaaagCCAGAAACATG | 174 |
| targeting15 | GTCGGCATACGGGACACACGCGT | |
| | TTCAGAGCTAAGCACAAGAGTGC | |
| non- | tcttgtggaaagCCAGAAACATG | 175 |
| targeting16 | GTCGGCTACAATCTTTGGCATGT | |
| | TTCAGAGCTAAGCACAAGAGTGC | |
| non- | tcttgtggaaagCCAGAAACATG | 176 |
| targeting17 | GTCGGCTACGGCGTGGAGAAGGT | |
| | TTCAGAGCTAAGCACAAGAGTGC | |
| non- | tcttgtggaaagCCAGAAACATG | 177 |
| targeting18 | GTCGGCTCCTGAAGCCAGTATGT | |
| | TTCAGAGCTAAGCACAAGAGTGC | |
| non- | tcttgtggaaagCCAGAAACATG | 178 |
| targeting19 | GTCGGGCAGTGAGTACAATACGT | |
| | TTCAGAGCTAAGCACAAGAGTGC | |
| non- | tcttgtggaaagCCAGAAACATG | 179 |
| targeting20 | GTCGGGGACCACCCACGATCCGT | |
| | TTCAGAGCTAAGCACAAGAGTGC | |
| non- | tcttgtggaaagCCAGAAACATG | 180 |
| targeting21 | GTCGTAAACACACGACCAAGTGT | |
| | TTCAGAGCTAAGCACAAGAGTGC | |
| non- | tcttgtggaaagCCAGAAACATG | 181 |
| targeting22 | GTCTAAAGCCGTCCTGATGTTGT | |
| | TTCAGAGCTAAGCACAAGAGTGC | |
| non- | tcttgtggaaagCCAGAAACATG | 182 |
| targeting23 | GTCTACCTATTGTGGAATTTGGT | |
| | TTCAGAGCTAAGCACAAGAGTGC | |
| non- | tcttgtggaaagCCAGAAACATG | 183 |
| targeting24 | GTCTACGTGTAGTTGTACATAGT | |
| | TTCAGAGCTAAGCACAAGAGTGC | |
| non- | tcttgtggaaagCCAGAAACATG | 184 |
| targeting25 | GTCTATTTTGTCTGCGCAGAAGT | |
| | TTCAGAGCTAAGCACAAGAGTGC | |
| non- | tcttgtggaaagCCAGAAACATG | 185 |
| targeting26 | GTCTCGTAGCCTAATGCGCCAGT | |
| | rrCAGAGCTAAGCACAAGAGTGC | |
| non- | tcttgtggaaagCCAGAAACATG | 186 |
| targeting27 | GTCTCTCGGAGTGGAGCAACAGT | |
| | TTCAGAGCTAAGCACAAGAGTGC | |
| non- | tcttgtggaaagCCAGAAACATG | 187 |
| targeting28 | GTCTGAAAAATAGGCCCAACCGT | |
| | TTCAGAGCTAAGCACAAGAGTGC | |
| non- | tcttgtggaaagCCAGAAACATG | 188 |
| targeting29 | GTCTGACGATTAATGCTTCTAGT | |
| | TTCAGAGCTAAGCACAAGAGTGC | |
| non- | tcttgtggaaagCCAGAAACATG | 189 |
| targeting30 | GTCTGGCTTGACACGACCGTTGT | |
| | TTCAGAGCTAAGCACAAGAGTGC | |
| non- | tcttgtggaaagCCAGAAACATG | 190 |
| targeting31 | GTCTGGCTTGCACCGTGTCATGT | |
| | TTCAGAGCTAAGCACAAGAGTGC | |
| non- | tcttgtggaaagCCAGAAACATG | 191 |
| targeting32 | GTGAACGCGTGTTTCCTTGCAGT | |
| | TTCAGAGCTAAGCACAAGAGTGC | |
| non- | tcttgtggaaagCCAGAAACATG | 192 |
| targeting33 | GTGAACGGTGAAGAGATAGGGGT | |
| | TTCAGAGCTAAGCACAAGAGTGC | |
| non- | tcttgtggaaagCCAGAAACATG | 193 |
| targeting34 | GTGAAGTGGGGCGTCGGACACGT | |
| | TTCAGAGCTAAGCACAAGAGTGC | |
| non- | tcttgtggaaagCCAGAAACATG | 194 |
| targeting35 | GTGAATCGAATACAAACGATGGT | |
| | TTCAGAGCTAAGCACAAGAGTGC | |
| non- | tcttgtggaaagCCAGAAACATG | 195 |
| targeting36 | GTGAATCGTAACCTCGCCATTGT | |
| | TTCAGAGCTAAGCACAAGAGTGC | |
| non- | tcttgtggaaagCCAGAAACATG | 196 |
| targeting37 | GTGACACATTGGCTGGGTGTTGT | |
| | TTCAGAGCTAAGCACAAGAGTGC | |
| non- | tcttgtggaaagCCAGAAACATG | 197 |
| targeting38 | GTGACCTCTGAGGAATTCACAGT | |
| | TTCAGAGCTAAGCACAAGAGTGC | |
| non- | tcttgtggaaagCCAGAAACATG | 198 |
| targeting39 | GTGACGCGATAGAGTTGGCTTGT | |
| | TTCAGAGCTAAGCACAAGAGTGC | |
| non- | tcttgtggaaagCCAGAAACATG | 199 |
| targeting40 | GTGACGCTCCACGTCCGGACCGT | |
| | TTCAGAGCTAAGCACAAGAGTGC | |
| non- | tcttgtggaaagCCAGAAACATG | 200 |
| targeting41 | GTGACTAGCTCTTACATATTCGT | |
| | TTCAGAGCTAAGCACAAGAGTGC | |
| non- | tcttgtggaaagCCAGAAACATG | 201 |
| targeting42 | GTGACTCGGGCAATATCGGTTGT | |
| | TTCAGAGCTAAGCACAAGAGTGC | |
| non- | tcttgtggaaagCCAGAAACATG | 202 |
| targeting43 | GTGAGCATGTCGGGAGTAACTGT | |
| | TTCAGAGCTAAGCACAAGAGTGC | |
| non- | tcttgtggaaagCCAGAAACATG | 203 |
| targeting44 | GTGAGCATTCGTAGCCCAGCAGT | |
| | TTCAGAGCTAAGCACAAGAGTGC | |
| non- | tcttgtggaaagCCAGAAACATG | 204 |
| targeting45 | GTGAGCGGCCTCTAATTAATCGT | |
| | TTCAGAGCTAAGCACAAGAGTGC | |
| non- | tcttgtggaaagCCAGAAACATG | 205 |
| targeting46 | GTGAGGATCATGTCGAGCGCCGT | |
| | TTCAGAGCTAAGCACAAGAGTGC | |
| non- | tcttgtggaaagCCAGAAACATG | 206 |
| targeting47 | GTGAGTCTTACTAGGTCCTGTGT | |
| | TTCAGAGCTAAGCACAAGAGTGC | |
| non- | tcttgtggaaagCCAGAAACATG | 207 |
| targeting48 | GTGCAACCTTCCTTTTCAGGAGT | |
| | TTCAGAGCTAAGCACAAGAGTGC | |
| non- | tcttgtggaaagCCAGAAACATG | 208 |
| targeting49 | GTGCAAGGACCTGGTATGAACGT | |
| | TTCAGAGCTAAGCACAAGAGTGC | |
| non- | tcttgtggaaagCCAGAAACATG | 209 |
| targeting50 | GTGCAGGTCTAGGTCCCAAAGGT | |
| | TTCAGAGCTAAGCACAAGAGTGC | |
| non- | tcttgtggaaagCCAGAAACATG | 210 |
| targeting5l | GTGCAGTCGCGCTGAGCGTCAGT | |
| | TTCAGAGCTAAGCACAAGAGTGC | |

### Dataset S2

sgRNA library for genetic screen of factors regulating RNA localization in the nucleus. This dataset lists the oligo sequences of the proto-spacers of 162 sgRNA targeting selected candidate genes for regulating RNA localization in the nucleus and 5 non-targeting sgRNAs.

| | | SEQ |
|---|---|---|
| | | ID |
| sgRNA | SEQUENCE | NO: |
| ALKBH5_1 | tcttgtggaaagCCAGAAACATG | 211 |
| | GATCAACGACTACCAGCCCGGGT | |
| | TTCAGAGCTAAGCACAAGAGTGC | |
| ALYREF_1 | tcttgtggaaagCCAGAAACATG | 212 |
| | GAGACGTGCACTTTGAGCGGAGT | |
| | TTCAGAGCTAAGCACAAGAGTGC | |
| DDX10_1 | tcttgtggaaagCCAGAAACATG | 213 |
| | GAGTGCAAGGATAGAAACACCGT | |
| | TTCAGAGCTAAGCACAAGAGTGC | |
| DDX18_1 | tcttgtggaaagCCAGAAACATG | 214 |
| | GAAACAAAAGCCCATGAATGTGT | |
| | TTCAGAGCTAAGCACAAGAGTGC | |
| DDX21_1 | tcttgtggaaagCCAGAAACATG | 215 |
| | GCAACATTAAATACCCAATGAGT | |
| | TTCAGAGCTAAGCACAAGAGTGC | |
| DDX24_1 | tcttgtggaaagCCAGAAACATG | 216 |
| | GCAACCACAATCTCAGGACGAGT | |
| | TTCAGAGCTAAGCACAAGAGTGC | |
| DDX27_1 | tcttgtggaaagCCAGAAACATG | 217 |
| | GGCAACTTGTCACAGACGCAGGT | |
| | TTCAGAGCTAAGCACAAGAGTGC | |
| DDX39B_1 | tcttgtggaaagCCAGAAACATG | 218 |
| | GCCTGCCAAGAAGGATGTCAAGT | |
| | TTCAGAGCTAAGCACAAGAGTGC | |
| DDX3X_1 | tcttgtggaaagCCAGAAACATG | 219 |
| | GCGTGGACGGAGTGATTACGAGT | |
| | TTCAGAGCTAAGCACAAGAGTGC | |
| DDX42_1 | tcttgtggaaagCCAGAAACATG | 220 |
| | GAGTGATTGTGTGTCCTACCAGT | |
| | TTCAGAGCTAAGCACAAGAGTGC | |
| DDX5_1 | tcttgtggaaagCCAGAAACATG | 221 |
| | GAAGTCTACTTGTATCTACGGGT | |
| | TTCAGAGCTAAGCACAAGAGTGC | |
| DDX50_1 | tcttgtggaaagCCAGAAACATG | 222 |
| | GAAGATTTAATAGCTCAAGCAGT | |
| | TTCAGAGCTAAGCACAAGAGTGC | |
| DDX51_1 | tcttgtggaaagCCAGAAACATG | 223 |
| | GACGTCAGGGATGTCCTCGATGT | |
| | TTCAGAGCTAAGCACAAGAGTGC | |
| DDX52_1 | tcttgtggaaagCCAGAAACATG | 224 |
| | GAAACACAAAATTCACGTCCAGT | |
| | TTCAGAGCTAAGCACAAGAGTGC | |
| DDX54_1 | tcttgtggaaagCCAGAAACATG | 225 |
| | GAGGTGCCAACACCCATCCAGGT | |
| | TTCAGAGCTAAGCACAAGAGTGC | |
| DDX56_1 | tcttgtggaaagCCAGAAACATG | 226 |
| | GATCATCTCACAGTTCAACCAGT | |
| | TTCAGAGCTAAGCACAAGAGTGC | |
| DHX15_1 | tcttgtggaaagCCAGAAACATG | 227 |
| | GCGCCCAGGAATAGTTAGGAGGT | |
| | TTCAGAGCTAAGCACAAGAGTGC | |
| DHX8_1 | tcttgtggaaagCCAGAAACATG | 228 |
| | GCCCAGGTCGAACATATCCAGGT | |
| | TTCAGAGCTAAGCACAAGAGTGC | |
| DHX9_1 | tcttgtggaaagCCAGAAACATG | 229 |
| | GCAAAACATTATACTGGCATGGT | |
| | TTCAGAGCTAAGCACAAGAGTGC | |
| FUS_1 | tcttgtggaaagCCAGAAACATG | 230 |
| | GAACACCACCGTACCTICCCGGT | |
| | TTCAGAGCTAAGCACAAGAGTGC | |
| HNRNPA0_1 | tcttgtggaaagCCAGAAACATG | 231 |
| | GCTTCGTGACCTACTCCAATGGT | |
| | PTCAGAGCTAAGCACAAGAGTGC | |
| HNRNPA1_1 | tcttgtggaaagCCAGAAACATG | 232 |
| | GCTTACTGACAATCTTATCCAGT | |
| | TTCAGAGCTAAGCACAAGAGTGC | |
| HNRNPA2B1_1 | tcttgtggaaagCCAGAAACATG | 233 |
| | GACTCTCCCATCAATTGAATGGT | |
| | TTCAGAGCTAAGCACAAGAGTGC | |
| HNRNPA3_1 | tcttgtggaaagCCAGAAACATG | 234 |
| | GAATGTGTGCTCGACCACACAGT | |
| | TTCAGAGCTAAGCACAAGAGTGC | |
| HNRNPAB_1 | tcttgtggaaagCCAGAAACATG | 235 |
| | GCCAACACTGGACGGTCAAGAGT | |
| | TTCAGAGCTAAGCACAAGAGTGC | |
| HNRNPC_1 | tcttgtggaaagCCAGAAACATG | 236 |
| | GAAATTCACTTACCTAAAACCGT | |
| | TTCAGAGCTAAGCACAAGAGTGC | |
| HNRNPCL1_1 | tcttgtggaaagCCAGAAACATG | 237 |
| | GAATTCTAAGAGTGGAAAGCGGT | |
| | TTCAGAGCTAAGCACAAGAGTGC | |
| HNRNPD_1 | tcttgtggaaagCCAGAAACATG | 238 |
| | GACAAGACCAATAAGAGGCGTGT | |
| | TTCAGAGCTAAGCACAAGAGTGC | |
| HNRNPDL_1 | tcttgtggaaagCCAGAAACATG | 239 |
| | GAGAGTACTTGTCTCGATTTGGT | |
| | TTCAGAGCTAAGCACAAGAGTGC | |
| HNRNPF_1 | tcttgtggaaagCCAGAAACATG | 240 |
| | GACTCCCATTTGGATGCACAAGT | |
| | TTCAGAGCTAAGCACAAGAGTGC | |
| HNRNPH1_1 | tcttgtggaaagCCAGAAACATG | 241 |
| | GAAATGGGATAACATTGCCGGGT | |
| | TTCAGAGCTAAGCACAAGAGTGC | |
| HNRNPH2_1 | tcttgtggaaagCCAGAAACATG | 242 |
| | GAGGTCCCTATGATAGGCCGGGT | |
| | TTCAGAGCTAAGCACAAGAGTGC | |
| HNRNPH3_1 | tcttgtggaaagCCAGAAACATG | 243 |
| | GACATTGACGATGGACTACCAGT | |
| | TTCAGAGCTAAGCACAAGAGTGC | |
| HXRXPK_1 | tcttgtggaaagCCAGAAACATG | 244 |
| | GATGATGTTTGATGACCGTCGGT | |
| | TTCAGAGCTAAGCACAAGAGTGC | |
| HNRNPL_1 | tcttgtggaaagCCAGAAACATG | 245 |
| | GACTCAGTTCAAAGTGCCCAGGT | |
| | TTCAGAGCTAAGCACAAGAGTGC | |
| HXRNPLL_1 | tcttgtggaaagCCAGAAACATG | 246 |
| | GAATACTGATGATCCATCAGGGT | |
| | TTCAGAGCTAAGCACAAGAGTGC | |
| HNRNPM_1 | tcttgtggaaagCCAGAAACATG | 247 |
| | GACCCCATGCCAATACCACCAGT | |
| | TTCAGAGCTAAGCACAAGAGTGC | |
| HNRNPR_1 | tcttgtggaaagCCAGAAACATG | 248 |
| | GACACTCCAAGGTGTTTACCAGT | |
| | TTCAGAGCTAAGCACAAGAGTGC | |
| HNRNPU_1 | tcttgtggaaagCCAGAAACATG | 249 |
| | GATCGAGTTAGAGGACCAAAGGT | |
| | TTCAGAGCTAAGCACAAGAGTGC | |
| HXRNPUL1_1 | tcttgtggaaagCCAGAAACATG | 250 |
| | GCCAGCCGATACGGACCACGTGT | |
| | TTCAGAGCTAAGCACAAGAGTGC | |
| HNRNPUL2_1 | tcttgtggaaagCCAGAAACATG | 251 |
| | GCAAGTGAGCAAAGACCGCTAGT | |
| | TTCAGAGCTAAGCACAAGAGTGC | |
| METTL16_1 | tcttgtggaaagCCAGAAACATG | 252 |
| | GAGATGGTATAGCTGCATGCTGT | |
| | TTCAGAGCTAAGCACAAGAGTGC | |
| NOXO_1 | tcttgtggaaagCCAGAAACATG | 253 |
| | GAGTC.GACCGCAACATCAAGGG | |
| | TTTCAGAGCTAAGCACAAGAGTG | |
| | C | |
| PCBP1_1 | tcttgtggaaagCCAGAAACATG | 254 |
| | GACACACTCGGTGACAGACTGGT | |
| | TTCAGAGCTAAGCACAAGAGTGC | |
| PCBP2_1 | tcttgtggaaagCCAGAAACATG | 255 |
| | GAGTTGGCAGTATCATCGGAAGT | |
| | TTCAGAGCTAAGCACAAGAGTGC | |
| PTBP1_1 | tcttgtggaaagCCAGAAACATG | 256 |
| | GCATGGTGAACTACTACACCTGT | |
| | TTCAGAGCTAAGCACAAGAGTGC | |
| RBMX_1 | tcttgtggaaagCCAGAAACATG | 257 |
| | GAAATATGGACGAATAGTGGAGT | |
| | TTCAGAGCTAAGCACAAGAGTGC | |
| SFPQ_1 | tcttgtggaaagCCAGAAACATG | 258 |
| | GATGATCGTGGAAGATCTACAGT | |
| | TTCAGAGCTAAGCACAAGAGTGC | |
| SYNCR1P_1 | tcttgtggaaagCCAGAAACATG | 259 |
| | GGATGACAAGAAAAAAAACAGGT | |
| | TTCAGAGCTAAGCACAAGAGTGC | |
| YTHDC1_1 | tcttgtggaaagCCAGAAACATG | 260 |
| | GATGAGTGCTAAAATGCTGGGGT | |
| | TTCAGAGCTAAGCACAAGAGTGC | |
| YTHDF2_1 | tcttgtggaaagCCAGAAACATG | 261 |
| | GATGGAGGGACTGTAGTAACTGT | |
| | TTCAGAGCTAAGCACAAGAGTGC | |
| METTL3_1 | tcttgtggaaagCCAGAAACATG | 262 |
| | GATTCTGTGACTATGGAACCAGT | |
| | TTCAGAGCTAAGCACAAGAGTGC | |
| METTL14_1 | tcttgtggaaagCCAGAAACATG | 263 |
| | GACCATCTTACCACTCTTCCAGT | |
| | TTCAGAGCTAAGCACAAGAGTGC | |
| SON_1 | tcttgtggaaagCCAGAAACATG | 264 |
| | GGCCAGTTGTAACAATGTCAGGT | |
| | TTCAGAGCTAAGCACAAGAGTGC | |
| ALKBH5_2 | tcttgtggaaagCCAGAAACATG | 265 |
| | GCCTGTACAACGAGCACACGGGT | |
| | TTCAGAGCTAAGCACAAGAGTGC | |
| ALYREF_2 | tcttgtggaaagCCAGAAACATG | 266 |
| | GGCCTCGATTCACTCGCGCGGGT | |
| | TTCAGAGCTAAGCACAAGAGTGC | |
| DDX10_2 | tcttgtggaaagCCAGAAACATG | 267 |
| | GCATGAACCCAGACATACTCAGT | |
| | TTCAGAGCTAAGCACAAGAGTGC | |
| DDX18_2 | tcttgtggaaagCCAGAAACATG | 268 |
| | GCTGATCGTATCTTGGATGTGGT | |
| | TTCAGAGCTAAGCACAAGAGTGC | |
| DDX21_2 | tcttgtggaaagCCAGAAACATG | 269 |
| | GCATCACAAAAAAGCTGTCAGGT | |
| | TTCAGAGCTAAGCACAAGAGTGC | |
| DDX24_2 | tcttgtggaaagCCAGAAACATG | 270 |
| | GCATGGTTTGTGATGATCCGGGT | |
| | TTCAGAGCTAAGCACAAGAGTGC | |
| DDX27_2 | tcttgtggaaagCCAGAAACATG | 271 |
| | GCTTAATCGGAACCATAGGCGGT | |
| | TTCAGAGCTAAGCACAAGAGTGC | |
| DDX39B_2 | tcttgtggaaagCCAGAAACATG | 272 |
| | GCTCAAAGCCACAGTCGACAAGT | |
| | ITCAGAGCTAAGCACAAGAGTGC | |
| DDX3X_2 | tcttgtggaaagCCAGAAACATG | 273 |
| | GGCACCACCATAAACCACGCAGT | |
| | TTCAGAGCTAAGCACAAGAGTGC | |
| DDX42_2 | tcttgtggaaagCCAGAAACATG | 274 |
| | GCCTGATCGACCCTATTCGAGGT | |
| | TTCAGAGCTAAGCACAAGAGTGC | |
| DDX5_2 | tcttgtggaaagCCAGAAACATG | 275 |
| | GCATTCCTAGAGAGAGGCGATGT | |
| | TTCAGAGCTAAGCACAAGAGTGC | |
| DDX50_2 | tcttgtggaaagCCAGAAACATG | 276 |
| | GAAGTGATAATAAACTAGAGGGT | |
| | TTCAGAGCTAAGCACAAGAGTGC | |
| DDX51_2 | tcttgtggaaagCCAGAAACATG | 277 |
| | GCTGCCTCGCAGAAAGCAACGGT | |
| | TTCAGAGCTAAGCACAAGAGTGC | |
| DDX52_2 | tcttgtggaaagCCAGAAACATG | 278 |
| | GTCAGTGTGTCCATTGGAGCAGT | |
| | TTCAGAGCTAAGCACAAGAGTGC | |
| DDX54_2 | tcttgtggaaagCCAGAAACATG | 279 |
| | GCAGGTAGGGGATTTCATCAGGT | |
| | TTCAGAGCTAAGCACAAGAGTGC | |
| DDX56_2 | tcttgtggaaagCCAGAAACATG | 280 |
| | GCCAGTTACAGCAGTTTCAGGGT | |
| | TTCAGAGCTAAGCACAAGAGTGC | |
| DHX15_2 | tcttgtggaaagCCAGAAACATG | 281 |
| | GTCATAGTATCGAGGAGTATGGT | |
| | TTCAGAGCTAAGCACAAGAGTGC | |
| DHX8_2 | tcttgtggaaagCCAGAAACATG | 282 |
| | GTAGACGGCGAAATCTTGTCGGT | |
| | TTCAGAGCTAAGCACAAGAGTGC | |
| DHX9_2 | tcttgtggaaagCCAGAAACATG | 283 |
| | GGGAGATITACCAACAACCATGT | |
| | TTCAGAGCTAAGCACAAGAGTGC | |
| FUS_2 | tcttgtggaaagCCAGAAACATG | 284 |
| | GCAAAGCTATAATCCCCCTCAGT | |
| | TTCAGAGCTAAGCACAAGAGTGC | |
| HNRNPA0_2 | tcttgtggaaagCCAGAAACATG | 285 |
| | GGGAGGATATCTACTCCGGTGGT | |
| | TTCAGAGCTAAGCACAAGAGTGC | |
| HNRNPA1_2 | tcttgtggaaagCCAGAAACATG | 286 |
| | GGGGAACGCTCACGGACTGTGGT | |
| | TTCAGAGCTAAGCACAAGAGTGC | |
| HNRNPA2B1_ | tcttgtggaaagCCAGAAACATG | 287 |
| 2 | GAGGAACTACTACGAACAATGGT | |
| | TTCAGAGCTAAGCACAAGAGTGC | |
| HNRNPA3_2 | tcttgtggaaagCCAGAAACATG | 288 |
| | GCTAATACCTCCTCCCCCCGGGT | |
| | TTCAGAGCTAAGCACAAGAGTGC | |
| HNRNPAB_2 | tcttgtggaaagCCAGAAACATG | 289 |
| | GCGCCACCGAGAACGGACATGGT | |
| | TTCAGAGCTAAGCACAAGAGTGC | |
| HNRNPC_2 | tcttgtggaaagCCAGAAACATG | 290 |
| | GAGGTGTGAAACGATCTGCAGGT | |
| | TTCAGAGCTAAGCACAAGAGTGC | |
| HNRNPCL1_2 | tcttgtggaaagCCAGAAACATG | 291 |
| | GAGGTGTGAAACGATCAGCAGGT | |
| | TTCAGAGCTAAGCACAAGAGTGC | |
| HNRNPD_2 | tcttgtggaaagCCAGAAACATG | 292 |
| | GCCATGGTGGCGGCGACACAGGT | |
| | TTCAGAGCTAAGCACAAGAGTGC | |
| HNRNPDL_2 | tcttgtggaaagCCAGAAACATG | 293 |
| | GCAACGCGAGCAAGAATCAGCGT | |
| | TTCAGAGCTAAGCACAAGAGTGC | |
| HNRNPF_2 | tcttgtggaaagCCAGAAACATG | 294 |
| | GCCATTTCATCTACACTAGAGGT | |
| | TTCAGAGCTAAGCACAAGAGTGC | |
| HNRNPH1_2 | tcttgtggaaagCCAGAAACATG | 295 |
| | GAATGTCTGATCACAGATACGGT | |
| | TTCAGAGCTAAGCACAAGAGTGC | |
| HNRNPH2_2 | tcttgtggaaagCCAGAAACATG | 296 |
| | GCTGTGAAGCAAACTGCACAAGT | |
| | TTCAGAGCTAAGCACAAGAGTGC | |
| HNRNPH3_2 | tcttgtggaaagCCAGAAACATG | 297 |
| | GAGATAGCAGAAAATGCTCTGGT | |
| | TTCAGAGCTAAGCACAAGAGTGC | |
| HNRNPK_2 | tcttgtggaaagCCAGAAACATG | 298 |
| | GCTGTTGGGACATACCGCTCGGT | |
| | TTCAGAGCTAAGCACAAGAGTGC | |
| HNRNPL_2 | tcttgtggaaagCCAGAAACATG | 299 |
| | GAGGATGGGTCCACCAGTGGGGT | |
| | TTCAGAGCTAAGCACAAGAGTGC | |
| HNRNPLL_2 | tcttgtggaaagCCAGAAACATG | 300 |
| | GAGGACTCTGTGAATCTGTGGGT | |
| | TTCAGAGCTAAGCACAAGAGTGC | |
| HNRNPM_2 | tcttgtggaaagCCAGAAACATG | 301 |
| | GAGGTGATGGCTACGACTGGTGT | |
| | TTCAGAGCTAAGCACAAGAGTGC | |
| HNRNPR_2 | tcttgtggaaagCCAGAAACATG | 302 |
| | GAGAACCAGATCCAGAAGTCAGT | |
| | TTCAGAGCTAAGCACAAGAGTGC | |
| HNRNPU_2 | tcttgtggaaagCCAGAAACATG | 303 |
| | GCACCGTCACCGCGAACAGCGGT | |
| | TTCAGAGCTAAGCACAAGAGTGC | |
| HNRNPUL1_2 | tcttgtggaaagCCAGAAACATG | 304 |
| | GCCCCGGATACGCTCACTAAGGT | |
| | TTCAGAGCTAAGCACAAGAGTGC | |
| HNRNPUL2_2 | tcttgtggaaagCCAGAAACATG | 305 |
| | GTCTTACGGTTTCGATGGACGGT | |
| | TTCAGAGCTAAGCACAAGAGTGC | |
| METTL16_2 | tcttgtggaaagCCAGAAACATG | 306 |
| | GCAGCATAAACGAGTTCCCTGGT | |
| | TTCAGAGCTAAGCACAAGAGTGC | |
| NONO_2 | tcttgtggaaagCCAGAAACATG | 307 |
| | GCTGGACAATATGCCACTCCGGT | |
| | TTCAGAGCTAAGCACAAGAGTGC | |
| PCBP1_2 | tcttgtggaaagCCAGAAACATG | 308 |
| | GAGAGATCCGCGAGAGTACGGGT | |
| | TTCAGAGCTAAGCACAAGAGTGC | |
| PCBP2_2 | tcttgtggaaagCCAGAAACATG | 309 |
| | GCAAGATCAAGGAAATACGAGGT | |
| | TTCAGAGCTAAGCACAAGAGTGC | |
| PTBP1_2 | tcttgtggaaagCCAGAAACATG | 310 |
| | GCCACGATGATCCTGAGCACGGT | |
| | TTCAGAGCTAAGCACAAGAGTGC | |
| RBMX_2 | tcttgtggaaagCCAGAAACATG | 311 |
| | GACATCTCTACGAGAGGGCAGGT | |
| | TTCAGAGCTAAGCACAAGAGTGC | |
| SFPQ_2 | tcttgtggaaagCCAGAAACATG | 312 |
| | GATGGGCCTCAATCAGAATCGGT | |
| | TTCAGAGCTAAGCACAAGAGTGC | |
| SYNCRIP_2 | tcttgtggaaagCCAGAAACATG | 313 |
| | GTATTCCTAAGAGTAAAACCAGT | |
| | TTCAGAGCTAAGCACAAGAGTGC | |
| YTHDC1_2 | tcttgtggaaagCCAGAAACATG | 314 |
| | GATTCTTATAAGGTTCTCTGGGT | |
| | TTCAGAGCTAAGCACAAGAGTGC | |
| YTHDF2_2 | tcttgtggaaagCCAGAAACATG | 315 |
| | GGAATACTATAGACCAAGGGAGT | |
| | TTCAGAGCTAAGCACAAGAGTGC | |
| METTL3_2 | tcttgtggaaagCCAGAAACATG | 316 |
| | GCTTGCTCTTACACAGAGTGTGT | |
| | TTCAGAGCTAAGCACAAGAGTGC | |
| METTL14_2 | tcttgtggaaagCCAGAAACATG | 317 |
| | GTAACACGGCACCAATGCTGTGT | |
| | TTCAGAGCTAAGCACAAGAGTGC | |
| SON_2 | tcttgtggaaagCCAGAAACATG | 318 |
| | GGGACATCATAGAGCGCTCGTGT | |
| | TTCAGAGCTAAGCACAAGAGTGC | |
| ALKBH5_3 | tcttgtggaaagCCAGAAACATG | 319 |
| | GGAGGCGCGCAAGGTGAAGAGGT | |
| | TTCAGAGCTAAGCACAAGAGTGC | |
| ALYREF_3 | tcttgtggaaagCCAGAAACATG | 320 |
| | GTTTCCCACCTGTCTCCACGCGT | |
| | TTCAGAGCTAAGCACAAGAGTGC | |
| DDX10_3 | tcttgtggaaagCCAGAAACATG | 321 |
| | GTGATAGGCCAGTTCTCTCGTGT | |
| | TTCAGAGCTAAGCACAAGAGTGC | |
| DDX18_3 | tcttgtggaaagCCAGAAACATG | 322 |
| | GTGATAAAGCGAATGCAACAGGT | |
| | TTCAGAGCTAAGCACAAGAGTGC | |
| DDX21_3 | tcttgtggaaagCCAGAAACATG | 323 |
| | GGATGTCATCCGAGTATATAGGT | |
| | TTCAGAGCTAAGCACAAGAGTGC | |
| DDX24_3 | tcttgtggaaagCCAGAAACATG | 324 |
| | GTGAGACTAGATCACCAGGCAGT | |
| | TTCAGAGCTAAGCACAAGAGTGC | |
| DDX27_3 | tcttgtggaaagCCAGAAACATG | 325 |
| | GTGATGAGTAGTCAGTCTCCGGT | |
| | TTCAGAGCTAAGCACAAGAGTGC | |
| DDX39B_3 | tcttgtggaaagCCAGAAACATG | 326 |
| | GGATAAGATGCTTGAACAGCTGT | |
| | TTCAGAGCTAAGCACAAGAGTGC | |
| DDX3X_3 | tcttgtggaaagCCAGAAACATG | 327 |
| | GTCTCGGTTCCTTAAATGAGGGT | |
| | TTCAGAGCTAAGCACAAGAGTGC | |
| DDX42_3 | tcttgtggaaagCCAGAAACATG | 328 |
| | GTCTACAAAATTGGATCTAAGGT | |
| | TTCAGAGCTAAGCACAAGAGTGC | |
| DDX5_3 | tcttgtggaaagCCAGAAACATG | 329 |
| | GCTGATAGGCAAACTCTAATGGT | |
| | TTCAGAGCTAAGCACAAGAGTGC | |
| DDX50_3 | tcttgtggaaagCCAGAAACATG | 330 |
| | GTATAACTAGGAAACTCAGCGGT | |
| | TTCAGAGCTAAGCACAAGAGTGC | |
| DDX51_3 | tcttgtggaaagCCAGAAACATG | 331 |
| | GnGACAGCATGCATCAGTCCGTT | |
| | TCAGAGCTAAGCACAAGAGTGC | |
| DDX52_3 | tcttgtggaaagCCAGAAACATG | 332 |
| | GTTATTAAAGCAAGATCCCCCGT | |
| | TTCAGAGCTAAGCACAAGAGTGC | |
| DDX54_3 | tcttgtggaaagCCAGAAACATG | 333 |
| | GGAGGCATCGCTGGAGCTACGCT | |
| | nTCAGAGCTAAGCACAAGAGTGC | |
| DDX56_3 | tcttgtggaaagCCAGAAACATG | 334 |
| | GTTGCTCCATAGGAAGGCGGTGT | |
| | TTCAGAGCTAAGCACAAGAGTGC | |
| DHX15_3 | tcttgtggaaagCCAGAAACATG | 335 |
| | GTTACACCATAACGCTCCAGGGT | |
| | TTCAGAGCTAAGCACAAGAGTGC | |
| DHX8_3 | tcttgtggaaagCCAGAAACATG | 336 |
| | GTGATGATCGCGTACTGAGTGGT | |
| | TTCAGAGCTAAGCACAAGAGTGC | |
| DHX9_3 | tcttgtggaaagCCAGAAACATG | 337 |
| | GTGGATGTGGGAAAACCACACGT | |
| | TTCAGAGCTAAGCACAAGAGTGC | |
| FUS_3 | tcttgtggaaagCCAGAAACATG | 338 |
| | GGCCACCACCACTACTCATGGGT | |
| | TTCAGAGCTAAGCACAAGAGTGC | |
| HNRNPA0_3 | tcttgtggaaagCCAGAAACATG | 339 |
| | GTGGGACTCTGACGGACTGCGGT | |
| | TTCAGAGCTAAGCACAAGAGTGC | |
| HXRXPA1_3 | tcttgtggaaagCCAGAAACATG | 340 |
| | GTTCATGTCTTAAGGTCGAAGGT | |
| | TTCAGAGCTAAGCACAAGAGTGC | |
| HNRNPA2B1_ | tcttgtggaaagCCAGAAACATG | 341 |
| 3 | GTTTAGGAATC1GGAAAACCAGT | |
| | TTCAGAGCTAAGCACAAGAGTGC | |
| HXRXPA3_3 | tcttgtggaaagCCAGAAACATG | 342 |
| | GTTCCACCAAAGTTTCCACCAGT | |
| | TTCAGAGCTAAGCACAAGAGTGC | |
| HNRNPAB_3 | tcttgtggaaagCCAGAAACATG | 343 |
| | GGTGAAGAAAATCTTCGTTGGGT | |
| | TTCAGAGCTAAGCACAAGAGTGC | |
| HNRNPC_3 | tcttgtggaaagCCAGAAACATG | 344 |
| | GTATCAGGAAACACTTCACGAGT | |
| | TTCAGAGCTAAGCACAAGAGTGC | |
| HNRNPCL1_3 | tcttgtggaaagCCAGAAACATG | 345 |
| | GTGTAGATATTAACCTGGCTGGT | |
| | TTCAGAGCTAAGCACAAGAGTGC | |
| HXRXPD_3 | tcttgtggaaagCCAGAAACATG | 346 |
| | GGTTTATAGGAGGCCTTAGCTGT | |
| | TTCAGAGCTAAGCACAAGAGTGC | |
| HXRXPDL_3 | tcttgtggaaagCCAGAAACATG | 347 |
| | GTGGAGCTGGATTTAAAATGGGT | |
| | TTCAGAGCTAAGCACAAGAGTGC | |
| HNRNPF_3 | tcttgtggaaagCCAGAAACATG | 348 |
| | GTGAAGCCGTAGCCATCACTGGT | |
| | TTCAGAGCTAAGCACAAGAGTGC | |
| HNRNPH1_3 | tcttgtggaaagCCAGAAACATG | 349 |
| | GTGGTCCAAATAGTCCTGACAGT | |
| | TTCAGAGCTAAGCACAAGAGTGC | |
| HNRNPH2_3 | tcttgtggaaagCCAGAAACATG | 350 |
| | GTCTGATCATAGATACGGAGAGT | |
| | TTCAGAGCTAAGCACAAGAGTGC | |
| HNRNPH3_3 | tcttgtggaaagCCAGAAACATG | 351 |
| | GTATGACAGAATGCGACGAGGGT | |
| | TTCAGAGCTAAGCACAAGAGTGC | |
| HNRNPK_3 | tcttgtggaaagCCAGAAACATG | 352 |
| | GTAAAATCAAAGAACTTCGAGGT | |
| | TTCAGAGCTAAGCACAAGAGTGC | |
| HNRNPL_3 | tcttgtggaaagCCAGAAACATG | 353 |
| | GTGCCCTCACCATATTCTGCGGT | |
| | TTCAGAGCTAAGCACAAGAGTGC | |
| HNRNPLL_3 | tcttgtggaaagCCAGAAACATG | 354 |
| | GTTAGGAATGACAATGACAGTGT | |
| | TTCAGAGCTAAGCACAAGAGTGC | |
| HNRNPM_3 | tcttgtggaaagCCAGAAACATG | 355 |
| | GGGTGGTGGTATGGAAAACATGT | |
| | TTCAGAGCTAAGCACAAGAGTGC | |
| HNRNPR_3 | tcttgtggaaagCCAGAAACATG | 356 |
| | GGGTCCGGCCTTCTCAAAAAGGT | |
| | TTCAGAGCTAAGCACAAGAGTGC | |
| HXRNPU_3 | tcttgtggaaagCCAGAAACATG | 357 |
| | GGGCTGGTCACTAACTACAAGGT | |
| | TTCAGAGCTAAGCACAAGAGTGC | |
| HXRXPUL1_3 | tcttgtggaaagCCAGAAACATG | 358 |
| | GGGGGTACTTTGAGCACCGAGGT | |
| | TTCAGAGCTAAGCACAAGAGTGC | |
| HXRXPUL2_3 | tcttgtggaaagCCAGAAACATG | 359 |
| | GTTACTATGAATTCCGAGAGGGT | |
| | TTCAGAGCTAAGCACAAGAGTGC | |
| METTL16_3 | tcttgtggaaagCCAGAAACATG | 360 |
| | GTCTGACGTGTACTCTCCTAAGT | |
| | TTCAGAGCTAAGCACAAGAGTGC | |
| NONO_3 | tcttgtggaaagCCAGAAACATG | 361 |
| | GGCTCTGGACAGATGCAGTGAGT | |
| | TTCAGAGCTAAGCACAAGAGTGC | |
| PCBP1_3 | tcttgtggaaagCCAGAAACATG | 362 |
| | GATGG1CATGACTCTCCCTTGGT | |
| | TTCAGAGCTAAGCACAAGAGTGC | |
| PCBP2_3 | tcttgtggaaagCCAGAAACATG | 363 |
| | GGTGTGTCAAACAGATCTGCGGT | |
| | TTCAGAGCTAAGCACAAGAGTGC | |
| PTBP1_3 | tcttgtggaaagCCAGAAACATG | 364 |
| | GCGTGGACGTTCGGAACGGAAGT | |
| | TTCAGAGCTAAGCACAAGAGTGC | |
| RBMX_3 | tcttgtggaaagCCAGAAACATG | 365 |
| | GCTCTAGTATCACGAGAACTTGT | |
| | TTCAGAGCTAAGCACAAGAGTGC | |
| SFPQ_3 | tcttgtggaaagCCAGAAACATG | 366 |
| | GTCTACCTGCTGATATCACGGGT | |
| | TTCAGAGCTAAGCACAAGAGTGC | |
| SYNCRIP_3 | tcttgtggaaagCCAGAAACATG | 367 |
| | GTGAGCGAGATGGTGCTGTCAGT | |
| | TTCAGAGCTAAGCACAAGAGTGC | |
| YTHDC1_3 | tcttgtggaaagCCAGAAACATG | 368 |
| | GTGATTATGACACTCGAAGTGGT | |
| | TTCAGAGCTAAGCACAAGAGTGC | |
| YTHDI-2_3 | tcttgtggaaagCCAGAAACATG | 369 |
| | GTGGGTTCGGTCATAATGGGGGT | |
| | TTCAGAGCTAAGCACAAGAGTGC | |
| METTL3_3 | tcttgtggaaagCCAGAAACATG | 370 |
| | GTATCTCCAGATCAACATCTGGT | |
| | TTCAGAGCTAAGCACAAGAGTGC | |
| METTL14_3 | tcttgtggaaagCCAGAAACATG | 371 |
| | GTTGAAGAATATCCTAAACTGGT | |
| | TTCAGAGCTAAGCACAAGAGTGC | |
| SON_3 | tcttgtggaaagCCAGAAACATG | 372 |
| | GGGTTGAAGGACTGACACCGCGT | |
| | TTCAGAGCTAAGCACAAGAGTGC | |
| non-targeting1 | tcttgtggaaagCCAGAAACATG | 373 |
| | GATCTTCAGGGTAACTACGAAGT | |
| | TTCAGAGCTAAGCACAAGAGTGC | |
| non-targeting2 | tcttgtggaaagCCAGAAACATG | 374 |
| | GATCTTCTCGACGAAAATGCGGT | |
| | TTCAGAGCTAAGCACAAGAGTGC | |
| non-targeting3 | tcttgtggaaagCCAGAAACATG | 375 |
| | GATGACATTGCGCGTCTACGGGT | |
| | TTCAGAGCTAAGCACAAGAGTGC | |
| non-targeting4 | tcttgtggaaagCCAGAAACATG | 376 |
| | GATGATATCTGACATGCAGCGGT | |
| | TTCAGAGCTAAGCACAAGAGTGC | |
| non-targeting5 | tcttgtggaaagCCAGAAACATG | 377 |
| | GATGCAAGACAGCCTCCCAGCGT | |
| | TTCAGAGCTAAGCACAAGAGTGC | |

### Dataset S3

Features quantified and gene hits identified in the screen for factors regulating nuclear RNA localization. This dataset lists the gene hits for individual phenotype features quantified in the screen.

| Phenotype | Increase | Decrease |
|---|---|---|
| SON cluster intensity | | SON |
| SON cluster area | | SON |
| SON cluster number | | SON |
| preribosome cluster intensity | DDX56, HNRNPM, | DDX10, DDX18, DDX52, HNRNPH1, |
| | SON | HNRNPK, HNRNPLL, SFPQ |
| preribosome cluster area | | DDX24 |
| Preribosome cluster number | | DDX21 |
| MRP cluster intensity | | HNRNPK, HNRNPH1 |
| MRP cluster area | | |
| MRP cluster number | HNRNPK | DDX21 |
| 7sk SON enrichment | | |
| U2 SON enrichment | HNRNPK | DDX42, DDX5 |
| polyA SON enrichment | HNRNPK | DDX5 |
| MALAT1 SON enrichment | HNRNPA1, HNRNPL, | DDX42, DHX15, |
| | PCBP1, HNRNPH3 | HNRNPH1, HNRNPK |

### Dataset S4

DNA oligo sequences used for library cloning and sequencing. This dataset lists the DNA oligo sequences used for library construction and next-generation sequencing (for the determination of barcode identity and barcode-sgRNA correspondence within the libraries, and for the quantification of proto-spacer and UMI abundance in recombination quantification).

| | | | SEQ |
|---|---|---|---|
| OLIGO | | | ID |
| NO. | NAME | SEQUENCE | NO: |
| 1 | trit1_value1 | cagaaacatggaagacgtgaccc | 378 |
| | trit2_value1 | ggAGATCCGATTGGAACCGTCCC | |
| | | AAGCGTTGCGAAACGAGACGGTC | |
| | | AATCGCGCTGCATACTTG | |
| 2 | trit1_value1 | cagaaacatggaagacgtgaccc | 379 |
| | trit2_value2 | ggAGATCCGATTGGAACCGTCCC | |
| | | AAGCGTTGCGAAGTGAATTGCTG | |
| | | CGCATCGGCGTACGACTT | |
| 3 | trit1_value1 | cagaaacatggaagacgtgaccc | 380 |
| | trit2_value3 | ggAGATCCGATTGGAACCGTCCC | |
| | | AAGCGTTGCGAAAGTTCAACGGG | |
| | | ACCGTGGCCGATGTTTCG | |
| 4 | Trit1_value2 | cagaaacatggaagacgtgaccc | 381 |
| | trit2_value1 | ggAGCTATCGTTCGTTCGAGGCC | |
| | | AGAGCATTCGAAACGAGACGGTC | |
| | | AATCGCGCTGCATACTTG | |
| 5 | trit1_value2 | cagaaacatggaagacgtgaccc | 382 |
| | trit2_value2 | ggAGCTATCGTTCGTTCGAGGCC | |
| | | AGAGCATTCGAAGTGAATTGCTG | |
| | | CGCATCGGCGTACGACTT | |
| 6 | trit1_value2 | cagaaacatggaagacgtgaccc | 383 |
| | trit2_value3 | ggAGCTATCGTTCGTTCGAGGCC | |
| | | AGAGCATTCGAAAGTTCAACGGG | |
| | | ACCGTGGCCGATGTTTCG | |
| 7 | trit1_value3 | cagaaacatggaagacgtgaccc | 384 |
| | trit2_value1 | ggACCCATGATCGTCCGATCTGG | |
| | | TCGGATTTGTAAACGAGACGGTC | |
| | | AATCGCGCTGCATACTTG | |
| 8 | trit1_value3 | cagaaacatggaagacgtgaccc | 385 |
| | trit2_value2 | ggACCCATGATCGTCCGATCTGG | |
| | | TCGGATTTGTAAGTGAATTGCTG | |
| | | CGCATCGGCGTACGACTT | |
| 9 | trit1_value3 | cagaaacatggaagacgtgaccc | 386 |
| | trit2_value3 | ggACCCATGATCGTCCGATCTGG | |
| | | TCGGATTTGTAAAGTTCAACGGG | |
| | | ACCGTGGCCGATGTTTCG | |
| 10 | trit2_value1 | CCGGTTCGACTGAAGGTTTACTA | 387 |
| | trit3_value1_RC | GGCGAGGTCAAGTATGCAGCGCG | |
| | | ATTGACCGTCTCGTTT | |
| 11 | trit2_value1 | GCTCGTCGGGCAACAATAGCTCT | 388 |
| | dit3_value2_RC | ACGCACCTCAAGTATGCAGCGCG | |
| | | ATTGACCGTCTCGTTT | |
| 12 | trit2_value1 | GCATGAGTTGCCTGGCGTTGCGA | 389 |
| | trit3_value3_RC | CGACTAATCAAGTATGCAGCGCG | |
| | | ATTGACCGTCTCGTTT | |
| 13 | trit2_value2 | CCGGTTCGACTGAAGGTTTACTA | 390 |
| | trit3_valuel_RC | GGCGAGGTAAGTCGTACGCCGAT | |
| | | GCGCAGCAATTcAcTT | |
| 14 | trit2_value2 | GCTCGTCGGGCAACAATAGCTCT | 391 |
| | trit3_value2_RC | ACGCACCTAAGTCGTACGCCGAT | |
| | | GCGCAGCAATTCACTT | |
| 15 | trit2_value2 | GCTCGTCGGGCAACAATAGCTCT | 392 |
| | trit3_value3_RC | ACGCACCTAAGTCGTACGCCGAT | |
| | | GCGCAGCAATTCACTT | |
| 16 | trit2_value3 | CCGGTTCGACTGAAGGTTTACTA | 393 |
| | trit3_valuel_RC | GGCGAGGTCGAAACATCGGCCAC | |
| | | GGTCCCGTTGAACTTT | |
| 17 | trit2_value3 | GCTCGTCGGGCAACAATAGCTCT | 394 |
| | trit3_value2_RC | ACGCACCTCGAAACATCGGCCAC | |
| | | GGTCCCGTTGAACTTT | |
| 18 | trit2_value3 | GCATGAGTTGCCTGGCGTTGCGA | 395 |
| | trit3_value3_RC | CGACTAATCGAAACATCGGCCAC | |
| | | GGTCCCGTTGAACTTT | |
| 19 | trit3_value1 | ACCTCGCCTAGTAAACCTTCAGT | 396 |
| | trit4_value1 | CGAACCGGAGTAAGCGCAACGGT | |
| | | GGACCGGAGACGACGG | |
| 20 | trit3_value1 | ACCTCGCCTAGTAAACCTTCAGT | 397 |
| | trit4_value2 | CGAACCGGAAAATTGCGTGACGG | |
| | | ACCTGGGCCATTGGCC | |
| 21 | trit3_value1 | ACCTCGCCTAGTAAACCTTCAGT | 398 |
| | trit4_value3 | CGAACCGGAGCAGATTCCGCTAC | |
| | | GCTCCGATTCGATCAA | |
| 22 | trit3_value2 | AGGTGCGTAGAGCTATTGTTGCC | 399 |
| | trit4_value1 | CGACGAGCAGTAAGCGCAACGGT | |
| | | GGACCGGAGACGACGG | |
| 23 | trit3_value2 | AGGTGCGTAGAGCTATTGTTGCC | 400 |
| | trit4_value2 | CGACGAGCAAAATTGCGTGACGG | |
| | | ACCTGGGCCATTGGCC | |
| 24 | trit3_value2 | AGGTGCGTAGAGCTATTGTTGCC | 401 |
| | trit4_value3 | CGACGAGCAGCAGATTCCGCTAC | |
| | | GCTCCGATTCGATCAA | |
| 25 | trit3_value3 | ATTAGTCGTCGCAACGCCAGGCA | 402 |
| | trit4_value1 | ACTCATGCAGTAAGCGCAACGGT | |
| | | GGACCGGAGACGACGG | |
| 26 | trit3_value3 | ATTAGTCGTCGCAACGCCAGGCA | 403 |
| | trit4_value2 | ACTCATGCAAAATTGCGTGACGG | |
| | | ACCTGGGCCATTGGCC | |
| 27 | trit3_value3 | ATTAGTCGTCGCAACGCCAGGCA | 404 |
| | trit4_value3 | ACTCATGCAGCAGATTCCGCTAC | |
| | | GCTCCGATTCGATCAA | |
| 28 | trit4_value1 | TTAGGTCCGGCGATTAGCGCTCG | 405 |
| | trit5_value1_RC | TGCGCGATCCGTCGTCTCCGGTC | |
| | | CACCGTTGCGCTTACT | |
| 29 | trit4_value1 | GCCTCGATTACGACGGATGTAAT | 406 |
| | trit5_value2_RC | TCGGCCGTCCGTCGTCTCCGGTC | |
| | | CACCGTTGCGCTTACT | |
| 30 | trit4_value1 | GCCCGTATTCCCGCTTGCGAGTA | 407 |
| | trit5_value3_RC | GGGCAATTCCGTCGTCTCCGGTC | |
| | | CACCGTTGCGCTTACT | |
| 31 | trit4_value2 | TTAGGTCCGGCGATTAGCGCTCG | 408 |
| | trit5_value1_RC | TGCGCGATGGCCAATGGCCCAGG | |
| | | TCCGTCACGCAATTTT | |
| 32 | trit4_value2 | GCCTCGATTACGACGGATGTAAT | 409 |
| | trit5_value2_RC | TCGGCCGTGGCCAATGGCCCAGG | |
| | | TCCGTCACGCAATTTT | |
| 33 | trit4_value2 | GCCCGTATTCCCGCTTGCGAGTA | 410 |
| | trit5_value3_RC | GGGCAATTGGCCAATGGCCCAGG | |
| | | TCCGTCACGCAATTTT | |
| 34 | trit4_value3 | TTAGGTCCGGCGATTAGCGCTCG | 411 |
| | trit5_value1_RC | TGCGCGATTTGATCGAATCGGAG | |
| | | CGTAGCGGAATCTGCT | |
| 35 | trit4_value3 | GCCTCGATTACGACGGATGTAAT | 412 |
| | trit5_value2_RC | TCGGCCGTTTGATCGAATCGGAG | |
| | | CGTAGCGGAATCTGCT | |
| 36 | trit4_value3 | GCCCGTATTCCCGCTTGCGAGTA | 413 |
| | trit5_value3_RC | GGGCAATTTTGATCGAATCGGAG | |
| | | CGTAGCGGAATCTGCT | |
| 37 | trit5_value1 | ATCGCGCACGAGCGCTAATCGCC | 414 |
| | trit6_value1 | GGACCTAAAGGTCGATGCCCTAA | |
| | | TCCACGTGCTTCCCGC | |
| 38 | trit5_value1 | ATCGCGCACGAGCGCTAATCGCC | 415 |
| | trit6_value2 | GGACCTAAACCATAACAACGGCT | |
| | | AGCACGGCACGCAAAT | |
| 39 | trit5_value1 | ATCGCGCACGAGCGCTAATCGCC | 416 |
| | trit6_value3 | GGACCTAAACCCTCGGTCCCACC | |
| | | TTACGGCGATGACCCT | |
| 40 | trit5_value2 | ACGGCCGAATTACATCCGTCGTA | 417 |
| | trit6_value1 | ATCGAGGCAGGTCGATGCCCTAA | |
| | | TCCACGTGCTTCCCGC | |
| 41 | trit5_value2 | ACGGCCGAATTACATCCGTCGTA | 418 |
| | trit6_value2 | ATCGAGGCACCATAACAACGGCT | |
| | | AGCACGGCACGCAAAT | |
| 42 | trit5_value2 | ACGGCCGAATTACATCCGTCGTA | 419 |
| | trit6_value3 | ATCGAGGCACCCTCGGTCCCACC | |
| | | TTACGGCGATGACCCT | |
| 43 | trit5_value3 | AATTGCCCTACTCGCAAGCGGGA | 420 |
| | trit6_value1 | ATACGGGCAGGTCGATGCCCTAA | |
| | | TCCACGTGCTTCCCGC | |
| 44 | trit5_value3 | AATTGCCCTACTCGCAAGCGGGA | 421 |
| | trit6_value2 | ATACGGGCACCATAACAACGGCT | |
| | | AGCACGGCACGCAAAT | |
| 45 | trit5_value3 | AATTGCCCTACTCGCAAGCGGGA | 422 |
| | trit6_value3 | ATACGGGCACCCTCGGTCCCACC | |
| | | TTACGGCGATGACCCT | |
| 46 | trit6_value1 | CAACGACTGGCGACCTTAGGGTT | 423 |
| | trit7_value1_RC | GGCTCGCTGCGGGAAGCACGTGG | |
| | | ATTAGGGCATCGACCT | |
| 47 | trit6_value1 | CGAGTAGGCCGCCTGAACAACTA | 424 |
| | trit7_value2_RC | GCCGGAGTGCGGGAAGCACGTGG | |
| | | ATTAGGGCATCGACCT | |
| 48 | trit6_value1 | GGACACGGGATCGACGGTAGTGG | 425 |
| | trit7_value3_RC | ATCGATTTGCGGGAAGCACGTGG | |
| | | ATTAGGGCATCGACCT | |
| 49 | trit6_value2 | CAACGACTGGCGACCTTAGGGTT | 426 |
| | trit7_value1_RC | GGCTCGCTATTTGCGTGCCGTGC | |
| | | TAGCCGTTGTTATGGT | |
| 50 | trit6_value2 | CGAGTAGGCCGCCTGAACAACTA | 427 |
| | trit7_value2_RC | GCCGGAGTATTTGCGTGCCGTGC | |
| | | TAGCCGTTGTTATGGT | |
| 51 | trit6_value2 | GGACACGGGATCGACGGTAGTGG | 428 |
| | trit7_value3_RC | ATCGATTTATTTGCGTGCCGTGC | |
| | | TAGCCGTTGTTATGGT | |
| 52 | trit6_value3 | CAACGACTGGCGACCTTAGGGTT | 429 |
| | trit7_value1_RC | GGCTCGCTAGGGTCATCGCCGTA | |
| | | AGGTGGGACCGAGGGT | |
| 53 | trit6_value3 | CGAGTAGGCCGCCTGAACAACTA | 430 |
| | trit7_value2_RC | GCCGGAGTAGGGTCATCGCCGTA | |
| | | AGGTGGGACCGAGGGT | |
| 54 | trit6_value3 | GGACACGGGATCGACGGTAGTGG | 431 |
| | tiit7_value3_RC | ATCGATTTAGGGTCATCGCCGTA | |
| | | AGGTGGGACCGAGGGT | |
| 55 | trit7_value1 | AGCGAGCCAACCCTAAGGTCGCC | 432 |
| | trit8_value1 | AGTCGTTGAAACCGGTACATGAC | |
| | | GCGGAACCTAAGGTCG | |
| 56 | trit7_value1 | AGCGAGCCAACCCTAAGGTCGCC | 433 |
| | trit8_value2 | AGTCGTTGAGTGAAATCGCAGCA | |
| | | GCCCACGTCGTAAACA | |
| 57 | trit7_value1 | AGCGAGCCAACCCTAAGGTCGCC | 434 |
| | trit8_value3 | AGTCGTTGATAGCAAAGCCGGTA | |
| | | GCGACAACCGTTTCCC | |
| 58 | trit7_value2 | ACTCCGGCTAGTTGTTCAGGCGG | 435 |
| | trit8_value1 | CCTACTCGAAACCGGTACATGAC | |
| | | GCGGAACCTAAGGTCG | |
| 59 | trit7_value2 | ACTCCGGCTAGTTGTTCAGGCGG | 436 |
| | trit8_value2 | CCTACTCGAGTGAAATCGCAGCA | |
| | | GCCCACGTCGTAAACA | |
| 60 | trit7_value2 | ACTCCGGCTAGTTGTTCAGGCGG | 437 |
| | tiit8_value3 | CCTACTCGATAGCAAAGCCGGTA | |
| | | GCGACAACCGTTTCCC | |
| 61 | trit7_value3 | AAATCGATCCACTACCGTCGATC | 438 |
| | trit8_value1 | CCGTGTCCAAACCGGTACATGAC | |
| | | GCGGAACCTAAGGTCG | |
| 62 | trit7_value3 | AAATCGATCCACFACCGTCGATC | 439 |
| | trit8_value2 | CCGTGTCCAGTGAAATCGCAGCA | |
| | | GCCCACGTCGTAAACA | |
| 63 | trit7_value3 | AAATCGATCCACTACCGTCGATC | 440 |
| | trit8_value3 | CCGTGTCCATAGCAAAGCCGGTA | |
| | | GCGACAACCGTTTCCC | |
| 64 | trit8_value1 | GATGGTCGACTGGCGGTCTTAAT | 441 |
| | tiit9_value1RC | ATGCCCATCGACCTTAGGTTCCG | |
| | | CGTCATGTACCGGTTT | |
| 65 | trit8_value1 | ATCCATATGACCGGCGGCCTTTT | 442 |
| | tiit9_value2_RC | CTCGGACTCGACCTTAGGTTCCG | |
| | | CGTCATGTACCGGTTT | |
| 66 | trit8_value1 | ATGATCCACGACCGAGCAGGTTA | 443 |
| | trit9_value3_RC | GTTGACGTCGACCTTAGGTTCCG | |
| | | CGTCATGTACCGGTTT | |
| 67 | trit8_value2 | GATGGTCGACTGGCGGTCTTAAT | 444 |
| | trit9_value1RC | ATGCCCATTGTTTACGACGTGGG | |
| | | CTGCTGCGATTTCACT | |
| 68 | trit8_value2 | ATCCATATGACCGGCGGCCTTTT | 445 |
| | trit9_value2RC | CTCGGACTTGTTTACGACGTGGG | |
| | | CTGCTGCGATTTCACT | |
| 69 | trit8_value2 | ATGATCCACGACCGAGCAGGTTA | 446 |
| | trit9_value3RC | GTTGACGTTGTTTACGACGTGGG | |
| | | CTGCTGCGATTTCACT | |
| 70 | trit8_value3 | GATGGTCGACTGGCGGTCTTAAT | 447 |
| | trit9_value1RC | ATGCCCATGGGAAACGGTTGTCG | |
| | | CTACCGGCTTTGCTAT | |
| 71 | trit8_value3 | ATCCATATGACCGGCGGCCTTTT | 448 |
| | trit9_value2_RC | CTCGGACTGGGAAACGGTTGTCG | |
| | | CTACCGGCTTTGCTAT | |
| 72 | trit8_value3 | ATGATCCACGACCGAGCAGGTTA | 449 |
| | trit9_value3_RC | GTTGACGTGGGAAACGGTTGTCG | |
| | | CTACCGGCTTTGCTAT | |
| 73 | trit9_value1 | ATGGGCATATTAAGACCGCCAGT | 450 |
| | trit10_value1 | CGACCATCAGTCCTGTTCTTGTC | |
| | | GAGCGTGGTGCGTCTA | |
| 74 | trit9_value1 | ATGGGCATATTAAGACCGCCAGT | 451 |
| | trit10_value2 | CGACCATCAATCCGTAGACCAAC | |
| | | CGGCCGTTAAACGAGT | |
| 75 | trit9_value1 | ATGGGCATATTAAGACCGCCAGT | 452 |
| | trit10_value3 | CGACCATCACTGAATCCGTCGTT | |
| | | CCAGACGCGAGTACCC | |
| 76 | trit9_value2 | AGTCCGAGAAAAGGCCGCCGGTC | 453 |
| | trit10_value1 | ATATGGATAGTCCTGTTdTGTCG | |
| | | AGCGTGGTGCGTCTA | |
| 77 | trit9_value2 | AGTCCGAGAAAAGGCCGCCGGTC | 454 |
| | trit10_value2 | ATATGGATAATCCGTAGACCAAC | |
| | | CGGCCGTTAAACGAGT | |
| 78 | trit9_value2 | AGTCCGAGAAAAGGCCGCCGGTC | 455 |
| | tri110_value3 | ATATGGATACTGAATCCGTCGTT | |
| | | CCAGACGCGAGTACCC | |
| 79 | trit9_value3 | ACGTCAACTAACCTGCTCGGTCG | 456 |
| | trit10_value1 | TGGATCATAGTCCTGTTCTTGTC | |
| | | GAGCGTGGTGCGTCTA | |
| 80 | trit9_value3 | ACGTCAACTAACCTGCTCGGTCG | 457 |
| | trit10_value2 | TGGATCATAATCCGTAGACCAAC | |
| | | CGGCCGTTAAACGAGT | |
| 81 | trit9_value3 | ACGTCAACTAACCTGCTCGGTCG | 458 |
| | trit10_value3 | TGGATCATACTGAATCCGTCGTT | |
| | | CCAGACGCGAGTACCC | |
| 82 | trit10_value1 | AACATCGGATCGGTGCGGTGGGA | 459 |
| | trit11_value1_RC | TGGATAATTAGACGCACCACGCT | |
| | | CGACAAGAACAGGACT | |
| 83 | trit10_value1 | AGACGACGCACGTTCGTACCGCG | 460 |
| | trit11_value2_RC | TACTTCGTTAGACGCACCACGCT | |
| | | CGACAAGAACAGGACT | |
| 84 | trit10_value1 | TTTGCTCGCAAGTGCGCACGAGT | 461 |
| | trit11_value3_RC | TGAACTGTTAGACGCACCACGCT | |
| | | CGACAAGAACAGGACT | |
| 85 | trit10_value2 | AACATCGGATCGGTGCGGTGGGA | 462 |
| | trit11_value1_RC | TGGATAATACTCGTTTAACGGCC | |
| | | GGTTGGTCTACGGATT | |
| 86 | trit10_value2 | AGACGACGCACGTTCGTACCGCG | 463 |
| | trit11value2RC | TACTTCGTACTCGTTTAACGGCC | |
| | | GGTTGGTCTACGGATT | |
| 87 | trit10_value2 | TTTGCTCGCAAGTGCGCACGAGT | 464 |
| | trit11_value3_RC | TGAACTGTACTCGTTTAACGGCC | |
| | | GGTTGGTCTACGGATT | |
| 88 | trit10_value3 | AACATCGGATCGGTGCGGTGGGA | 465 |
| | trit11_value1_RC | TGGATAATGGGTACTCGCGTCTG | |
| | | GAACGACGGATTCAGT | |
| 89 | trit10_value3 | AGACGACGCACGTTCGTACCGCG | 466 |
| | trit11_value2_RC | TACTTCGTGGGTACTCGCGTCTG | |
| | | GAACGACGGATTCAGT | |
| 90 | trit10_value3 | TTTGCTCGCAAGTGCGCACGAGT | 467 |
| | trit11_value3_RC | TGAACTGTGGGTACTCGCGTCTG | |
| | | GAACGACGGATTCAGT | |
| 91 | trit11_value1 | ATTATCCATCCCACCGCACCGAT | 468 |
| | trit12_value1 | CCGATGTTAAGCCCGTCTAAACG | |
| | | CGGCGAGGTACATTGGgccgtcc | |
| | | gtcatctcgttttcgatg | |
| 92 | trit11_value1 | ATTATCCATCCCACCGCACCGAT | 469 |
| | trit12_value2 | CCGATGTTAGCCGCGATATTCAC | |
| | | GCCGCGTATGGAAACTgccgtcc | |
| | | gtcatctcgttttcgatg | |
| 93 | trit11_value1 | ATTATCCATCCCACCGCACCGAT | 470 |
| | trit12_value3 | CCGATGTTAATGAAACGTCTGGG | |
| | | CCCGTAACGCTGTAGCgccgtcc | |
| | | gtcatctcgttttcgatg | |
| 94 | trit11_value2 | ACGAAGTACGCGGTACGAACGTG | 471 |
| | trit12_value1 | CGTCGTCTAAGCCCGTCTAAACG | |
| | | CGGCGAGGTACATTGGgccgtcc | |
| | | gtcatctcgttttcgatg | |
| 95 | trit11_value2 | ACGAAGTACGCGGTACGAACGTG | 472 |
| | trit12_value2 | CGTCGTCTAGCCGCGATATTCAC | |
| | | GCCGCGTATGGAAACTgccgtcc | |
| | | gtcatctcgttttcgatg | |
| % | trit11_value2 | ACGAAGTACGCGGTACGAACGTG | 473 |
| | trit12_value3 | CGTCGTCTAATGAAACGTCTGGG | |
| | | CCCGTAACGCTGTAGCgccgtcc | |
| | | gtcatctcgttttcgatg | |
| 97 | trit11_value3 | ACAGTTCAACTCGTGCGCACTTG | 474 |
| | trit12_value1 | CGAGCAAAAAGCCCGTCTAAACG | |
| | | CGGCGAGGTACATiGGgccgtcc | |
| | | gtcatctcgttttcgatg | |
| 98 | trit11_value3 | ACAGTTCAACTCGTGCGCACTTG | 475 |
| | trit12_value2 | CGAGCAAAAGCCGCGATATTCAC | |
| | | GCCGCGTATGGAAACTgccgtcc | |
| | | gtcatctcgttttcgatg | |
| 99 | trit11_value3 | ACAGTTCAACTCGTGCG.CACTT | 476 |
| | trit12_value3 | GCGAGCAAAAATGAAACGTCTGG | |
| | | GCCCGTAACGCTGTAGCgccgtc | |
| | | cgtcatctcgttttcgatg | |
| 100 | Barcode assem | catcgaaaacgagatgacggacg | 477 |
| | rev | gc | |
| 101 | Barcode assem | ggctttatatatcttgtggaaag | 478 |
| | fwd | CCAGAAACATGGAAGACGtgacc | |
| | | cgg | |
| 102 | UMI rev | CCAGAGGTTGATTATCGTAATGG | 479 |
| | | ATCCTNNNNNNNNNNNNNNNNNN | |
| | | NNtctagacatcgaaaacgagat | |
| | | gacggacggc | |
| 103 | reporter insert | ggctttatatatcttgtggaaag | 480 |
| | fwd | CCAGAAACAaGGaccaggatggg | |
| | | caccacccGTTTCAGAGCTAAGC | |
| | | ACAAGAGTGC | |
| 104 | reporter insert | ccgggtcaCGTCTTCCATGTTTC | 481 |
| | rev | TGGctatcaTTACACCTTGCGCT | |
| | | TCTTCTTGGG | |
| 105 | New 1 step | GTGGCACCCGAGTCGGGTGCTTT | 482 |
| | trit1_value1 | TTTTtcgggTAccAGATCCGATT | |
| | trit2_value1 | GGAACCGTCCCAAGCGTTGCGAA | |
| | | ACGAGACGGTCAATCGCGCTGCA | |
| | | TACTTG | |
| 106 | New 1 step | GTGGCACCCGAGTCGGGTGCTTT | 483 |
| | trit1_value1 | TTTTtcgggTAccAGATCCGATT | |
| | trit2_value2 | GGAACCGTCCCAAGCGTTGCGAA | |
| | | GTGAATTGCTGCGCATCGGCGTA | |
| | | CGACTT | |
| 107 | New 1 step | GTGGCACCCGAGTCGGGTGCTTT | 484 |
| | trit1_value1 | TTTTtcgggTAccAGATCCGATT | |
| | tri t2_value3 | GGAACCGTCCCAAGCGTTGCGAA | |
| | | AGTTCAACGGGACCGTGGCCGAT | |
| | | GTTi’CG | |
| 108 | New 1 step | GTGGCACCCGAGTCGGGTGCTTT | 485 |
| | trit1_value2 | iTTTtcgggTAccAGCTATCGTT | |
| | trit2_value 1 | CGTTCGAGGCCAGAGCATTCGAA | |
| | | ACGAGACGGTCAATCGCGCTGCA | |
| | | TACTTG | |
| 109 | New 1 step | GTGGCACCCGAGTCGGGTGCTTT | 486 |
| | trit1_value2 | TTTTtcgggTAccAGCTATCGTT | |
| | trit2_value2 | CGTTCGAGGCCAGAGCATTCGAA | |
| | | GTGAATTGCTGCGCATCGGCGTA | |
| | | CGACTT | |
| 110 | New 1 step | GTGGCACCCGAGTCGGGTGCTTT | 487 |
| | trit1_value2 | TTTTtcgggTAccAGCTATCGTT | |
| | tri t2_value3 | CGTTCGAGGCCAGAGCATTCGAA | |
| | | AGTTCAACGGGACCGTGGCCGAT | |
| | | GTTTCG | |
| 111 | New 1 step | GTGGCACCCGAGTCGGGTGCTTT | 488 |
| | trit1_value3 | TTTTtcgggTAccACCCATGATC | |
| | trit2_value1 | GTCCGATCTGGTCGGATTTGTAA | |
| | | ACGAGACGGTCAATCGCGCTGCA | |
| | | TACTTG | |
| 112 | New 1 step | GTGGCACCCGAGTCGGGTGCTTT | 489 |
| | trit1_value3 | TTTTtcgggTAccACCCATGATC | |
| | trit2_value2 | GTCCGATCTGGTCGGATTTGTAA | |
| | | GTGAATTGCTGCGCATCGGCGTA | |
| | | CGACTT | |
| 113 | New 1 step | GTGGCACCCGAGTCGGGTGCTTT | 490 |
| | trit1_value3 | TTTTtcgggTAccACCCATGATC | |
| | trit2_value3 | GTCCGATCTGGTCGGATTTGTAA | |
| | | AGTTCAACGGGACCGTGGCCGAT | |
| | | GTTTCG | |
| 114 | gRNA constant | GTTTCAGAGCTAAGCACAAGAGT | 491 |
| | fwd | GC | |
| 115 | gRNA constant | GCACCCGACTCGGGTGCCAC | 492 |
| | rev | | |
| 116 | PS fwd | gctttatatatcttgtggaaagC | 493 |
| | | CAGAAACATGG | |
| 117 | PS rev | GCACTCTTGTGCTTAGCTCTGAA | 494 |
| | | AC | |
| 118 | PS UMI rev | GTCGACGGTATttatcgtaatgg | 495 |
| | | atcctNNNNNNNNNNNNNNNNNN | |
| | | NNtctagacatcgaaaacgagat | |
| | | gacggacggc | |
| 119 | 501 F12 | AATGATACGGCGACCACCGAGAT | 496 |
| | ligation | CTACACTATAGCCTACACTCTTT | |
| | | CCCTACACGACGCTCTTCCGATC | |
| | | TNNNNNNNNNNNNCCAGAAACAT | |
| | | GGgcatgtGGATCC | |
| 120 | 501 F11 | AATGATACGGCGACCACCGAGAT | 497 |
| | ligation | CTACACTATAGCCTACACTCTTT | |
| | | CCCTACACGACGCTCTTCCGATC | |
| | | TNNNNNNNNNNNCCAGAAACATG | |
| | | GgcatgtGGATCC | |
| 121 | 501 F10 | AATGATACGGCGACCACCGAGAT | 498 |
| | ligation | CTACACTATAGCCTACACTCTTT | |
| | | CCCTACACGACGCTCTTCCGATC | |
| | | TNNNNNNNNNNCCAGAAACATGG | |
| | | gcatgtGGATCC | |
| 122 | 701R 12 | CAAGCAGAAGACGGCATACGAGA | 499 |
| | ligation | TCGAGTAATGTGACTGGAGTTCA | |
| | | GACGTGTGCTCTTCCGATCTNNN | |
| | | NNNNNNNNNGGATCCacatgcCC | |
| | | ATGTTTCTGG | |
| 123 | 701R 11 | CAAGCAGAAGACGGCATACGAGA | 500 |
| | ligation | TCGAGTAATGTGACTGGAGTTCA | |
| | | GACGTGTGCTCTTCCGATCTNNN | |
| | | NNNNNNNNGGATCCacatgcCCA | |
| | | TGTTTCTGG | |
| 124 | 701R10 | CAAGCAGAAGACGGCATACGAGA | 501 |
| | ligation | TCGAGTAATGTGACTGGAGTTCA | |
| | | GACGTGTGCTCTTCCGATCTNNN | |
| | | NNNNNNNGGATCCacatgcCCAT | |
| | | GTTTCTGG | |
| 125 | 502 F12 ligation | AATGATACGGCGACCACCGAGAT | 502 |
| | | CTACACATAGAGGCACACTCTTT | |
| | | CCCTACACGACGCTCTTCCGATC | |
| | | TNNNNNNNNNNNNCCAGAAACAT | |
| | | GGgcatgtGGATCC | |
| 126 | 502 F11 ligation | AATGATACGGCGACCACCGAGAT | 503 |
| | | CTACACATAGAGGCACACTCTTT | |
| | | CCCTACACGACGCTCTTCCGATC | |
| | | TNNNNNNNNNNNCCAGAAACATG | |
| | | GgcatgtGGATCC | |
| 127 | 502 F10 ligation | AATGATACGGCGACCACCGAGAT | 504 |
| | | CTACACATAGAGGCACACTCTTT | |
| | | CCCTACACGACGCTCTTCCGATC | |
| | | TNNNNNNNNNNCCAGAAACATGG | |
| | | gcatgtGGATCC | |
| 128 | 702R 12 ligation | CAAGCAGAAGACGGCATACGAGA | 505 |
| | | TTCTCCGGAGTGACTGGAGTTCA | |
| | | GACGTGTGCTCTTCCGATCTNNN | |
| | | NNNNNNNNNGGATCCacatgcCC | |
| | | ATGTTTCTGG | |
| 129 | 702R 11 ligation | CAAGCAGAAGACGGCATACGAGA | 506 |
| | | TTCTCCGGAGTGACTGGAGTTCA | |
| | | GACGTGTGCTCTTCCGATCTNNN | |
| | | NNNNNNNNGGATCCacatgcCCA | |
| | | TGTTTCTGG | |
| 130 | 702R10 ligation | CAAGCAGAAGACGGCATACGAGA | 507 |
| | | TTCTCCGGAGTGACTGGAGTTCA | |
| | | GACGTGTGCTCTTCCGATCTNNN | |
| | | NNNNNNNGGATCCacatgcCCAT | |
| | | GTTTCTGG | |
| 131 | 501 F12 ligation | AATGATACGGCGACCACCGAGAT | 508 |
| | KpnI | CTACACTATAGCCTACACTCTTT | |
| | | CCCTACACGACGCTCTTCCGATC | |
| | | TNNNNNNNNNNNNcacggTAccg | |
| | | tactgcaGGATCCtgc | |
| 132 | 501 F11 ligation | AATGATACGGCGACCACCGAGAT | 509 |
| | KpnI | CTACACTATAGCCTACACTCTTT | |
| | | CCCTACACGACGCTCTTCCGATC | |
| | | TNNNNNNNNNNNcacggTAccgt | |
| | | actgcaGGATCCtgc | |
| 133 | 501 F10 ligation | AATGATACGGCGACCACCGAGAT | 510 |
| | KpnI | CTACACT’ATAGCCTACACTCTT | |
| | | TCCCTACACGACGCTCTTCCGAT | |
| | | CTNNNNNNNNNNcacggTAccgt | |
| | | actgcaGGATCCtgc | |
| 134 | 701R 12 ligation | CAAGCAGAAGACGGCATACGAGA | 511 |
| | KpnI | TCGAGTAATGTGACTGGAGTTCA | |
| | | GACGTGTGCTCTTCCGATCTNNN | |
| | | NNNNNNNNNgcaGGATCCtgcag | |
| | | tacggTAccgtg | |
| 135 | 701R 11 ligation | CAAGCAGAAGACGGCATACGAGA | 512 |
| | KpnI | TCGAGTAATGTGACTGGAGTTCA | |
| | | GACGTGTGCTCTTCCGATCTNNN | |
| | | NNNNNNNNgcaGGATCCtgcagt | |
| | | acggTAccgtg | |
| 136 | 701R10 ligation | CAAGCAGAAGACGGCATACGAGA | 513 |
| | KpnI | TCGAGTAATGTGACTGGAGTTCA | |
| | | GACGTGTGCTCTTCCGATCTNNN | |
| | | NNNNNNNgcaGGATCCtgcagta | |
| | | cggTAccgtg | |
| 137 | 501 F12 gRNA | AATGATACGGCGACCACCGAGAT | 514 |
| | fwd | CTACACTATAGCCTACACTCTTT | |
| | | CCCTACACGACGCTCTTCCGATC | |
| | | TNNNNNNNNNNNNgctttatata | |
| | | tcttgtggaaagCCAGAAACATG | |
| | | G | |
| 138 | 501 F11 gRNA | AATGATACGGCGACCACCGAGAT | 515 |
| | fwd | CTACACTATAGCCTACACTCTTT | |
| | | CCCTACACGACGCTCTTCCGATC | |
| | | TNNNNNNNNNNNgctttatatat | |
| | | cttgtggaaagCCAGAAACATGG | |
| 139 | 501 F10 gRNA | AATGATACGGCGACCACCGAGAT | 516 |
| | fwd | CTACACTATAGCCTACACTCTTT | |
| | | CCCTACACGACGCTCTTCCGATC | |
| | | TNNNNNNNNNNgctttatatatc | |
| | | ttgtggaaagCCAGAAACATGG | |
| 140 | 701 R gRNA | CAAGCAGAAGACGGCAfACGAGA | 517 |
| | rev | TCGAGTAATGTGACTGGAGTTCA | |
| | | GACGTGTGCTCTTCCGATCTNNN | |
| | | NNNNNNNNNGCACCCGACTCGGG | |
| | | TGCCAC | |
| 141 | 502 F12 UMI | AATGATACGGCGACCACCGAGAT | 518 |
| | fwd | CTACACATAGAGGCACACTCTTT | |
| | | CCCTACACGACGCTCTTCCGATC | |
| | | TNNNNNNNNNNNNgccgtccgtc | |
| | | atctcgttttcg | |
| 142 | 502 F11 | AATGATACGGCGACCACCGAGAT | 519 |
| | UMII fwd | CTACACATAGAGGCACACTCTTT | |
| | | CCCTACACGACGCTCTTCCGATC | |
| | | TNNNNNNNNNNNgccgtccgtca | |
| | | tctcgttttcg | |
| 143 | 502 F10 | AATGATACGGCGACCACCGAGAT | 520 |
| | UMI fwd | CTACACATAGAGGCACACTCTTT | |
| | | CCCTACACGACGCTCTTCCGATC | |
| | | TNNNNNNNNNNgccgtccgtcat | |
| | | ctcgttttcg | |
| 144 | 702 R UMI | CAAGCAGAAGACGGCATACGAGA | 521 |
| | rev | T TCTCCGGAGTGACTGG AGTT | |
| | | CAG ACGTGTGCTCTTCCG ATC | |
| | | TNNNNNNNNNNNNTCCCTAGTTA | |
| | | GCCAGAGAGCTCC | |

### Dataset S5

FISH probe sequences for barcode, reporter gene and phenotype imaging. This dataset includes the following separate lists of oligonucleotide probes:

The primary amplification probes for barcode imaging

The secondary amplification probes for barcode imaging

The FISH probes for polyA-containing RNA

The FISH probes for MALAT1

The FISH probes for 7SK

The FISH probes for MRP

The FISH probes for pre-ribosome

The FISH probes for U2 snRNA

The FISH probes for the reporter genes Puro-T2A-mCherry or mCherry-luciferase

The oligonucleotide probe attached to antibodies for SON

The readout probes for all of the above targets

Some probes were modified, and the modifications are shown in the probe sequences.

| | | | | SEQ |
|---|---|---|---|---|
| | | | ORDER | ID |
| READOUT | SEQUENCE | DYE | FORM | NO |
| 1 | ATCCTCCTTC | Cy5 | Cy5-S-S-AT | 522 |
| | AATACATCCC | | CCTCCTTCAA | |
| | | | TACATCCC | |
| 2 | ACACTACCAC | Alexa750 | Alexa750-S | 523 |
| | CATTTCCTAT | | -S-ACACTAC | |
| | | | CACCATTTCC | |
| | | | TAT | |
| 3 | ACTCCACTAC | Alexa750 | Alexa750-S | 524 |
| | TACTCACTCT | | -S-ACTCCAC | |
| | | | TACTACTCAC | |
| | | | TCT | |
| 4 | ACCCTCTAAC | Cy5 | Cy5-S-S-AC | 525 |
| | TTCCATCACA | | CCTCTAACIT | |
| | | | CCATCACA | |
| 5 | ACCACAACCC | Cy5 | Cy5-S-S-AC | 526 |
| | ATTCCTTTCA | | CACAACCCAT | |
| | | | TCCTTTCA | |
| 6 | TTTCTACCAC | Alexa750 | Alexa750-S | 527 |
| | TAATCAACCC | | -S-TTTCTAC | |
| | | | CACTAATCAA | |
| | | | CCC | |
| 7 | ACCCTTTACA | Cy5 | Cy5-S-S-AC | 528 |
| | AACACACCCT | | CCTTTACAAA | |
| | | | CACaccct | |
| 8 | TCCTATTCTC | Alexa750 | Alexa750-S | 529 |
| | AACCTAACCT | | -S-TCCTATT | |
| | | | CTCAACCTAA | |
| | | | CCT | |
| 9 | TATCCTTCAA | Alexa750 | Alexa750-S | 530 |
| | TCCCTCCACA | | -S-TATCCTT | |
| | | | CAATCCCTCC | |
| | | | ACA | |
| 10 | ACATTACACC | Cy5 | Cy5-S-S-AC | 531 |
| | TCATTCTCCC | | ATTACACCTC | |
| | | | ATTCTCCC | |
| 11 | TTTACTCCCT | Cy5 | Cy5-S-S-TT | 532 |
| | ACACCTCCAA | | TACTCCCTAC | |
| | | | ACCTCCAA | |
| 12 | TTCTCCCTCT | Alexa750 | Alexa750-S | 533 |
| | ATCAACTCTA | | -S-TTCTCCC | |
| | | | TCTATCAACT | |
| | | | CTA | |
| 13 | ACCCTTACTA | Cy5 | Cy5-S-S-AC | 534 |
| | CTACATCATC | | CCTTACTACT | |
| | | | ACATCATC | |
| 14 | TCCTAACAAC | Alexa750 | Alexa750-S | 535 |
| | CAACTACTCC | | -S-TCCTAAC | |
| | | | AACCAACTAC | |
| | | | TCC | |
| 15 | TCTATCATTA | Alexa750 | Alexa750-S | 536 |
| | CCCTCCTCCT | | -S-TCTATCA | |
| | | | TTACCCTCCT | |
| | | | CCT | |
| 16 | TATTCACCTT | Cy5 | Cy5-S-S-TA | 537 |
| | ACAAACCCTC | | TTCACCTTAC | |
| | | | AAACCCTC | |
| 17 | AAACACACAC | Cy5 | Cy5-S-S-AA | 538 |
| | TAAACCACCC | | ACACACACTA | |
| | | | AACCACCC | |
| 18 | AACTCATCTC | Alexa750 | Alexa750-S | 539 |
| | AATCCTCCCA | | -S-AACTCAT | |
| | | | CTCAATCCTC | |
| | | | CCA | |
| 19 | TATCTCATCA | Cy5 | Cy5-S-S-TA | 540 |
| | ATCCCACACT | | TCTCATCAAT | |
| | | | CCCACACT | |
| 20 | TCTATCATCT | Alexa750 | Alexa750-S | 541 |
| | CCAAACCACA | | -S-TCTATCA | |
| | | | TCTCCAAACC | |
| | | | ACA | |
| 21 | TCCAACTCAT | Alexa750 | Alexa750-S | 542 |
| | CTCTAATCTC | | -S-TCCAACT | |
| | | | CATCTCTAAT | |
| | | | CTC | |
| 22 | AATACTCTCC | Cy5 | Cy5-S-S-AA | 543 |
| | CACCTCAACT | | TACTCTCCCA | |
| | | | CCTCAACT | |
| 23 | ATAAATCATT | Cy5 | Cy5-S-S-AT | 544 |
| | CCCACTACCC | | AAATCATTCC | |
| | | | CACTACCC | |
| 24 | ACCCAACACT | Alexa750 | Alexa750-S | 545 |
| | CATAACATCC | | -S-ACCCAAC | |
| | | | ACTCATAACA | |
| | | | TCC | |
| 25 | TTCCTAACAA | Cy5 | Cy5-S-S-TT | 546 |
| | ATCACATCCC | | CCTAACAAAT | |
| | | | CACATCCC | |
| 26 | TTCTTCCCTC | Alexa750 | Alexa750-S | 547 |
| | AATCTTCATC | | -S-TTCTTCC | |
| | | | CTCAATCTTC | |
| | | | ATC | |
| 27 | TACTACAAAC | Cy5 | Cy5-S-S-TA | 548 |
| | CCATAATCCC | | CTACAAACCC | |
| | | | ATAATCCC | |
| 28 | TCCTCATCTT | Alexa750 | Alexa750-S | 549 |
| | ACTCCCTCTA | | -S-TCCTCAT | |
| | | | CTTACTCCCT | |
| | | | CTA | |
| 29 | AATCTCACCT | Cy5 | Cy5-S-S-AA | 550 |
| | TCCACTTCAC | | TCTCACcTTc | |
| | | | cAcTTcAC | |
| 30 | TTACCTCTAA | Alexa750 | Alexa750-S | 551 |
| | CCCTCCATTC | | -S-TTACCTC | |
| | | | TAACCCTCCA | |
| | | | TTC | |
| 31 | ACTTTCCACA | Cy5 | Cy5-S-S-AC | 552 |
| | TACTATCCCA | | TTTCCACATA | |
| | | | CTATCCCA | |
| 32 | ACCTTTCTCC | Alexa750 | Alexa750-S | 553 |
| | ATACCCAACT | | -S-ACCTTTC | |
| | | | TCCATACCCA | |
| | | | ACT | |
| 33 | TCAAACTTTC | Cy5 | Cy5-S-S-Tc | 554 |
| | CAACCACCTC | | aaacTTTccA | |
| | | | ACCACcTc | |
| 34 | ACACCATTTA | Alexa750 | Alexa750-S | 555 |
| | TCCACTCCTC | | -S-ACACCAT | |
| | | | TTATCCACTC | |
| | | | CTC | |
| 35 | TCCCAACTAA | Cy5 | Cy5-S-S-TC | 556 |
| | CCTAACATTC | | CCAACTAACC | |
| | | | TAACATTC | |
| 36 | ACATCCTAAC | Alexa750 | Alexa750-S | 557 |
| | TACAACCTTC | | -S-ACATCCT | |
| | | | AACTACAACC | |
| | | | TTC | |
| Phenotype | TTCCACAATC | Alexa488 | Alexa488-S | 558 |
| readout 1 | ACTTCCACAA | | -S-TTCCACA | |
| | | | ATCACTTCCA | |
| | | | CAA | |
| Phenotype | TCCCATACAA | Alexa488 | Alexa488-S | 559 |
| readout 2 | ATCCAAACCT | | -S-TCCCATA | |
| | | | CAAATCCAAA | |
| | | | CCT | |
| Phenotype | TTCTCTACTC | Alexa488 | Alexa488-S | 560 |
| readout 3 | ATTCCCTCAA | | -S-TTCTCTA | |
| | | | CTCATTCCCT | |
| | | | CAA | |
| Phenotype | AAACTCCACA | Alexa488 | Alexa488-S | 561 |
| readout 4 | TCCATCTCAT | | -S-AAACTCC | |
| | | | ACATCCATCT | |
| | | | CAT | |
| Phenotype | AATCTCCTCC | Alexa488 | Alexa488-S | 562 |
| readout 5 | AACACTTCTA | | -S-AATCTCC | |
| | | | TCCAACACTT | |
| | | | CTA | |
| Phenotype | TAAACATAAC | Alexa488 | Alexa488-S | 563 |
| readout 6 | ACCCTTTCCC | | -S-TAAACAT | |
| | | | AACACCCTTT | |
| | | | CCC | |
| Phenotype | TTATCCATCC | Alexa488 | Alexa488-S | 564 |
| readout 7 | CTCTTCCTAC | | -S-TTATCCA | |
| | | | TCCCTCTTCC | |
| | | | TAC | |
| Phenotype | AACTACATAC | Alexa488 | Alexa488-S | 565 |
| readout 8 | TCCCTACCTC | | -S-AACTACA | |
| | | | TACTCCCTAC | |
| | | | CTC | |
| Reporter | ATTACACTCC | ATT0565 | ATT0565-AT | 566 |
| gene | ATCCACTCAA | | TACACTCCAT | |
| smFISH | | | CCACTCAA | |
| readout | | | | |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
(a) introducing, into a plurality of cells, DNA comprising a guide portion comprising a recognition sequence, a reporter portion, and an identification portion comprising read sequences;
(b) determining positions of RNA molecules expressed from the reporter portion of the introduced DNA within the plurality of cells by determining the reporter portions;
(c) determining a read sequence on the RNA molecules expressed from the introduced DNA comprising the reporter portion and the identification portion within the plurality of cells by exposing the cells to a readout probe able to bind to the read sequence;
(d) colocalizing the binding of the readout probe with the positions of the RNA molecules expressed from the reporter portion of the introduced DNA;
(e) repeating (b), (c), and (d) a plurality of times using different read sequences; and
(f) creating codewords corresponding to the binding of the colocalized readout probes, wherein the values of the digits of the codewords are based on the binding of the readout probes to the read sequences.

2. The method of claim 1, further comprising identifying the guide portion for individual cells based on the measured codewords.

3. The method of any one of claim 1 or 2, wherein the recognition sequence recognizes a DNA sequence.

4. The method of any one of claims 1-3, wherein the recognition sequence recognizes an RNA sequence.

5. The method of any one of claims 1-4, wherein the introduced DNA arises from a library of nucleic acids.

6. The method of claim 5, wherein the library is generated by pooled cloning.

7. The method of any one of claims 1-6, wherein the identities of the associated pairs of guide portion and identification portion on the DNA are determined by sequencing.

8. The method of any one of claims 1-7, wherein the introduced DNA allows association of the guide portion and identification portion to occur.

9. The method of any one of claims 1-8, wherein each read sequence represents a value of a position within a codeword.

10. The method of any one of claims 1-9, wherein the read sequences are determined sequentially.

11. The method of any one of claims 1-10, wherein the guide portion further comprises a Cas protein binding sequence.

12. The method of any one of claims 1-11, wherein the guide portion allows the targeting of the Cas protein to DNA or RNA to perturb the sequence or expression of a gene.

13. The method of any one of claims 1-12, wherein the reporter portion encodes a protein detectable by fluorescence.

14. The method of any one of claims 1-13, wherein the reporter portion encodes a fluorescent protein.

15. The method of any one of claims 1-14, wherein the reporter portion encodes luciferase.

16. The method of any one of claims 1-15, wherein the reporter portion encodes a protein detectable by immunoprecipitation.

17. The method of any one of claims 1-16, wherein the reporter portion encodes a protein detectable by immunofluorescence.

18. The method of any one of claims 1-17, wherein the reporter portion encodes a Myc tag.

19. The method of any one of claims 1-18, wherein the reporter portion encodes a HA tag.

20. The method of any one of claims 1-19, wherein the reporter portion comprises a reporter gene.

21. The method of any one of claims 1-20, wherein the identification portion is present within a 3′ UTR of the reporter gene.

22. The method of any one of claims 1-21, wherein the reporter portion comprises a first promoter.

23. The method of claim 22, wherein the promoter is a promoter that drives transcription.

24. The method of any one of claim 22 or 23, wherein the promoter comprises a CMV promoter.

25. The method of any one of claims 22-24, wherein the recognition sequence comprises a second promoter separate from the first promoter.

26. The method of any one of claims 1-25, wherein the recognition sequence and the reporter portion are separated by less than 1000 bases.

27. The method of any one of claims 1-26, wherein the recognition sequence and the reporter portion within the DNA are separated by less than 100 bases.

28. The method of any one of claims 1-27, comprising determining the one or more read sequences using fluorescence.

29. The method of any one of claims 1-28, comprising determining the positions of the RNA comprising the reporter portion and the identification portion using smFISH targeting the reporter portion.

30. The method of any one of claims 1-29, comprising introducing the DNA into the plurality of cells using a virus.

31. The method of claim 30, wherein the virus is a lentivirus.

32. The method of claim 31, wherein the guide portion and the identification portion are positioned adjacent to the 3′ of the polypurine tract sequence within the lentivirus.

33. The method of claim 32, wherein the guide portion is duplicated within the 5′ region of the lentivirus.

34. The method of any one of claims 1-33, wherein introducing the DNA into the plurality of cells comprises electroporating the DNA into the plurality of cells.

35. The method of any one of claims 1-34, wherein the cells comprise cells in tissue.

36. The method of any one of claims 1-35, comprising introducing the DNA into the plurality of cells such that at least 50% of the cells contains no more than one type of introduced DNA.

37. The method of any one of claims 1-36, comprising introducing the DNA into the plurality of cells such that at least 90% of the cells contains no more than one type of introduced DNA.

38. The method of any one of claims 1-37, comprising introducing the DNA into the genome of the cells.

39. The method of any one of claims 1-38, wherein the DNA further comprise a promotor.

40. The method of any one of claims 1-39, wherein the read sequences defines a binary space of digits for the codewords.

41. The method of any one of claims 1-40, wherein the read sequences defines a ternary space of digits for the codewords.

42. The method of any one of claims 1-41, wherein determining the one or more read sequences comprises:
for each digit of the codeword, applying a readout probe corresponding to the digit of the codeword to the plurality of cells.

43. The method of claim 42, comprising sequentially applying and removing each of the readout probes to the plurality of the cells.

44. The method of any one of claims 1-43, wherein for at least some of the created codewords, matching the codeword to valid codewords wherein, if no match is found, either discarding the codeword or applying error correction to the codeword to form a valid codeword.

45. The method of claim 44, wherein the values of the digits of the codewords defines a set of potential codewords.

46. The method of claim 45, wherein the set of potential codewords comprises at least 10² unique sequences.

47. The method of claim 46, wherein the set of potential codewords comprises at least 10³ unique sequences.

48. The method of any one of claim 46 or 47, wherein the set of potential codewords comprises at least 10⁴ unique sequences.

49. The method of any one of claims 46-48, wherein the set of potential codewords comprises at least 10⁵ unique sequences.

50. The method of any one of claims 46-49, wherein the set of potential codewords comprises at least 10⁶ unique sequences.

51. The method of any one of claims 44-50, wherein a portion of the potential codewords are valid codewords.

52. The method of claim 51, comprising comparing the measured codewords to the valid codewords to determine errors in codeword measurement.

53. The method of any one of claims 44-52, wherein the valid codewords are a randomly selected subset from the possible codewords.

54. The method of claim 53, wherein less than 10% of possible codewords are valid codewords.

55. The method of any one of claim 53 or 54, wherein less than 5% of possible codewords are the valid codewords.

56. The method of any one of claims 53-55, wherein less than 1% of possible codewords are the valid codewords.

57. The method of any one of claims 53-56, wherein less than 0.5% of possible codewords are the valid codewords.

58. The method of any one of claims 1-57, wherein the identification portion comprises N variable portions, N being at least 3, each variable portion being of at least two possibilities.

59. The method of claim 58, wherein N is at least 5.

60. The method of any one of claim 58 or 59, wherein N is at least 10.

61. The method of any one of claims 58-60, wherein N is at least 15.

62. The method of any one of claims 58-61, wherein N is at least 20.

63. The method of any one of claims 1-62, wherein the variable portions within the identification portion are each the same length.

64. The method of any one of claims 1-63, wherein the variable portions within the identification portion each have a length of between 5 and 50 nt.

65. The method of any one of claims 1-64, wherein the variable portions within the identification portion each have a length of between 15 and 25 nt.

66. The method of any one of claims 1-65, wherein the identification portion comprises an error-detectable code.

67. The method of any one of claims 1-66, wherein the identification portion comprises an error-correcting code.

68. The method of any one of claim 66 or 67, wherein the error-detectable or error-correcting code comprises a Hamming code.

69. The method of any one of claims 66-68, wherein the error-detectable or error-correcting code comprises an extended Hamming code.

70. The method of any one of claims 66-69, wherein the error-detectable or error-correcting code comprises a Reed-Solomon code.

71. The method of any one of claims 66-70, wherein the error-detectable or error-correcting code is not uniform for all members of the code.

72. The method of any one of claim 1-71, wherein the nucleic acid probes comprises at least 8 possible read sequences.

73. The method of any one of claims 1-72, wherein the nucleic acid probes comprises at least 16 possible read sequences.

74. The method of any one of claims 1-73, wherein the nucleic acid probes comprises no more than 32 possible read sequences.

75. The method of any one of claims 1-74, wherein the nucleic acid probes comprises at least 32 possible read sequences.

76. A method, comprising:
introducing, into a plurality of cells, DNA comprising a guide portion comprising a recognition sequence, a reporter portion, and an identification portion comprising read sequences;
determining positions of RNA molecules expressed from the reporter portion of the introduced DNA within the plurality of cells by determining the reporter portions;
determining the read sequences within the plurality of cells by exposing the cells to a plurality of readout probes each able to bind to a read sequence, colocalizing the binding of the readout probes with the positions of the RNA molecules expressed from the reporter portion of the introduced DNA; and
creating codewords corresponding to the binding of the colocalized readout probes, wherein the values of the digits of the codewords are based on the binding of the readout probes to the read sequences.

77. A method, comprising:
introducing DNA into a plurality of cells using a lentivirus, wherein the DNA comprises a guide portion comprising a recognition sequence, a reporter portion, and an identification portion comprising read sequences;
determining phenotype of the plurality of cells; and
determining genotype of the plurality of cells; and
determining the correspondence between the genotype and the phenotype.

78. The method of claim 77, wherein determining phenotype comprises determining a cell property.

79. The method of any one of claim 77 or 78, wherein determining phenotype comprises determining morphology of a cellular structure.

80. The method of claim 79, wherein the morphology is whole cell morphology.

81. The method of any one of claim 79 or 80, wherein the morphology is subcompartment morphology.

82. The method of any one of claims 77-81, wherein determining phenotype comprises determining the morphology of more then one cellular structure.

83. The method of any one of claims 77-82, wherein determining phenotype comprises determining a protein using immunofluorescence.

84. The method of any one of claims 77-83, wherein determining phenotype comprises determining a protein using fluorescence.

85. The method of any one of claims 77-84, wherein determining phenotype comprises determining a protein using a fluorescent protein.

86. The method of any one of claims 77-85, wherein determining phenotype comprises determining a protein using an organic dye.

87. The method of any one of claims 77-86, wherein determining phenotype comprises determining cell dynamic behavior.

88. The method of any one of claims 77-87, wherein determining phenotype comprises determining a cell-cell interaction.

89. The method of any one of claims 77-88, wherein determining phenotype comprises determining a cell state.

90. The method of any one of claims 77-89, wherein determining phenotype comprises determining a cellular RNA or RNAs using smFISH.

91. The method of any one of claims 77-90, wherein determining phenotype comprises determining a gene expression profile using multiplexed FISH.

92. The method of any one of claims 77-91, wherein determining phenotype comprises determining a gene expression profile using MERFISH.

93. The method of any one of claims 77-92, wherein determining phenotype comprises determining a RNA or a plurality of RNAs spatially.

94. The method of any one of claims 77-93, wherein determining phenotype comprises determining at least portion of a proteome.

95. The method of any one of claims 77-94, wherein determining phenotype comprises determining at least a portion of a chromosome using DNA FISH.

96. The method of any one of claims 77-95, wherein determining phenotype comprises determining at least a portion of a chromosome using multiplexed DNA FISH.

97. The method of any one of claims 77-96, wherein determining phenotype comprises determining at least a portion of a chromosome using CASFISH.

98. The method of any one of claims 77-97, wherein determining phenotype comprises determining protein modification cells using immunofluorescence.

99. The method of any one of claims 77-98, wherein determining phenotype comprises determining a protein interaction with RNA or DNA.

100. The method of any one of claims 77-99, wherein determining phenotype comprises determining an epigenetic modification.

101. The method of any one of claims 77-100, wherein determining phenotype comprises determining cell growth.

102. The method of any one of claims 77-101, wherein determining phenotype comprises determining a change of cell growth.

103. The method of any one of claims 77-102 wherein determining phenotype and determining genotype use a common imaging technique.

104. The method of any one of claims 77-103, wherein determining phenotype and determining genotype use different imaging techniques.

105. The method of any one of claim 103 or 104, wherein the imaging technique has a resolution better than 300 nm.

106. The method of any one of claims 103-105, wherein determining phenotype uses multicolor fluorescence imaging.

107. The method of any one of claims 103-106, wherein determining phenotype uses confocal imaging.

108. The method of any one of claims 103-107, wherein determining phenotype uses TIRF imaging.

109. The method of any one of claims 103-108, wherein determining phenotype uses two-photon imaging.

110. The method of any one of claims 103-109, wherein determining phenotype uses STORM.

111. The method of any one of claims 103-110, wherein determining phenotype uses a superresolution technique.

112. The method of claim 111, wherein the superresolution technique is PALM, FPALM, STED, SIM, and/or RESOLFT.

113. The method of any one of claims 77-112, wherein determining genotype comprises determining the sequence of the identification portion of the introduced DNA.

114. The method of any one of claims 77-113, wherein determining genotype comprises determining the genotype using smFISH.

115. The method of any one of claims 77-114, wherein determining genotype comprises determining the genotype using multiplexed FISH

116. The method of any one of claims 77-115, wherein determining genotype comprises determining the genotype using MERFISH.

117. The method of any one of claims 77-116, wherein determining genotype comprises determining the genotype using in situ hybridization.

118. The method of any one of claims 77-117, wherein determining genotype comprises determining the genotype using sequential FISH.

119. The method of any one of claims 77-118, wherein determining genotype comprises determining the genotype using CASFISH.

120. The method of any one of claims 77-119, wherein determining genotype comprises determining the genotype using in situ sequencing.

121. A method, comprising:
introducing nucleic acids into a plurality of cells, wherein the nucleic acids comprise a guide portion comprising a recognition sequence, a reporter portion, and an identification portion comprising read sequences;
imaging the plurality of cells, wherein the cells exhibit imagable differences in phenotype due to expression of the guide portion; and
acquiring a plurality of images of the plurality of cells, wherein the images of the cells exhibit differences due to differences in the identification portions of the nucleic acids within the cells.

122. The method of claim 121, wherein based on the correspondence between the guide portion and the identification portion, the guide portion introduced to the plurality of cells is identified.

123. The method of any one of claim 121 or 122, wherein cells having different phenotypes have different appearances when imaged.

124. The method of any one of claims 121-123, wherein cells having different phenotypes have different fluorescences when imaged.

125. The method of any one of claims 121-124, wherein imaging the plurality of cells comprises acquiring images of the plurality of cells using a single imaging modality.

126. The method of any one of claims 121-125, wherein imaging the plurality of cells comprises acquiring images of the plurality of cells using a plurality of imaging modalities.

127. The method of any one of claims 121-126, wherein imaging the plurality of cells comprises acquiring a single image.

128. The method of any one of claims 121-127, wherein imaging the plurality of cells comprises acquiring a plurality of images.

129. The method of any one of claims 121-128, wherein imaging the plurality of cells comprises imaging the plurality of cells using smFISH.

130. The method of any one of claims 121-129, wherein imaging the plurality of cells comprises imaging the plurality of cells using MERFISH.

131. The method of any one of claims 121-130, further comprising determining morphology of the cells.

132. The method of claim 131, comprising determining the morphology changes of the cells over time.

133. The method of any one of claim 131 or 132, comprising determining the morphology changes of the organelles of the cells.

134. The method of any one of claims 131-133, comprising determining the morphology changes during cell growth.

135. A method, comprising:
introducing DNA into a plurality of cells using a lentivirus, wherein the DNA comprises a guide portion comprising a recognition sequence and an identification portion comprising read sequences;
determining phenotype of the plurality of cells;
determining genotype of the plurality of cells; and
determining the correspondence between genotypes and phenotypes.

136. The method of claim 135, wherein determining phenotype comprises determining a cell property.

137. The method of any one of claim 135 or 136, wherein determining phenotype comprises determining morphology of a cellular structure.

138. The method of claim 137, wherein the morphology is whole cell morphology.

139. The method of any one of claim 137 or 138, wherein the morphology is subcompartment morphology.

140. The method of any one of claims 137-139, wherein determining phenotype comprises determining morphology of more then one cellular structure.

141. The method of any one of claims 135-140, wherein determining phenotype comprises determining a protein using immunofluorescence.

142. The method of any one of claims 135-141, wherein determining phenotype comprises determining a protein using fluorescence.

143. The method of any one of claims 135-142, wherein determining phenotype comprises determining a protein using a fluorescent protein.

144. The method of any one of claims 135-143, wherein determining phenotype comprises determining a protein using an organic dye.

145. The method of any one of claims 135-144, wherein determining phenotype comprises determining cell dynamic behavior.

146. The method of any one of claims 135-145, wherein determining phenotype comprises determining a cell-cell interaction.

147. The method of any one of claims 135-146, wherein determining phenotype comprises determining a cell state.

148. The method of any one of claims 135-147, wherein determining phenotype comprises determining a RNA or multiple RNAs using smFISH.

149. The method of any one of claims 135-148, wherein determining phenotype comprises determining a gene expression profile using multiplexed FISH.

150. The method of any one of claims 135-149, wherein determining phenotype comprises determining a gene expression profile using MERFISH.

151. The method of any one of claims 135-150, wherein determining phenotype comprises determining a RNA or multiple RNAs spatially.

152. The method of any one of claims 135-151, wherein determining phenotype comprises determining at least portion of a proteome.

153. The method of any one of claims 135-152, wherein determining phenotype comprises determining at least a portion of a chromosome using DNA FISH.

154. The method of any one of claims 135-153, wherein determining phenotype comprises determining at least a portion of a chromosome using multiplexed DNA FISH.

155. The method of any one of claims 135-154, wherein determining phenotype comprises determining at least a portion of a chromosome using CASFISH.

156. The method of any one of claims 135-155, wherein determining phenotype comprises determining protein modification cells using immunofluorescence.

157. The method of any one of claims 135-156, wherein determining phenotype comprises determining a protein interaction with RNA or DNA.

158. The method of any one of claims 135-157, wherein determining phenotype comprises determining an epigenetic modification.

159. The method of any one of claims 135-158, wherein determining phenotype comprises determining cell growth.

160. The method of any one of claims 135-159, wherein determining phenotype comprises determining a change of cell growth.

161. The method of any one of claims 135-160, wherein determining phenotype and determining genotype use a common imaging technique.

162. The method of claim 161, wherein the imaging technique has a resolution better than 300 nm.

163. The method of any one of claims 135-160, wherein determining phenotype and determining genotype use different imaging techniques.

164. The method of claim 163, wherein at least one of the imaging techniques has a resolution better than 300 nm.

165. The method of any one of claims 135-164, wherein determining phenotype uses multicolor fluorescence imaging.

166. The method of any one of claims 135-165, wherein determining phenotype uses confocal imaging.

167. The method of any one of claims 135-166, wherein determining phenotype uses TIRF imaging.

168. The method of any one of claims 135-167, wherein determining phenotype uses two-photon imaging.

169. The method of any one of claims 135-168, wherein determining phenotype uses STORM.

170. The method of any one of claims 135-169, wherein determining phenotype uses a superresolution technique.

171. The method of any one of claims 135-170, wherein the superresolution technique is PALM, FPALM, STED, SIM, and/or RESOLFT.

172. The method of any one of claims 135-171, wherein determining genotype comprises determining the sequence of the identification protion of the introduced DNA.

173. The method of any one of claims 135-172, wherein determining genotype comprises determining the genotype using smFISH.

174. The method of any one of claims 135-173, wherein determining genotype comprises determining the genotype using multiplexed FISH.

175. The method of any one of claims 135-174, wherein determining genotype comprises determining the genotype using MERFISH.

176. The method of any one of claims 135-175, wherein determining genotype comprises determining the genotype using in situ hybridization.

177. The method of any one of claims 135-176, wherein determining genotype comprises determining the genotype using sequential FISH.

178. The method of any one of claims 135-177, wherein determining genotype comprises determining the genotype using CASFISH.

179. The method of any one of claims 135-178, wherein determining genotype comprises determining the genotype using in situ sequencing.
